SEQUENCE LISTING (973 sequences)

SEQ ID NO: 1 - DNA, 4590 bp, Homo sapiens
  source 1..4590: mol_type = other DNA
aggagagcctcccggtgtatttgaataaaccaggttggcaaatcatactatagctgaaagaattggcaggaactgaaaatgactaggaagaggacatactgggtgcccaactcttctggtggcctcgtgaatcgtggcatcgacataggcgatgacatggtttcaggacttatttataaaacctatactctccaagatggcccctggagtcagcaagagagaaatcctgaggctccagggagggcagctgtcccaccgtgggggaagtatgatgctgccttgagaaccatgattcccttccgtcccaagccgaggtttcctgccccccagcccctggacaatgctggcctgttctcctacctcaccgtgtcatggctcaccccgctcatgatccaaagcttacggagtcgcttagatgagaacaccatccctccactgtcagtccatgatgcctcagacaaaaatgtccaaaggcttcaccgcctttgggaagaagaagtctcaaggcgagggattgaaaaagcttcagtgcttctggtgatgctgaggttccagagaacaaggttgattttcgatgcacttctgggcatctgcttctgcattgccagtgtactcgggccaatattgattataccaaagatcctggaatattcagaagagcagttggggaatgctgtccatggagtgggactctgctttgccctttttctctccgaatgcgtgaagtctctgagtttctcctccagttggatcatcaaccaacgcacagccatcaggttccgagcagctgtttcctcctttgcctttgagaagctcatccaatttaagtctgtaatacacatcacctcaggagaggccatcagcttcttcaccggtgatgtaaactacctgtttgaaggggtgtgctatggacccctagtactgatcacctgcgcatcgctggtcatctgcagcatttcttcctacttcattattggatacactgcatttattgccatcttatgctatctcctggttttcccactggcggtattcatgacaagaatggctgtgaaggctcagcatcacacatctgaggtcagcgaccagcgcatccgtgtgaccagtgaagttctcacttgcattaagctgattaaaatgtacacatgggagaaaccatttgcagaaatcattgaagacctaagaaggaaggaaaggaaactattggagaagtgcgggcttgtccagagcctgacaagtataaccttgttcatcatccccacagtggccacagcggtctgggttctcatccacacatccttaaagctgaaactcacagcgtcaatggccttcagcatgctggcctccttgaatctccttcggctgtcagtgttctttgtgcctattgcagtcaaaggtctcacgaattccaagtctgcagtgatgaggttcaagaagtttttcctccaggagagccctgttttctatgtccagacattacaagaccccagcaaagctctggtctttgaggaggccaccttgtcatggcaacagacctgtcccgggatcgtcaatggggcactggagctggagaggaacgggcatgcttctgaggggatgaccaggcctagagatgccctcgggccagaggaagaagggaacagcctgggcccagagttgcacaagatcaacctggtggtgtccaaggggatgatgttaggggtctgcggcaacacggggagtggtaagagcagcctgttgtcagccatcctggaggagatgcacttgctcgagggctcggtgggggtgcagggaagcctggcctatgtcccccagcaggcctggatcgtcagcgggaacatcagggagaacatcctcatgggaggcgcatatgacaaggcccgatacctccaggtgctccactgctgctccctgaatcgggacctggaacttctgccctttggagacatgacagagattggagagcggggcctcaacctctctggggggcagaaacagaggatcagcctggcccgcgccgtctattccgaccgtcagatctacctgctggacgaccccctgtctgctgtggacgcccacgtggggaagcacatttttgaggagtgcattaagaagacactcagggggaagacggtcgtcctggtgacccaccagctgcagtacttagaattttgtggccagatcattttgttggaaaatgggaaaatctgtgaaaatggaactcacagtgagttaatgcagaaaaaggggaaatatgcccaacttatccagaagatgcacaaggaagccacttcggacatgctgcaggacacagcaaagatagcagagaagccaaaggtagaaagtcaggctctggccacctccctggaagagtctctcaacggaaatgctgtgccggagcatcagctcacacaggaggaggagatggaagaaggctccttgagttggagggtctaccaccactacatccaggcagctggaggttacatggtctcttgcataattttcttcttcgtggtgctgatcgtcttcttaacgatcttcagcttctggtggctgagctactggttggagcagggctcggggaccaatagcagccgagagagcaatggaaccatggcagacctgggcaacattgcagacaatcctcaactgtccttctaccagctggtgtacgggctcaacgccctgctcctcatctgtgtgggggtctgctcctcagggattttcaccaaggtcacgaggaaggcatccacggccctgcacaacaagctctttaacaaggttttccgctgccccatgagtttctttgacaccatcccaataggccggcttttgaactgcttcgcaggggacttggaacagctggaccagctcttgcccatcttttcagagcagttcctggtcctgtccttaatggtgatcgccgtcctgttgattgtcagtgtgctgtctccatatatcctgttaatgggagccataatcatggttatttgcttcatttattatatgatgttcaagaaggccatcggtgtgttcaagagactggagaactatagccggtctcctttattctcccacatcctcaattctctgcaaggcctgagctccatccatgtctatggaaaaactgaagacttcatcagccagtttaagaggctgactgatgcgcagaataactacctgctgttgtttctatcttccacacgatggatggcattgaggctggagatcatgaccaaccttgtgaccttggctgttgccctgttcgtggcttttggcatttcctccaccccctactcctttaaagtcatggctgtcaacatcgtgctgcagctggcgtccagcttccaggccactgcccggattggcttggagacagaggcacagttcacggctgtagagaggatactgcagtacatgaagatgtgtgtctcggaagctcctttacacatggaaggcacaagttgtccccaggggtggccacagcatggggaaatcatatttcaggattatcacatgaaatacagagacaacacacccaccgtgcttcacggcatcaacctgaccatccgcggccacgaagtggtgggcatcgtgggaaggacgggctctgggaagtcctccttgggcatggctctcttccgcctggtggagcccatggcaggccggattctcattgacggcgtggacatttgcagcatcggcctggaggacttgcggtccaagctctcagtgatccctcaagatccagtgctgctctcaggaaccatcagattcaacctagatccctttgaccgtcacactgaccagcagatctgggatgccttggagaggacattcctgaccaaggccatctcaaagttccccaaaaagctgcatacagatgtggtggaaaacggtggaaacttctctgtgggggagaggcagctgctctgcattgccagggctgtgcttcgcaactccaagatcatccttatcgatgaagccacagcctccattgacatggagacagacaccctgatccagcgcacaatccgtgaagccttccagggctgcaccgtgctcgtcattgcccaccgtgtcaccactgtgctgaactgtgaccacatcctggttatgggcaatgggaaggtggtagaatttgatcggccggaggtactgcggaagaagcctgggtcattgttcgcagccctcatggccacagccacttcttcactgagataaggagatgtggagacttcatggaggctggcagctgagctcagaggttcacacaggtgcagcttcgaggcccacagtctgcgaccttcttgtttggagatgagaacttctcctggaagcaggggtaaatgtagggggggtggggattgctggatggaaaccctggaataggctacttgatggctctcaagaccttagaaccccagaaccatctaagacatgggattcagtgatcatgtggttctccttttaacttacatgctgaataattttataataaggtaaaagcttatagttttctgatctgtgttagaagtgttgcaaatgctgtactgactttgtaaaatataaaactaaggaaaactc

SEQ ID NO: 2 - DNA, 23 bp, Homo sapiens
  source 1..23: mol_type = other DNA
ccggtgtatttgaataaaccagg

SEQ ID NO: 3 - DNA, 23 bp, Homo sapiens
  source 1..23: mol_type = other DNA
aggttggcaaatcatactatagc

SEQ ID NO: 4 - DNA, 23 bp, Homo sapiens
  source 1..23: mol_type = other DNA
ttggcaaatcatactatagctga

SEQ ID NO: 5 - DNA, 23 bp, Homo sapiens
  source 1..23: mol_type = other DNA
ggcaaatcatactatagctgaaa

SEQ ID NO: 6 - DNA, 23 bp, Homo sapiens
  source 1..23: mol_type = other DNA
atcatactatagctgaaagaatt

SEQ ID NO: 7 - DNA, 23 bp, Homo sapiens
  source 1..23: mol_type = other DNA
ctgaaagaattggcaggaactga

SEQ ID NO: 8 - DNA, 23 bp, Homo sapiens
  source 1..23: mol_type = other DNA
aagaattggcaggaactgaaaat

SEQ ID NO: 9 - DNA, 23 bp, Homo sapiens
  source 1..23: mol_type = other DNA
tggcaggaactgaaaatgactag

SEQ ID NO: 10 - DNA, 23 bp, Homo sapiens
  source 1..23: mol_type = other DNA
ggcaggaactgaaaatgactagg

SEQ ID NO: 11 - DNA, 23 bp, Homo sapiens
  source 1..23: mol_type = other DNA
aggaactgaaaatgactaggaag

SEQ ID NO: 12 - DNA, 23 bp, Homo sapiens
  source 1..23: mol_type = other DNA
aactgaaaatgactaggaagagg

SEQ ID NO: 13 - DNA, 23 bp, Homo sapiens
  source 1..23: mol_type = other DNA
tcgtgaatcgtggcatcgacata

SEQ ID NO: 14 - DNA, 23 bp, Homo sapiens
  source 1..23: mol_type = other DNA
gtgaatcgtggcatcgacatagg

SEQ ID NO: 15 - DNA, 23 bp, synthetic construct
  misc_feature 1..23: note = Synthetic sequence
  source 1..23: mol_type = other DNA
ggcgatgacatggtttcaggact

SEQ ID NO: 16 - DNA, 23 bp, Homo sapiens
  source 1..23: mol_type = other DNA
atgacatggtttcaggacttatt

SEQ ID NO: 17 - DNA, 23 bp, Homo sapiens
  source 1..23: mol_type = other DNA
gacatggtttcaggacttattta

SEQ ID NO: 18 - DNA, 23 bp, Homo sapiens
  source 1..23: mol_type = other DNA
atggtttcaggacttatttataa

SEQ ID NO: 19 - DNA, 23 bp, Homo sapiens
  source 1..23: mol_type = other DNA
tggtttcaggacttatttataaa

SEQ ID NO: 20 - DNA, 23 bp, Homo sapiens
  source 1..23: mol_type = other DNA
caggacttatttataaaacctat

SEQ ID NO: 21 - DNA, 23 bp, Homo sapiens
  source 1..23: mol_type = other DNA
aggacttatttataaaacctata

SEQ ID NO: 22 - DNA, 23 bp, Homo sapiens
  source 1..23: mol_type = other DNA
gacttatttataaaacctatact

SEQ ID NO: 23 - DNA, 23 bp, Homo sapiens
  source 1..23: mol_type = other DNA
ctggagtcagcaagagagaaatc

SEQ ID NO: 24 - DNA, 23 bp, Homo sapiens
  source 1..23: mol_type = other DNA
aagtatgatgctgccttgagaac

SEQ ID NO: 25 - DNA, 23 bp, Homo sapiens
  source 1..23: mol_type = other DNA
tggacaatgctggcctgttctcc

SEQ ID NO: 26 - DNA, 23 bp, Homo sapiens
  source 1..23: mol_type = other DNA
cccgctcatgatccaaagcttac

SEQ ID NO: 27 - DNA, 23 bp, Homo sapiens
  source 1..23: mol_type = other DNA
ccgctcatgatccaaagcttacg

SEQ ID NO: 28 - DNA, 23 bp, Homo sapiens
  source 1..23: mol_type = other DNA
aagcttacggagtcgcttagatg

SEQ ID NO: 29 - DNA, 23 bp, Homo sapiens
  source 1..23: mol_type = other DNA
agcttacggagtcgcttagatga

SEQ ID NO: 30 - DNA, 23 bp, Homo sapiens
  source 1..23: mol_type = other DNA
tcgcttagatgagaacaccatcc

SEQ ID NO: 31 - DNA, 23 bp, Homo sapiens
  source 1..23: mol_type = other DNA
gtccatgatgcctcagacaaaaa

SEQ ID NO: 32 - DNA, 23 bp, Homo sapiens
  source 1..23: mol_type = other DNA
tccatgatgcctcagacaaaaat

SEQ ID NO: 33 - DNA, 23 bp, Homo sapiens
  source 1..23: mol_type = other DNA
tgcctcagacaaaaatgtccaaa

SEQ ID NO: 34 - DNA, 23 bp, Homo sapiens
  source 1..23: mol_type = other DNA
gcctcagacaaaaatgtccaaag

SEQ ID NO: 35 - DNA, 23 bp, Homo sapiens
  source 1..23: mol_type = other DNA
gacaaaaatgtccaaaggcttca

SEQ ID NO: 36 - DNA, 23 bp, Homo sapiens
  source 1..23: mol_type = other DNA
aggcttcaccgcctttgggaaga

SEQ ID NO: 37 - DNA, 23 bp, Homo sapiens
  source 1..23: mol_type = other DNA
caccgcctttgggaagaagaagt

SEQ ID NO: 38 - DNA, 23 bp, Homo sapiens
  source 1..23: mol_type = other DNA
ccgcctttgggaagaagaagtct

SEQ ID NO: 39 - DNA, 23 bp, Homo sapiens
  source 1..23: mol_type = other DNA
gcctttgggaagaagaagtctca

SEQ ID NO: 40 - DNA, 23 bp, Homo sapiens
  source 1..23: mol_type = other DNA
agggattgaaaaagcttcagtgc

SEQ ID NO: 41 - DNA, 23 bp, Homo sapiens
  source 1..23: mol_type = other DNA
cagtgcttctggtgatgctgagg

SEQ ID NO: 42 - DNA, 23 bp, Homo sapiens
  source 1..23: mol_type = other DNA
tggtgatgctgaggttccagaga

SEQ ID NO: 43 - DNA, 23 bp, Homo sapiens
  source 1..23: mol_type = other DNA
tgctgaggttccagagaacaagg

SEQ ID NO: 44 - DNA, 23 bp, Homo sapiens
  source 1..23: mol_type = other DNA
gaggttccagagaacaaggttga

SEQ ID NO: 45 - DNA, 23 bp, Homo sapiens
  source 1..23: mol_type = other DNA
ttccagagaacaaggttgatttt

SEQ ID NO: 46 - DNA, 23 bp, Homo sapiens
  source 1..23: mol_type = other DNA
tccagagaacaaggttgattttc

SEQ ID NO: 47 - DNA, 23 bp, Homo sapiens
  source 1..23: mol_type = other DNA
gagaacaaggttgattttcgatg

SEQ ID NO: 48 - DNA, 23 bp, Homo sapiens
  source 1..23: mol_type = other DNA
aggttgattttcgatgcacttct

SEQ ID NO: 49 - DNA, 23 bp, Homo sapiens
  source 1..23: mol_type = other DNA
atgcacttctgggcatctgcttc

SEQ ID NO: 50 - DNA, 23 bp, synthetic construct
  misc_feature 1..23: note = Synthetic sequence
  source 1..23: mol_type = other DNA
tgcacttctgggcatctgcttct

SEQ ID NO: 51 - DNA, 23 bp, synthetic construct
  misc_feature 1..23: note = Synthetic sequence
  source 1..23: mol_type = other DNA
cagtgtactcgggccaatattga

SEQ ID NO: 52 - DNA, 23 bp, Homo sapiens
  source 1..23: mol_type = other DNA
gggccaatattgattataccaaa

SEQ ID NO: 53 - DNA, 23 bp, Homo sapiens
  source 1..23: mol_type = other DNA
ggccaatattgattataccaaag

SEQ ID NO: 54 - DNA, 23 bp, Homo sapiens
  source 1..23: mol_type = other DNA
gccaatattgattataccaaaga

SEQ ID NO: 55 - DNA, 23 bp, Homo sapiens
  source 1..23: mol_type = other DNA
taccaaagatcctggaatattca

SEQ ID NO: 56 - DNA, 23 bp, Homo sapiens
  source 1..23: mol_type = other DNA
ggggaatgctgtccatggagtgg

SEQ ID NO: 57 - DNA, 23 bp, Homo sapiens
  source 1..23: mol_type = other DNA
ctctccgaatgcgtgaagtctct

SEQ ID NO: 58 - DNA, 23 bp, synthetic construct
  misc_feature 1..23: note = Synthetic sequence
  source 1..23: mol_type = other DNA
ctccgaatgcgtgaagtctctga

SEQ ID NO: 59 - DNA, 23 bp, Homo sapiens
  source 1..23: mol_type = other DNA
ccgaatgcgtgaagtctctgagt

SEQ ID NO: 60 - DNA, 23 bp, Homo sapiens
  source 1..23: mol_type = other DNA
atgcgtgaagtctctgagtttct

SEQ ID NO: 61 - DNA, 23 bp, Homo sapiens
  source 1..23: mol_type = other DNA
gcgtgaagtctctgagtttctcc

SEQ ID NO: 62 - DNA, 23 bp, Homo sapiens
  source 1..23: mol_type = other DNA
gagtttctcctccagttggatca

SEQ ID NO: 63 - DNA, 23 bp, Homo sapiens
  source 1..23: mol_type = other DNA
ctccagttggatcatcaaccaac

SEQ ID NO: 64 - DNA, 23 bp, Homo sapiens
  source 1..23: mol_type = other DNA
tccagttggatcatcaaccaacg

SEQ ID NO: 65 - DNA, 23 bp, Homo sapiens
  source 1..23: mol_type = other DNA
cagctgtttcctcctttgccttt

SEQ ID NO: 66 - DNA, 23 bp, Homo sapiens
  source 1..23: mol_type = other DNA
agctgtttcctcctttgcctttg

SEQ ID NO: 67 - DNA, 23 bp, Homo sapiens
  source 1..23: mol_type = other DNA
ttcctcctttgcctttgagaagc

SEQ ID NO: 68 - DNA, 23 bp, Homo sapiens
  source 1..23: mol_type = other DNA
tgcctttgagaagctcatccaat

SEQ ID NO: 69 - DNA, 23 bp, Homo sapiens
  source 1..23: mol_type = other DNA
ttgagaagctcatccaatttaag

SEQ ID NO: 70 - DNA, 23 bp, Homo sapiens
  source 1..23: mol_type = other DNA
aagctcatccaatttaagtctgt

SEQ ID NO: 71 - DNA, 23 bp, Homo sapiens
  source 1..23: mol_type = other DNA
ctcatccaatttaagtctgtaat

SEQ ID NO: 72 - DNA, 23 bp, Homo sapiens
  source 1..23: mol_type = other DNA
atccaatttaagtctgtaataca

SEQ ID NO: 73 - DNA, 23 bp, Homo sapiens
  source 1..23: mol_type = other DNA
cagcttcttcaccggtgatgtaa

SEQ ID NO: 74 - DNA, 23 bp, Homo sapiens
  source 1..23: mol_type = other DNA
agcttcttcaccggtgatgtaaa

SEQ ID NO: 75 - DNA, 23 bp, Homo sapiens
  source 1..23: mol_type = other DNA
ccggtgatgtaaactacctgttt

SEQ ID NO: 76 - DNA, 23 bp, Homo sapiens
  source 1..23: mol_type = other DNA
cggtgatgtaaactacctgtttg

SEQ ID NO: 77 - DNA, 23 bp, Homo sapiens
  source 1..23: mol_type = other DNA
ctgtttgaaggggtgtgctatgg

SEQ ID NO: 78 - DNA, 23 bp, Homo sapiens
  source 1..23: mol_type = other DNA
gtgctatggacccctagtactga

SEQ ID NO: 79 - DNA, 23 bp, Homo sapiens
  source 1..23: mol_type = other DNA
cgctggtcatctgcagcatttct

SEQ ID NO: 80 - DNA, 23 bp, Homo sapiens
  source 1..23: mol_type = other DNA
ctggtcatctgcagcatttcttc

SEQ ID NO: 81 - DNA, 23 bp, Homo sapiens
  source 1..23: mol_type = other DNA
tggtcatctgcagcatttcttcc

SEQ ID NO: 82 - DNA, 23 bp, Homo sapiens
  source 1..23: mol_type = other DNA
ctgcagcatttcttcctacttca

SEQ ID NO: 83 - DNA, 23 bp, Homo sapiens
  source 1..23: mol_type = other DNA
cagcatttcttcctacttcatta

SEQ ID NO: 84 - DNA, 23 bp, Homo sapiens
  source 1..23: mol_type = other DNA
agcatttcttcctacttcattat

SEQ ID NO: 85 - DNA, 23 bp, Homo sapiens
  source 1..23: mol_type = other DNA
ttcctacttcattattggataca

SEQ ID NO: 86 - DNA, 23 bp, Homo sapiens
  source 1..23: mol_type = other DNA
tacttcattattggatacactgc

SEQ ID NO: 87 - DNA, 23 bp, Homo sapiens
  source 1..23: mol_type = other DNA
ctgcatttattgccatcttatgc

SEQ ID NO: 88 - DNA, 23 bp, Homo sapiens
  source 1..23: mol_type = other DNA
tgccatcttatgctatctcctgg

SEQ ID NO: 89 - DNA, 23 bp, Homo sapiens
  source 1..23: mol_type = other DNA
tgctatctcctggttttcccact

SEQ ID NO: 90 - DNA, 23 bp, Homo sapiens
  source 1..23: mol_type = other DNA
tggcggtattcatgacaagaatg

SEQ ID NO: 91 - DNA, 23 bp, Homo sapiens
  source 1..23: mol_type = other DNA
ggcggtattcatgacaagaatgg

SEQ ID NO: 92 - DNA, 23 bp, Homo sapiens
  source 1..23: mol_type = other DNA
ggctgtgaaggctcagcatcaca

SEQ ID NO: 93 - DNA, 23 bp, Homo sapiens
  source 1..23: mol_type = other DNA
ggctcagcatcacacatctgagg

SEQ ID NO: 94 - DNA, 23 bp, Homo sapiens
  source 1..23: mol_type = other DNA
cagtgaagttctcacttgcatta

SEQ ID NO: 95 - DNA, 23 bp, Homo sapiens
  source 1..23: mol_type = other DNA
gtgaagttctcacttgcattaag

SEQ ID NO: 96 - DNA, 23 bp, Homo sapiens
  source 1..23: mol_type = other DNA
ttctcacttgcattaagctgatt

SEQ ID NO: 97 - DNA, 23 bp, Homo sapiens
  source 1..23: mol_type = other DNA
ctcacttgcattaagctgattaa

SEQ ID NO: 98 - DNA, 23 bp, Homo sapiens
  source 1..23: mol_type = other DNA
cacttgcattaagctgattaaaa

SEQ ID NO: 99 - DNA, 23 bp, Homo sapiens
  source 1..23: mol_type = other DNA
ttgcattaagctgattaaaatgt

SEQ ID NO: 100 - DNA, 23 bp, Homo sapiens
  source 1..23: mol_type = other DNA
aagctgattaaaatgtacacatg

SEQ ID NO: 101 - DNA, 23 bp, Homo sapiens
  source 1..23: mol_type = other DNA
agctgattaaaatgtacacatgg

SEQ ID NO: 102 - DNA, 23 bp, Homo sapiens
  source 1..23: mol_type = other DNA
atgtacacatgggagaaaccatt

SEQ ID NO: 103 - DNA, 23 bp, Homo sapiens
  source 1..23: mol_type = other DNA
atgggagaaaccatttgcagaaa

SEQ ID NO: 104 - DNA, 23 bp, Homo sapiens
  source 1..23: mol_type = other DNA
tgggagaaaccatttgcagaaat

SEQ ID NO: 105 - DNA, 23 bp, Homo sapiens
  source 1..23: mol_type = other DNA
gggagaaaccatttgcagaaatc

SEQ ID NO: 106 - DNA, 23 bp, Homo sapiens
  source 1..23: mol_type = other DNA
accatttgcagaaatcattgaag

SEQ ID NO: 107 - DNA, 23 bp, Homo sapiens
  source 1..23: mol_type = other DNA
cagaaatcattgaagacctaaga

SEQ ID NO: 108 - DNA, 23 bp, Homo sapiens
  source 1..23: mol_type = other DNA
aagacctaagaaggaaggaaagg

SEQ ID NO: 109 - DNA, 23 bp, Homo sapiens
  source 1..23: mol_type = other DNA
acctaagaaggaaggaaaggaaa

SEQ ID NO: 110 - DNA, 23 bp, Homo sapiens
  source 1..23: mol_type = other DNA
aagaaggaaggaaaggaaactat

SEQ ID NO: 111 - DNA, 23 bp, Homo sapiens
  source 1..23: mol_type = other DNA
aaggaaggaaaggaaactattgg

SEQ ID NO: 112 - DNA, 23 bp, Homo sapiens
  source 1..23: mol_type = other DNA
aggaaaggaaactattggagaag

SEQ ID NO: 113 - DNA, 23 bp, Homo sapiens
  source 1..23: mol_type = other DNA
gcctgacaagtataaccttgttc

SEQ ID NO: 114 - DNA, 23 bp, Homo sapiens
  source 1..23: mol_type = other DNA
gacaagtataaccttgttcatca

SEQ ID NO: 115 - DNA, 23 bp, Homo sapiens
  source 1..23: mol_type = other DNA
aagtataaccttgttcatcatcc

SEQ ID NO: 116 - DNA, 23 bp, Homo sapiens
  source 1..23: mol_type = other DNA
gggttctcatccacacatcctta

SEQ ID NO: 117 - DNA, 23 bp, Homo sapiens
  source 1..23: mol_type = other DNA
tccacacatccttaaagctgaaa

SEQ ID NO: 118 - DNA, 23 bp, Homo sapiens
  source 1..23: mol_type = other DNA
cacacatccttaaagctgaaact

SEQ ID NO: 119 - DNA, 23 bp, Homo sapiens
  source 1..23: mol_type = other DNA
cacatccttaaagctgaaactca

SEQ ID NO: 120 - DNA, 23 bp, Homo sapiens
  source 1..23: mol_type = other DNA
tccttaaagctgaaactcacagc

SEQ ID NO: 121 - DNA, 23 bp, Homo sapiens
  source 1..23: mol_type = other DNA
cagcgtcaatggccttcagcatg

SEQ ID NO: 122 - DNA, 23 bp, Homo sapiens
  source 1..23: mol_type = other DNA
agcgtcaatggccttcagcatgc

SEQ ID NO: 123 - DNA, 23 bp, Homo sapiens
  source 1..23: mol_type = other DNA
cagcatgctggcctccttgaatc

SEQ ID NO: 124 - DNA, 23 bp, Homo sapiens
  source 1..23: mol_type = other DNA
gtgttctttgtgcctattgcagt

SEQ ID NO: 125 - DNA, 23 bp, Homo sapiens
  source 1..23: mol_type = other DNA
gtgcctattgcagtcaaaggtct

SEQ ID NO: 126 - DNA, 23 bp, Homo sapiens
  source 1..23: mol_type = other DNA
gcctattgcagtcaaaggtctca

SEQ ID NO: 127 - DNA, 23 bp, Homo sapiens
  source 1..23: mol_type = other DNA
cagtcaaaggtctcacgaattcc

SEQ ID NO: 128 - DNA, 23 bp, Homo sapiens
  source 1..23: mol_type = other DNA
ctgcagtgatgaggttcaagaag

SEQ ID NO: 129 - DNA, 23 bp, Homo sapiens
  source 1..23: mol_type = other DNA
tgcagtgatgaggttcaagaagt

SEQ ID NO: 130 - DNA, 23 bp, Homo sapiens
  source 1..23: mol_type = other DNA
cagtgatgaggttcaagaagttt

SEQ ID NO: 131 - DNA, 23 bp, Homo sapiens
  source 1..23: mol_type = other DNA
gtgatgaggttcaagaagttttt

SEQ ID NO: 132 - DNA, 23 bp, Homo sapiens
  source 1..23: mol_type = other DNA
gaggttcaagaagtttttcctcc

SEQ ID NO: 133 - DNA, 23 bp, Homo sapiens
  source 1..23: mol_type = other DNA
ttctatgtccagacattacaaga

SEQ ID NO: 134 - DNA, 23 bp, Homo sapiens
  source 1..23: mol_type = other DNA
cccagcaaagctctggtctttga

SEQ ID NO: 135 - DNA, 23 bp, Homo sapiens
  source 1..23: mol_type = other DNA
cagcaaagctctggtctttgagg

SEQ ID NO: 136 - DNA, 23 bp, Homo sapiens
  source 1..23: mol_type = other DNA
gagaggaacgggcatgcttctga

SEQ ID NO: 137 - DNA, 23 bp, Homo sapiens
  source 1..23: mol_type = other DNA
gggatgaccaggcctagagatgc

SEQ ID NO: 138 - DNA, 23 bp, Homo sapiens
  source 1..23: mol_type = other DNA
gcccagagttgcacaagatcaac

SEQ ID NO: 139 - DNA, 23 bp, Homo sapiens
  source 1..23: mol_type = other DNA
cccagagttgcacaagatcaacc

SEQ ID NO: 140 - DNA, 23 bp, Homo sapiens
  source 1..23: mol_type = other DNA
tggtgtccaaggggatgatgtta

SEQ ID NO: 141 - DNA, 23 bp, Homo sapiens
  source 1..23: mol_type = other DNA
cggcaacacggggagtggtaaga

SEQ ID NO: 142 - DNA, 23 bp, Homo sapiens
  source 1..23: mol_type = other DNA
gtggtaagagcagcctgttgtca

SEQ ID NO: 143 - DNA, 23 bp, Homo sapiens
  source 1..23: mol_type = other DNA
cgggaacatcagggagaacatcc

SEQ ID NO: 144 - DNA, 23 bp, Homo sapiens
  source 1..23: mol_type = other DNA
ctggaacttctgccctttggaga

SEQ ID NO: 145 - DNA, 23 bp, Homo sapiens
  source 1..23: mol_type = other DNA
ctgccctttggagacatgacaga

SEQ ID NO: 146 - DNA, 23 bp, Homo sapiens
  source 1..23: mol_type = other DNA
gccctttggagacatgacagaga

SEQ ID NO: 147 - DNA, 23 bp, Homo sapiens
  source 1..23: mol_type = other DNA
cacatttttgaggagtgcattaa

SEQ ID NO: 148 - DNA, 23 bp, Homo sapiens
  source 1..23: mol_type = other DNA
agctgcagtacttagaattttgt

SEQ ID NO: 149 - DNA, 23 bp, Homo sapiens
  source 1..23: mol_type = other DNA
ctgcagtacttagaattttgtgg

SEQ ID NO: 150 - DNA, 23 bp, Homo sapiens
  source 1..23: mol_type = other DNA
tagaattttgtggccagatcatt

SEQ ID NO: 151 - DNA, 23 bp, Homo sapiens
  source 1..23: mol_type = other DNA
tggccagatcattttgttggaaa

SEQ ID NO: 152 - DNA, 23 bp, Homo sapiens
  source 1..23: mol_type = other DNA
ggccagatcattttgttggaaaa

SEQ ID NO: 153 - DNA, 23 bp, Homo sapiens
  source 1..23: mol_type = other DNA
gccagatcattttgttggaaaat

SEQ ID NO: 154 - DNA, 23 bp, Homo sapiens
  source 1..23: mol_type = other DNA
cagatcattttgttggaaaatgg

SEQ ID NO: 155 - DNA, 23 bp, Homo sapiens
  source 1..23: mol_type = other DNA
ttggaaaatgggaaaatctgtga

SEQ ID NO: 156 - DNA, 23 bp, Homo sapiens
  source 1..23: mol_type = other DNA
gggaaaatctgtgaaaatggaac

SEQ ID NO: 157 - DNA, 23 bp, Homo sapiens
  source 1..23: mol_type = other DNA
atggaactcacagtgagttaatg

SEQ ID NO: 158 - DNA, 23 bp, Homo sapiens
  source 1..23: mol_type = other DNA
tggaactcacagtgagttaatgc

SEQ ID NO: 159 - DNA, 23 bp, Homo sapiens
  source 1..23: mol_type = other DNA
aactcacagtgagttaatgcaga

SEQ ID NO: 160 - DNA, 23 bp, Homo sapiens
  source 1..23: mol_type = other DNA
ctcacagtgagttaatgcagaaa

SEQ ID NO: 161 - DNA, 23 bp, Homo sapiens
  source 1..23: mol_type = other DNA
cacagtgagttaatgcagaaaaa

SEQ ID NO: 162 - DNA, 23 bp, Homo sapiens
  source 1..23: mol_type = other DNA
cagtgagttaatgcagaaaaagg

SEQ ID NO: 163 - DNA, 23 bp, Homo sapiens
  source 1..23: mol_type = other DNA
atgcagaaaaaggggaaatatgc

SEQ ID NO: 164 - DNA, 23 bp, Homo sapiens
  source 1..23: mol_type = other DNA
aggggaaatatgcccaacttatc

SEQ ID NO: 165 - DNA, 23 bp, Homo sapiens
  source 1..23: mol_type = other DNA
ggggaaatatgcccaacttatcc

SEQ ID NO: 166 - DNA, 23 bp, Homo sapiens
  source 1..23: mol_type = other DNA
tgcccaacttatccagaagatgc

SEQ ID NO: 167 - DNA, 23 bp, Homo sapiens
  source 1..23: mol_type = other DNA
atccagaagatgcacaaggaagc

SEQ ID NO: 168 - DNA, 23 bp, Homo sapiens
  source 1..23: mol_type = other DNA
caggacacagcaaagatagcaga

SEQ ID NO: 169 - DNA, 23 bp, Homo sapiens
  source 1..23: mol_type = other DNA
agcagagaagccaaaggtagaaa

SEQ ID NO: 170 - DNA, 23 bp, Homo sapiens
  source 1..23: mol_type = other DNA
gagaagccaaaggtagaaagtca

SEQ ID NO: 171 - DNA, 23 bp, Homo sapiens
  source 1..23: mol_type = other DNA
gagtctctcaacggaaatgctgt

SEQ ID NO: 172 - DNA, 23 bp, Homo sapiens
  source 1..23: mol_type = other DNA
gtctctcaacggaaatgctgtgc

SEQ ID NO: 173 - DNA, 23 bp, Homo sapiens
  source 1..23: mol_type = other DNA
atggaagaaggctccttgagttg

SEQ ID NO: 174 - DNA, 23 bp, Homo sapiens
  source 1..23: mol_type = other DNA
tggaagaaggctccttgagttgg

SEQ ID NO: 175 - DNA, 23 bp, Homo sapiens
  source 1..23: mol_type = other DNA
gaggttacatggtctcttgcata

SEQ ID NO: 176 - DNA, 23 bp, Homo sapiens
  source 1..23: mol_type = other DNA
aggttacatggtctcttgcataa

SEQ ID NO: 177 - DNA, 23 bp, Homo sapiens
  source 1..23: mol_type = other DNA
tacatggtctcttgcataatttt

SEQ ID NO: 178 - DNA, 23 bp, synthetic construct
  misc_feature 1..23: note = Synthetic sequence
  source 1..23: mol_type = other DNA
tggtctcttgcataattttcttc

SEQ ID NO: 179 - DNA, 23 bp, Homo sapiens
  source 1..23: mol_type = other DNA
ctcttgcataattttcttcttcg

SEQ ID NO: 180 - DNA, 23 bp, Homo sapiens
  source 1..23: mol_type = other DNA
ttgcataattttcttcttcgtgg

SEQ ID NO: 181 - DNA, 23 bp, Homo sapiens
  source 1..23: mol_type = other DNA
tggtgctgatcgtcttcttaacg

SEQ ID NO: 182 - DNA, 23 bp, Homo sapiens
  source 1..23: mol_type = other DNA
tgctgatcgtcttcttaacgatc

SEQ ID NO: 183 - DNA, 23 bp, Homo sapiens
  source 1..23: mol_type = other DNA
tcgtcttcttaacgatcttcagc

SEQ ID NO: 184 - DNA, 23 bp, Homo sapiens
  source 1..23: mol_type = other DNA
ggcaacattgcagacaatcctca

SEQ ID NO: 185 - DNA, 23 bp, Homo sapiens
  source 1..23: mol_type = other DNA
aacattgcagacaatcctcaact

SEQ ID NO: 186 - DNA, 23 bp, Homo sapiens
  source 1..23: mol_type = other DNA
ttgcagacaatcctcaactgtcc

SEQ ID NO: 187 - DNA, 23 bp, Homo sapiens
  source 1..23: mol_type = other DNA
tcctcaactgtccttctaccagc

SEQ ID NO: 188 - DNA, 23 bp, Homo sapiens
  source 1..23: mol_type = other DNA
cagggattttcaccaaggtcacg

SEQ ID NO: 189 - DNA, 23 bp, Homo sapiens
  source 1..23: mol_type = other DNA
ccctgcacaacaagctctttaac

SEQ ID NO: 190 - DNA, 23 bp, Homo sapiens
  source 1..23: mol_type = other DNA
tgcacaacaagctctttaacaag

SEQ ID NO: 191 - DNA, 23 bp, Homo sapiens
  source 1..23: mol_type = other DNA
aacaagctctttaacaaggtttt

SEQ ID NO: 192 - DNA, 23 bp, Homo sapiens
  source 1..23: mol_type = other DNA
gccccatgagtttctttgacacc

SEQ ID NO: 193 - DNA, 23 bp, Homo sapiens
  source 1..23: mol_type = other DNA
ttctttgacaccatcccaatagg

SEQ ID NO: 194 - DNA, 23 bp, Homo sapiens
  source 1..23: mol_type = other DNA
tcccaataggccggcttttgaac

SEQ ID NO: 195 - DNA, 23 bp, Homo sapiens
  source 1..23: mol_type = other DNA
cccaataggccggcttttgaact

SEQ ID NO: 196 - DNA, 23 bp, Homo sapiens
  source 1..23: mol_type = other DNA
accagctcttgcccatcttttca

SEQ ID NO: 197 - DNA, 23 bp, Homo sapiens
  source 1..23: mol_type = other DNA
cagctcttgcccatcttttcaga

SEQ ID NO: 198 - DNA, 23 bp, Homo sapiens
  source 1..23: mol_type = other DNA
ctgtctccatatatcctgttaat

SEQ ID NO: 199 - DNA, 23 bp, Homo sapiens
  source 1..23: mol_type = other DNA
gtctccatatatcctgttaatgg

SEQ ID NO: 200 - DNA, 23 bp, Homo sapiens
  source 1..23: mol_type = other DNA
tcctgttaatgggagccataatc

SEQ ID NO: 201 - DNA, 23 bp, Homo sapiens
  source 1..23: mol_type = other DNA
gggagccataatcatggttattt

SEQ ID NO: 202 - DNA, 23 bp, Homo sapiens
  source 1..23: mol_type = other DNA
gagccataatcatggttatttgc

SEQ ID NO: 203 - DNA, 23 bp, Homo sapiens
  source 1..23: mol_type = other DNA
atcatggttatttgcttcattta

SEQ ID NO: 204 - DNA, 23 bp, Homo sapiens
  source 1..23: mol_type = other DNA
atggttatttgcttcatttatta

SEQ ID NO: 205 - DNA, 23 bp, Homo sapiens
  source 1..23: mol_type = other DNA
tggttatttgcttcatttattat

SEQ ID NO: 206 - DNA, 23 bp, Homo sapiens
  source 1..23: mol_type = other DNA
ttgcttcatttattatatgatgt

SEQ ID NO: 207 - DNA, 23 bp, Homo sapiens
  source 1..23: mol_type = other DNA
ttcaagagactggagaactatag

SEQ ID NO: 208 - DNA, 23 bp, Homo sapiens
  source 1..23: mol_type = other DNA
aactatagccggtctcctttatt

SEQ ID NO: 209 - DNA, 23 bp, Homo sapiens
  source 1..23: mol_type = other DNA
tcctttattctcccacatcctca

SEQ ID NO: 210 - DNA, 23 bp, Homo sapiens
  source 1..23: mol_type = other DNA
ctcccacatcctcaattctctgc

SEQ ID NO: 211 - DNA, 23 bp, Homo sapiens
  source 1..23: mol_type = other DNA
ctccatccatgtctatggaaaaa

SEQ ID NO: 212 - DNA, 23 bp, Homo sapiens
  source 1..23: mol_type = other DNA
tccatccatgtctatggaaaaac

SEQ ID NO: 213 - DNA, 23 bp, Homo sapiens
  source 1..23: mol_type = other DNA
atccatgtctatggaaaaactga

SEQ ID NO: 214 - DNA, 23 bp, Homo sapiens
  source 1..23: mol_type = other DNA
atggaaaaactgaagacttcatc

SEQ ID NO: 215 - DNA, 23 bp, Homo sapiens
  source 1..23: mol_type = other DNA
tggaaaaactgaagacttcatca

SEQ ID NO: 216 - DNA, 23 bp, Homo sapiens
  source 1..23: mol_type = other DNA
aagacttcatcagccagtttaag

SEQ ID NO: 217 - DNA, 23 bp, Homo sapiens
  source 1..23: mol_type = other DNA
cagtttaagaggctgactgatgc

SEQ ID NO: 218 - DNA, 23 bp, Homo sapiens
  source 1..23: mol_type = other DNA
ggctgactgatgcgcagaataac

SEQ ID NO: 219 - DNA, 23 bp, Homo sapiens
  source 1..23: mol_type = other DNA
acctgctgttgtttctatcttcc

SEQ ID NO: 220 - DNA, 23 bp, Homo sapiens
  source 1..23: mol_type = other DNA
tgctgttgtttctatcttccaca

SEQ ID NO: 221 - DNA, 23 bp, Homo sapiens
  source 1..23: mol_type = other DNA
ctgttgtttctatcttccacacg

SEQ ID NO: 222 - DNA, 23 bp, Homo sapiens
  source 1..23: mol_type = other DNA
ggcattgaggctggagatcatga

SEQ ID NO: 223 - DNA, 23 bp, Homo sapiens
  source 1..23: mol_type = other DNA
ccctgttcgtggcttttggcatt

SEQ ID NO: 224 - DNA, 23 bp, Homo sapiens
  source 1..23: mol_type = other DNA
tcgtggcttttggcatttcctcc

SEQ ID NO: 225 - DNA, 23 bp, Homo sapiens
  source 1..23: mol_type = other DNA
ccccctactcctttaaagtcatg

SEQ ID NO: 226 - DNA, 23 bp, Homo sapiens
  source 1..23: mol_type = other DNA
cccctactcctttaaagtcatgg

SEQ ID NO: 227 - DNA, 23 bp, Homo sapiens
  source 1..23: mol_type = other DNA
tggagacagaggcacagttcacg

SEQ ID NO: 228 - DNA, 23 bp, Homo sapiens
  source 1..23: mol_type = other DNA
ggcacagttcacggctgtagaga

SEQ ID NO: 229 - DNA, 23 bp, Homo sapiens
  source 1..23: mol_type = other DNA
cacagttcacggctgtagagagg

SEQ ID NO: 230 - DNA, 23 bp, Homo sapiens
  source 1..23: mol_type = other DNA
ggctgtagagaggatactgcagt

SEQ ID NO: 231 - DNA, 23 bp, Homo sapiens
  source 1..23: mol_type = other DNA
gaggatactgcagtacatgaaga

SEQ ID NO: 232 - DNA, 23 bp, Homo sapiens
  source 1..23: mol_type = other DNA
tactgcagtacatgaagatgtgt

SEQ ID NO: 233 - skipped

SEQ ID NO: 234 - DNA, 23 bp, Homo sapiens
  source 1..23: mol_type = other DNA
ctgcagtacatgaagatgtgtgt

SEQ ID NO: 235 - DNA, 23 bp, Homo sapiens
  source 1..23: mol_type = other DNA
tacacatggaaggcacaagttgt

SEQ ID NO: 236 - DNA, 23 bp, Homo sapiens
  source 1..23: mol_type = other DNA
cacatggaaggcacaagttgtcc

SEQ ID NO: 237 - DNA, 23 bp, Homo sapiens
  source 1..23: mol_type = other DNA
gccacagcatggggaaatcatat

SEQ ID NO: 238 - DNA, 23 bp, Homo sapiens
  source 1..23: mol_type = other DNA
tggggaaatcatatttcaggatt

SEQ ID NO: 239 - DNA, 23 bp, Homo sapiens
  source 1..23: mol_type = other DNA
ggggaaatcatatttcaggatta

SEQ ID NO: 240 - DNA, 23 bp, Homo sapiens
  source 1..23: mol_type = other DNA
gggaaatcatatttcaggattat

SEQ ID NO: 241 - DNA, 23 bp, Homo sapiens
  source 1..23: mol_type = other DNA
ttcaggattatcacatgaaatac

SEQ ID NO: 242 - DNA, 23 bp, Homo sapiens
  source 1..23: mol_type = other DNA
aggattatcacatgaaatacaga

SEQ ID NO: 243 - DNA, 23 bp, Homo sapiens
  source 1..23: mol_type = other DNA
atcacatgaaatacagagacaac

SEQ ID NO: 244 - DNA, 23 bp, Homo sapiens
  source 1..23: mol_type = other DNA
atgaaatacagagacaacacacc

SEQ ID NO: 245 - DNA, 23 bp, Homo sapiens
  source 1..23: mol_type = other DNA
ctcattgacggcgtggacatttg

SEQ ID NO: 246 - DNA, 23 bp, Homo sapiens
  source 1..23: mol_type = other DNA
aggacttgcggtccaagctctca

SEQ ID NO: 247 - DNA, 23 bp, Homo sapiens
  source 1..23: mol_type = other DNA
gcggtccaagctctcagtgatcc

SEQ ID NO: 248 - DNA, 23 bp, Homo sapiens
  source 1..23: mol_type = other DNA
ctctcagtgatccctcaagatcc

SEQ ID NO: 249 - DNA, 23 bp, Homo sapiens
  source 1..23: mol_type = other DNA
ctgctctcaggaaccatcagatt

SEQ ID NO: 250 - DNA, 23 bp, Homo sapiens
  source 1..23: mol_type = other DNA
aggaaccatcagattcaacctag

SEQ ID NO: 251 - DNA, 23 bp, Homo sapiens
  source 1..23: mol_type = other DNA
aaccatcagattcaacctagatc

SEQ ID NO: 252 - DNA, 23 bp, Homo sapiens
  source 1..23: mol_type = other DNA
accatcagattcaacctagatcc

SEQ ID NO: 253 - DNA, 23 bp, Homo sapiens
  source 1..23: mol_type = other DNA
tccctttgaccgtcacactgacc

SEQ ID NO: 254 - DNA, 23 bp, Homo sapiens
  source 1..23: mol_type = other DNA
tgccttggagaggacattcctga

SEQ ID NO: 255 - DNA, 23 bp, Homo sapiens
  source 1..23: mol_type = other DNA
tcctgaccaaggccatctcaaag

SEQ ID NO: 256 - DNA, 23 bp, Homo sapiens
  source 1..23: mol_type = other DNA
ctcaaagttccccaaaaagctgc

SEQ ID NO: 257 - DNA, 23 bp, Homo sapiens
  source 1..23: mol_type = other DNA
ttccccaaaaagctgcatacaga

SEQ ID NO: 258 - DNA, 23 bp, Homo sapiens
  source 1..23: mol_type = other DNA
ccccaaaaagctgcatacagatg

SEQ ID NO: 259 - DNA, 23 bp, Homo sapiens
  source 1..23: mol_type = other DNA
cccaaaaagctgcatacagatgt

SEQ ID NO: 260 - DNA, 23 bp, Homo sapiens
  source 1..23: mol_type = other DNA
tggtggaaaacggtggaaacttc

SEQ ID NO: 261 - DNA, 23 bp, Homo sapiens
  source 1..23: mol_type = other DNA
tggaaaacggtggaaacttctct

SEQ ID NO: 262 - DNA, 23 bp, Homo sapiens
  source 1..23: mol_type = other DNA
ggctgtgcttcgcaactccaaga

SEQ ID NO: 263 - DNA, 23 bp, Homo sapiens
  source 1..23: mol_type = other DNA
tgcttcgcaactccaagatcatc

SEQ ID NO: 264 - DNA, 23 bp, Homo sapiens
  source 1..23: mol_type = other DNA
tcgcaactccaagatcatcctta

SEQ ID NO: 265 - DNA, 23 bp, Homo sapiens
  source 1..23: mol_type = other DNA
cgcaactccaagatcatccttat

SEQ ID NO: 266 - DNA, 23 bp, Homo sapiens
  source 1..23: mol_type = other DNA
ctccaagatcatccttatcgatg

SEQ ID NO: 267 - DNA, 23 bp, Homo sapiens
  source 1..23: mol_type = other DNA
tccaagatcatccttatcgatga

SEQ ID NO: 268 - DNA, 23 bp, Homo sapiens
  source 1..23: mol_type = other DNA
aagatcatccttatcgatgaagc

SEQ ID NO: 269 - DNA, 23 bp, Homo sapiens
  source 1..23: mol_type = other DNA
ctccattgacatggagacagaca

SEQ ID NO: 270 - DNA, 23 bp, Homo sapiens
  source 1..23: mol_type = other DNA
caccactgtgctgaactgtgacc

SEQ ID NO: 271 - DNA, 23 bp, Homo sapiens
  source 1..23: mol_type = other DNA
tcctggttatgggcaatgggaag

SEQ ID NO: 272 - DNA, 23 bp, Homo sapiens
  source 1..23: mol_type = other DNA
gggcaatgggaaggtggtagaat

SEQ ID NO: 273 - DNA, 23 bp, Homo sapiens
  source 1..23: mol_type = other DNA
ggcaatgggaaggtggtagaatt

SEQ ID NO: 274 - DNA, 23 bp, Homo sapiens
  source 1..23: mol_type = other DNA
tgggaaggtggtagaatttgatc

SEQ ID NO: 275 - DNA, 23 bp, Homo sapiens
  source 1..23: mol_type = other DNA
cagccacttcttcactgagataa

SEQ ID NO: 276 - DNA, 23 bp, Homo sapiens
  source 1..23: mol_type = other DNA
agccacttcttcactgagataag

SEQ ID NO: 277 - DNA, 23 bp, Homo sapiens
  source 1..23: mol_type = other DNA
gccacttcttcactgagataagg

SEQ ID NO: 278 - DNA, 23 bp, Homo sapiens
  source 1..23: mol_type = other DNA
ttcttcactgagataaggagatg

SEQ ID NO: 279 - DNA, 23 bp, Homo sapiens
  source 1..23: mol_type = other DNA
ttcactgagataaggagatgtgg

SEQ ID NO: 280 - DNA, 23 bp, Homo sapiens
  source 1..23: mol_type = other DNA
aaggagatgtggagacttcatgg

SEQ ID NO: 281 - DNA, 23 bp, Homo sapiens
  source 1..23: mol_type = other DNA
gagatgtggagacttcatggagg

SEQ ID NO: 282 - DNA, 23 bp, Homo sapiens
  source 1..23: mol_type = other DNA
cagcttcgaggcccacagtctgc

SEQ ID NO: 283 - DNA, 23 bp, Homo sapiens
  source 1..23: mol_type = other DNA
cccacagtctgcgaccttcttgt

SEQ ID NO: 284 - DNA, 23 bp, Homo sapiens
  source 1..23: mol_type = other DNA
gcgaccttcttgtttggagatga

SEQ ID NO: 285 - DNA, 23 bp, Homo sapiens
  source 1..23: mol_type = other DNA
gaccttcttgtttggagatgaga

SEQ ID NO: 286 - DNA, 23 bp, Homo sapiens
  source 1..23: mol_type = other DNA
ttggagatgagaacttctcctgg

SEQ ID NO: 287 - DNA, 23 bp, Homo sapiens
  source 1..23: mol_type = other DNA
ctcctggaagcaggggtaaatgt

SEQ ID NO: 288 - skipped

SEQ ID NO: 289 - DNA, 23 bp, Homo sapiens
  source 1..23: mol_type = other DNA
ctggaagcaggggtaaatgtagg

SEQ ID NO: 290 - DNA, 23 bp, Homo sapiens
  source 1..23: mol_type = other DNA
gtggggattgctggatggaaacc

SEQ ID NO: 291 - DNA, 23 bp, Homo sapiens
  source 1..23: mol_type = other DNA
ctggatggaaaccctggaatagg

SEQ ID NO: 292 - DNA, 23 bp, Homo sapiens
  source 1..23: mol_type = other DNA
tggaaaccctggaataggctact

SEQ ID NO: 293 - DNA, 23 bp, Homo sapiens
  source 1..23: mol_type = other DNA
accctggaataggctacttgatg

SEQ ID NO: 294 - DNA, 23 bp, Homo sapiens
  source 1..23: mol_type = other DNA
ccctggaataggctacttgatgg

SEQ ID NO: 295 - DNA, 23 bp, Homo sapiens
  source 1..23: mol_type = other DNA
gaccttagaaccccagaaccatc

SEQ ID NO: 296 - DNA, 23 bp, Homo sapiens
  source 1..23: mol_type = other DNA
accttagaaccccagaaccatct

SEQ ID NO: 297 - DNA, 23 bp, Homo sapiens
  source 1..23: mol_type = other DNA
accccagaaccatctaagacatg

SEQ ID NO: 298 - DNA, 23 bp, Homo sapiens
  source 1..23: mol_type = other DNA
ccccagaaccatctaagacatgg

SEQ ID NO: 299 - DNA, 23 bp, Homo sapiens
  source 1..23: mol_type = other DNA
aaccatctaagacatgggattca

SEQ ID NO: 300 - DNA, 23 bp, Homo sapiens
  source 1..23: mol_type = other DNA
atctaagacatgggattcagtga

SEQ ID NO: 301 - DNA, 23 bp, Homo sapiens
  source 1..23: mol_type = other DNA
gacatgggattcagtgatcatgt

SEQ ID NO: 302 - DNA, 23 bp, Homo sapiens
  source 1..23: mol_type = other DNA
gggattcagtgatcatgtggttc

SEQ ID NO: 303 - DNA, 23 bp, Homo sapiens
  source 1..23: mol_type = other DNA
gtgatcatgtggttctcctttta

SEQ ID NO: 304 - DNA, 23 bp, Homo sapiens
  source 1..23: mol_type = other DNA
atcatgtggttctccttttaact

SEQ ID NO: 305 - DNA, 23 bp, Homo sapiens
  source 1..23: mol_type = other DNA
atgtggttctccttttaacttac

SEQ ID NO: 306 - DNA, 23 bp, Homo sapiens
  source 1..23: mol_type = other DNA
tggttctccttttaacttacatg

SEQ ID NO: 307 - DNA, 23 bp, Homo sapiens
  source 1..23: mol_type = other DNA
tccttttaacttacatgctgaat

SEQ ID NO: 308 - DNA, 23 bp, Homo sapiens
  source 1..23: mol_type = other DNA
aacttacatgctgaataatttta

SEQ ID NO: 309 - DNA, 23 bp, Homo sapiens
  source 1..23: mol_type = other DNA
tacatgctgaataattttataat

SEQ ID NO: 310 - DNA, 23 bp, Homo sapiens
  source 1..23: mol_type = other DNA
atgctgaataattttataataag

SEQ ID NO: 311 - DNA, 23 bp, Homo sapiens
  source 1..23: mol_type = other DNA
tgctgaataattttataataagg

SEQ ID NO: 312 - DNA, 23 bp, Homo sapiens
  source 1..23: mol_type = other DNA
aaggtaaaagcttatagttttct

SEQ ID NO: 313 - DNA, 23 bp, Homo sapiens
  source 1..23: mol_type = other DNA
aagcttatagttttctgatctgt

SEQ ID NO: 314 - DNA, 23 bp, Homo sapiens
  source 1..23: mol_type = other DNA
ctgatctgtgttagaagtgttgc

SEQ ID NO: 315 - DNA, 23 bp, Homo sapiens
  source 1..23: mol_type = other DNA
ctgtgttagaagtgttgcaaatg

SEQ ID NO: 316 - DNA, 23 bp, Homo sapiens
  source 1..23: mol_type = other DNA
tagaagtgttgcaaatgctgtac

SEQ ID NO: 317 - DNA, 23 bp, Homo sapiens
  source 1..23: mol_type = other DNA
gtgttgcaaatgctgtactgact

SEQ ID NO: 318 - DNA, 23 bp, Homo sapiens
  source 1..23: mol_type = other DNA
ttgcaaatgctgtactgactttg

SEQ ID NO: 319 - DNA, 23 bp, Homo sapiens
  source 1..23: mol_type = other DNA
tgcaaatgctgtactgactttgt

SEQ ID NO: 320 - DNA, 23 bp, Homo sapiens
  source 1..23: mol_type = other DNA
atgctgtactgactttgtaaaat

SEQ ID NO: 321 - DNA, 23 bp, Homo sapiens
  source 1..23: mol_type = other DNA
tgctgtactgactttgtaaaata

SEQ ID NO: 322 - DNA, 23 bp, Homo sapiens
  source 1..23: mol_type = other DNA
ctgtactgactttgtaaaatata

SEQ ID NO: 323 - DNA, 23 bp, Homo sapiens
  source 1..23: mol_type = other DNA
tactgactttgtaaaatataaaa

SEQ ID NO: 324 - DNA, 23 bp, Homo sapiens
  source 1..23: mol_type = other DNA
ctgactttgtaaaatataaaact

SEQ ID NO: 325 - DNA, 23 bp, Homo sapiens
  source 1..23: mol_type = other DNA
gactttgtaaaatataaaactaa

SEQ ID NO: 326 - RNA, 21 nt, synthetic construct
  misc_feature 1..21: note = Synthetic sequence
  source 1..21: mol_type = other RNA
tggtttattcaaatacaccgg

SEQ ID NO: 327 - RNA, 21 nt, synthetic construct
  misc_feature 1..21: note = Synthetic sequence
  source 1..21: mol_type = other RNA
ggtgtatttgaataaaccagg

SEQ ID NO: 328 - RNA, 21 nt, synthetic construct
  misc_feature 1..21: note = Synthetic sequence
  source 1..21: mol_type = other RNA
tatagtatgatttgccaacct

SEQ ID NO: 329 - RNA, 21 nt, synthetic construct
  misc_feature 1..21: note = Synthetic sequence
  source 1..21: mol_type = other RNA
gttggcaaatcatactatagc

SEQ ID NO: 330 - RNA, 21 nt, synthetic construct
  misc_feature 1..21: note = Synthetic sequence
  source 1..21: mol_type = other RNA
agctatagtatgatttgccaa

SEQ ID NO: 331 - RNA, 21 nt, synthetic construct
  misc_feature 1..21: note = Synthetic sequence
  source 1..21: mol_type = other RNA
ggcaaatcatactatagctga

SEQ ID NO: 332 - RNA, 21 nt, synthetic construct
  misc_feature 1..21: note = Synthetic sequence
  source 1..21: mol_type = other RNA
tcagctatagtatgatttgcc

SEQ ID NO: 333 - RNA, 21 nt, synthetic construct
  misc_feature 1..21: note = Synthetic sequence
  source 1..21: mol_type = other RNA
caaatcatactatagctgaaa

SEQ ID NO: 334 - RNA, 21 nt, synthetic construct
  misc_feature 1..21: note = Synthetic sequence
  source 1..21: mol_type = other RNA
ttctttcagctatagtatgat

SEQ ID NO: 335 - RNA, 21 nt, synthetic construct
  misc_feature 1..21: note = Synthetic sequence
  source 1..21: mol_type = other RNA
catactatagctgaaagaatt

SEQ ID NO: 336 - RNA, 21 nt, synthetic construct
  misc_feature 1..21: note = Synthetic sequence
  source 1..21: mol_type = other RNA
agttcctgccaattctttcag

SEQ ID NO: 337 - RNA, 21 nt, synthetic construct
  misc_feature 1..21: note = Synthetic sequence
  source 1..21: mol_type = other RNA
gaaagaattggcaggaactga

SEQ ID NO: 338 - RNA, 21 nt, synthetic construct
  misc_feature 1..21: note = Synthetic sequence
  source 1..21: mol_type = other RNA
tttcagttcctgccaattctt

SEQ ID NO: 339 - RNA, 21 nt, synthetic construct
  misc_feature 1..21: note = Synthetic sequence
  source 1..21: mol_type = other RNA
gaattggcaggaactgaaaat

SEQ ID NO: 340 - RNA, 21 nt, synthetic construct
  misc_feature 1..21: note = Synthetic sequence
  source 1..21: mol_type = other RNA
agtcattttcagttcctgcca

SEQ ID NO: 341 - RNA, 21 nt, synthetic construct
  misc_feature 1..21: note = Synthetic sequence
  source 1..21: mol_type = other RNA
gcaggaactgaaaatgactag

SEQ ID NO: 342 - RNA, 21 nt, synthetic construct
  misc_feature 1..21: note = Synthetic sequence
  source 1..21: mol_type = other RNA
tagtcattttcagttcctgcc

SEQ ID NO: 343 - RNA, 21 nt, synthetic construct
  misc_feature 1..21: note = Synthetic sequence
  source 1..21: mol_type = other RNA
caggaactgaaaatgactagg

SEQ ID NO: 344 - RNA, 21 nt, synthetic construct
  misc_feature 1..21: note = Synthetic sequence
  source 1..21: mol_type = other RNA
tcctagtcattttcagttcct

SEQ ID NO: 345 - RNA, 21 nt, synthetic construct
  misc_feature 1..21: note = Synthetic sequence
  source 1..21: mol_type = other RNA
gaactgaaaatgactaggaag

SEQ ID NO: 346 - RNA, 21 nt, synthetic construct
  misc_feature 1..21: note = Synthetic sequence
  source 1..21: mol_type = other RNA
tcttcctagtcattttcagtt

SEQ ID NO: 347 - RNA, 21 nt, synthetic construct
  misc_feature 1..21: note = Synthetic sequence
  source 1..21: mol_type = other RNA
ctgaaaatgactaggaagagg

SEQ ID NO: 348 - RNA, 21 nt, synthetic construct
  misc_feature 1..21: note = Synthetic sequence
  source 1..21: mol_type = other RNA
tgtcgatgccacgattcacga

SEQ ID NO: 349 - RNA, 21 nt, synthetic construct
  misc_feature 1..21: note = Synthetic sequence
  source 1..21: mol_type = other RNA
gtgaatcgtggcatcgacata

SEQ ID NO: 350 - RNA, 21 nt, synthetic construct
  misc_feature 1..21: note = Synthetic sequence
  source 1..21: mol_type = other RNA
tatgtcgatgccacgattcac

SEQ ID NO: 351 - RNA, 21 nt, synthetic construct
  misc_feature 1..21: note = Synthetic sequence
  source 1..21: mol_type = other RNA
gaatcgtggcatcgacatagg

SEQ ID NO: 352 - RNA, 21 nt, synthetic construct
  misc_feature 1..21: note = Synthetic sequence
  source 1..21: mol_type = other RNA
tcctgaaaccatgtcatcgcc

SEQ ID NO: 353 - RNA, 21 nt, synthetic construct
  misc_feature 1..21: note = Synthetic sequence
  source 1..21: mol_type = other RNA
cgatgacatggtttcaggact

SEQ ID NO: 354 - RNA, 21 nt, synthetic construct
  misc_feature 1..21: note = Synthetic sequence
  source 1..21: mol_type = other RNA
taagtcctgaaaccatgtcat

SEQ ID NO: 355 - RNA, 21 nt, synthetic construct
  misc_feature 1..21: note = Synthetic sequence
  source 1..21: mol_type = other RNA
gacatggtttcaggacttatt

SEQ ID NO: 356 - RNA, 21 nt, synthetic construct
  misc_feature 1..21: note = Synthetic sequence
  source 1..21: mol_type = other RNA
aataagtcctgaaaccatgtc

SEQ ID NO: 357 - RNA, 21 nt, synthetic construct
  misc_feature 1..21: note = Synthetic sequence
  source 1..21: mol_type = other RNA
catggtttcaggacttattta

SEQ ID NO: 358 - RNA, 21 nt, synthetic construct
  misc_feature 1..21: note = Synthetic sequence
  source 1..21: mol_type = other RNA
ataaataagtcctgaaaccat

SEQ ID NO: 359 - RNA, 21 nt, synthetic construct
  misc_feature 1..21: note = Synthetic sequence
  source 1..21: mol_type = other RNA
ggtttcaggacttatttataa

SEQ ID NO: 360 - RNA, 21 nt, synthetic construct
  misc_feature 1..21: note = Synthetic sequence
  source 1..21: mol_type = other RNA
tataaataagtcctgaaacca

SEQ ID NO: 361 - RNA, 21 nt, synthetic construct
  misc_feature 1..21: note = Synthetic sequence
  source 1..21: mol_type = other RNA
gtttcaggacttatttataaa

SEQ ID NO: 362 - RNA, 21 nt, synthetic construct
  misc_feature 1..21: note = Synthetic sequence
  source 1..21: mol_type = other RNA
aggttttataaataagtcctg

SEQ ID NO: 363 - RNA, 21 nt, synthetic construct
  misc_feature 1..21: note = Synthetic sequence
  source 1..21: mol_type = other RNA
ggacttatttataaaacctat

SEQ ID NO: 364 - RNA, 21 nt, synthetic construct
  misc_feature 1..21: note = Synthetic sequence
  source 1..21: mol_type = other RNA
taggttttataaataagtcct

SEQ ID NO: 365 - RNA, 21 nt, synthetic construct
  misc_feature 1..21: note = Synthetic sequence
  source 1..21: mol_type = other RNA
gacttatttataaaacctata

SEQ ID NO: 366 - RNA, 21 nt, synthetic construct
  misc_feature 1..21: note = Synthetic sequence
  source 1..21: mol_type = other RNA
tataggttttataaataagtc

SEQ ID NO: 367 - RNA, 21 nt, synthetic construct
  misc_feature 1..21: note = Synthetic sequence
  source 1..21: mol_type = other RNA
cttatttataaaacctatact

SEQ ID NO: 368 - RNA, 21 nt, synthetic construct
  misc_feature 1..21: note = Synthetic sequence
  source 1..21: mol_type = other RNA
tttctctcttgctgactccag

SEQ ID NO: 369 - RNA, 21 nt, synthetic construct
  misc_feature 1..21: note = Synthetic sequence
  source 1..21: mol_type = other RNA
ggagtcagcaagagagaaatc

SEQ ID NO: 370 - RNA, 21 nt, synthetic construct
  misc_feature 1..21: note = Synthetic sequence
  source 1..21: mol_type = other RNA
tctcaaggcagcatcatactt

SEQ ID NO: 371 - RNA, 21 nt, synthetic construct
  misc_feature 1..21: note = Synthetic sequence
  source 1..21: mol_type = other RNA
gtatgatgctgccttgagaac

SEQ ID NO: 372 - RNA, 21 nt, synthetic construct
  misc_feature 1..21: note = Synthetic sequence
  source 1..21: mol_type = other RNA
agaacaggccagcattgtcca

SEQ ID NO: 373 - RNA, 21 nt, synthetic construct
  misc_feature 1..21: note = Synthetic sequence
  source 1..21: mol_type = other RNA
gacaatgctggcctgttctcc

SEQ ID NO: 374 - RNA, 21 nt, synthetic construct
  misc_feature 1..21: note = Synthetic sequence
  source 1..21: mol_type = other RNA
aagctttggatcatgagcggg

SEQ ID NO: 375 - RNA, 21 nt, synthetic construct
  misc_feature 1..21: note = Synthetic sequence
  source 1..21: mol_type = other RNA
cgctcatgatccaaagcttac

SEQ ID NO: 376 - RNA, 21 nt, synthetic construct
  misc_feature 1..21: note = Synthetic sequence
  source 1..21: mol_type = other RNA
taagctttggatcatgagcgg

SEQ ID NO: 377 - RNA, 21 nt, synthetic construct
  misc_feature 1..21: note = Synthetic sequence
  source 1..21: mol_type = other RNA
gctcatgatccaaagcttacg

SEQ ID NO: 378 - RNA, 21 nt, synthetic construct
  misc_feature 1..21: note = Synthetic sequence
  source 1..21: mol_type = other RNA
tctaagcgactccgtaagctt

SEQ ID NO: 379 - RNA, 21 nt, synthetic construct
  misc_feature 1..21: note = Synthetic sequence
  source 1..21: mol_type = other RNA
gcttacggagtcgcttagatg

SEQ ID NO: 380 - RNA, 21 nt, synthetic construct
  misc_feature 1..21: note = Synthetic sequence
  source 1..21: mol_type = other RNA
atctaagcgactccgtaagct

SEQ ID NO: 381 - RNA, 21 nt, synthetic construct
  misc_feature 1..21: note = Synthetic sequence
  source 1..21: mol_type = other RNA
cttacggagtcgcttagatga

SEQ ID NO: 382 - RNA, 21 nt, synthetic construct
  misc_feature 1..21: note = Synthetic sequence
  source 1..21: mol_type = other RNA
atggtgttctcatctaagcga

SEQ ID NO: 383 - RNA, 21 nt, synthetic construct
  misc_feature 1..21: note = Synthetic sequence
  source 1..21: mol_type = other RNA
gcttagatgagaacaccatcc

SEQ ID NO: 384 - RNA, 21 nt, synthetic construct
  misc_feature 1..21: note = Synthetic sequence
  source 1..21: mol_type = other RNA
tttgtctgaggcatcatggac

SEQ ID NO: 385 - RNA, 21 nt, synthetic construct
  misc_feature 1..21: note = Synthetic sequence
  source 1..21: mol_type = other RNA
ccatgatgcctcagacaaaaa

SEQ ID NO: 386 - RNA, 21 nt, synthetic construct
  misc_feature 1..21: note = Synthetic sequence
  source 1..21: mol_type = other RNA
ttttgtctgaggcatcatgga

SEQ ID NO: 387 - RNA, 21 nt, synthetic construct
  misc_feature 1..21: note = Synthetic sequence
  source 1..21: mol_type = other RNA
catgatgcctcagacaaaaat

SEQ ID NO: 388 - RNA, 21 nt, synthetic construct
  misc_feature 1..21: note = Synthetic sequence
  source 1..21: mol_type = other RNA
tggacatttttgtctgaggca

SEQ ID NO: 389 - RNA, 21 nt, synthetic construct
  misc_feature 1..21: note = Synthetic sequence
  source 1..21: mol_type = other RNA
cctcagacaaaaatgtccaaa

SEQ ID NO: 390 - RNA, 21 nt, synthetic construct
  misc_feature 1..21: note = Synthetic sequence
  source 1..21: mol_type = other RNA
ttggacatttttgtctgaggc

SEQ ID NO: 391 - RNA, 21 nt, synthetic construct
  misc_feature 1..21: note = Synthetic sequence
  source 1..21: mol_type = other RNA
ctcagacaaaaatgtccaaag

SEQ ID NO: 392 - RNA, 21 nt, synthetic construct
  misc_feature 1..21: note = Synthetic sequence
  source 1..21: mol_type = other RNA
aagcctttggacatttttgtc

SEQ ID NO: 393 - RNA, 21 nt, synthetic construct
  misc_feature 1..21: note = Synthetic sequence
  source 1..21: mol_type = other RNA
caaaaatgtccaaaggcttca

SEQ ID NO: 394 - RNA, 21 nt, synthetic construct
  misc_feature 1..21: note = Synthetic sequence
  source 1..21: mol_type = other RNA
ttcccaaaggcggtgaagcct

SEQ ID NO: 395 - RNA, 21 nt, synthetic construct
  misc_feature 1..21: note = Synthetic sequence
  source 1..21: mol_type = other RNA
gcttcaccgcctttgggaaga

SEQ ID NO: 396 - RNA, 21 nt, synthetic construct
  misc_feature 1..21: note = Synthetic sequence
  source 1..21: mol_type = other RNA
ttcttcttcccaaaggcggtg

SEQ ID NO: 397 - RNA, 21 nt, synthetic construct
  misc_feature 1..21: note = Synthetic sequence
  source 1..21: mol_type = other RNA
ccgcctttgggaagaagaagt

SEQ ID NO: 398 - RNA, 21 nt, synthetic construct
  misc_feature 1..21: note = Synthetic sequence
  source 1..21: mol_type = other RNA
acttcttcttcccaaaggcgg

SEQ ID NO: 399 - RNA, 21 nt, synthetic construct
  misc_feature 1..21: note = Synthetic sequence
  source 1..21: mol_type = other RNA
gcctttgggaagaagaagtct

SEQ ID NO: 400 - RNA, 21 nt, synthetic construct
  misc_feature 1..21: note = Synthetic sequence
  source 1..21: mol_type = other RNA
agacttcttcttcccaaaggc

SEQ ID NO: 401 - RNA, 21 nt, synthetic construct
  misc_feature 1..21: note = Synthetic sequence
  source 1..21: mol_type = other RNA
ctttgggaagaagaagtctca

SEQ ID NO: 402 - RNA, 21 nt, synthetic construct
  misc_feature 1..21: note = Synthetic sequence
  source 1..21: mol_type = other RNA
actgaagctttttcaatccct

SEQ ID NO: 403 - RNA, 21 nt, synthetic construct
  misc_feature 1..21: note = Synthetic sequence
  source 1..21: mol_type = other RNA
ggattgaaaaagcttcagtgc

SEQ ID NO: 404 - RNA, 21 nt, synthetic construct
  misc_feature 1..21: note = Synthetic sequence
  source 1..21: mol_type = other RNA
tcagcatcaccagaagcactg

SEQ ID NO: 405 - RNA, 21 nt, synthetic construct
  misc_feature 1..21: note = Synthetic sequence
  source 1..21: mol_type = other RNA
gtgcttctggtgatgctgagg

SEQ ID NO: 406 - RNA, 21 nt, synthetic construct
  misc_feature 1..21: note = Synthetic sequence
  source 1..21: mol_type = other RNA
tctggaacctcagcatcacca

SEQ ID NO: 407 - RNA, 21 nt, synthetic construct
  misc_feature 1..21: note = Synthetic sequence
  source 1..21: mol_type = other RNA
gtgatgctgaggttccagaga

SEQ ID NO: 408 - RNA, 21 nt, synthetic construct
  misc_feature 1..21: note = Synthetic sequence
  source 1..21: mol_type = other RNA
ttgttctctggaacctcagca

SEQ ID NO: 409 - RNA, 21 nt, synthetic construct
  misc_feature 1..21: note = Synthetic sequence
  source 1..21: mol_type = other RNA
ctgaggttccagagaacaagg

SEQ ID NO: 410 - RNA, 21 nt, synthetic construct
  misc_feature 1..21: note = Synthetic sequence
  source 1..21: mol_type = other RNA
aaccttgttctctggaacctc

SEQ ID NO: 411 - RNA, 21 nt, synthetic construct
  misc_feature 1..21: note = Synthetic sequence
  source 1..21: mol_type = other RNA
ggttccagagaacaaggttga

SEQ ID NO: 412 - RNA, 21 nt, synthetic construct
  misc_feature 1..21: note = Synthetic sequence
  source 1..21: mol_type = other RNA
aatcaaccttgttctctggaa

SEQ ID NO: 413 - RNA, 21 nt, synthetic construct
  misc_feature 1..21: note = Synthetic sequence
  source 1..21: mol_type = other RNA
ccagagaacaaggttgatttt

SEQ ID NO: 414 - RNA, 21 nt, synthetic construct
  misc_feature 1..21: note = Synthetic sequence
  source 1..21: mol_type = other RNA
aaatcaaccttgttctctgga

SEQ ID NO: 415 - RNA, 21 nt, synthetic construct
  misc_feature 1..21: note = Synthetic sequence
  source 1..21: mol_type = other RNA
cagagaacaaggttgattttc

SEQ ID NO: 416 - RNA, 21 nt, synthetic construct
  misc_feature 1..21: note = Synthetic sequence
  source 1..21: mol_type = other RNA
tcgaaaatcaaccttgttctc

SEQ ID NO: 417 - RNA, 21 nt, synthetic construct
  misc_feature 1..21: note = Synthetic sequence
  source 1..21: mol_type = other RNA
gaacaaggttgattttcgatg

SEQ ID NO: 418 - RNA, 21 nt, synthetic construct
  misc_feature 1..21: note = Synthetic sequence
  source 1..21: mol_type = other RNA
aagtgcatcgaaaatcaacct

SEQ ID NO: 419 - RNA, 21 nt, synthetic construct
  misc_feature 1..21: note = Synthetic sequence
  source 1..21: mol_type = other RNA
gttgattttcgatgcacttct

SEQ ID NO: 420 - RNA, 21 nt, synthetic construct
  misc_feature 1..21: note = Synthetic sequence
  source 1..21: mol_type = other RNA
agcagatgcccagaagtgcat

SEQ ID NO: 421 - RNA, 21 nt, synthetic construct
  misc_feature 1..21: note = Synthetic sequence
  source 1..21: mol_type = other RNA
gcacttctgggcatctgcttc

SEQ ID NO: 422 - RNA, 21 nt, synthetic construct
  misc_feature 1..21: note = Synthetic sequence
  source 1..21: mol_type = other RNA
aagcagatgcccagaagtgca

SEQ ID NO: 423 - RNA, 21 nt, synthetic construct
  misc_feature 1..21: note = Synthetic sequence
  source 1..21: mol_type = other RNA
cacttctgggcatctgcttct

SEQ ID NO: 424 - RNA, 21 nt, synthetic construct
  misc_feature 1..21: note = Synthetic sequence
  source 1..21: mol_type = other RNA
aatattggcccgagtacactg

SEQ ID NO: 425 - RNA, 21 nt, synthetic construct
  misc_feature 1..21: note = Synthetic sequence
  source 1..21: mol_type = other RNA
gtgtactcgggccaatattga

SEQ ID NO: 426 - RNA, 21 nt, synthetic construct
  misc_feature 1..21: note = Synthetic sequence
  source 1..21: mol_type = other RNA
tggtataatcaatattggccc

SEQ ID NO: 427 - RNA, 21 nt, synthetic construct
  misc_feature 1..21: note = Synthetic sequence
  source 1..21: mol_type = other RNA
gccaatattgattataccaaa

SEQ ID NO: 428 - RNA, 21 nt, synthetic construct
  misc_feature 1..21: note = Synthetic sequence
  source 1..21: mol_type = other RNA
ttggtataatcaatattggcc

SEQ ID NO: 429 - RNA, 21 nt, synthetic construct
  misc_feature 1..21: note = Synthetic sequence
  source 1..21: mol_type = other RNA
ccaatattgattataccaaag

SEQ ID NO: 430 - RNA, 21 nt, synthetic construct
  misc_feature 1..21: note = Synthetic sequence
  source 1..21: mol_type = other RNA
tttggtataatcaatattggc

SEQ ID NO: 431 - RNA, 21 nt, synthetic construct
  misc_feature 1..21: note = Synthetic sequence
  source 1..21: mol_type = other RNA
caatattgattataccaaaga

SEQ ID NO: 432 - RNA, 21 nt, synthetic construct
  misc_feature 1..21: note = Synthetic sequence
  source 1..21: mol_type = other RNA
aatattccaggatctttggta

SEQ ID NO: 433 - RNA, 21 nt, synthetic construct
  misc_feature 1..21: note = Synthetic sequence
  source 1..21: mol_type = other RNA
ccaaagatcctggaatattca

SEQ ID NO: 434 - RNA, 21 nt, synthetic construct
  misc_feature 1..21: note = Synthetic sequence
  source 1..21: mol_type = other RNA
actccatggacagcattcccc

SEQ ID NO: 435 - RNA, 21 nt, synthetic construct
  misc_feature 1..21: note = Synthetic sequence
  source 1..21: mol_type = other RNA
ggaatgctgtccatggagtgg

SEQ ID NO: 436 - RNA, 21 nt, synthetic construct
  misc_feature 1..21: note = Synthetic sequence
  source 1..21: mol_type = other RNA
agacttcacgcattcggagag

SEQ ID NO: 437 - RNA, 21 nt, synthetic construct
  misc_feature 1..21: note = Synthetic sequence
  source 1..21: mol_type = other RNA
ctccgaatgcgtgaagtctct

SEQ ID NO: 438 - RNA, 21 nt, synthetic construct
  misc_feature 1..21: note = Synthetic sequence
  source 1..21: mol_type = other RNA
agagacttcacgcattcggag

SEQ ID NO: 439 - RNA, 21 nt, synthetic construct
  misc_feature 1..21: note = Synthetic sequence
  source 1..21: mol_type = other RNA
ccgaatgcgtgaagtctctga

SEQ ID NO: 440 - RNA, 21 nt, synthetic construct
  misc_feature 1..21: note = Synthetic sequence
  source 1..21: mol_type = other RNA
tcagagacttcacgcattcgg

SEQ ID NO: 441 - RNA, 21 nt, synthetic construct
  misc_feature 1..21: note = Synthetic sequence
  source 1..21: mol_type = other RNA
gaatgcgtgaagtctctgagt

SEQ ID NO: 442 - RNA, 21 nt, synthetic construct
  misc_feature 1..21: note = Synthetic sequence
  source 1..21: mol_type = other RNA
aaactcagagacttcacgcat

SEQ ID NO: 443 - RNA, 21 nt, synthetic construct
  misc_feature 1..21: note = Synthetic sequence
  source 1..21: mol_type = other RNA
gcgtgaagtctctgagtttct

SEQ ID NO: 444 - RNA, 21 nt, synthetic construct
  misc_feature 1..21: note = Synthetic sequence
  source 1..21: mol_type = other RNA
agaaactcagagacttcacgc

SEQ ID NO: 445 - RNA, 21 nt, synthetic construct
  misc_feature 1..21: note = Synthetic sequence
  source 1..21: mol_type = other RNA
gtgaagtctctgagtttctcc

SEQ ID NO: 446 - RNA, 21 nt, synthetic construct
  misc_feature 1..21: note = Synthetic sequence
  source 1..21: mol_type = other RNA
atccaactggaggagaaactc

SEQ ID NO: 447 - RNA, 21 nt, synthetic construct
  misc_feature 1..21: note = Synthetic sequence
  source 1..21: mol_type = other RNA
gtttctcctccagttggatca

SEQ ID NO: 448 - RNA, 21 nt, synthetic construct
  misc_feature 1..21: note = Synthetic sequence
  source 1..21: mol_type = other RNA
tggttgatgatccaactggag

SEQ ID NO: 449 - RNA, 21 nt, synthetic construct
  misc_feature 1..21: note = Synthetic sequence
  source 1..21: mol_type = other RNA
ccagttggatcatcaaccaac

SEQ ID NO: 450 - RNA, 21 nt, synthetic construct
  misc_feature 1..21: note = Synthetic sequence
  source 1..21: mol_type = other RNA
ttggttgatgatccaactgga

SEQ ID NO: 451 - RNA, 21 nt, synthetic construct
  misc_feature 1..21: note = Synthetic sequence
  source 1..21: mol_type = other RNA
cagttggatcatcaaccaacg

SEQ ID NO: 452 - RNA, 21 nt, synthetic construct
  misc_feature 1..21: note = Synthetic sequence
  source 1..21: mol_type = other RNA
aggcaaaggaggaaacagctg

SEQ ID NO: 453 - RNA, 21 nt, synthetic construct
  misc_feature 1..21: note = Synthetic sequence
  source 1..21: mol_type = other RNA
gctgtttcctcctttgccttt

SEQ ID NO: 454 - RNA, 21 nt, synthetic construct
  misc_feature 1..21: note = Synthetic sequence
  source 1..21: mol_type = other RNA
aaggcaaaggaggaaacagct

SEQ ID NO: 455 - RNA, 21 nt, synthetic construct
  misc_feature 1..21: note = Synthetic sequence
  source 1..21: mol_type = other RNA
ctgtttcctcctttgcctttg

SEQ ID NO: 456 - RNA, 21 nt, synthetic construct
  misc_feature 1..21: note = Synthetic sequence
  source 1..21: mol_type = other RNA
ttctcaaaggcaaaggaggaa

SEQ ID NO: 457 - RNA, 21 nt, synthetic construct
  misc_feature 1..21: note = Synthetic sequence
  source 1..21: mol_type = other RNA
cctcctttgcctttgagaagc

SEQ ID NO: 458 - RNA, 21 nt, synthetic construct
  misc_feature 1..21: note = Synthetic sequence
  source 1..21: mol_type = other RNA
tggatgagcttctcaaaggca

SEQ ID NO: 459 - RNA, 21 nt, synthetic construct
  misc_feature 1..21: note = Synthetic sequence
  source 1..21: mol_type = other RNA
cctttgagaagctcatccaat

SEQ ID NO: 460 - RNA, 21 nt, synthetic construct
  misc_feature 1..21: note = Synthetic sequence
  source 1..21: mol_type = other RNA
taaattggatgagcttctcaa

SEQ ID NO: 461 - RNA, 21 nt, synthetic construct
  misc_feature 1..21: note = Synthetic sequence
  source 1..21: mol_type = other RNA
gagaagctcatccaatttaag

SEQ ID NO: 462 - RNA, 21 nt, synthetic construct
  misc_feature 1..21: note = Synthetic sequence
  source 1..21: mol_type = other RNA
agacttaaattggatgagctt

SEQ ID NO: 463 - RNA, 21 nt, synthetic construct
  misc_feature 1..21: note = Synthetic sequence
  source 1..21: mol_type = other RNA
gctcatccaatttaagtctgt

SEQ ID NO: 464 - RNA, 21 nt, synthetic construct
  misc_feature 1..21: note = Synthetic sequence
  source 1..21: mol_type = other RNA
tacagacttaaattggatgag

SEQ ID NO: 465 - RNA, 21 nt, synthetic construct
  misc_feature 1..21: note = Synthetic sequence
  source 1..21: mol_type = other RNA
catccaatttaagtctgtaat

SEQ ID NO: 466 - RNA, 21 nt, synthetic construct
  misc_feature 1..21: note = Synthetic sequence
  source 1..21: mol_type = other RNA
tattacagacttaaattggat

SEQ ID NO: 467 - RNA, 21 nt, synthetic construct
  misc_feature 1..21: note = Synthetic sequence
  source 1..21: mol_type = other RNA
ccaatttaagtctgtaataca

SEQ ID NO: 468 - RNA, 21 nt, synthetic construct
  misc_feature 1..21: note = Synthetic sequence
  source 1..21: mol_type = other RNA
acatcaccggtgaagaagctg

SEQ ID NO: 469 - RNA, 21 nt, synthetic construct
  misc_feature 1..21: note = Synthetic sequence
  source 1..21: mol_type = other RNA
gcttcttcaccggtgatgtaa

SEQ ID NO: 470 - RNA, 21 nt, synthetic construct
  misc_feature 1..21: note = Synthetic sequence
  source 1..21: mol_type = other RNA
tacatcaccggtgaagaagct

SEQ ID NO: 471 - RNA, 21 nt, synthetic construct
  misc_feature 1..21: note = Synthetic sequence
  source 1..21: mol_type = other RNA
cttcttcaccggtgatgtaaa

SEQ ID NO: 472 - RNA, 21 nt, synthetic construct
  misc_feature 1..21: note = Synthetic sequence
  source 1..21: mol_type = other RNA
acaggtagtttacatcaccgg

SEQ ID NO: 473 - RNA, 21 nt, synthetic construct
  misc_feature 1..21: note = Synthetic sequence
  source 1..21: mol_type = other RNA
ggtgatgtaaactacctgttt

SEQ ID NO: 474 - RNA, 21 nt, synthetic construct
  misc_feature 1..21: note = Synthetic sequence
  source 1..21: mol_type = other RNA
aacaggtagtttacatcaccg

SEQ ID NO: 475 - RNA, 21 nt, synthetic construct
  misc_feature 1..21: note = Synthetic sequence
  source 1..21: mol_type = other RNA
gtgatgtaaactacctgtttg

SEQ ID NO: 476 - RNA, 21 nt, synthetic construct
  misc_feature 1..21: note = Synthetic sequence
  source 1..21: mol_type = other RNA
atagcacaccccttcaaacag

SEQ ID NO: 477 - RNA, 21 nt, synthetic construct
  misc_feature 1..21: note = Synthetic sequence
  source 1..21: mol_type = other RNA
gtttgaaggggtgtgctatgg

SEQ ID NO: 478 - RNA, 21 nt, synthetic construct
  misc_feature 1..21: note = Synthetic sequence
  source 1..21: mol_type = other RNA
agtactaggggtccatagcac

SEQ ID NO: 479 - RNA, 21 nt, synthetic construct
  misc_feature 1..21: note = Synthetic sequence
  source 1..21: mol_type = other RNA
gctatggacccctagtactga

SEQ ID NO: 480 - RNA, 21 nt, synthetic construct
  misc_feature 1..21: note = Synthetic sequence
  source 1..21: mol_type = other RNA
aaatgctgcagatgaccagcg

SEQ ID NO: 481 - RNA, 21 nt, synthetic construct
  misc_feature 1..21: note = Synthetic sequence
  source 1..21: mol_type = other RNA
ctggtcatctgcagcatttct

SEQ ID NO: 482 - RNA, 21 nt, synthetic construct
  misc_feature 1..21: note = Synthetic sequence
  source 1..21: mol_type = other RNA
agaaatgctgcagatgaccag

SEQ ID NO: 483 - RNA, 21 nt, synthetic construct
  misc_feature 1..21: note = Synthetic sequence
  source 1..21: mol_type = other RNA
ggtcatctgcagcatttcttc

SEQ ID NO: 484 - RNA, 21 nt, synthetic construct
  misc_feature 1..21: note = Synthetic sequence
  source 1..21: mol_type = other RNA
aagaaatgctgcagatgacca

SEQ ID NO: 485 - RNA, 21 nt, synthetic construct
  misc_feature 1..21: note = Synthetic sequence
  source 1..21: mol_type = other RNA
gtcatctgcagcatttcttcc

SEQ ID NO: 486 - RNA, 21 nt, synthetic construct
  misc_feature 1..21: note = Synthetic sequence
  source 1..21: mol_type = other RNA
aagtaggaagaaatgctgcag

SEQ ID NO: 487 - RNA, 21 nt, synthetic construct
  misc_feature 1..21: note = Synthetic sequence
  source 1..21: mol_type = other RNA
gcagcatttcttcctacttca

SEQ ID NO: 488 - RNA, 21 nt, synthetic construct
  misc_feature 1..21: note = Synthetic sequence
  source 1..21: mol_type = other RNA
atgaagtaggaagaaatgctg

SEQ ID NO: 489 - RNA, 21 nt, synthetic construct
  misc_feature 1..21: note = Synthetic sequence
  source 1..21: mol_type = other RNA
gcatttcttcctacttcatta

SEQ ID NO: 490 - RNA, 21 nt, synthetic construct
  misc_feature 1..21: note = Synthetic sequence
  source 1..21: mol_type = other RNA
aatgaagtaggaagaaatgct

SEQ ID NO: 491 - RNA, 21 nt, synthetic construct
  misc_feature 1..21: note = Synthetic sequence
  source 1..21: mol_type = other RNA
catttcttcctacttcattat

SEQ ID NO: 492 - RNA, 21 nt, synthetic construct
  misc_feature 1..21: note = Synthetic sequence
  source 1..21: mol_type = other RNA
tatccaataatgaagtaggaa

SEQ ID NO: 493 - RNA, 21 nt, synthetic construct
  misc_feature 1..21: note = Synthetic sequence
  source 1..21: mol_type = other RNA
cctacttcattattggataca

SEQ ID NO: 494 - RNA, 21 nt, synthetic construct
  misc_feature 1..21: note = Synthetic sequence
  source 1..21: mol_type = other RNA
agtgtatccaataatgaagta

SEQ ID NO: 495 - RNA, 21 nt, synthetic construct
  misc_feature 1..21: note = Synthetic sequence
  source 1..21: mol_type = other RNA
cttcattattggatacactgc

SEQ ID NO: 496 - RNA, 21 nt, synthetic construct
  misc_feature 1..21: note = Synthetic sequence
  source 1..21: mol_type = other RNA
ataagatggcaataaatgcag

SEQ ID NO: 497 - RNA, 21 nt, synthetic construct
  misc_feature 1..21: note = Synthetic sequence
  source 1..21: mol_type = other RNA
gcatttattgccatcttatgc

SEQ ID NO: 498 - RNA, 21 nt, synthetic construct
  misc_feature 1..21: note = Synthetic sequence
  source 1..21: mol_type = other RNA
aggagatagcataagatggca

SEQ ID NO: 499 - RNA, 21 nt, synthetic construct
  misc_feature 1..21: note = Synthetic sequence
  source 1..21: mol_type = other RNA
ccatcttatgctatctcctgg

SEQ ID NO: 500 - RNA, 21 nt, synthetic construct
  misc_feature 1..21: note = Synthetic sequence
  source 1..21: mol_type = other RNA
tgggaaaaccaggagatagca

SEQ ID NO: 501 - RNA, 21 nt, synthetic construct
  misc_feature 1..21: note = Synthetic sequence
  source 1..21: mol_type = other RNA
ctatctcctggttttcccact

SEQ ID NO: 502 - RNA, 21 nt, synthetic construct
  misc_feature 1..21: note = Synthetic sequence
  source 1..21: mol_type = other RNA
ttcttgtcatgaataccgcca

SEQ ID NO: 503 - RNA, 21 nt, synthetic construct
  misc_feature 1..21: note = Synthetic sequence
  source 1..21: mol_type = other RNA
gcggtattcatgacaagaatg

SEQ ID NO: 504 - RNA, 21 nt, synthetic construct
  misc_feature 1..21: note = Synthetic sequence
  source 1..21: mol_type = other RNA
attcttgtcatgaataccgcc

SEQ ID NO: 505 - RNA, 21 nt, synthetic construct
  misc_feature 1..21: note = Synthetic sequence
  source 1..21: mol_type = other RNA
cggtattcatgacaagaatgg

SEQ ID NO: 506 - RNA, 21 nt, synthetic construct
  misc_feature 1..21: note = Synthetic sequence
  source 1..21: mol_type = other RNA
tgatgctgagccttcacagcc

SEQ ID NO: 507 - RNA, 21 nt, synthetic construct
  misc_feature 1..21: note = Synthetic sequence
  source 1..21: mol_type = other RNA
ctgtgaaggctcagcatcaca

SEQ ID NO: 508 - RNA, 21 nt, synthetic construct
  misc_feature 1..21: note = Synthetic sequence
  source 1..21: mol_type = other RNA
tcagatgtgtgatgctgagcc

SEQ ID NO: 509 - RNA, 21 nt, synthetic construct
  misc_feature 1..21: note = Synthetic sequence
  source 1..21: mol_type = other RNA
ctcagcatcacacatctgagg

SEQ ID NO: 510 - RNA, 21 nt, synthetic construct
  misc_feature 1..21: note = Synthetic sequence
  source 1..21: mol_type = other RNA
atgcaagtgagaacttcactg

SEQ ID NO: 511 - RNA, 21 nt, synthetic construct
  misc_feature 1..21: note = Synthetic sequence
  source 1..21: mol_type = other RNA
gtgaagttctcacttgcatta

SEQ ID NO: 512 - RNA, 21 nt, synthetic construct
  misc_feature 1..21: note = Synthetic sequence
  source 1..21: mol_type = other RNA
taatgcaagtgagaacttcac

SEQ ID NO: 513 - RNA, 21 nt, synthetic construct
  misc_feature 1..21: note = Synthetic sequence
  source 1..21: mol_type = other RNA
gaagttctcacttgcattaag

SEQ ID NO: 514 - RNA, 21 nt, synthetic construct
  misc_feature 1..21: note = Synthetic sequence
  source 1..21: mol_type = other RNA
tcagcttaatgcaagtgagaa

SEQ ID NO: 515 - RNA, 21 nt, synthetic construct
  misc_feature 1..21: note = Synthetic sequence
  source 1..21: mol_type = other RNA
ctcacttgcattaagctgatt

SEQ ID NO: 516 - RNA, 21 nt, synthetic construct
  misc_feature 1..21: note = Synthetic sequence
  source 1..21: mol_type = other RNA
aatcagcttaatgcaagtgag

SEQ ID NO: 517 - RNA, 21 nt, synthetic construct
  misc_feature 1..21: note = Synthetic sequence
  source 1..21: mol_type = other RNA
cacttgcattaagctgattaa

SEQ ID NO: 518 - RNA, 21 nt, synthetic construct
  misc_feature 1..21: note = Synthetic sequence
  source 1..21: mol_type = other RNA
ttaatcagcttaatgcaagtg

SEQ ID NO: 519 - RNA, 21 nt, synthetic construct
  misc_feature 1..21: note = Synthetic sequence
  source 1..21: mol_type = other RNA
cttgcattaagctgattaaaa

SEQ ID NO: 520 - RNA, 21 nt, synthetic construct
  misc_feature 1..21: note = Synthetic sequence
  source 1..21: mol_type = other RNA
attttaatcagcttaatgcaa

SEQ ID NO: 521 - RNA, 21 nt, synthetic construct
  misc_feature 1..21: note = Synthetic sequence
  source 1..21: mol_type = other RNA
gcattaagctgattaaaatgt

SEQ ID NO: 522 - RNA, 21 nt, synthetic construct
  misc_feature 1..21: note = Synthetic sequence
  source 1..21: mol_type = other RNA
tgtgtacattttaatcagctt

SEQ ID NO: 523 - RNA, 21 nt, synthetic construct
  misc_feature 1..21: note = Synthetic sequence
  source 1..21: mol_type = other RNA
gctgattaaaatgtacacatg

SEQ ID NO: 524 - RNA, 21 nt, synthetic construct
  misc_feature 1..21: note = Synthetic sequence
  source 1..21: mol_type = other RNA
atgtgtacattttaatcagct

SEQ ID NO: 525 - RNA, 21 nt, synthetic construct
  misc_feature 1..21: note = Synthetic sequence
  source 1..21: mol_type = other RNA
ctgattaaaatgtacacatgg

SEQ ID NO: 526 - RNA, 21 nt, synthetic construct
  misc_feature 1..21: note = Synthetic sequence
  source 1..21: mol_type = other RNA
tggtttctcccatgtgtacat

SEQ ID NO: 527 - RNA, 21 nt, synthetic construct
  misc_feature 1..21: note = Synthetic sequence
  source 1..21: mol_type = other RNA
gtacacatgggagaaaccatt

SEQ ID NO: 528 - RNA, 21 nt, synthetic construct
  misc_feature 1..21: note = Synthetic sequence
  source 1..21: mol_type = other RNA
tctgcaaatggtttctcccat

SEQ ID NO: 529 - RNA, 21 nt, synthetic construct
  misc_feature 1..21: note = Synthetic sequence
  source 1..21: mol_type = other RNA
gggagaaaccatttgcagaaa

SEQ ID NO: 530 - RNA, 21 nt, synthetic construct
  misc_feature 1..21: note = Synthetic sequence
  source 1..21: mol_type = other RNA
ttctgcaaatggtttctccca

SEQ ID NO: 531 - RNA, 21 nt, synthetic construct
  misc_feature 1..21: note = Synthetic sequence
  source 1..21: mol_type = other RNA
ggagaaaccatttgcagaaat

SEQ ID NO: 532 - RNA, 21 nt, synthetic construct
  misc_feature 1..21: note = Synthetic sequence
  source 1..21: mol_type = other RNA
tttctgcaaatggtttctccc

SEQ ID NO: 533 - RNA, 21 nt, synthetic construct
  misc_feature 1..21: note = Synthetic sequence
  source 1..21: mol_type = other RNA
gagaaaccatttgcagaaatc

SEQ ID NO: 534 - RNA, 21 nt, synthetic construct
  misc_feature 1..21: note = Synthetic sequence
  source 1..21: mol_type = other RNA
tcaatgatttctgcaaatggt

SEQ ID NO: 535 - RNA, 21 nt, synthetic construct
  misc_feature 1..21: note = Synthetic sequence
  source 1..21: mol_type = other RNA
catttgcagaaatcattgaag

SEQ ID NO: 536 - RNA, 21 nt, synthetic construct
  misc_feature 1..21: note = Synthetic sequence
  source 1..21: mol_type = other RNA
ttaggtcttcaatgatttctg

SEQ ID NO: 537 - RNA, 21 nt, synthetic construct
  misc_feature 1..21: note = Synthetic sequence
  source 1..21: mol_type = other RNA
gaaatcattgaagacctaaga

SEQ ID NO: 538 - RNA, 21 nt, synthetic construct
  misc_feature 1..21: note = Synthetic sequence
  source 1..21: mol_type = other RNA
tttccttccttcttaggtctt

SEQ ID NO: 539 - RNA, 21 nt, synthetic construct
  misc_feature 1..21: note = Synthetic sequence
  source 1..21: mol_type = other RNA
gacctaagaaggaaggaaagg

SEQ ID NO: 540 - RNA, 21 nt, synthetic construct
  misc_feature 1..21: note = Synthetic sequence
  source 1..21: mol_type = other RNA
tcctttccttccttcttaggt

SEQ ID NO: 541 - RNA, 21 nt, synthetic construct
  misc_feature 1..21: note = Synthetic sequence
  source 1..21: mol_type = other RNA
ctaagaaggaaggaaaggaaa

SEQ ID NO: 542 - RNA, 21 nt, synthetic construct
  misc_feature 1..21: note = Synthetic sequence
  source 1..21: mol_type = other RNA
agtttcctttccttccttctt

SEQ ID NO: 543 - RNA, 21 nt, synthetic construct
  misc_feature 1..21: note = Synthetic sequence
  source 1..21: mol_type = other RNA
gaaggaaggaaaggaaactat

SEQ ID NO: 544 - RNA, 21 nt, synthetic construct
  misc_feature 1..21: note = Synthetic sequence
  source 1..21: mol_type = other RNA
aatagtttcctttccttcctt

SEQ ID NO: 545 - RNA, 21 nt, synthetic construct
  misc_feature 1..21: note = Synthetic sequence
  source 1..21: mol_type = other RNA
ggaaggaaaggaaactattgg

SEQ ID NO: 546 - RNA, 21 nt, synthetic construct
  misc_feature 1..21: note = Synthetic sequence
  source 1..21: mol_type = other RNA
tctccaatagtttcctttcct

SEQ ID NO: 547 - RNA, 21 nt, synthetic construct
  misc_feature 1..21: note = Synthetic sequence
  source 1..21: mol_type = other RNA
gaaaggaaactattggagaag

SEQ ID NO: 548 - RNA, 21 nt, synthetic construct
  misc_feature 1..21: note = Synthetic sequence
  source 1..21: mol_type = other RNA
acaaggttatacttgtcaggc

SEQ ID NO: 549 - RNA, 21 nt, synthetic construct
  misc_feature 1..21: note = Synthetic sequence
  source 1..21: mol_type = other RNA
ctgacaagtataaccttgttc

SEQ ID NO: 550 - RNA, 21 nt, synthetic construct
  misc_feature 1..21: note = Synthetic sequence
  source 1..21: mol_type = other RNA
atgaacaaggttatacttgtc

SEQ ID NO: 551 - RNA, 21 nt, synthetic construct
  misc_feature 1..21: note = Synthetic sequence
  source 1..21: mol_type = other RNA
caagtataaccttgttcatca

SEQ ID NO: 552 - RNA, 21 nt, synthetic construct
  misc_feature 1..21: note = Synthetic sequence
  source 1..21: mol_type = other RNA
atgatgaacaaggttatactt

SEQ ID NO: 553 - RNA, 21 nt, synthetic construct
  misc_feature 1..21: note = Synthetic sequence
  source 1..21: mol_type = other RNA
gtataaccttgttcatcatcc

SEQ ID NO: 554 - RNA, 21 nt, synthetic construct
  misc_feature 1..21: note = Synthetic sequence
  source 1..21: mol_type = other RNA
aggatgtgtggatgagaaccc

SEQ ID NO: 555 - RNA, 21 nt, synthetic construct
  misc_feature 1..21: note = Synthetic sequence
  source 1..21: mol_type = other RNA
gttctcatccacacatcctta

SEQ ID NO: 556 - RNA, 21 nt, synthetic construct
  misc_feature 1..21: note = Synthetic sequence
  source 1..21: mol_type = other RNA
tcagctttaaggatgtgtgga

SEQ ID NO: 557 - RNA, 21 nt, synthetic construct
  misc_feature 1..21: note = Synthetic sequence
  source 1..21: mol_type = other RNA
cacacatccttaaagctgaaa

SEQ ID NO: 558 - RNA, 21 nt, synthetic construct
  misc_feature 1..21: note = Synthetic sequence
  source 1..21: mol_type = other RNA
tttcagctttaaggatgtgtg

SEQ ID NO: 559 - RNA, 21 nt, synthetic construct
  misc_feature 1..21: note = Synthetic sequence
  source 1..21: mol_type = other RNA
cacatccttaaagctgaaact

SEQ ID NO: 560 - RNA, 21 nt, synthetic construct
  misc_feature 1..21: note = Synthetic sequence
  source 1..21: mol_type = other RNA
agtttcagctttaaggatgtg

SEQ ID NO: 561 - RNA, 21 nt, synthetic construct
  misc_feature 1..21: note = Synthetic sequence
  source 1..21: mol_type = other RNA
catccttaaagctgaaactca

SEQ ID NO: 562 - RNA, 21 nt, synthetic construct
  misc_feature 1..21: note = Synthetic sequence
  source 1..21: mol_type = other RNA
tgtgagtttcagctttaagga

SEQ ID NO: 563 - RNA, 21 nt, synthetic construct
  misc_feature 1..21: note = Synthetic sequence
  source 1..21: mol_type = other RNA
cttaaagctgaaactcacagc

SEQ ID NO: 564 - RNA, 21 nt, synthetic construct
  misc_feature 1..21: note = Synthetic sequence
  source 1..21: mol_type = other RNA
tgctgaaggccattgacgctg

SEQ ID NO: 565 - RNA, 21 nt, synthetic construct
  misc_feature 1..21: note = Synthetic sequence
  source 1..21: mol_type = other RNA
gcgtcaatggccttcagcatg

SEQ ID NO: 566 - RNA, 21 nt, synthetic construct
  misc_feature 1..21: note = Synthetic sequence
  source 1..21: mol_type = other RNA
atgctgaaggccattgacgct

SEQ ID NO: 567 - RNA, 21 nt, synthetic construct
  misc_feature 1..21: note = Synthetic sequence
  source 1..21: mol_type = other RNA
cgtcaatggccttcagcatgc

SEQ ID NO: 568 - RNA, 21 nt, synthetic construct
  misc_feature 1..21: note = Synthetic sequence
  source 1..21: mol_type = other RNA
ttcaaggaggccagcatgctg

SEQ ID NO: 569 - RNA, 21 nt, synthetic construct
  misc_feature 1..21: note = Synthetic sequence
  source 1..21: mol_type = other RNA
gcatgctggcctccttgaatc

SEQ ID NO: 570 - RNA, 21 nt, synthetic construct
  misc_feature 1..21: note = Synthetic sequence
  source 1..21: mol_type = other RNA
tgcaataggcacaaagaacac

SEQ ID NO: 571 - RNA, 21 nt, synthetic construct
  misc_feature 1..21: note = Synthetic sequence
  source 1..21: mol_type = other RNA
gttctttgtgcctattgcagt

SEQ ID NO: 572 - RNA, 21 nt, synthetic construct
  misc_feature 1..21: note = Synthetic sequence
  source 1..21: mol_type = other RNA
acctttgactgcaataggcac

SEQ ID NO: 573 - RNA, 21 nt, synthetic construct
  misc_feature 1..21: note = Synthetic sequence
  source 1..21: mol_type = other RNA
gcctattgcagtcaaaggtct

SEQ ID NO: 574 - RNA, 21 nt, synthetic construct
  misc_feature 1..21: note = Synthetic sequence
  source 1..21: mol_type = other RNA
agacctttgactgcaataggc

SEQ ID NO: 575 - RNA, 21 nt, synthetic construct
  misc_feature 1..21: note = Synthetic sequence
  source 1..21: mol_type = other RNA
ctattgcagtcaaaggtctca

SEQ ID NO: 576 - RNA, 21 nt, synthetic construct
  misc_feature 1..21: note = Synthetic sequence
  source 1..21: mol_type = other RNA
aattcgtgagacctttgactg

SEQ ID NO: 577 - RNA, 21 nt, synthetic construct
  misc_feature 1..21: note = Synthetic sequence
  source 1..21: mol_type = other RNA
gtcaaaggtctcacgaattcc

SEQ ID NO: 578 - RNA, 21 nt, synthetic construct
  misc_feature 1..21: note = Synthetic sequence
  source 1..21: mol_type = other RNA
tcttgaacctcatcactgcag

SEQ ID NO: 579 - RNA, 21 nt, synthetic construct
  misc_feature 1..21: note = Synthetic sequence
  source 1..21: mol_type = other RNA
gcagtgatgaggttcaagaag

SEQ ID NO: 580 - RNA, 21 nt, synthetic construct
  misc_feature 1..21: note = Synthetic sequence
  source 1..21: mol_type = other RNA
ttcttgaacctcatcactgca

SEQ ID NO: 581 - RNA, 21 nt, synthetic construct
  misc_feature 1..21: note = Synthetic sequence
  source 1..21: mol_type = other RNA
cagtgatgaggttcaagaagt

SEQ ID NO: 582 - RNA, 21 nt, synthetic construct
  misc_feature 1..21: note = Synthetic sequence
  source 1..21: mol_type = other RNA
acttcttgaacctcatcactg

SEQ ID NO: 583 - RNA, 21 nt, synthetic construct
  misc_feature 1..21: note = Synthetic sequence
  source 1..21: mol_type = other RNA
gtgatgaggttcaagaagttt

SEQ ID NO: 584 - RNA, 21 nt, synthetic construct
  misc_feature 1..21: note = Synthetic sequence
  source 1..21: mol_type = other RNA
aaacttcttgaacctcatcac

SEQ ID NO: 585 - RNA, 21 nt, synthetic construct
  misc_feature 1..21: note = Synthetic sequence
  source 1..21: mol_type = other RNA
gatgaggttcaagaagttttt

SEQ ID NO: 586 - RNA, 22 nt, synthetic construct
  misc_feature 1..22: note = Synthetic sequence
  source 1..22: mol_type = other RNA
aggaaaaacttcttcaaccctc

SEQ ID NO: 587 - RNA, 21 nt, synthetic construct
  misc_feature 1..21: note = Synthetic sequence
  source 1..21: mol_type = other RNA
ggttcaagaagtttttcctcc

SEQ ID NO: 588 - RNA, 21 nt, synthetic construct
  misc_feature 1..21: note = Synthetic sequence
  source 1..21: mol_type = other RNA
ttgtaatgtctggacatagaa

SEQ ID NO: 589 - RNA, 21 nt, synthetic construct
  misc_feature 1..21: note = Synthetic sequence
  source 1..21: mol_type = other RNA
ctatgtccagacattacaaga

SEQ ID NO: 590 - RNA, 21 nt, synthetic construct
  misc_feature 1..21: note = Synthetic sequence
  source 1..21: mol_type = other RNA
aaagaccagagctttgctggg

SEQ ID NO: 591 - RNA, 21 nt, synthetic construct
  misc_feature 1..21: note = Synthetic sequence
  source 1..21: mol_type = other RNA
cagcaaagctctggtctttga

SEQ ID NO: 592 - RNA, 21 nt, synthetic construct
  misc_feature 1..21: note = Synthetic sequence
  source 1..21: mol_type = other RNA
tcaaagaccagagctttgctg

SEQ ID NO: 593 - RNA, 21 nt, synthetic construct
  misc_feature 1..21: note = Synthetic sequence
  source 1..21: mol_type = other RNA
gcaaagctctggtctttgagg

SEQ ID NO: 594 - RNA, 21 nt, synthetic construct
  misc_feature 1..21: note = Synthetic sequence
  source 1..21: mol_type = other RNA
agaagcatgcccgttcctctc

SEQ ID NO: 595 - RNA, 21 nt, synthetic construct
  misc_feature 1..21: note = Synthetic sequence
  source 1..21: mol_type = other RNA
gaggaacgggcatgcttctga

SEQ ID NO: 596 - RNA, 21 nt, synthetic construct
  misc_feature 1..21: note = Synthetic sequence
  source 1..21: mol_type = other RNA
atctctaggcctggtcatccc

SEQ ID NO: 597 - RNA, 21 nt, synthetic construct
  misc_feature 1..21: note = Synthetic sequence
  source 1..21: mol_type = other RNA
gatgaccaggcctagagatgc

SEQ ID NO: 598 - RNA, 21 nt, synthetic construct
  misc_feature 1..21: note = Synthetic sequence
  source 1..21: mol_type = other RNA
tgatcttgtgcaactctgggc

SEQ ID NO: 599 - RNA, 21 nt, synthetic construct
  misc_feature 1..21: note = Synthetic sequence
  source 1..21: mol_type = other RNA
ccagagttgcacaagatcaac

SEQ ID NO: 600 - RNA, 21 nt, synthetic construct
  misc_feature 1..21: note = Synthetic sequence
  source 1..21: mol_type = other RNA
ttgatcttgtgcaactctggg

SEQ ID NO: 601 - RNA, 21 nt, synthetic construct
  misc_feature 1..21: note = Synthetic sequence
  source 1..21: mol_type = other RNA
cagagttgcacaagatcaacc

SEQ ID NO: 602 - RNA, 21 nt, synthetic construct
  misc_feature 1..21: note = Synthetic sequence
  source 1..21: mol_type = other RNA
acatcatccccttggacacca

SEQ ID NO: 603 - RNA, 21 nt, synthetic construct
  misc_feature 1..21: note = Synthetic sequence
  source 1..21: mol_type = other RNA
gtgtccaaggggatgatgtta

SEQ ID NO: 604 - RNA, 21 nt, synthetic construct
  misc_feature 1..21: note = Synthetic sequence
  source 1..21: mol_type = other RNA
ttaccactccccgtgttgccg

SEQ ID NO: 605 - RNA, 21 nt, synthetic construct
  misc_feature 1..21: note = Synthetic sequence
  source 1..21: mol_type = other RNA
gcaacacggggagtggtaaga

SEQ ID NO: 606 - RNA, 21 nt, synthetic construct
  misc_feature 1..21: note = Synthetic sequence
  source 1..21: mol_type = other RNA
acaacaggctgctcttaccac

SEQ ID NO: 607 - RNA, 21 nt, synthetic construct
  misc_feature 1..21: note = Synthetic sequence
  source 1..21: mol_type = other RNA
ggtaagagcagcctgttgtca

SEQ ID NO: 608 - RNA, 21 nt, synthetic construct
  misc_feature 1..21: note = Synthetic sequence
  source 1..21: mol_type = other RNA
atgttctccctgatgttcccg

SEQ ID NO: 609 - RNA, 21 nt, synthetic construct
  misc_feature 1..21: note = Synthetic sequence
  source 1..21: mol_type = other RNA
ggaacatcagggagaacatcc

SEQ ID NO: 610 - RNA, 21 nt, synthetic construct
  misc_feature 1..21: note = Synthetic sequence
  source 1..21: mol_type = other RNA
tccaaagggcagaagttccag

SEQ ID NO: 611 - RNA, 21 nt, synthetic construct
  misc_feature 1..21: note = Synthetic sequence
  source 1..21: mol_type = other RNA
ggaacttctgccctttggaga

SEQ ID NO: 612 - RNA, 21 nt, synthetic construct
  misc_feature 1..21: note = Synthetic sequence
  source 1..21: mol_type = other RNA
tgtcatgtctccaaagggcag

SEQ ID NO: 613 - RNA, 21 nt, synthetic construct
  misc_feature 1..21: note = Synthetic sequence
  source 1..21: mol_type = other RNA
gccctttggagacatgacaga

SEQ ID NO: 614 - RNA, 21 nt, synthetic construct
  misc_feature 1..21: note = Synthetic sequence
  source 1..21: mol_type = other RNA
tctgtcatgtctccaaagggc

SEQ ID NO: 615 - RNA, 21 nt, synthetic construct
  misc_feature 1..21: note = Synthetic sequence
  source 1..21: mol_type = other RNA
cctttggagacatgacagaga

SEQ ID NO: 616 - RNA, 21 nt, synthetic construct
  misc_feature 1..21: note = Synthetic sequence
  source 1..21: mol_type = other RNA
aatgcactcctcaaaaatgtg

SEQ ID NO: 617 - RNA, 21 nt, synthetic construct
  misc_feature 1..21: note = Synthetic sequence
  source 1..21: mol_type = other RNA
catttttgaggagtgcattaa

SEQ ID NO: 618 - RNA, 21 nt, synthetic construct
  misc_feature 1..21: note = Synthetic sequence
  source 1..21: mol_type = other RNA
aaaattctaagtactgcagct

SEQ ID NO: 619 - RNA, 21 nt, synthetic construct
  misc_feature 1..21: note = Synthetic sequence
  source 1..21: mol_type = other RNA
ctgcagtacttagaattttgt

SEQ ID NO: 620 - RNA, 21 nt, synthetic construct
  misc_feature 1..21: note = Synthetic sequence
  source 1..21: mol_type = other RNA
acaaaattctaagtactgcag

SEQ ID NO: 621 - RNA, 21 nt, synthetic construct
  misc_feature 1..21: note = Synthetic sequence
  source 1..21: mol_type = other RNA
gcagtacttagaattttgtgg

SEQ ID NO: 622 - RNA, 21 nt, synthetic construct
  misc_feature 1..21: note = Synthetic sequence
  source 1..21: mol_type = other RNA
tgatctggccacaaaattcta

SEQ ID NO: 623 - RNA, 21 nt, synthetic construct
  misc_feature 1..21: note = Synthetic sequence
  source 1..21: mol_type = other RNA
gaattttgtggccagatcatt

SEQ ID NO: 624 - RNA, 21 nt, synthetic construct
  misc_feature 1..21: note = Synthetic sequence
  source 1..21: mol_type = other RNA
tccaacaaaatgatctggcca

SEQ ID NO: 625 - RNA, 21 nt, synthetic construct
  misc_feature 1..21: note = Synthetic sequence
  source 1..21: mol_type = other RNA
gccagatcattttgttggaaa

SEQ ID NO: 626 - RNA, 21 nt, synthetic construct
  misc_feature 1..21: note = Synthetic sequence
  source 1..21: mol_type = other RNA
ttccaacaaaatgatctggcc

SEQ ID NO: 627 - RNA, 21 nt, synthetic construct
  misc_feature 1..21: note = Synthetic sequence
  source 1..21: mol_type = other RNA
ccagatcattttgttggaaaa

SEQ ID NO: 628 - RNA, 21 nt, synthetic construct
  misc_feature 1..21: note = Synthetic sequence
  source 1..21: mol_type = other RNA
tttccaacaaaatgatctggc

SEQ ID NO: 629 - RNA, 21 nt, synthetic construct
  misc_feature 1..21: note = Synthetic sequence
  source 1..21: mol_type = other RNA
cagatcattttgttggaaaat

SEQ ID NO: 630 - RNA, 21 nt, synthetic construct
  misc_feature 1..21: note = Synthetic sequence
  source 1..21: mol_type = other RNA
attttccaacaaaatgatctg

SEQ ID NO: 631 - RNA, 21 nt, synthetic construct
  misc_feature 1..21: note = Synthetic sequence
  source 1..21: mol_type = other RNA
gatcattttgttggaaaatgg

SEQ ID NO: 632 - RNA, 21 nt, synthetic construct
  misc_feature 1..21: note = Synthetic sequence
  source 1..21: mol_type = other RNA
acagattttcccattttccaa

SEQ ID NO: 633 - RNA, 21 nt, synthetic construct
  misc_feature 1..21: note = Synthetic sequence
  source 1..21: mol_type = other RNA
ggaaaatgggaaaatctgtga

SEQ ID NO: 634 - RNA, 21 nt, synthetic construct
  misc_feature 1..21: note = Synthetic sequence
  source 1..21: mol_type = other RNA
tccattttcacagattttccc

SEQ ID NO: 635 - RNA, 21 nt, synthetic construct
  misc_feature 1..21: note = Synthetic sequence
  source 1..21: mol_type = other RNA
gaaaatctgtgaaaatggaac

SEQ ID NO: 636 - RNA, 21 nt, synthetic construct
  misc_feature 1..21: note = Synthetic sequence
  source 1..21: mol_type = other RNA
ttaactcactgtgagttccat

SEQ ID NO: 637 - RNA, 21 nt, synthetic construct
  misc_feature 1..21: note = Synthetic sequence
  source 1..21: mol_type = other RNA
ggaactcacagtgagttaatg

SEQ ID NO: 638 - RNA, 21 nt, synthetic construct
  misc_feature 1..21: note = Synthetic sequence
  source 1..21: mol_type = other RNA
attaactcactgtgagttcca

SEQ ID NO: 639 - RNA, 21 nt, synthetic construct
  misc_feature 1..21: note = Synthetic sequence
  source 1..21: mol_type = other RNA
gaactcacagtgagttaatgc

SEQ ID NO: 640 - RNA, 21 nt, synthetic construct
  misc_feature 1..21: note = Synthetic sequence
  source 1..21: mol_type = other RNA
tgcattaactcactgtgagtt

SEQ ID NO: 641 - RNA, 21 nt, synthetic construct
  misc_feature 1..21: note = Synthetic sequence
  source 1..21: mol_type = other RNA
ctcacagtgagttaatgcaga

SEQ ID NO: 642 - RNA, 21 nt, synthetic construct
  misc_feature 1..21: note = Synthetic sequence
  source 1..21: mol_type = other RNA
tctgcattaactcactgtgag

SEQ ID NO: 643 - RNA, 21 nt, synthetic construct
  misc_feature 1..21: note = Synthetic sequence
  source 1..21: mol_type = other RNA
cacagtgagttaatgcagaaa

SEQ ID NO: 644 - RNA, 21 nt, synthetic construct
  misc_feature 1..21: note = Synthetic sequence
  source 1..21: mol_type = other RNA
tttctgcattaactcactgtg

SEQ ID NO: 645 - RNA, 21 nt, synthetic construct
  misc_feature 1..21: note = Synthetic sequence
  source 1..21: mol_type = other RNA
cagtgagttaatgcagaaaaa

SEQ ID NO: 646 - RNA, 21 nt, synthetic construct
  misc_feature 1..21: note = Synthetic sequence
  source 1..21: mol_type = other RNA
tttttctgcattaactcactg

SEQ ID NO: 647 - RNA, 21 nt, synthetic construct
  misc_feature 1..21: note = Synthetic sequence
  source 1..21: mol_type = other RNA
gtgagttaatgcagaaaaagg

SEQ ID NO: 648 - RNA, 21 nt, synthetic construct
  misc_feature 1..21: note = Synthetic sequence
  source 1..21: mol_type = other RNA
atatttcccctttttctgcat

SEQ ID NO: 649 - RNA, 21 nt, synthetic construct
  misc_feature 1..21: note = Synthetic sequence
  source 1..21: mol_type = other RNA
gcagaaaaaggggaaatatgc

SEQ ID NO: 650 - RNA, 21 nt, synthetic construct
  misc_feature 1..21: note = Synthetic sequence
  source 1..21: mol_type = other RNA
taagttgggcatatttcccct

SEQ ID NO: 651 - RNA, 21 nt, synthetic construct
  misc_feature 1..21: note = Synthetic sequence
  source 1..21: mol_type = other RNA
gggaaatatgcccaacttatc

SEQ ID NO: 652 - RNA, 21 nt, synthetic construct
  misc_feature 1..21: note = Synthetic sequence
  source 1..21: mol_type = other RNA
ataagttgggcatatttcccc

SEQ ID NO: 653 - RNA, 21 nt, synthetic construct
  misc_feature 1..21: note = Synthetic sequence
  source 1..21: mol_type = other RNA
ggaaatatgcccaacttatcc

SEQ ID NO: 654 - RNA, 21 nt, synthetic construct
  misc_feature 1..21: note = Synthetic sequence
  source 1..21: mol_type = other RNA
atcttctggataagttgggca

SEQ ID NO: 655 - RNA, 21 nt, synthetic construct
  misc_feature 1..21: note = Synthetic sequence
  source 1..21: mol_type = other RNA
cccaacttatccagaagatgc

SEQ ID NO: 656 - RNA, 21 nt, synthetic construct
  misc_feature 1..21: note = Synthetic sequence
  source 1..21: mol_type = other RNA
ttccttgtgcatcttctggat

SEQ ID NO: 657 - RNA, 21 nt, synthetic construct
  misc_feature 1..21: note = Synthetic sequence
  source 1..21: mol_type = other RNA
ccagaagatgcacaaggaagc

SEQ ID NO: 658 - RNA, 21 nt, synthetic construct
  misc_feature 1..21: note = Synthetic sequence
  source 1..21: mol_type = other RNA
tgctatctttgctgtgtcctg

SEQ ID NO: 659 - RNA, 21 nt, synthetic construct
  misc_feature 1..21: note = Synthetic sequence
  source 1..21: mol_type = other RNA
ggacacagcaaagatagcaga

SEQ ID NO: 660 - RNA, 21 nt, synthetic construct
  misc_feature 1..21: note = Synthetic sequence
  source 1..21: mol_type = other RNA
tctacctttggcttctctgct

SEQ ID NO: 661 - RNA, 21 nt, synthetic construct
  misc_feature 1..21: note = Synthetic sequence
  source 1..21: mol_type = other RNA
cagagaagccaaaggtagaaa

SEQ ID NO: 662 - RNA, 21 nt, synthetic construct
  misc_feature 1..21: note = Synthetic sequence
  source 1..21: mol_type = other RNA
actttctacctttggcttctc

SEQ ID NO: 663 - RNA, 21 nt, synthetic construct
  misc_feature 1..21: note = Synthetic sequence
  source 1..21: mol_type = other RNA
gaagccaaaggtagaaagtca

SEQ ID NO: 664 - RNA, 21 nt, synthetic construct
  misc_feature 1..21: note = Synthetic sequence
  source 1..21: mol_type = other RNA
agcatttccgttgagagactc

SEQ ID NO: 665 - RNA, 21 nt, synthetic construct
  misc_feature 1..21: note = Synthetic sequence
  source 1..21: mol_type = other RNA
gtctctcaacggaaatgctgt

SEQ ID NO: 666 - RNA, 21 nt, synthetic construct
  misc_feature 1..21: note = Synthetic sequence
  source 1..21: mol_type = other RNA
acagcatttccgttgagagac

SEQ ID NO: 667 - RNA, 21 nt, synthetic construct
  misc_feature 1..21: note = Synthetic sequence
  source 1..21: mol_type = other RNA
ctctcaacggaaatgctgtgc

SEQ ID NO: 668 - RNA, 21 nt, synthetic construct
  misc_feature 1..21: note = Synthetic sequence
  source 1..21: mol_type = other RNA
actcaaggagccttcttccat

SEQ ID NO: 669 - RNA, 21 nt, synthetic construct
  misc_feature 1..21: note = Synthetic sequence
  source 1..21: mol_type = other RNA
ggaagaaggctccttgagttg

SEQ ID NO: 670 - RNA, 21 nt, synthetic construct
  misc_feature 1..21: note = Synthetic sequence
  source 1..21: mol_type = other RNA
aactcaaggagccttcttcca

SEQ ID NO: 671 - RNA, 21 nt, synthetic construct
  misc_feature 1..21: note = Synthetic sequence
  source 1..21: mol_type = other RNA
gaagaaggctccttgagttgg

SEQ ID NO: 672 - RNA, 21 nt, synthetic construct
  misc_feature 1..21: note = Synthetic sequence
  source 1..21: mol_type = other RNA
tgcaagagaccatgtaacctc

SEQ ID NO: 673 - RNA, 21 nt, synthetic construct
  misc_feature 1..21: note = Synthetic sequence
  source 1..21: mol_type = other RNA
ggttacatggtctcttgcata

SEQ ID NO: 674 - RNA, 21 nt, synthetic construct
  misc_feature 1..21: note = Synthetic sequence
  source 1..21: mol_type = other RNA
atgcaagagaccatgtaacct

SEQ ID NO: 675 - RNA, 21 nt, synthetic construct
  misc_feature 1..21: note = Synthetic sequence
  source 1..21: mol_type = other RNA
gttacatggtctcttgcataa

SEQ ID NO: 676 - RNA, 21 nt, synthetic construct
  misc_feature 1..21: note = Synthetic sequence
  source 1..21: mol_type = other RNA
aattatgcaagagaccatgta

SEQ ID NO: 677 - RNA, 21 nt, synthetic construct
  misc_feature 1..21: note = Synthetic sequence
  source 1..21: mol_type = other RNA
catggtctcttgcataatttt

SEQ ID NO: 678 - RNA, 21 nt, synthetic construct
  misc_feature 1..21: note = Synthetic sequence
  source 1..21: mol_type = other RNA
agaaaattatgcaagagacca

SEQ ID NO: 679 - RNA, 21 nt, synthetic construct
  misc_feature 1..21: note = Synthetic sequence
  source 1..21: mol_type = other RNA
gtctcttgcataattttcttc

SEQ ID NO: 680 - RNA, 21 nt, synthetic construct
  misc_feature 1..21: note = Synthetic sequence
  source 1..21: mol_type = other RNA
aagaagaaaattatgcaagag

SEQ ID NO: 681 - RNA, 21 nt, synthetic construct
  misc_feature 1..21: note = Synthetic sequence
  source 1..21: mol_type = other RNA
cttgcataattttcttcttcg

SEQ ID NO: 682 - RNA, 21 nt, synthetic construct
  misc_feature 1..21: note = Synthetic sequence
  source 1..21: mol_type = other RNA
acgaagaagaaaattatgcaa

SEQ ID NO: 683 - RNA, 21 nt, synthetic construct
  misc_feature 1..21: note = Synthetic sequence
  source 1..21: mol_type = other RNA
gcataattttcttcttcgtgg

SEQ ID NO: 684 - RNA, 21 nt, synthetic construct
  misc_feature 1..21: note = Synthetic sequence
  source 1..21: mol_type = other RNA
ttaagaagacgatcagcacca

SEQ ID NO: 685 - RNA, 21 nt, synthetic construct
  misc_feature 1..21: note = Synthetic sequence
  source 1..21: mol_type = other RNA
gtgctgatcgtcttcttaacg

SEQ ID NO: 686 - RNA, 21 nt, synthetic construct
  misc_feature 1..21: note = Synthetic sequence
  source 1..21: mol_type = other RNA
tcgttaagaagacgatcagca

SEQ ID NO: 687 - RNA, 21 nt, synthetic construct
  misc_feature 1..21: note = Synthetic sequence
  source 1..21: mol_type = other RNA
ctgatcgtcttcttaacgatc

SEQ ID NO: 688 - RNA, 21 nt, synthetic construct
  misc_feature 1..21: note = Synthetic sequence
  source 1..21: mol_type = other RNA
tgaagatcgttaagaagacga

SEQ ID NO: 689 - RNA, 21 nt, synthetic construct
  misc_feature 1..21: note = Synthetic sequence
  source 1..21: mol_type = other RNA
gtcttcttaacgatcttcagc

SEQ ID NO: 690 - RNA, 21 nt, synthetic construct
  misc_feature 1..21: note = Synthetic sequence
  source 1..21: mol_type = other RNA
aggattgtctgcaatgttgcc

SEQ ID NO: 691 - RNA, 21 nt, synthetic construct
  misc_feature 1..21: note = Synthetic sequence
  source 1..21: mol_type = other RNA
caacattgcagacaatcctca

SEQ ID NO: 692 - RNA, 21 nt, synthetic construct
  misc_feature 1..21: note = Synthetic sequence
  source 1..21: mol_type = other RNA
ttgaggattgtctgcaatgtt

SEQ ID NO: 693 - RNA, 21 nt, synthetic construct
  misc_feature 1..21: note = Synthetic sequence
  source 1..21: mol_type = other RNA
cattgcagacaatcctcaact

SEQ ID NO: 694 - RNA, 21 nt, synthetic construct
  misc_feature 1..21: note = Synthetic sequence
  source 1..21: mol_type = other RNA
acagttgaggattgtctgcaa

SEQ ID NO: 695 - RNA, 21 nt, synthetic construct
  misc_feature 1..21: note = Synthetic sequence
  source 1..21: mol_type = other RNA
gcagacaatcctcaactgtcc

SEQ ID NO: 696 - RNA, 21 nt, synthetic construct
  misc_feature 1..21: note = Synthetic sequence
  source 1..21: mol_type = other RNA
tggtagaaggacagttgagga

SEQ ID NO: 697 - RNA, 21 nt, synthetic construct
  misc_feature 1..21: note = Synthetic sequence
  source 1..21: mol_type = other RNA
ctcaactgtccttctaccagc

SEQ ID NO: 698 - RNA, 21 nt, synthetic construct
  misc_feature 1..21: note = Synthetic sequence
  source 1..21: mol_type = other RNA
tgaccttggtgaaaatccctg

SEQ ID NO: 699 - RNA, 21 nt, synthetic construct
  misc_feature 1..21: note = Synthetic sequence
  source 1..21: mol_type = other RNA
gggattttcaccaaggtcacg

SEQ ID NO: 700 - RNA, 21 nt, synthetic construct
  misc_feature 1..21: note = Synthetic sequence
  source 1..21: mol_type = other RNA
taaagagcttgttgtgcaggg

SEQ ID NO: 701 - RNA, 21 nt, synthetic construct
  misc_feature 1..21: note = Synthetic sequence
  source 1..21: mol_type = other RNA
ctgcacaacaagctctttaac

SEQ ID NO: 702 - RNA, 21 nt, synthetic construct
  misc_feature 1..21: note = Synthetic sequence
  source 1..21: mol_type = other RNA
tgttaaagagcttgttgtgca

SEQ ID NO: 703 - RNA, 21 nt, synthetic construct
  misc_feature 1..21: note = Synthetic sequence
  source 1..21: mol_type = other RNA
cacaacaagctctttaacaag

SEQ ID NO: 704 - RNA, 21 nt, synthetic construct
  misc_feature 1..21: note = Synthetic sequence
  source 1..21: mol_type = other RNA
aaccttgttaaagagcttgtt

SEQ ID NO: 705 - RNA, 21 nt, synthetic construct
  misc_feature 1..21: note = Synthetic sequence
  source 1..21: mol_type = other RNA
caagctctttaacaaggtttt

SEQ ID NO: 706 - RNA, 21 nt, synthetic construct
  misc_feature 1..21: note = Synthetic sequence
  source 1..21: mol_type = other RNA
tgtcaaagaaactcatggggc

SEQ ID NO: 707 - RNA, 21 nt, synthetic construct
  misc_feature 1..21: note = Synthetic sequence
  source 1..21: mol_type = other RNA
cccatgagtttctttgacacc

SEQ ID NO: 708 - RNA, 21 nt, synthetic construct
  misc_feature 1..21: note = Synthetic sequence
  source 1..21: mol_type = other RNA
tattgggatggtgtcaaagaa

SEQ ID NO: 709 - RNA, 21 nt, synthetic construct
  misc_feature 1..21: note = Synthetic sequence
  source 1..21: mol_type = other RNA
ctttgacaccatcccaatagg

SEQ ID NO: 710 - RNA, 21 nt, synthetic construct
  misc_feature 1..21: note = Synthetic sequence
  source 1..21: mol_type = other RNA
tcaaaagccggcctattggga

SEQ ID NO: 711 - RNA, 21 nt, synthetic construct
  misc_feature 1..21: note = Synthetic sequence
  source 1..21: mol_type = other RNA
ccaataggccggcttttgaac

SEQ ID NO: 712 - RNA, 21 nt, synthetic construct
  misc_feature 1..21: note = Synthetic sequence
  source 1..21: mol_type = other RNA
ttcaaaagccggcctattggg

SEQ ID NO: 713 - RNA, 21 nt, synthetic construct
  misc_feature 1..21: note = Synthetic sequence
  source 1..21: mol_type = other RNA
caataggccggcttttgaact

SEQ ID NO: 714 - RNA, 21 nt, synthetic construct
  misc_feature 1..21: note = Synthetic sequence
  source 1..21: mol_type = other RNA
aaaagatgggcaagagctggt

SEQ ID NO: 715 - RNA, 21 nt, synthetic construct
  misc_feature 1..21: note = Synthetic sequence
  source 1..21: mol_type = other RNA
cagctcttgcccatcttttca

SEQ ID NO: 716 - RNA, 21 nt, synthetic construct
  misc_feature 1..21: note = Synthetic sequence
  source 1..21: mol_type = other RNA
tgaaaagatgggcaagagctg

SEQ ID NO: 717 - RNA, 21 nt, synthetic construct
  misc_feature 1..21: note = Synthetic sequence
  source 1..21: mol_type = other RNA
gctcttgcccatcttttcaga

SEQ ID NO: 718 - RNA, 21 nt, synthetic construct
  misc_feature 1..21: note = Synthetic sequence
  source 1..21: mol_type = other RNA
taacaggatatatggagacag

SEQ ID NO: 719 - RNA, 21 nt, synthetic construct
  misc_feature 1..21: note = Synthetic sequence
  source 1..21: mol_type = other RNA
gtctccatatatcctgttaat

SEQ ID NO: 720 - RNA, 21 nt, synthetic construct
  misc_feature 1..21: note = Synthetic sequence
  source 1..21: mol_type = other RNA
attaacaggatatatggagac

SEQ ID NO: 721 - RNA, 21 nt, synthetic construct
  misc_feature 1..21: note = Synthetic sequence
  source 1..21: mol_type = other RNA
ctccatatatcctgttaatgg

SEQ ID NO: 722 - RNA, 21 nt, synthetic construct
  misc_feature 1..21: note = Synthetic sequence
  source 1..21: mol_type = other RNA
ttatggctcccattaacagga

SEQ ID NO: 723 - RNA, 21 nt, synthetic construct
  misc_feature 1..21: note = Synthetic sequence
  source 1..21: mol_type = other RNA
ctgttaatgggagccataatc

SEQ ID NO: 724 - RNA, 21 nt, synthetic construct
  misc_feature 1..21: note = Synthetic sequence
  source 1..21: mol_type = other RNA
ataaccatgattatggctccc

SEQ ID NO: 725 - RNA, 21 nt, synthetic construct
  misc_feature 1..21: note = Synthetic sequence
  source 1..21: mol_type = other RNA
gagccataatcatggttattt

SEQ ID NO: 726 - RNA, 21 nt, synthetic construct
  misc_feature 1..21: note = Synthetic sequence
  source 1..21: mol_type = other RNA
aaataaccatgattatggctc

SEQ ID NO: 727 - RNA, 21 nt, synthetic construct
  misc_feature 1..21: note = Synthetic sequence
  source 1..21: mol_type = other RNA
gccataatcatggttatttgc

SEQ ID NO: 728 - RNA, 21 nt, synthetic construct
  misc_feature 1..21: note = Synthetic sequence
  source 1..21: mol_type = other RNA
aatgaagcaaataaccatgat

SEQ ID NO: 729 - RNA, 21 nt, synthetic construct
  misc_feature 1..21: note = Synthetic sequence
  source 1..21: mol_type = other RNA
catggttatttgcttcattta

SEQ ID NO: 730 - RNA, 21 nt, synthetic construct
  misc_feature 1..21: note = Synthetic sequence
  source 1..21: mol_type = other RNA
ataaatgaagcaaataaccat

SEQ ID NO: 731 - RNA, 21 nt, synthetic construct
  misc_feature 1..21: note = Synthetic sequence
  source 1..21: mol_type = other RNA
ggttatttgcttcatttatta

SEQ ID NO: 732 - RNA, 21 nt, synthetic construct
  misc_feature 1..21: note = Synthetic sequence
  source 1..21: mol_type = other RNA
aataaatgaagcaaataacca

SEQ ID NO: 733 - RNA, 21 nt, synthetic construct
  misc_feature 1..21: note = Synthetic sequence
  source 1..21: mol_type = other RNA
gttatttgcttcatttattat

SEQ ID NO: 734 - RNA, 21 nt, synthetic construct
  misc_feature 1..21: note = Synthetic sequence
  source 1..21: mol_type = other RNA
atcatataataaatgaagcaa

SEQ ID NO: 735 - RNA, 21 nt, synthetic construct
  misc_feature 1..21: note = Synthetic sequence
  source 1..21: mol_type = other RNA
gcttcatttattatatgatgt

SEQ ID NO: 736 - RNA, 21 nt, synthetic construct
  misc_feature 1..21: note = Synthetic sequence
  source 1..21: mol_type = other RNA
atagttctccagtctcttgaa

SEQ ID NO: 737 - RNA, 21 nt, synthetic construct
  misc_feature 1..21: note = Synthetic sequence
  source 1..21: mol_type = other RNA
caagagactggagaactatag

SEQ ID NO: 738 - RNA, 21 nt, synthetic construct
  misc_feature 1..21: note = Synthetic sequence
  source 1..21: mol_type = other RNA
taaaggagaccggctatagtt

SEQ ID NO: 739 - RNA, 21 nt, synthetic construct
  misc_feature 1..21: note = Synthetic sequence
  source 1..21: mol_type = other RNA
ctatagccggtctcctttatt

SEQ ID NO: 740 - RNA, 21 nt, synthetic construct
  misc_feature 1..21: note = Synthetic sequence
  source 1..21: mol_type = other RNA
aggatgtgggagaataaagga

SEQ ID NO: 741 - RNA, 21 nt, synthetic construct
  misc_feature 1..21: note = Synthetic sequence
  source 1..21: mol_type = other RNA
ctttattctcccacatcctca

SEQ ID NO: 742 - RNA, 21 nt, synthetic construct
  misc_feature 1..21: note = Synthetic sequence
  source 1..21: mol_type = other RNA
agagaattgaggatgtgggag

SEQ ID NO: 743 - RNA, 21 nt, synthetic construct
  misc_feature 1..21: note = Synthetic sequence
  source 1..21: mol_type = other RNA
cccacatcctcaattctctgc

SEQ ID NO: 744 - RNA, 21 nt, synthetic construct
  misc_feature 1..21: note = Synthetic sequence
  source 1..21: mol_type = other RNA
tttccatagacatggatggag

SEQ ID NO: 745 - RNA, 21 nt, synthetic construct
  misc_feature 1..21: note = Synthetic sequence
  source 1..21: mol_type = other RNA
ccatccatgtctatggaaaaa

SEQ ID NO: 746 - RNA, 21 nt, synthetic construct
  misc_feature 1..21: note = Synthetic sequence
  source 1..21: mol_type = other RNA
ttttccatagacatggatgga

SEQ ID NO: 747 - RNA, 21 nt, synthetic construct
  misc_feature 1..21: note = Synthetic sequence
  source 1..21: mol_type = other RNA
catccatgtctatggaaaaac

SEQ ID NO: 748 - RNA, 21 nt, synthetic construct
  misc_feature 1..21: note = Synthetic sequence
  source 1..21: mol_type = other RNA
agtttttccatagacatggat

SEQ ID NO: 749 - RNA, 21 nt, synthetic construct
  misc_feature 1..21: note = Synthetic sequence
  source 1..21: mol_type = other RNA
ccatgtctatggaaaaactga

SEQ ID NO: 750 - RNA, 21 nt, synthetic construct
  misc_feature 1..21: note = Synthetic sequence
  source 1..21: mol_type = other RNA
tgaagtcttcagtttttccat

SEQ ID NO: 751 - RNA, 21 nt, synthetic construct
  misc_feature 1..21: note = Synthetic sequence
  source 1..21: mol_type = other RNA
ggaaaaactgaagacttcatc

SEQ ID NO: 752 - RNA, 21 nt, synthetic construct
  misc_feature 1..21: note = Synthetic sequence
  source 1..21: mol_type = other RNA
atgaagtcttcagtttttcca

SEQ ID NO: 753 - RNA, 21 nt, synthetic construct
  misc_feature 1..21: note = Synthetic sequence
  source 1..21: mol_type = other RNA
gaaaaactgaagacttcatca

SEQ ID NO: 754 - RNA, 21 nt, synthetic construct
  misc_feature 1..21: note = Synthetic sequence
  source 1..21: mol_type = other RNA
taaactggctgatgaagtctt

SEQ ID NO: 755 - RNA, 21 nt, synthetic construct
  misc_feature 1..21: note = Synthetic sequence
  source 1..21: mol_type = other RNA
gacttcatcagccagtttaag

SEQ ID NO: 756 - RNA, 21 nt, synthetic construct
  misc_feature 1..21: note = Synthetic sequence
  source 1..21: mol_type = other RNA
atcagtcagcctcttaaactg

SEQ ID NO: 757 - RNA, 21 nt, synthetic construct
  misc_feature 1..21: note = Synthetic sequence
  source 1..21: mol_type = other RNA
gtttaagaggctgactgatgc

SEQ ID NO: 758 - RNA, 21 nt, synthetic construct
  misc_feature 1..21: note = Synthetic sequence
  source 1..21: mol_type = other RNA
tattctgcgcatcagtcagcc

SEQ ID NO: 759 - RNA, 21 nt, synthetic construct
  misc_feature 1..21: note = Synthetic sequence
  source 1..21: mol_type = other RNA
ctgactgatgcgcagaataac

SEQ ID NO: 760 - RNA, 21 nt, synthetic construct
  misc_feature 1..21: note = Synthetic sequence
  source 1..21: mol_type = other RNA
aagatagaaacaacagcaggt

SEQ ID NO: 761 - RNA, 21 nt, synthetic construct
  misc_feature 1..21: note = Synthetic sequence
  source 1..21: mol_type = other RNA
ctgctgttgtttctatcttcc

SEQ ID NO: 762 - RNA, 21 nt, synthetic construct
  misc_feature 1..21: note = Synthetic sequence
  source 1..21: mol_type = other RNA
tggaagatagaaacaacagca

SEQ ID NO: 763 - RNA, 21 nt, synthetic construct
  misc_feature 1..21: note = Synthetic sequence
  source 1..21: mol_type = other RNA
ctgttgtttctatcttccaca

SEQ ID NO: 764 - RNA, 21 nt, synthetic construct
  misc_feature 1..21: note = Synthetic sequence
  source 1..21: mol_type = other RNA
tgtggaagatagaaacaacag

SEQ ID NO: 765 - RNA, 21 nt, synthetic construct
  misc_feature 1..21: note = Synthetic sequence
  source 1..21: mol_type = other RNA
gttgtttctatcttccacacg

SEQ ID NO: 766 - RNA, 21 nt, synthetic construct
  misc_feature 1..21: note = Synthetic sequence
  source 1..21: mol_type = other RNA
atgatctccagcctcaatgcc

SEQ ID NO: 767 - RNA, 21 nt, synthetic construct
  misc_feature 1..21: note = Synthetic sequence
  source 1..21: mol_type = other RNA
cattgaggctggagatcatga

SEQ ID NO: 768 - RNA, 21 nt, synthetic construct
  misc_feature 1..21: note = Synthetic sequence
  source 1..21: mol_type = other RNA
tgccaaaagccacgaacaggg

SEQ ID NO: 769 - RNA, 21 nt, synthetic construct
  misc_feature 1..21: note = Synthetic sequence
  source 1..21: mol_type = other RNA
ctgttcgtggcttttggcatt

SEQ ID NO: 770 - RNA, 21 nt, synthetic construct
  misc_feature 1..21: note = Synthetic sequence
  source 1..21: mol_type = other RNA
aggaaatgccaaaagccacga

SEQ ID NO: 771 - RNA, 21 nt, synthetic construct
  misc_feature 1..21: note = Synthetic sequence
  source 1..21: mol_type = other RNA
gtggcttttggcatttcctcc

SEQ ID NO: 772 - RNA, 21 nt, synthetic construct
  misc_feature 1..21: note = Synthetic sequence
  source 1..21: mol_type = other RNA
tgactttaaaggagtaggggg

SEQ ID NO: 773 - RNA, 21 nt, synthetic construct
  misc_feature 1..21: note = Synthetic sequence
  source 1..21: mol_type = other RNA
ccctactcctttaaagtcatg

SEQ ID NO: 774 - RNA, 21 nt, synthetic construct
  misc_feature 1..21: note = Synthetic sequence
  source 1..21: mol_type = other RNA
atgactttaaaggagtagggg

SEQ ID NO: 775 - RNA, 21 nt, synthetic construct
  misc_feature 1..21: note = Synthetic sequence
  source 1..21: mol_type = other RNA
cctactcctttaaagtcatgg

SEQ ID NO: 776 - RNA, 21 nt, synthetic construct
  misc_feature 1..21: note = Synthetic sequence
  source 1..21: mol_type = other RNA
tgaactgtgcctctgtctcca

SEQ ID NO: 777 - RNA, 21 nt, synthetic construct
  misc_feature 1..21: note = Synthetic sequence
  source 1..21: mol_type = other RNA
gagacagaggcacagttcacg

SEQ ID NO: 778 - RNA, 21 nt, synthetic construct
  misc_feature 1..21: note = Synthetic sequence
  source 1..21: mol_type = other RNA
tctacagccgtgaactgtgcc

SEQ ID NO: 779 - RNA, 21 nt, synthetic construct
  misc_feature 1..21: note = Synthetic sequence
  source 1..21: mol_type = other RNA
cacagttcacggctgtagaga

SEQ ID NO: 780 - RNA, 21 nt, synthetic construct
  misc_feature 1..21: note = Synthetic sequence
  source 1..21: mol_type = other RNA
tctctacagccgtgaactgtg

SEQ ID NO: 781 - RNA, 21 nt, synthetic construct
  misc_feature 1..21: note = Synthetic sequence
  source 1..21: mol_type = other RNA
cagttcacggctgtagagagg

SEQ ID NO: 782 - RNA, 21 nt, synthetic construct
  misc_feature 1..21: note = Synthetic sequence
  source 1..21: mol_type = other RNA
tgcagtatcctctctacagcc

SEQ ID NO: 783 - RNA, 21 nt, synthetic construct
  misc_feature 1..21: note = Synthetic sequence
  source 1..21: mol_type = other RNA
ctgtagagaggatactgcagt

SEQ ID NO: 784 - RNA, 21 nt, synthetic construct
  misc_feature 1..21: note = Synthetic sequence
  source 1..21: mol_type = other RNA
ttcatgtactgcagtatcctc

SEQ ID NO: 785 - RNA, 21 nt, synthetic construct
  misc_feature 1..21: note = Synthetic sequence
  source 1..21: mol_type = other RNA
ggatactgcagtacatgaaga

SEQ ID NO: 786 - RNA, 21 nt, synthetic construct
  misc_feature 1..21: note = Synthetic sequence
  source 1..21: mol_type = other RNA
acatcttcatgtactgcagta

SEQ ID NO: 787 - RNA, 21 nt, synthetic construct
  misc_feature 1..21: note = Synthetic sequence
  source 1..21: mol_type = other RNA
ctgcagtacatgaagatgtgt

SEQ ID NO: 788 - RNA, 21 nt, synthetic construct
  misc_feature 1..21: note = Synthetic sequence
  source 1..21: mol_type = other RNA
acacatcttcatgtactgcag

SEQ ID NO: 789 - RNA, 21 nt, synthetic construct
  misc_feature 1..21: note = Synthetic sequence
  source 1..21: mol_type = other RNA
gcagtacatgaagatgtgtgt

SEQ ID NO: 790 - RNA, 21 nt, synthetic construct
  misc_feature 1..21: note = Synthetic sequence
  source 1..21: mol_type = other RNA
aacttgtgccttccatgtgta

SEQ ID NO: 791 - RNA, 21 nt, synthetic construct
  misc_feature 1..21: note = Synthetic sequence
  source 1..21: mol_type = other RNA
cacatggaaggcacaagttgt

SEQ ID NO: 792 - RNA, 21 nt, synthetic construct
  misc_feature 1..21: note = Synthetic sequence
  source 1..21: mol_type = other RNA
acaacttgtgccttccatgtg

SEQ ID NO: 793 - RNA, 21 nt, synthetic construct
  misc_feature 1..21: note = Synthetic sequence
  source 1..21: mol_type = other RNA
catggaaggcacaagttgtcc

SEQ ID NO: 794 - RNA, 21 nt, synthetic construct
  misc_feature 1..21: note = Synthetic sequence
  source 1..21: mol_type = other RNA
atgatttccccatgctgtggc

SEQ ID NO: 795 - RNA, 21 nt, synthetic construct
  misc_feature 1..21: note = Synthetic sequence
  source 1..21: mol_type = other RNA
cacagcatggggaaatcatat

SEQ ID NO: 796 - RNA, 21 nt, synthetic construct
  misc_feature 1..21: note = Synthetic sequence
  source 1..21: mol_type = other RNA
tcctgaaatatgatttcccca

SEQ ID NO: 797 - RNA, 21 nt, synthetic construct
  misc_feature 1..21: note = Synthetic sequence
  source 1..21: mol_type = other RNA
gggaaatcatatttcaggatt

SEQ ID NO: 798 - RNA, 21 nt, synthetic construct
  misc_feature 1..21: note = Synthetic sequence
  source 1..21: mol_type = other RNA
atcctgaaatatgatttcccc

SEQ ID NO: 799 - RNA, 21 nt, synthetic construct
  misc_feature 1..21: note = Synthetic sequence
  source 1..21: mol_type = other RNA
ggaaatcatatttcaggatta

SEQ ID NO: 800 - RNA, 21 nt, synthetic construct
  misc_feature 1..21: note = Synthetic sequence
  source 1..21: mol_type = other RNA
aatcctgaaatatgatttccc

SEQ ID NO: 801 - RNA, 21 nt, synthetic construct
  misc_feature 1..21: note = Synthetic sequence
  source 1..21: mol_type = other RNA
gaaatcatatttcaggattat

SEQ ID NO: 802 - RNA, 21 nt, synthetic construct
  misc_feature 1..21: note = Synthetic sequence
  source 1..21: mol_type = other RNA
atttcatgtgataatcctgaa

SEQ ID NO: 803 - RNA, 21 nt, synthetic construct
  misc_feature 1..21: note = Synthetic sequence
  source 1..21: mol_type = other RNA
caggattatcacatgaaatac

SEQ ID NO: 804 - RNA, 21 nt, synthetic construct
  misc_feature 1..21: note = Synthetic sequence
  source 1..21: mol_type = other RNA
tgtatttcatgtgataatcct

SEQ ID NO: 805 - RNA, 21 nt, synthetic construct
  misc_feature 1..21: note = Synthetic sequence
  source 1..21: mol_type = other RNA
gattatcacatgaaatacaga

SEQ ID NO: 806 - RNA, 21 nt, synthetic construct
  misc_feature 1..21: note = Synthetic sequence
  source 1..21: mol_type = other RNA
tgtctctgtatttcatgtgat

SEQ ID NO: 807 - RNA, 21 nt, synthetic construct
  misc_feature 1..21: note = Synthetic sequence
  source 1..21: mol_type = other RNA
cacatgaaatacagagacaac

SEQ ID NO: 808 - RNA, 21 nt, synthetic construct
  misc_feature 1..21: note = Synthetic sequence
  source 1..21: mol_type = other RNA
tgtgttgtctctgtatttcat

SEQ ID NO: 809 - RNA, 21 nt, synthetic construct
  misc_feature 1..21: note = Synthetic sequence
  source 1..21: mol_type = other RNA
gaaatacagagacaacacacc

SEQ ID NO: 810 - RNA, 21 nt, synthetic construct
  misc_feature 1..21: note = Synthetic sequence
  source 1..21: mol_type = other RNA
aatgtccacgccgtcaatgag

SEQ ID NO: 811 - RNA, 21 nt, synthetic construct
  misc_feature 1..21: note = Synthetic sequence
  source 1..21: mol_type = other RNA
cattgacggcgtggacatttg

SEQ ID NO: 812 - RNA, 21 nt, synthetic construct
  misc_feature 1..21: note = Synthetic sequence
  source 1..21: mol_type = other RNA
agagcttggaccgcaagtcct

SEQ ID NO: 813 - RNA, 21 nt, synthetic construct
  misc_feature 1..21: note = Synthetic sequence
  source 1..21: mol_type = other RNA
gacttgcggtccaagctctca

SEQ ID NO: 814 - RNA, 21 nt, synthetic construct
  misc_feature 1..21: note = Synthetic sequence
  source 1..21: mol_type = other RNA
atcactgagagcttggaccgc

SEQ ID NO: 815 - RNA, 21 nt, synthetic construct
  misc_feature 1..21: note = Synthetic sequence
  source 1..21: mol_type = other RNA
ggtccaagctctcagtgatcc

SEQ ID NO: 816 - RNA, 21 nt, synthetic construct
  misc_feature 1..21: note = Synthetic sequence
  source 1..21: mol_type = other RNA
atcttgagggatcactgagag

SEQ ID NO: 817 - RNA, 21 nt, synthetic construct
  misc_feature 1..21: note = Synthetic sequence
  source 1..21: mol_type = other RNA
ctcagtgatccctcaagatcc

SEQ ID NO: 818 - RNA, 21 nt, synthetic construct
  misc_feature 1..21: note = Synthetic sequence
  source 1..21: mol_type = other RNA
tctgatggttcctgagagcag

SEQ ID NO: 819 - RNA, 21 nt, synthetic construct
  misc_feature 1..21: note = Synthetic sequence
  source 1..21: mol_type = other RNA
gctctcaggaaccatcagatt

SEQ ID NO: 820 - RNA, 21 nt, synthetic construct
  misc_feature 1..21: note = Synthetic sequence
  source 1..21: mol_type = other RNA
aggttgaatctgatggttcct

SEQ ID NO: 821 - RNA, 21 nt, synthetic construct
  misc_feature 1..21: note = Synthetic sequence
  source 1..21: mol_type = other RNA
gaaccatcagattcaacctag

SEQ ID NO: 822 - RNA, 21 nt, synthetic construct
  misc_feature 1..21: note = Synthetic sequence
  source 1..21: mol_type = other RNA
tctaggttgaatctgatggtt

SEQ ID NO: 823 - RNA, 21 nt, synthetic construct
  misc_feature 1..21: note = Synthetic sequence
  source 1..21: mol_type = other RNA
ccatcagattcaacctagatc

SEQ ID NO: 824 - RNA, 21 nt, synthetic construct
  misc_feature 1..21: note = Synthetic sequence
  source 1..21: mol_type = other RNA
atctaggttgaatctgatggt

SEQ ID NO: 825 - RNA, 21 nt, synthetic construct
  misc_feature 1..21: note = Synthetic sequence
  source 1..21: mol_type = other RNA
catcagattcaacctagatcc

SEQ ID NO: 826 - RNA, 21 nt, synthetic construct
  misc_feature 1..21: note = Synthetic sequence
  source 1..21: mol_type = other RNA
tcagtgtgacggtcaaaggga

SEQ ID NO: 827 - RNA, 21 nt, synthetic construct
  misc_feature 1..21: note = Synthetic sequence
  source 1..21: mol_type = other RNA
cctttgaccgtcacactgacc

SEQ ID NO: 828 - RNA, 21 nt, synthetic construct
  misc_feature 1..21: note = Synthetic sequence
  source 1..21: mol_type = other RNA
aggaatgtcctctccaaggca

SEQ ID NO: 829 - RNA, 21 nt, synthetic construct
  misc_feature 1..21: note = Synthetic sequence
  source 1..21: mol_type = other RNA
ccttggagaggacattcctga

SEQ ID NO: 830 - RNA, 21 nt, synthetic construct
  misc_feature 1..21: note = Synthetic sequence
  source 1..21: mol_type = other RNA
ttgagatggccttggtcagga

SEQ ID NO: 831 - RNA, 21 nt, synthetic construct
  misc_feature 1..21: note = Synthetic sequence
  source 1..21: mol_type = other RNA
ctgaccaaggccatctcaaag

SEQ ID NO: 832 - RNA, 21 nt, synthetic construct
  misc_feature 1..21: note = Synthetic sequence
  source 1..21: mol_type = other RNA
agctttttggggaactttgag

SEQ ID NO: 833 - RNA, 21 nt, synthetic construct
  misc_feature 1..21: note = Synthetic sequence
  source 1..21: mol_type = other RNA
caaagttccccaaaaagctgc

SEQ ID NO: 834 - RNA, 21 nt, synthetic construct
  misc_feature 1..21: note = Synthetic sequence
  source 1..21: mol_type = other RNA
tgtatgcagctttttggggaa

SEQ ID NO: 835 - RNA, 21 nt, synthetic construct
  misc_feature 1..21: note = Synthetic sequence
  source 1..21: mol_type = other RNA
ccccaaaaagctgcatacaga

SEQ ID NO: 836 - RNA, 21 nt, synthetic construct
  misc_feature 1..21: note = Synthetic sequence
  source 1..21: mol_type = other RNA
tctgtatgcagctttttgggg

SEQ ID NO: 837 - RNA, 21 nt, synthetic construct
  misc_feature 1..21: note = Synthetic sequence
  source 1..21: mol_type = other RNA
ccaaaaagctgcatacagatg

SEQ ID NO: 838 - RNA, 21 nt, synthetic construct
  misc_feature 1..21: note = Synthetic sequence
  source 1..21: mol_type = other RNA
atctgtatgcagctttttggg

SEQ ID NO: 839 - RNA, 21 nt, synthetic construct
  misc_feature 1..21: note = Synthetic sequence
  source 1..21: mol_type = other RNA
caaaaagctgcatacagatgt

SEQ ID NO: 840 - RNA, 21 nt, synthetic construct
  misc_feature 1..21: note = Synthetic sequence
  source 1..21: mol_type = other RNA
agtttccaccgttttccacca

SEQ ID NO: 841 - RNA, 21 nt, synthetic construct
  misc_feature 1..21: note = Synthetic sequence
  source 1..21: mol_type = other RNA
gtggaaaacggtggaaacttc

SEQ ID NO: 842 - RNA, 21 nt, synthetic construct
  misc_feature 1..21: note = Synthetic sequence
  source 1..21: mol_type = other RNA
agaagtttccaccgttttcca

SEQ ID NO: 843 - RNA, 21 nt, synthetic construct
  misc_feature 1..21: note = Synthetic sequence
  source 1..21: mol_type = other RNA
gaaaacggtggaaacttctct

SEQ ID NO: 844 - RNA, 21 nt, synthetic construct
  misc_feature 1..21: note = Synthetic sequence
  source 1..21: mol_type = other RNA
ttggagttgcgaagcacagcc

SEQ ID NO: 845 - RNA, 21 nt, synthetic construct
  misc_feature 1..21: note = Synthetic sequence
  source 1..21: mol_type = other RNA
ctgtgcttcgcaactccaaga

SEQ ID NO: 846 - RNA, 21 nt, synthetic construct
  misc_feature 1..21: note = Synthetic sequence
  source 1..21: mol_type = other RNA
tgatcttggagttgcgaagca

SEQ ID NO: 847 - RNA, 21 nt, synthetic construct
  misc_feature 1..21: note = Synthetic sequence
  source 1..21: mol_type = other RNA
cttcgcaactccaagatcatc

SEQ ID NO: 848 - RNA, 21 nt, synthetic construct
  misc_feature 1..21: note = Synthetic sequence
  source 1..21: mol_type = other RNA
aggatgatcttggagttgcga

SEQ ID NO: 849 - RNA, 21 nt, synthetic construct
  misc_feature 1..21: note = Synthetic sequence
  source 1..21: mol_type = other RNA
gcaactccaagatcatcctta

SEQ ID NO: 850 - RNA, 21 nt, synthetic construct
  misc_feature 1..21: note = Synthetic sequence
  source 1..21: mol_type = other RNA
aaggatgatcttggagttgcg

SEQ ID NO: 851 - RNA, 21 nt, synthetic construct
  misc_feature 1..21: note = Synthetic sequence
  source 1..21: mol_type = other RNA
caactccaagatcatccttat

SEQ ID NO: 852 - RNA, 21 nt, synthetic construct
  misc_feature 1..21: note = Synthetic sequence
  source 1..21: mol_type = other RNA
tcgataaggatgatcttggag

SEQ ID NO: 853 - RNA, 21 nt, synthetic construct
  misc_feature 1..21: note = Synthetic sequence
  source 1..21: mol_type = other RNA
ccaagatcatccttatcgatg

SEQ ID NO: 854 - RNA, 21 nt, synthetic construct
  misc_feature 1..21: note = Synthetic sequence
  source 1..21: mol_type = other RNA
atcgataaggatgatcttgga

SEQ ID NO: 855 - RNA, 21 nt, synthetic construct
  misc_feature 1..21: note = Synthetic sequence
  source 1..21: mol_type = other RNA
caagatcatccttatcgatga

SEQ ID NO: 856 - RNA, 21 nt, synthetic construct
  misc_feature 1..21: note = Synthetic sequence
  source 1..21: mol_type = other RNA
ttcatcgataaggatgatctt

SEQ ID NO: 857 - RNA, 21 nt, synthetic construct
  misc_feature 1..21: note = Synthetic sequence
  source 1..21: mol_type = other RNA
gatcatccttatcgatgaagc

SEQ ID NO: 858 - RNA, 21 nt, synthetic construct
  misc_feature 1..21: note = Synthetic sequence
  source 1..21: mol_type = other RNA
tctgtctccatgtcaatggag

SEQ ID NO: 859 - RNA, 21 nt, synthetic construct
  misc_feature 1..21: note = Synthetic sequence
  source 1..21: mol_type = other RNA
ccattgacatggagacagaca

SEQ ID NO: 860 - RNA, 21 nt, synthetic construct
  misc_feature 1..21: note = Synthetic sequence
  source 1..21: mol_type = other RNA
tcacagttcagcacagtggtg

SEQ ID NO: 861 - RNA, 21 nt, synthetic construct
  misc_feature 1..21: note = Synthetic sequence
  source 1..21: mol_type = other RNA
ccactgtgctgaactgtgacc

SEQ ID NO: 862 - RNA, 21 nt, synthetic construct
  misc_feature 1..21: note = Synthetic sequence
  source 1..21: mol_type = other RNA
tcccattgcccataaccagga

SEQ ID NO: 863 - RNA, 21 nt, synthetic construct
  misc_feature 1..21: note = Synthetic sequence
  source 1..21: mol_type = other RNA
ctggttatgggcaatgggaag

SEQ ID NO: 864 - RNA, 21 nt, synthetic construct
  misc_feature 1..21: note = Synthetic sequence
  source 1..21: mol_type = other RNA
tctaccaccttcccattgccc

SEQ ID NO: 865 - RNA, 21 nt, synthetic construct
  misc_feature 1..21: note = Synthetic sequence
  source 1..21: mol_type = other RNA
gcaatgggaaggtggtagaat

SEQ ID NO: 866 - RNA, 21 nt, synthetic construct
  misc_feature 1..21: note = Synthetic sequence
  source 1..21: mol_type = other RNA
ttctaccaccttcccattgcc

SEQ ID NO: 867 - RNA, 21 nt, synthetic construct
  misc_feature 1..21: note = Synthetic sequence
  source 1..21: mol_type = other RNA
caatgggaaggtggtagaatt

SEQ ID NO: 868 - RNA, 21 nt, synthetic construct
  misc_feature 1..21: note = Synthetic sequence
  source 1..21: mol_type = other RNA
tcaaattctaccaccttccca

SEQ ID NO: 869 - RNA, 21 nt, synthetic construct
  misc_feature 1..21: note = Synthetic sequence
  source 1..21: mol_type = other RNA
ggaaggtggtagaatttgatc

SEQ ID NO: 870 - RNA, 21 nt, synthetic construct
  misc_feature 1..21: note = Synthetic sequence
  source 1..21: mol_type = other RNA
atctcagtgaagaagtggctg

SEQ ID NO: 871 - RNA, 21 nt, synthetic construct
  misc_feature 1..21: note = Synthetic sequence
  source 1..21: mol_type = other RNA
gccacttcttcactgagataa

SEQ ID NO: 872 - RNA, 21 nt, synthetic construct
  misc_feature 1..21: note = Synthetic sequence
  source 1..21: mol_type = other RNA
tatctcagtgaagaagtggct

SEQ ID NO: 873 - RNA, 21 nt, synthetic construct
  misc_feature 1..21: note = Synthetic sequence
  source 1..21: mol_type = other RNA
ccacttcttcactgagataag

SEQ ID NO: 874 - RNA, 21 nt, synthetic construct
  misc_feature 1..21: note = Synthetic sequence
  source 1..21: mol_type = other RNA
ttatctcagtgaagaagtggc

SEQ ID NO: 875 - RNA, 21 nt, synthetic construct
  misc_feature 1..21: note = Synthetic sequence
  source 1..21: mol_type = other RNA
cacttcttcactgagataagg

SEQ ID NO: 876 - RNA, 21 nt, synthetic construct
  misc_feature 1..21: note = Synthetic sequence
  source 1..21: mol_type = other RNA
tctccttatctcagtgaagaa

SEQ ID NO: 877 - RNA, 21 nt, synthetic construct
  misc_feature 1..21: note = Synthetic sequence
  source 1..21: mol_type = other RNA
cttcactgagataaggagatg

SEQ ID NO: 878 - RNA, 21 nt, synthetic construct
  misc_feature 1..21: note = Synthetic sequence
  source 1..21: mol_type = other RNA
acatctccttatctcagtgaa

SEQ ID NO: 879 - RNA, 21 nt, synthetic construct
  misc_feature 1..21: note = Synthetic sequence
  source 1..21: mol_type = other RNA
cactgagataaggagatgtgg

SEQ ID NO: 880 - RNA, 21 nt, synthetic construct
  misc_feature 1..21: note = Synthetic sequence
  source 1..21: mol_type = other RNA
atgaagtctccacatctcctt

SEQ ID NO: 881 - RNA, 21 nt, synthetic construct
  misc_feature 1..21: note = Synthetic sequence
  source 1..21: mol_type = other RNA
ggagatgtggagacttcatgg

SEQ ID NO: 882 - RNA, 21 nt, synthetic construct
  misc_feature 1..21: note = Synthetic sequence
  source 1..21: mol_type = other RNA
tccatgaagtctccacatctc

SEQ ID NO: 883 - RNA, 21 nt, synthetic construct
  misc_feature 1..21: note = Synthetic sequence
  source 1..21: mol_type = other RNA
gatgtggagacttcatggagg

SEQ ID NO: 884 - RNA, 21 nt, synthetic construct
  misc_feature 1..21: note = Synthetic sequence
  source 1..21: mol_type = other RNA
agactgtgggcctcgaagctg

SEQ ID NO: 885 - RNA, 21 nt, synthetic construct
  misc_feature 1..21: note = Synthetic sequence
  source 1..21: mol_type = other RNA
gcttcgaggcccacagtctgc

SEQ ID NO: 886 - RNA, 21 nt, synthetic construct
  misc_feature 1..21: note = Synthetic sequence
  source 1..21: mol_type = other RNA
aagaaggtcgcagactgtggg

SEQ ID NO: 887 - RNA, 21 nt, synthetic construct
  misc_feature 1..21: note = Synthetic sequence
  source 1..21: mol_type = other RNA
cacagtctgcgaccttcttgt

SEQ ID NO: 888 - RNA, 21 nt, synthetic construct
  misc_feature 1..21: note = Synthetic sequence
  source 1..21: mol_type = other RNA
atctccaaacaagaaggtcgc

SEQ ID NO: 889 - RNA, 21 nt, synthetic construct
  misc_feature 1..21: note = Synthetic sequence
  source 1..21: mol_type = other RNA
gaccttcttgtttggagatga

SEQ ID NO: 890 - RNA, 21 nt, synthetic construct
  misc_feature 1..21: note = Synthetic sequence
  source 1..21: mol_type = other RNA
tcatctccaaacaagaaggtc

SEQ ID NO: 891 - RNA, 21 nt, synthetic construct
  misc_feature 1..21: note = Synthetic sequence
  source 1..21: mol_type = other RNA
ccttcttgtttggagatgaga

SEQ ID NO: 892 - RNA, 21 nt, synthetic construct
  misc_feature 1..21: note = Synthetic sequence
  source 1..21: mol_type = other RNA
aggagaagttctcatctccaa

SEQ ID NO: 893 - RNA, 21 nt, synthetic construct
  misc_feature 1..21: note = Synthetic sequence
  source 1..21: mol_type = other RNA
ggagatgagaacttctcctgg

SEQ ID NO: 894 - RNA, 21 nt, synthetic construct
  misc_feature 1..21: note = Synthetic sequence
  source 1..21: mol_type = other RNA
atttacccctgcttccaggag

SEQ ID NO: 895 - RNA, 21 nt, synthetic construct
  misc_feature 1..21: note = Synthetic sequence
  source 1..21: mol_type = other RNA
cctggaagcaggggtaaatgt

SEQ ID NO: 896 - RNA, 21 nt, synthetic construct
  misc_feature 1..21: note = Synthetic sequence
  source 1..21: mol_type = other RNA
tacatttacccctgcttccag

SEQ ID NO: 897 - RNA, 21 nt, synthetic construct
  misc_feature 1..21: note = Synthetic sequence
  source 1..21: mol_type = other RNA
ggaagcaggggtaaatgtagg

SEQ ID NO: 898 - RNA, 21 nt, synthetic construct
  misc_feature 1..21: note = Synthetic sequence
  source 1..21: mol_type = other RNA
tttccatccagcaatccccac

SEQ ID NO: 899 - RNA, 21 nt, synthetic construct
  misc_feature 1..21: note = Synthetic sequence
  source 1..21: mol_type = other RNA
ggggattgctggatggaaacc

SEQ ID NO: 900 - RNA, 21 nt, synthetic construct
  misc_feature 1..21: note = Synthetic sequence
  source 1..21: mol_type = other RNA
tattccagggtttccatccag

SEQ ID NO: 901 - RNA, 21 nt, synthetic construct
  misc_feature 1..21: note = Synthetic sequence
  source 1..21: mol_type = other RNA
ggatggaaaccctggaatagg

SEQ ID NO: 902 - RNA, 21 nt, synthetic construct
  misc_feature 1..21: note = Synthetic sequence
  source 1..21: mol_type = other RNA
tagcctattccagggtttcca

SEQ ID NO: 903 - RNA, 21 nt, synthetic construct
  misc_feature 1..21: note = Synthetic sequence
  source 1..21: mol_type = other RNA
gaaaccctggaataggctact

SEQ ID NO: 904 - RNA, 21 nt, synthetic construct
  misc_feature 1..21: note = Synthetic sequence
  source 1..21: mol_type = other RNA
tcaagtagcctattccagggt

SEQ ID NO: 905 - RNA, 21 nt, synthetic construct
  misc_feature 1..21: note = Synthetic sequence
  source 1..21: mol_type = other RNA
cctggaataggctacttgatg

SEQ ID NO: 906 - RNA, 21 nt, synthetic construct
  misc_feature 1..21: note = Synthetic sequence
  source 1..21: mol_type = other RNA
atcaagtagcctattccaggg

SEQ ID NO: 907 - RNA, 21 nt, synthetic construct
  misc_feature 1..21: note = Synthetic sequence
  source 1..21: mol_type = other RNA
ctggaataggctacttgatgg

SEQ ID NO: 908 - RNA, 21 nt, synthetic construct
  misc_feature 1..21: note = Synthetic sequence
  source 1..21: mol_type = other RNA
tggttctggggttctaaggtc

SEQ ID NO: 909 - RNA, 21 nt, synthetic construct
  misc_feature 1..21: note = Synthetic sequence
  source 1..21: mol_type = other RNA
ccttagaaccccagaaccatc

SEQ ID NO: 910 - RNA, 21 nt, synthetic construct
  misc_feature 1..21: note = Synthetic sequence
  source 1..21: mol_type = other RNA
atggttctggggttctaaggt

SEQ ID NO: 911 - RNA, 21 nt, synthetic construct
  misc_feature 1..21: note = Synthetic sequence
  source 1..21: mol_type = other RNA
cttagaaccccagaaccatct

SEQ ID NO: 912 - RNA, 21 nt, synthetic construct
  misc_feature 1..21: note = Synthetic sequence
  source 1..21: mol_type = other RNA
tgtcttagatggttctggggt

SEQ ID NO: 913 - RNA, 21 nt, synthetic construct
  misc_feature 1..21: note = Synthetic sequence
  source 1..21: mol_type = other RNA
cccagaaccatctaagacatg

SEQ ID NO: 914 - RNA, 21 nt, synthetic construct
  misc_feature 1..21: note = Synthetic sequence
  source 1..21: mol_type = other RNA
atgtcttagatggttctgggg

SEQ ID NO: 915 - RNA, 21 nt, synthetic construct
  misc_feature 1..21: note = Synthetic sequence
  source 1..21: mol_type = other RNA
ccagaaccatctaagacatgg

SEQ ID NO: 916 - RNA, 21 nt, synthetic construct
  misc_feature 1..21: note = Synthetic sequence
  source 1..21: mol_type = other RNA
aatcccatgtcttagatggtt

SEQ ID NO: 917 - RNA, 21 nt, synthetic construct
  misc_feature 1..21: note = Synthetic sequence
  source 1..21: mol_type = other RNA
ccatctaagacatgggattca

SEQ ID NO: 918 - RNA, 21 nt, synthetic construct
  misc_feature 1..21: note = Synthetic sequence
  source 1..21: mol_type = other RNA
actgaatcccatgtcttagat

SEQ ID NO: 919 - RNA, 21 nt, synthetic construct
  misc_feature 1..21: note = Synthetic sequence
  source 1..21: mol_type = other RNA
ctaagacatgggattcagtga

SEQ ID NO: 920 - RNA, 21 nt, synthetic construct
  misc_feature 1..21: note = Synthetic sequence
  source 1..21: mol_type = other RNA
atgatcactgaatcccatgtc

SEQ ID NO: 921 - RNA, 21 nt, synthetic construct
  misc_feature 1..21: note = Synthetic sequence
  source 1..21: mol_type = other RNA
catgggattcagtgatcatgt

SEQ ID NO: 922 - RNA, 21 nt, synthetic construct
  misc_feature 1..21: note = Synthetic sequence
  source 1..21: mol_type = other RNA
accacatgatcactgaatccc

SEQ ID NO: 923 - RNA, 21 nt, synthetic construct
  misc_feature 1..21: note = Synthetic sequence
  source 1..21: mol_type = other RNA
gattcagtgatcatgtggttc

SEQ ID NO: 924 - RNA, 21 nt, synthetic construct
  misc_feature 1..21: note = Synthetic sequence
  source 1..21: mol_type = other RNA
aaaggagaaccacatgatcac

SEQ ID NO: 925 - RNA, 21 nt, synthetic construct
  misc_feature 1..21: note = Synthetic sequence
  source 1..21: mol_type = other RNA
gatcatgtggttctcctttta

SEQ ID NO: 926 - RNA, 21 nt, synthetic construct
  misc_feature 1..21: note = Synthetic sequence
  source 1..21: mol_type = other RNA
ttaaaaggagaaccacatgat

SEQ ID NO: 927 - RNA, 21 nt, synthetic construct
  misc_feature 1..21: note = Synthetic sequence
  source 1..21: mol_type = other RNA
catgtggttctccttttaact

SEQ ID NO: 928 - RNA, 21 nt, synthetic construct
  misc_feature 1..21: note = Synthetic sequence
  source 1..21: mol_type = other RNA
aagttaaaaggagaaccacat

SEQ ID NO: 929 - RNA, 21 nt, synthetic construct
  misc_feature 1..21: note = Synthetic sequence
  source 1..21: mol_type = other RNA
gtggttctccttttaacttac

SEQ ID NO: 930 - RNA, 21 nt, synthetic construct
  misc_feature 1..21: note = Synthetic sequence
  source 1..21: mol_type = other RNA
tgtaagttaaaaggagaacca

SEQ ID NO: 931 - RNA, 21 nt, synthetic construct
  misc_feature 1..21: note = Synthetic sequence
  source 1..21: mol_type = other RNA
gttctccttttaacttacatg

SEQ ID NO: 932 - RNA, 21 nt, synthetic construct
  misc_feature 1..21: note = Synthetic sequence
  source 1..21: mol_type = other RNA
tcagcatgtaagttaaaagga

SEQ ID NO: 933 - RNA, 21 nt, synthetic construct
  misc_feature 1..21: note = Synthetic sequence
  source 1..21: mol_type = other RNA
cttttaacttacatgctgaat

SEQ ID NO: 934 - RNA, 21 nt, synthetic construct
  misc_feature 1..21: note = Synthetic sequence
  source 1..21: mol_type = other RNA
aaattattcagcatgtaagtt

SEQ ID NO: 935 - RNA, 21 nt, synthetic construct
  misc_feature 1..21: note = Synthetic sequence
  source 1..21: mol_type = other RNA
cttacatgctgaataatttta

SEQ ID NO: 936 - RNA, 21 nt, synthetic construct
  misc_feature 1..21: note = Synthetic sequence
  source 1..21: mol_type = other RNA
tataaaattattcagcatgta

SEQ ID NO: 937 - RNA, 21 nt, synthetic construct
  misc_feature 1..21: note = Synthetic sequence
  source 1..21: mol_type = other RNA
catgctgaataattttataat

SEQ ID NO: 938 - RNA, 21 nt, synthetic construct
  misc_feature 1..21: note = Synthetic sequence
  source 1..21: mol_type = other RNA
tattataaaattattcagcat

SEQ ID NO: 939 - RNA, 21 nt, synthetic construct
  misc_feature 1..21: note = Synthetic sequence
  source 1..21: mol_type = other RNA
gctgaataattttataataag

SEQ ID NO: 940 - RNA, 21 nt, synthetic construct
  misc_feature 1..21: note = Synthetic sequence
  source 1..21: mol_type = other RNA
ttattataaaattattcagca

SEQ ID NO: 941 - RNA, 21 nt, synthetic construct
  misc_feature 1..21: note = Synthetic sequence
  source 1..21: mol_type = other RNA
ctgaataattttataataagg

SEQ ID NO: 942 - RNA, 21 nt, synthetic construct
  misc_feature 1..21: note = Synthetic sequence
  source 1..21: mol_type = other RNA
aaaactataagcttttacctt

SEQ ID NO: 943 - RNA, 21 nt, synthetic construct
  misc_feature 1..21: note = Synthetic sequence
  source 1..21: mol_type = other RNA
ggtaaaagcttatagttttct

SEQ ID NO: 944 - RNA, 21 nt, synthetic construct
  misc_feature 1..21: note = Synthetic sequence
  source 1..21: mol_type = other RNA
agatcagaaaactataagctt

SEQ ID NO: 945 - RNA, 21 nt, synthetic construct
  misc_feature 1..21: note = Synthetic sequence
  source 1..21: mol_type = other RNA
gcttatagttttctgatctgt

SEQ ID NO: 946 - RNA, 21 nt, synthetic construct
  misc_feature 1..21: note = Synthetic sequence
  source 1..21: mol_type = other RNA
aacacttctaacacagatcag

SEQ ID NO: 947 - RNA, 21 nt, synthetic construct
  misc_feature 1..21: note = Synthetic sequence
  source 1..21: mol_type = other RNA
gatctgtgttagaagtgttgc

SEQ ID NO: 948 - RNA, 21 nt, synthetic construct
  misc_feature 1..21: note = Synthetic sequence
  source 1..21: mol_type = other RNA
tttgcaacacttctaacacag

SEQ ID NO: 949 - RNA, 21 nt, synthetic construct
  misc_feature 1..21: note = Synthetic sequence
  source 1..21: mol_type = other RNA
gtgttagaagtgttgcaaatg

SEQ ID NO: 950 - RNA, 21 nt, synthetic construct
  misc_feature 1..21: note = Synthetic sequence
  source 1..21: mol_type = other RNA
acagcatttgcaacacttcta

SEQ ID NO: 951 - RNA, 21 nt, synthetic construct
  misc_feature 1..21: note = Synthetic sequence
  source 1..21: mol_type = other RNA
gaagtgttgcaaatgctgtac

SEQ ID NO: 952 - RNA, 21 nt, synthetic construct
  misc_feature 1..21: note = Synthetic sequence
  source 1..21: mol_type = other RNA
tcagtacagcatttgcaacac

SEQ ID NO: 953 - RNA, 21 nt, synthetic construct
  misc_feature 1..21: note = Synthetic sequence
  source 1..21: mol_type = other RNA
gttgcaaatgctgtactgact

SEQ ID NO: 954 - RNA, 21 nt, synthetic construct
  misc_feature 1..21: note = Synthetic sequence
  source 1..21: mol_type = other RNA
aagtcagtacagcatttgcaa

SEQ ID NO: 955 - RNA, 21 nt, synthetic construct
  misc_feature 1..21: note = Synthetic sequence
  source 1..21: mol_type = other RNA
gcaaatgctgtactgactttg

SEQ ID NO: 956 - RNA, 21 nt, synthetic construct
  misc_feature 1..21: note = Synthetic sequence
  source 1..21: mol_type = other RNA
aaagtcagtacagcatttgca

SEQ ID NO: 957 - RNA, 21 nt, synthetic construct
  misc_feature 1..21: note = Synthetic sequence
  source 1..21: mol_type = other RNA
caaatgctgtactgactttgt

SEQ ID NO: 958 - RNA, 21 nt, synthetic construct
  misc_feature 1..21: note = Synthetic sequence
  source 1..21: mol_type = other RNA
tttacaaagtcagtacagcat

SEQ ID NO: 959 - RNA, 21 nt, synthetic construct
  misc_feature 1..21: note = Synthetic sequence
  source 1..21: mol_type = other RNA
gctgtactgactttgtaaaat

SEQ ID NO: 960 - RNA, 21 nt, synthetic construct
  misc_feature 1..21: note = Synthetic sequence
  source 1..21: mol_type = other RNA
ttttacaaagtcagtacagca

SEQ ID NO: 961 - RNA, 21 nt, synthetic construct
  misc_feature 1..21: note = Synthetic sequence
  source 1..21: mol_type = other RNA
ctgtactgactttgtaaaata

SEQ ID NO: 962 - RNA, 21 nt, synthetic construct
  misc_feature 1..21: note = Synthetic sequence
  source 1..21: mol_type = other RNA
tattttacaaagtcagtacag

SEQ ID NO: 963 - RNA, 21 nt, synthetic construct
  misc_feature 1..21: note = Synthetic sequence
  source 1..21: mol_type = other RNA
gtactgactttgtaaaatata

SEQ ID NO: 964 - RNA, 21 nt, synthetic construct
  misc_feature 1..21: note = Synthetic sequence
  source 1..21: mol_type = other RNA
ttatattttacaaagtcagta

SEQ ID NO: 965 - RNA, 21 nt, synthetic construct
  misc_feature 1..21: note = Synthetic sequence
  source 1..21: mol_type = other RNA
ctgactttgtaaaatataaaa

SEQ ID NO: 966 - RNA, 21 nt, synthetic construct
  misc_feature 1..21: note = Synthetic sequence
  source 1..21: mol_type = other RNA
ttttatattttacaaagtcag

SEQ ID NO: 967 - RNA, 21 nt, synthetic construct
  misc_feature 1..21: note = Synthetic sequence
  source 1..21: mol_type = other RNA
gactttgtaaaatataaaact

SEQ ID NO: 968 - RNA, 21 nt, synthetic construct
  misc_feature 1..21: note = Synthetic sequence
  source 1..21: mol_type = other RNA
agttttatattttacaaagtc

SEQ ID NO: 969 - RNA, 21 nt, synthetic construct
  misc_feature 1..21: note = Synthetic sequence
  source 1..21: mol_type = other RNA
ctttgtaaaatataaaactaa

SEQ ID NO: 970 - RNA, 19 nt, synthetic construct
  misc_feature 1..19: note = Synthetic siRNA
  source 1..19: mol_type = other RNA
gtttcaggactattttata

SEQ ID NO: 971 - RNA, 19 nt, synthetic construct
  misc_feature 1..19: note = Synthetic siRNA
  source 1..19: mol_type = other RNA
cctacttcattattggata

SEQ ID NO: 972 - RNA, 19 nt, synthetic construct
  misc_feature 1..19: note = Synthetic siRNA
  source 1..19: mol_type = other RNA
gtcctgtccttaatggtga

SEQ ID NO: 973 - RNA, 19 nt, synthetic construct
  misc_feature 1..19: note = Synthetic siRNA
  source 1..19: mol_type = other RNA
caaagatcctggaatattc

(19) United States
(12) Patent Application Publication
(10) Pub. No.: US 2025/0188461 A1
(43) Pub. Date: Jun. 12, 2025

(54) METHODS OF TREATING OSMIDROSIS

(71) Applicants: Roger L. Kaspar, Santa Cruz, CA (US); Thomas V. Barker, Aptos, CA (US)

(72) Inventors: Roger L. Kaspar, Santa Cruz, CA (US); Thomas V. Barker, Aptos, CA (US)

(21) Appl. No.: 18/959,170

(22) Filed: Nov. 25, 2024

Related U.S. Application Data
(63) Continuation of application No. 18/131,509, filed on Apr. 6, 2023, now abandoned.
Application No. 18/131,509 is a continuation of application No. 16/321,583, filed on Jan. 29, 2019, now abandoned.
(60) Provisional application No. 62/368,896, filed on Jul. 29, 2016.

(51) Int. Cl. C12N 15/113; A61K 45/06; A61K 48/00

(52) U.S. Cl. CPC C12N 15/113; A61K 45/06; A61K 48/0075; A61K 48/0083; C12N 2310/11; C12N 2310/141; C12N 2310/3181; C12N 2310/3233

(57) ABSTRACT

A method of treating an osmidrosis condition in a subject can include administering a therapeutic agent in an amount that is effective to inhibit expression of an ABCC11 gene in a target cell of the subject to an osmidrosis-reducing level. A therapeutic composition for treating an osmidrosis condition in a subject can include a therapeutically effective amount of an ABCC11 gene-inhibiting agent and a pharmaceutically acceptable carrier.

### PRIORITY DATA

This application is a continuation of U.S. patent application Ser. No. 18/131,509, filed Apr. 6, 2023, which is a continuation of U.S. patent application Ser. No. 16/321,583, filed Jan. 29, 2019, which is a 371 U.S. Nationalization of Patent Cooperation Treaty Application Serial No. PCT/US2017/044731, filed on Jul. 31, 2017, which claims the benefit of U.S. Provisional Patent Application Ser. No. 62/368,896, filed on Jul. 29, 2016, each of which is incorporated herein by reference.

### SEQUENCE LISTING

This application contains a sequence listing which is incorporated herein by reference in ST.26 XML format named 4093-001.PCT.US.CON2_Sequence_Listing.xml, created Mar. 3, 2025, and is 1.06 MB in size. The sequences contained in the sequence listing are found throughout the originally filed application.

### BACKGROUND

Sweating is an important physiological function that helps protect the body from overheating. There are millions of sweat glands distributed over the human body. Human sweat glands are primarily divided into two types: eccrine and apocrine. The majority of sweat glands are “eccrine” sweat glands, which are distributed over the entire skin surface and found in large numbers on the soles of the feet, the palms of the hands, the face, and in the armpits. Eccrine glands secrete an odorless, clear fluid that helps the body control its temperature by promoting heat loss through evaporation. However, in some cases, eccrine sweat can cause body odor. As one non-limiting example, in some circumstances, eccrine sweat can soften keratin, which can lead to bacterial degradation of the keratin and a corresponding foul smell. Another type of sweat gland is called the “apocrine” gland. Apocrine glands have a more limited distribution on the human body and are found most abundantly in the axilla, genital skin, and breasts. They produce a thick, oily fluid that produces a characteristic body odor when it comes into contact with bacteria on the surface of the skin.

While body odor can typically be controlled or masked using standard antiperspirants/deodorants, some individuals suffer from excessively foul-smelling sweat, which is considered pathologic and termed osmidrosis (also known as bromhidrosis or bromidrosis). Osmidrosis can be challenging to treat or prevent using standard antiperspirants/deodorants. As such, many patients suffering from this condition resort to alternative treatments such as microwave destruction of apocrine glands, botulinum toxin injections, and/or laser destruction of apocrine glands. In some instances, surgical removal of the apocrine glands by a radical surgical procedure is viewed as the best solution for osmidrosis.

### BRIEF DESCRIPTION OF THE DRAWINGS

For a fuller understanding of the nature and advantage of the present invention, reference is being made to the following detailed description of preferred embodiments and in connection with the accompanying drawings, in which:

FIG. 1 is a graph illustrating siRNA-mediated inhibition of ABCC11a gene expression in human HepG2 cells, in accordance with one aspect of the present disclosure.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” include express support for plural referents unless the context clearly dictates otherwise. Thus, for example, reference to “a polymer” can include a plurality of such polymers.

As used herein, “subject” refers to a mammal that can benefit from treatment with an ABCC11 inhibitor. A benefit can be obtained if the subject has a disease or condition, or is at risk of developing a disease or condition for which an ABCC11 inhibitor is a therapeutically effective treatment or preventative measure. In some aspects, such subject may be a human.

As used herein, the terms “treat,” “treatment,” or “treating” when used in conjunction with the administration of an ABCC11 inhibitor, such as an siRNA that targets the ABCC11 gene, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition. Treatment outcomes can be expected or unexpected. In one specific aspect, a treatment outcome can be a delay in occurrence or onset of a disease or conditions or the signs or symptoms thereof. In another aspect, a treatment can be reducing, ameliorating, eliminating, or otherwise providing a subject with relief from (i.e. relieving) the condition with which they are afflicted, or providing relief from signs or symptoms of the condition.

As used herein a “therapeutic agent,” “drug,” or “active agent” refers to an agent or compound that has a desired or intended biological effect (e.g. beneficial or positive) on a subject when administered to the subject in an appropriate or effective amount. In one aspect, an ABCC11 inhibitor can be a therapeutic agent.

The terms “ABCC11 inhibitor” or “ABCC11 gene-inhibiting agent” refer to agents or compounds that are effective in inhibiting expression of the ABCC11 protein (e.g. the wild type ABCC11 protein). ABCC11 is the human ATP-binding cassette (ABC) transport gene and encodes an ATP-driven efflux pump protein. ABCC11 is involved in cellular export of precursor odorants. Examples of ABCC11 inhibitors include but are not limited to siRNAs, miRNAs, antisense oligonucleotides, ribozymes, peptide nucleic acids, morpholinos, small molecule inhibitors, the like, or combinations thereof. Expression of the wildtype ABCC11 gene could alternatively be blocked by permanent genetic manipulations including homologous recombination, CRISPR/Cas9 gene editing and the like.

As used herein, the terms “inhibit” or “inhibiting” are used to refer to a variety of inhibition techniques. For example, the terms “inhibit” or “inhibiting” can refer to pre-and/or post-transcriptional inhibition. With respect to pre-transcription inhibition, “inhibit” or “inhibiting” can refer to preventing or reducing transcription of a gene, inducing altered transcription of a gene, and/or reducing a rate of transcription of a gene, whether permanent, semi-permanent, or transient. Thus, in some examples, “inhibit” or “inhibiting” can refer to permanent changes to the DNA, whereas in other examples no permanent change to the DNA is made. With respect to post-transcriptional inhibition, “inhibit” or “inhibiting” can refer to preventing or reducing translation of a genetic sequence to a protein, inducing an altered translation of a genetic sequence to an altered protein (e.g. as misfolded protein, etc.), and/or reducing a rate of translation of a genetic sequence to a protein, whether permanent, semi-permanent, or transient. In some specific examples, “inhibit” or “inhibiting” can refer to pre-transcriptional inhibition. In other specific examples, “inhibit” or “inhibiting” can refer to post-transcriptional inhibition. Of course, the type of inhibition can depend on the specific type(s) of inhibitor(s) or therapeutic agent(s) employed. Thus, “inhibit” or “inhibiting” can include any decrease in expression of a gene as compared to native expression, whether pre-or post-transcriptional, partial or complete.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a preparation that releases or otherwise administers an ABCC11 inhibitor.

The phrase “effective amount,” “therapeutically effective amount,” or “therapeutically effective rate(s)” of an active ingredient refer to a non-toxic, but sufficient amount or delivery rate of the active ingredient or therapeutic agent, to achieve therapeutic results in treating a disease or condition for which the drug or therapeutic is being delivered. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, “osmidrosis-reducing amount” or “odor-reducing amount” of an ABCC11 inhibitor, such as siRNA, and/or other suitable therapeutic agent refers to a sufficient amount or concentration of an ABCC11 inhibitor and/or other suitable therapeutic agent in a formulation or composition to provide an intended effect and/or achieve an intended result when administered to a subject. For example, an “osmidrosis-reducing amount” or “odor-reducing amount” of an ABCC11 inhibitor and/or other suitable therapeutic agent may be an amount sufficient to treat a particular target indication, e.g. osmidrosis or other condition for which the ABCC11 inhibitor and/or other suitable therapeutic agent can be used. In some non-limiting examples, an “osmidrosis-reducing amount” or “odor-reducing amount” can be an amount that induces inhibition of expression of the ABCC11 gene in a target cell by at least a target amount. In some non-limiting examples, an “osmidrosis-reducing amount” or “odor-reducing amount” can be an amount that reduces apocrine sweat production and/or output in a subject by at least a target amount. In some non-limiting examples, an “osmidrosis-reducing amount” or “odor-reducing amount” can be an amount that reduces bacterial loading (e.g. colony forming units [CFU] per unit area) and/or activity on a skin surface by at least a target amount.

As used herein, “skin,” “skin surface,” “derma,” “epidermis,” and similar terms are used interchangeably, and refer to not only the outer skin of a subject comprising the epidermis, but also to underlying layers and to mucosal surfaces.

As used herein, a “dosing regimen” or “regimen” such as “treatment dosing regimen,” or a “prophylactic dosing regimen,” refers to how, when, how much, and for how long a dose of a composition can or should be administered to a subject in order to achieve an intended treatment or effect.

As used herein the term “topical formulation” refers to a formulation that may be applied to skin or a mucosa. Topical formulations may, for example, be used to treat a subject by delivering an active agent or drug, such as an ABCC11 inhibitor. Topical formulations can be used for both topical and transdermal administration of substances. Examples of topical formulations include but are not limited to ointments, creams, lotions, gels, and pastes.

As used herein, “topical administration” is used in its conventional sense to mean delivery of a substance, such as a therapeutically active agent, to the skin or a localized region of a subject's body. Topical administration of a drug, such as an ABCC11 inhibitor may often be advantageously applied in, for example, the treatment of osmidrosis in a subject's skin. While topical administration can be for the purpose of treating a local area or region of tissue, such as skin, topical administration can also be for the purpose of providing transdermal administration.

As used herein, “transdermal administration” refers to administration through the skin. Transdermal administration is often applied where systemic delivery of an active is desired, although it may also be useful for delivering an active to tissues underlying the skin with minimal systemic absorption.

As used herein, “carrier,” and “pharmaceutically acceptable carrier” may be used interchangeably, and refer to any liquid, gel, salve, solvent, liquid, diluent, fluid ointment base, liposome, micelle, giant micelle, or the like, or any other suitable carrier that is suitable for delivery of a therapeutic agent to and/or into a target cell (e.g. an apocrine cell) and for use in contact with a subject or the subject's tissue without causing adverse physiological responses, and which does not interact with the other components of the composition in a deleterious manner. A number of carrier ingredients are known for use in making topical formulations, such as gelatin, polymers, fats and oils, lecithin, collagens, alcohols, water, etc.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference in this application may be made to compositions, systems, or methods that provide “improved” or “enhanced” performance. It is to be understood that unless otherwise stated, such “improvement” or “enhancement” is a measure of a benefit obtained based on a comparison to compositions, systems or methods in the prior art. Furthermore, it is to be understood that the degree of improved or enhanced performance may vary between disclosed embodiments and that no equality or consistency in the amount, degree, or realization of improvement or enhancement is to be assumed as universally applicable.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The human ATP-binding cassette (ABC) transport gene (ABCC11), having the gene sequence of SEQ ID NO: 1, encodes an ATP-driven efflux pump protein that has a key role in secretion of components of cerumen (earwax) and body odor precursors from apocrine glands. Expression of wildtype ABCC11 results in wet type earwax and osmidrosis while expression of a single-nucleotide polymorphism (SNP) version (538G→A, Gly180Arg, rs17822931) results in the dry type earwax and no osmidrosis. There is a strong association of the ABCC11 SNP with both osmidrosis and the earwax type. For example, a dominant inheritance pattern of the GG or GA genotypes is a wet type earwax phenotype and osmidrosis, while the recessive AA genotype results in the dry type earwax phenotype and no osmidrosis. More specifically, the wildtype ABCC11 protein is N-linked glycosylated, whereas the SNP version is not. Therefore, the lack of N-linked glycosylation results in recognition of the SNP-encoded version as a misfolded protein, with resultant ubiquitination and proteosomal degradation. However, no apparent deleterious effects result from homozygous expression of the SNP version of the ABCC11 gene (the protein product that is degraded), which suggests that the wildtype version can be eliminated safely with no side effects. As such, certain SNP's can lead to targeted degradation of the ABCC11 protein. In the absence of the ABCC11 protein, excretion of odor substances or odor precursors is blocked or limited and thus osmidrosis is reduced.

The present disclosure describes methods and compositions for treating osmidrosis. In some examples, a method of treating osmidrosis can include inhibiting ABCC11 gene expression. In some examples, inhibiting ABCC11 gene expression (and therefore reducing or eliminating odor) can include administration of inhibitors such as small interfering RNAs (siRNAs), micro RNAs (miRNAs), morpholinos, antisense oligonucleotides (ASOs), peptide nucleic acids, small molecule inhibitors, the like, or combinations thereof that temporarily inhibit ABCC11 expression. In some additional examples, a method of inhibiting ABCC11 gene expression can include gene therapy. Gene therapy (e.g. homologous recombination, CRISPR/Cas9 gene editing, etc.) can be used to permanently alter the DNA to prevent expression of ABCC11. In some examples, a method of treating osmidrosis can include both administering an inhibitor and gene therapy.

In one embodiment, the present invention provides a method of treating a subject with osmidrosis by administering to the subject an RNA sequence that inhibits the expression of the gene encoding the ABCC11 protein (e.g. wildtype ABCC11). As described above, it has been discovered that it is possible to suppress expression of wildtype ABCC11 without causing unwanted side effects as homozygous expression of the SNP-containing gene product is degraded without any apparent adverse unwanted effects. In other words, it may be possible to remove expression of ABCC11 protein and reduce osmidrosis without any unwanted side effects.

In some examples, methods of treating osmidrosis can include identifying a gene that contributes to osmidrosis and inhibiting gene expression contributing to osmidrosis in a target cell. In some additional examples, methods of treating osmidrosis can further include preparing an inhibitor to be administered to a subject having osmidrosis. In some specific examples, the gene that contributes to osmidrosis can be or include ABCC11.

A variety of segments or sequences of the ABCC11 gene can be targeted using a therapeutic agent to inhibit expression of the ABCC11 gene, whether the inhibition is permanent, semi-permanent, or transient. For example, one or more of the gene sequences listed in Table 1 below can be targeted to inhibit ABCC11 gene expression:

TABLE 1

| Position | ABCC11 Target Sequence | SEQ ID NO: |
|---|---|---|
| 12-34 | CCGGTGTATTTGAATAAACCAGG | 2 |
| 32-54 | AGGTTGGCAAATCATACTATAGC | 3 |
| 35-57 | TTGGCAAATCATACTATAGCTGA | 4 |
| 37-59 | GGCAAATCATACTATAGCTGAAA | 5 |
| 42-64 | ATCATACTATAGCTGAAAGAATT | 6 |
| 54-76 | CTGAAAGAATTGGCAGGAACTGA | 7 |
| 58-80 | AAGAATTGGCAGGAACTGAAAAT | 8 |
| 64-86 | TGGCAGGAACTGAAAATGACTAG | 9 |
| 65-87 | GGCAGGAACTGAAAATGACTAGG | 10 |
| 68-90 | AGGAACTGAAAATGACTAGGAAG | 11 |
| 71-93 | AACTGAAAATGACTAGGAAGAGG | 12 |
| 125-147 | TCGTGAATCGTGGCATCGACATA | 13 |
| 127-149 | GTGAATCGTGGCATCGACATAGG | 14 |
| 148-170 | GGCGATGACATGGTTTCAGGACT | 15 |
| 152-174 | ATGACATGGTTTCAGGACTTATT | 16 |
| 154-176 | GACATGGTTTCAGGACTTATTTA | 17 |
| 157-179 | ATGGTTTCAGGACTTATTTATAA | 18 |
| 158-180 | TGGTTTCAGGACTTATTTATAAA | 19 |
| 164-186 | CAGGACTTATTTATAAAACCTAT | 20 |
| 165-187 | AGGACTTATTTATAAAACCTATA | 21 |
| 167-189 | GACTTATTTATAAAACCTATACT | 22 |
| 204-226 | CTGGAGTCAGCAAGAGAGAAATC | 23 |
| 265-287 | AAGTATGATGCTGCCTTGAGAAC | 24 |
| 335-357 | TGGACAATGCTGGCCTGTTCTCC | 25 |
| 381-403 | CCCGCTCATGATCCAAAGCTTAC | 26 |
| 382-404 | CCGCTCATGATCCAAAGCTTACG | 27 |
| 396-418 | AAGCTTACGGAGTCGCTTAGATG | 28 |
| 397-419 | AGCTTACGGAGTCGCTTAGATGA | 29 |
| 408-430 | TCGCTTAGATGAGAACACCATCC | 30 |
| 442-464 | GTCCATGATGCCTCAGACAAAAA | 31 |
| 443-465 | TCCATGATGCCTCAGACAAAAAT | 32 |
| 450-472 | TGCCTCAGACAAAAATGTCCAAA | 33 |
| 451-473 | GCCTCAGACAAAAATGTCCAAAG | 34 |
| 457-479 | GACAAAAATGTCCAAAGGCTTCA | 35 |
| 472-494 | AGGCTTCACCGCCTTTGGGAAGA | 36 |
| 478-500 | CACCGCCTTTGGGAAGAAGAAGT | 37 |
| 480-502 | CCGCCTTTGGGAAGAAGAAGTCT | 38 |
| 482-504 | GCCTTTGGGAAGAAGAAGTCTCA | 39 |
| 510-532 | AGGGATTGAAAAAGCTTCAGTGC | 40 |
| 527-549 | CAGTGCTTCTGGTGATGCTGAGG | 41 |
| 536-558 | TGGTGATGCTGAGGTTCCAGAGA | 42 |
| 542-564 | TGCTGAGGTTCCAGAGAACAAGG | 43 |
| 546-568 | GAGGTTCCAGAGAACAAGGTTGA | 44 |
| 550-572 | TTCCAGAGAACAAGGTTGATTTT | 45 |
| 551-573 | TCCAGAGAACAAGGTTGATTTTC | 46 |
| 555-577 | GAGAACAAGGTTGATTTTCGATG | 47 |
| 562-584 | AGGTTGATTTTCGATGCACTTCT | 48 |
| 575-597 | ATGCACTTCTGGGCATCTGCTTC | 49 |
| 576-598 | TGCACTTCTGGGCATCTGCTTCT | 50 |
| 606-628 | CAGTGTACTCGGGCCAATATTGA | 51 |
| 616-638 | GGGCCAATATTGATTATACCAAA | 52 |
| 617-639 | GGCCAATATTGATTATACCAAAG | 53 |
| 618-640 | GCCAATATTGATTATACCAAAGA | 54 |
| 632-654 | TACCAAAGATCCTGGAATATTCA | 55 |
| 666-688 | GGGGAATGCTGTCCATGGAGTGG | 56 |
| 709-731 | CTCTCCGAATGCGTGAAGTCTCT | 57 |
| 711-733 | CTCCGAATGCGTGAAGTCTCTGA | 58 |
| 713-735 | CCGAATGCGTGAAGTCTCTGAGT | 59 |
| 717-739 | ATGCGTGAAGTCTCTGAGTTTCT | 60 |
| 719-741 | GCGTGAAGTCTCTGAGTTTCTCC | 61 |
| 732-754 | GAGTTTCTCCTCCAGTTGGATCA | 62 |
| 741-763 | CTCCAGTTGGATCATCAACCAAC | 63 |
| 742-764 | TCCAGTTGGATCATCAACCAACG | 64 |
| 785-807 | CAGCTGTTTCCTCCTTTGCCTTT | 65 |
| 786-808 | AGCTGTTTCCTCCTTTGCCTTTG | 66 |
| 792-814 | TTCCTCCTTTGCCTTTGAGAAGC | 67 |
| 801-823 | TGCCTTTGAGAAGCTCATCCAAT | 68 |
| 806-828 | TTGAGAAGCTCATCCAATTTAAG | 69 |
| 811-833 | AAGCTCATCCAATTTAAGTCTGT | 70 |
| 814-836 | CTCATCCAATTTAAGTCTGTAAT | 71 |
| 817-839 | ATCCAATTTAAGTCTGTAATACA | 72 |
| 861-883 | CAGCTTCTTCACCGGTGATGTAA | 73 |
| 862-884 | AGCTTCTTCACCGGTGATGTAAA | 74 |
| 872-894 | CCGGTGATGTAAACTACCTGTTT | 75 |
| 873-895 | CGGTGATGTAAACTACCTGTTTG | 76 |
| 889-911 | CTGTTTGAAGGGGTGTGCTATGG | 77 |
| 903-925 | GTGCTATGGACCCCTAGTACTGA | 78 |
| 938-960 | CGCTGGTCATCTGCAGCATTTCT | 79 |
| 940-962 | CTGGTCATCTGCAGCATTTCTTC | 80 |
| 941-963 | TGGTCATCTGCAGCATTTCTTCC | 81 |
| 948-970 | CTGCAGCATTTCTTCCTACTTCA | 82 |
| 951-973 | CAGCATTTCTTCCTACTTCATTA | 83 |
| 952-974 | AGCATTTCTTCCTACTTCATTAT | 84 |
| 960-982 | TTCCTACTTCATTATTGGATACA | 85 |
| 964-986 | TACTTCATTATTGGATACACTGC | 86 |
| 983-1005 | CTGCATTTATTGCCATCTTATGC | 87 |
| 993-1015 | TGCCATCTTATGCTATCTCCTGG | 88 |
| 1003-1025 | TGCTATCTCCTGGTTTTCCCACT | 89 |
| 1025-1047 | TGGCGGTATTCATGACAAGAATG | 90 |
| 1026-1048 | GGCGGTATTCATGACAAGAATGG | 91 |
| 1047-1069 | GGCTGTGAAGGCTCAGCATCACA | 92 |
| 1056-1078 | GGCTCAGCATCACACATCTGAGG | 93 |
| 1104-1126 | CAGTGAAGTTCTCACTTGCATTA | 94 |
| 1106-1128 | GTGAAGTTCTCACTTGCATTAAG | 95 |
| 1112-1134 | TTCTCACTTGCATTAAGCTGATT | 96 |
| 1114-1136 | CTCACTTGCATTAAGCTGATTAA | 97 |
| 1116-1138 | CACTTGCATTAAGCTGATTAAAA | 98 |
| 1119-1141 | TTGCATTAAGCTGATTAAAATGT | 99 |
| 1126-1148 | AAGCTGATTAAAATGTACACATG | 100 |
| 1127-1149 | AGCTGATTAAAATGTACACATGG | 101 |
| 1138-1160 | ATGTACACATGGGAGAAACCATT | 102 |
| 1146-1168 | ATGGGAGAAACCATTTGCAGAAA | 103 |
| 1147-1169 | TGGGAGAAACCATTTGCAGAAAT | 104 |
| 1148-1170 | GGGAGAAACCATTTGCAGAAATC | 105 |
| 1155-1177 | ACCATTTGCAGAAATCATTGAAG | 106 |
| 1163-1185 | CAGAAATCATTGAAGACCTAAGA | 107 |
| 1175-1197 | AAGACCTAAGAAGGAAGGAAAGG | 108 |
| 1178-1200 | ACCTAAGAAGGAAGGAAAGGAAA | 109 |
| 1182-1204 | AAGAAGGAAGGAAAGGAAACTAT | 110 |
| 1185-1207 | AAGGAAGGAAAGGAAACTATTGG | 111 |
| 1190-1212 | AGGAAAGGAAACTATTGGAGAAG | 112 |
| 1229-1251 | GCCTGACAAGTATAACCTTGTTC | 113 |
| 1233-1255 | GACAAGTATAACCTTGTTCATCA | 114 |
| 1236-1258 | AAGTATAACCTTGTTCATCATCC | 115 |
| 1280-1302 | GGGTTCTCATCCACACATCCTTA | 116 |
| 1289-1311 | TCCACACATCCTTAAAGCTGAAA | 117 |
| 1291-1313 | CACACATCCTTAAAGCTGAAACT | 118 |
| 1293-1315 | CACATCCTTAAAGCTGAAACTCA | 119 |
| 1297-1319 | TCCTTAAAGCTGAAACTCACAGC | 120 |
| 1316-1338 | CAGCGTCAATGGCCTTCAGCATG | 121 |
| 1317-1339 | AGCGTCAATGGCCTTCAGCATGC | 122 |
| 1332-1354 | CAGCATGCTGGCCTCCTTGAATC | 123 |
| 1369-1391 | GTGTTCTTTGTGCCTATTGCAGT | 124 |
| 1378-1400 | GTGCCTATTGCAGTCAAAGGTCT | 125 |
| 1380-1402 | GCCTATTGCAGTCAAAGGTCTCA | 126 |
| 1388-1410 | CAGTCAAAGGTCTCACGAATTCC | 127 |
| 1415-1437 | CTGCAGTGATGAGGTTCAAGAAG | 128 |
| 1416-1438 | TGCAGTGATGAGGTTCAAGAAGT | 129 |
| 1418-1440 | CAGTGATGAGGTTCAAGAAGTTT | 130 |
| 1420-1442 | GTGATGAGGTTCAAGAAGTTTTT | 131 |
| 1425-1447 | GAGGTTCAAGAAGTTTTTCCTCC | 132 |
| 1462-1484 | TTCTATGTCCAGACATTACAAGA | 133 |
| 1486-1508 | CCCAGCAAAGCTCTGGTCTTTGA | 134 |
| 1488-1510 | CAGCAAAGCTCTGGTCTTTGAGG | 135 |
| 1570-1592 | GAGAGGAACGGGCATGCTTCTGA | 136 |
| 1594-1616 | GGGATGACCAGGCCTAGAGATGC | 137 |
| 1649-1671 | GCCCAGAGTTGCACAAGATCAAC | 138 |
| 1650-1672 | CCCAGAGTTGCACAAGATCAACC | 139 |
| 1676-1698 | TGGTGTCCAAGGGGATGATGTTA | 140 |
| 1707-1729 | CGGCAACACGGGGAGTGGTAAGA | 141 |
| 1721-1743 | GTGGTAAGAGCAGCCTGTTGTCA | 142 |
| 1833-1855 | CGGGAACATCAGGGAGAACATCC | 143 |
| 1924-1946 | CTGGAACTTCTGCCCTTTGGAGA | 144 |
| 1933-1955 | CTGCCCTTTGGAGACATGACAGA | 145 |
| 1935-1957 | GCCCTTTGGAGACATGACAGAGA | 146 |
| 2089-2111 | CACATTTTTGAGGAGTGCATTAA | 147 |
| 2153-2175 | AGCTGCAGTACTTAGAATTTTGT | 148 |
| 2155-2177 | CTGCAGTACTTAGAATTTTGTGG | 149 |
| 2165-2187 | TAGAATTTTGTGGCCAGATCATT | 150 |
| 2175-2197 | TGGCCAGATCATTTTGTTGGAAA | 151 |
| 2176-2198 | GGCCAGATCATTTTGTTGGAAAA | 152 |
| 2177-2199 | GCCAGATCATTTTGTTGGAAAAT | 153 |
| 2179-2201 | CAGATCATTTTGTTGGAAAATGG | 154 |
| 2191-2213 | TTGGAAAATGGGAAAATCTGTGA | 155 |
| 2200-2222 | GGGAAAATCTGTGAAAATGGAAC | 156 |
| 2216-2238 | ATGGAACTCACAGTGAGTTAATG | 157 |
| 2217-2239 | TGGAACTCACAGTGAGTTAATGC | 158 |
| 2220-2242 | AACTCACAGTGAGTTAATGCAGA | 159 |
| 2222-2244 | CTCACAGTGAGTTAATGCAGAAA | 160 |
| 2224-2246 | CACAGTGAGTTAATGCAGAAAAA | 161 |
| 2226-2248 | CAGTGAGTTAATGCAGAAAAAGG | 162 |
| 2236-2258 | ATGCAGAAAAAGGGGAAATATGC | 163 |
| 2246-2268 | AGGGGAAATATGCCCAACTTATC | 164 |
| 2247-2269 | GGGGAAATATGCCCAACTTATCC | 165 |
| 2256-2278 | TGCCCAACTTATCCAGAAGATGC | 166 |
| 2266-2288 | ATCCAGAAGATGCACAAGGAAGC | 167 |
| 2305-2327 | CAGGACACAGCAAAGATAGCAGA | 168 |
| 2322-2344 | AGCAGAGAAGCCAAAGGTAGAAA | 169 |
| 2326-2348 | GAGAAGCCAAAGGTAGAAAGTCA | 170 |
| 2371-2393 | GAGTCTCTCAACGGAAATGCTGT | 171 |
| 2373-2395 | GTCTCTCAACGGAAATGCTGTGC | 172 |
| 2425-2447 | ATGGAAGAAGGCTCCTTGAGTTG | 173 |
| 2426-2448 | TGGAAGAAGGCTCCTTGAGTTGG | 174 |
| 2480-2502 | GAGGTTACATGGTCTCTTGCATA | 175 |
| 2481-2503 | AGGTTACATGGTCTCTTGCATAA | 176 |
| 2485-2507 | TACATGGTCTCTTGCATAATTTT | 177 |
| 2489-2511 | TGGTCTCTTGCATAATTTTCTTC | 178 |
| 2493-2515 | CTCTTGCATAATTTTCTTCTTCG | 179 |
| 2496-2518 | TTGCATAATTTTCTTCTTCGTGG | 180 |
| 2516-2538 | TGGTGCTGATCGTCTTCTTAACG | 181 |
| 2519-2541 | TGCTGATCGTCTTCTTAACGATC | 182 |
| 2525-2547 | TCGTCTTCTTAACGATCTTCAGC | 183 |
| 2629-2651 | GGCAACATTGCAGACAATCCTCA | 184 |
| 2632-2654 | AACATTGCAGACAATCCTCAACT | 185 |
| 2636-2658 | TTGCAGACAATCCTCAACTGTCC | 186 |
| 2646-2668 | TCCTCAACTGTCCTTCTACCAGC | 187 |
| 2720-2742 | CAGGGATTTTCACCAAGGTCACG | 188 |
| 2759-2781 | CCCTGCACAACAAGCTCTTTAAC | 189 |
| 2762-2784 | TGCACAACAAGCTCTTTAACAAG | 190 |
| 2767-2789 | AACAAGCTCTTTAACAAGGTTTT | 191 |
| 2795-2817 | GCCCCATGAGTTTCTTTGACACC | 192 |
| 2806-2828 | TTCTTTGACACCATCCCAATAGG | 193 |
| 2819-2841 | TCCCAATAGGCCGGCTTTTGAAC | 194 |
| 2820-2842 | CCCAATAGGCCGGCTTTTGAACT | 195 |
| 2870-2892 | ACCAGCTCTTGCCCATCTTTTCA | 196 |
| 2872-2894 | CAGCTCTTGCCCATCTTTTCAGA | 197 |
| 2950-2972 | CTGTCTCCATATATCCTGTTAAT | 198 |
| 2952-2974 | GTCTCCATATATCCTGTTAATGG | 199 |
| 2963-2985 | TCCTGTTAATGGGAGCCATAATC | 200 |
| 2973-2995 | GGGAGCCATAATCATGGTTATTT | 201 |
| 2975-2997 | GAGCCATAATCATGGTTATTTGC | 202 |
| 2983-3005 | ATCATGGTTATTTGCTTCATTTA | 203 |
| 2986-3008 | ATGGTTATTTGCTTCATTTATTA | 204 |
| 2987-3009 | TGGTTATTTGCTTCATTTATTAT | 205 |
| 2994-3016 | TTGCTTCATTTATTATATGATGT | 206 |
| 3037-3059 | TTCAAGAGACTGGAGAACTATAG | 207 |
| 3052-3074 | AACTATAGCCGGTCTCCTTTATT | 208 |
| 3066-3088 | TCCTTTATTCTCCCACATCCTCA | 209 |
| 3075-3097 | CTCCCACATCCTCAATTCTCTGC | 210 |
| 3108-3130 | CTCCATCCATGTCTATGGAAAAA | 211 |
| 3109-3131 | TCCATCCATGTCTATGGAAAAAC | 212 |
| 3112-3134 | ATCCATGTCTATGGAAAAACTGA | 213 |
| 3122-3144 | ATGGAAAAACTGAAGACTTCATC | 214 |
| 3123-3145 | TGGAAAAACTGAAGACTTCATCA | 215 |
| 3134-3156 | AAGACTTCATCAGCCAGTTTAAG | 216 |
| 3148-3170 | CAGTTTAAGAGGCTGACTGATGC | 217 |
| 3158-3180 | GGCTGACTGATGCGCAGAATAAC | 218 |
| 3182-3204 | ACCTGCTGTTGTTTCTATCTTCC | 219 |
| 3185-3207 | TGCTGTTGTTTCTATCTTCCACA | 220 |
| 3187-3209 | CTGTTGTTTCTATCTTCCACACG | 221 |
| 3216-3238 | GGCATTGAGGCTGGAGATCATGA | 222 |
| 3263-3285 | CCCTGTTCGTGGCTTTTGGCATT | 223 |
| 3269-3291 | TCGTGGCTTTTGGCATTTCCTCC | 224 |
| 3293-3315 | CCCCCTACTCCTTTAAAGTCATG | 225 |
| 3294-3316 | CCCCTACTCCTTTAAAGTCATGG | 226 |
| 3374-3396 | TGGAGACAGAGGCACAGTTCACG | 227 |
| 3384-3406 | GGCACAGTTCACGGCTGTAGAGA | 228 |
| 3386-3408 | CACAGTTCACGGCTGTAGAGAGG | 229 |
| 3396-3418 | GGCTGTAGAGAGGATACTGCAGT | 230 |
| 3405-3427 | GAGGATACTGCAGTACATGAAGA | 231 |
| 3410-3432 | TACTGCAGTACATGAAGATGTGT | 232 |
| 3412-3434 | CTGCAGTACATGAAGATGTGTGT | 234 |
| 3449-3471 | TACACATGGAAGGCACAAGTTGT | 235 |
| 3451-3473 | CACATGGAAGGCACAAGTIGTCC | 236 |
| 3483-3505 | GCCACAGCATGGGGAAATCATAT | 237 |
| 3492-3514 | TGGGGAAATCATATTTCAGGATT | 238 |
| 3493-3515 | GGGGAAATCATATTTCAGGATTA | 239 |
| 3494-3516 | GGGAAATCATATTTCAGGATTAT | 240 |
| 3506-3528 | TTCAGGATTATCACATGAAATAC | 241 |
| 3509-3531 | AGGATTATCACATGAAATACAGA | 242 |
| 3515-3537 | ATCACATGAAATACAGAGACAAC | 243 |
| 3520-3542 | ATGAAATACAGAGACAACACACC | 244 |
| 3676-3698 | CTCATTGACGGCGTGGACATTTG | 245 |
| 3713-3735 | AGGACTTGCGGTCCAAGCTCTCA | 246 |
| 3720-3742 | GCGGTCCAAGCTCTCAGTGATCC | 247 |
| 3730-3752 | CTCTCAGTGATCCCTCAAGATCC | 248 |
| 3757-3779 | CTGCTCTCAGGAACCATCAGATT | 249 |
| 3765-3787 | AGGAACCATCAGATTCAACCTAG | 250 |
| 3768-3790 | AACCATCAGATTCAACCTAGATC | 251 |
| 3769-3791 | ACCATCAGATTCAACCTAGATCC | 252 |
| 3789-3811 | TCCCTTTGACCGTCACACTGACC | 253 |
| 3825-3847 | TGCCTTGGAGAGGACATTCCTGA | 254 |
| 3842-3864 | TCCTGACCAAGGCCATCTCAAAG | 255 |
| 3858-3880 | CTCAAAGTTCCCCAAAAAGCTGC | 256 |
| 3865-3887 | TTCCCCAAAAAGCTGCATACAGA | 257 |
| 3867-3889 | CCCCAAAAAGCTGCATACAGATG | 258 |
| 3868-3890 | CCCAAAAAGCTGCATACAGATGT | 259 |
| 3890-3912 | TGGTGGAAAACGGTGGAAACTTC | 260 |
| 3893-3915 | TGGAAAACGGTGGAAACTTCTCT | 261 |
| 3948-3970 | GGCTGTGCTTCGCAACTCCAAGA | 262 |
| 3953-3975 | TGCTTCGCAACTCCAAGATCATC | 263 |
| 3957-3979 | TCGCAACTCCAAGATCATCCTTA | 264 |
| 3958-3980 | CGCAACTCCAAGATCATCCTTAT | 265 |
| 3963-3985 | CTCCAAGATCATCCTTATCGATG | 266 |
| 3964-3986 | TCCAAGATCATCCTTATCGATGA | 267 |
| 3967-3989 | AAGATCATCCTTATCGATGAAGC | 268 |
| 3996-4018 | CTCCATTGACATGGAGACAGACA | 269 |
| 4086-4108 | CACCACTGTGCTGAACTGTGACC | 270 |
| 4112-4134 | TCCTGGTTATGGGCAATGGGAAG | 271 |
| 4122-4144 | GGGCAATGGGAAGGTGGTAGAAT | 272 |
| 4123-4145 | GGCAATGGGAAGGTGGTAGAATT | 273 |
| 4128-4150 | TGGGAAGGTGGTAGAATTTGATC | 274 |
| 4205-4227 | CAGCCACTTCTTCACTGAGATAA | 275 |
| 4206-4228 | AGCCACTTCTTCACTGAGATAAG | 276 |
| 4207-4229 | GCCACTTCTTCACTGAGATAAGG | 277 |
| 4212-4234 | TTCTTCACTGAGATAAGGAGATG | 278 |
| 4215-4237 | TTCACTGAGATAAGGAGATGTGG | 279 |
| 4226-4248 | AAGGAGATGTGGAGACTTCATGG | 280 |
| 4229-4251 | GAGATGTGGAGACTTCATGGAGG | 281 |
| 4284-4306 | CAGCTTCGAGGCCCACAGTCTGC | 282 |
| 4295-4317 | CCCACAGTCTGCGACCTTCTTGT | 283 |
| 4305-4327 | GCGACCTTCTTGTTTGGAGATGA | 284 |
| 4307-4329 | GACCTTCTTGTTTGGAGATGAGA | 285 |
| 4318-4340 | TTGGAGATGAGAACTTCTCCTGG | 286 |
| 4334-4356 | CTCCTGGAAGCAGGGGTAAATGT | 287 |
| 4337-4359 | CTGGAAGCAGGGGTAAATGTAGG | 289 |
| 4364-4386 | GTGGGGATTGCTGGATGGAAACC | 290 |
| 4374-4396 | CTGGATGGAAACCCTGGAATAGG | 291 |
| 4379-4401 | TGGAAACCCTGGAATAGGCTACT | 292 |
| 4384-4406 | ACCCTGGAATAGGCTACTTGATG | 293 |
| 4385-4407 | CCCTGGAATAGGCTACTTGATGG | 294 |
| 4415-4437 | GACCTTAGAACCCCAGAACCATC | 295 |
| 4416-4438 | ACCTTAGAACCCCAGAACCATCT | 296 |
| 4424-4446 | ACCCCAGAACCATCTAAGACATG | 297 |
| 4425-4447 | CCCCAGAACCATCTAAGACATGG | 298 |
| 4431-4453 | AACCATCTAAGACATGGGATTCA | 299 |
| 4435-4457 | ATCTAAGACATGGGATTCAGTGA | 300 |
| 4441-4463 | GACATGGGATTCAGTGATCATGT | 301 |
| 4446-4468 | GGGATTCAGTGATCATGTGGTTC | 302 |
| 4454-4476 | GTGATCATGTGGTTCTCCTTTTA | 303 |
| 4457-4479 | ATCATGTGGTTCTCCTTTTAACT | 304 |
| 4460-4482 | ATGTGGTTCTCCTTTTAACTTAC | 305 |
| 4463-4485 | TGGTTCTCCTTTTAACTTACATG | 306 |
| 4469-4491 | TCCTTTTAACTTACATGCTGAAT | 307 |
| 4476-4498 | AACTTACATGCTGAATAATTTTA | 308 |
| 4480-4502 | TACATGCTGAATAATTTTATAAT | 309 |
| 4483-4505 | ATGCTGAATAATTTTATAATAAG | 310 |
| 4484-4506 | TGCTGAATAATTTTATAATAAGG | 311 |
| 4503-4525 | AAGGTAAAAGCTTATAGTTTTCT | 312 |
| 4510-4532 | AAGCTTATAGTTTTCTGATCTGT | 313 |
| 4524-4546 | CTGATCTGTGTTAGAAGTGTTGC | 314 |
| 4529-4551 | CTGTGTTAGAAGTGTTGCAAATG | 315 |
| 4535-4557 | TAGAAGTGTTGCAAATGCTGTAC | 316 |
| 4540-4562 | GTGTTGCAAATGCTGTACTGACT | 317 |
| 4543-4565 | TTGCAAATGCTGTACTGACTTTG | 318 |
| 4544-4566 | TGCAAATGCTGTACTGACTTTGT | 319 |
| 4549-4571 | ATGCTGTACTGACTTTGTAAAAT | 320 |
| 4550-4572 | TGCTGTACTGACTTTGTAAAATA | 321 |
| 4552-4574 | CTGTACTGACTTTGTAAAATATA | 322 |
| 4555-4577 | TACTGACTTTGTAAAATATAAAA | 323 |
| 4557-4579 | CTGACTTTGTAAAATATAAAACT | 324 |
| 4559-4581 | GACTTTGTAAAATATAAAACTAA | 325 |

As described above, in some examples, one or more of SEQ ID NOs: 2-325, or portions thereof, can be targeted to inhibit expression of the ABCC11 gene. In yet other examples, two or more of SEQ ID NOs: 2-325, or portions thereof, can be targeted to inhibit expression of the ABCC11 gene. In still other examples, three or more, four or more, five or more, or ten or more of SEQ ID NOs: 2-325, or portions thereof, can be targeted to inhibit expression of the ABCC11 gene. In some examples, each of SEQ ID NOs: 2-325, or portions thereof, can be targeted to inhibit expression of the ABCC11 gene.

In some examples, SEQ ID NO: 2, or a portion thereof, can be targeted. In some examples, SEQ ID NO: 3, or a portion thereof, can be targeted. In some examples, SEQ ID NO: 4, or a portion thereof, can be targeted. In some examples, SEQ ID NO: 5, or a portion thereof, can be targeted. In some examples, SEQ ID NO: 6, or a portion thereof, can be targeted. In some examples, SEQ ID NO: 7, or a portion thereof, can be targeted. In some examples, SEQ ID NO: 8, or a portion thereof, can be targeted. In some examples, SEQ ID NO: 9, or a portion thereof, can be targeted. In some examples, SEQ ID NO: 10, or a portion thereof, can be targeted. In some examples, SEQ ID NO: 11, or a portion thereof, can be targeted. In some examples, SEQ ID NO: 12, or a portion thereof, can be targeted. In some examples, SEQ ID NO: 13, or a portion thereof, can be targeted. In some examples, SEQ ID NO: 14, or a portion thereof, can be targeted. In some examples, SEQ ID NO: 15, or a portion thereof, can be targeted. In some examples, SEQ ID NO: 16, or a portion thereof, can be targeted. In some examples, SEQ ID NO: 17, or a portion thereof, can be targeted. In some examples, SEQ ID NO: 18, or a portion thereof, can be targeted. In some examples, SEQ ID NO: 19, or a portion thereof, can be targeted. In some examples, SEQ ID NO: 20, or a portion thereof, can be targeted. In some examples, SEQ ID NO: 21, or a portion thereof, can be targeted. In some examples, SEQ ID NO: 22, or a portion thereof, can be targeted. In some examples, SEQ ID NO: 23, or a portion thereof, can be targeted. In some examples, SEQ ID NO: 24, or a portion thereof, can be targeted. In some examples, SEQ ID NO: 25, or a portion thereof, can be targeted. In some examples, SEQ ID NO: 26, or a portion thereof, can be targeted. In some examples, SEQ ID NO: 27, or a portion thereof, can be targeted. In some examples, SEQ ID NO: 28, or a portion thereof, can be targeted. In some examples, SEQ ID NO: 29, or a portion thereof, can be targeted. In some examples, SEQ ID NO: 30, or a portion thereof, can be targeted. In some examples, SEQ ID NO: 31, or a portion thereof, can be targeted. In some examples, SEQ ID NO: 32, or a portion thereof, can be targeted. In some examples, SEQ ID NO: 33, or a portion thereof, can be targeted. In some examples, SEQ ID NO: 34, or a portion thereof, can be targeted. In some examples, SEQ ID NO: 35, or a portion thereof, can be targeted. In some examples, SEQ ID NO: 36, or a portion thereof, can be targeted. In some examples, SEQ ID NO: 37, or a portion thereof, can be targeted. In some examples, SEQ ID NO: 38, or a portion thereof, can be targeted. In some examples, SEQ ID NO: 39, or a portion thereof, can be targeted. In some examples, SEQ ID NO: 40, or a portion thereof, can be targeted. In some examples, SEQ ID NO: 41, or a portion thereof, can be targeted. In some examples, SEQ ID NO: 42, or a portion thereof, can be targeted. In some examples, SEQ ID NO: 43, or a portion thereof, can be targeted. In some examples, SEQ ID NO: 44, or a portion thereof, can be targeted. In some examples, SEQ ID NO: 45, or a portion thereof, can be targeted. In some examples, SEQ ID NO: 46, or a portion thereof, can be targeted. In some examples, SEQ ID NO: 47, or a portion thereof, can be targeted. In some examples, SEQ ID NO: 48, or a portion thereof, can be targeted. In some examples, SEQ ID NO: 49, or a portion thereof, can be targeted. In some examples, SEQ ID NO: 50, or a portion thereof, can be targeted. In some examples, SEQ ID NO: 51, or a portion thereof, can be targeted. In some examples, SEQ ID NO: 52, or a portion thereof, can be targeted. In some examples, SEQ ID NO: 53, or a portion thereof, can be targeted. In some examples, SEQ ID NO: 54, or a portion thereof, can be targeted. In some examples, SEQ ID NO: 55, or a portion thereof, can be targeted. In some examples, SEQ ID NO: 56, or a portion thereof, can be targeted. In some examples, SEQ ID NO: 57, or a portion thereof, can be targeted. In some examples, SEQ ID NO: 58, or a portion thereof, can be targeted. In some examples, SEQ ID NO: 59, or a portion thereof, can be targeted. In some examples, SEQ ID NO: 60, or a portion thereof, can be targeted. In some examples, SEQ ID NO: 61, or a portion thereof, can be targeted. In some examples, SEQ ID NO: 62, or a portion thereof, can be targeted. In some examples, SEQ ID NO: 63, or a portion thereof, can be targeted. In some examples, SEQ ID NO: 64, or a portion thereof, can be targeted. In some examples, SEQ ID NO: 65, or a portion thereof, can be targeted. In some examples, SEQ ID NO: 66, or a portion thereof, can be targeted. In some examples, SEQ ID NO: 67, or a portion thereof, can be targeted. In some examples, SEQ ID NO: 68, or a portion thereof, can be targeted. In some examples, SEQ ID NO: 69, or a portion thereof, can be targeted. In some examples, SEQ ID NO: 70, or a portion thereof, can be targeted. In some examples, SEQ ID NO: 71, or a portion thereof, can be targeted. In some examples, SEQ ID NO: 72, or a portion thereof, can be targeted. In some examples, SEQ ID NO: 73, or a portion thereof, can be targeted. In some examples, SEQ ID NO: 74, or a portion thereof, can be targeted. In some examples, SEQ ID NO: 75, or a portion thereof, can be targeted. In some examples, SEQ ID NO: 76, or a portion thereof, can be targeted. In some examples, SEQ ID NO: 77, or a portion thereof, can be targeted. In some examples, SEQ ID NO: 78, or a portion thereof, can be targeted. In some examples, SEQ ID NO: 79, or a portion thereof, can be targeted. In some examples, SEQ ID NO: 80, or a portion thereof, can be targeted. In some examples, SEQ ID NO: 81, or a portion thereof, can be targeted. In some examples, SEQ ID NO: 82, or a portion thereof, can be targeted. In some examples, SEQ ID NO: 83, or a portion thereof, can be targeted. In some examples, SEQ ID NO: 84, or a portion thereof, can be targeted. In some examples, SEQ ID NO: 85, or a portion thereof, can be targeted. In some examples, SEQ ID NO: 86, or a portion thereof, can be targeted. In some examples, SEQ ID NO: 87, or a portion thereof, can be targeted. In some examples, SEQ ID NO: 88, or a portion thereof, can be targeted. In some examples, SEQ ID NO: 89, or a portion thereof, can be targeted. In some examples, SEQ ID NO: 90, or a portion thereof, can be targeted. In some examples, SEQ ID NO: 91, or a portion thereof, can be targeted. In some examples, SEQ ID NO: 92, or a portion thereof, can be targeted. In some examples, SEQ ID NO: 93, or a portion thereof, can be targeted. In some examples, SEQ ID NO: 94, or a portion thereof, can be targeted. In some examples, SEQ ID NO: 95, or a portion thereof, can be targeted. In some examples, SEQ ID NO: 96, or a portion thereof, can be targeted. In some examples, SEQ ID NO: 97, or a portion thereof, can be targeted. In some examples, SEQ ID NO: 98, or a portion thereof, can be targeted. In some examples, SEQ ID NO: 99, or a portion thereof, can be targeted. In some examples, SEQ ID NO: 100, or a portion thereof, can be targeted. In some examples, SEQ ID NO: 101, or a portion thereof, can be targeted. In some examples, SEQ ID NO: 102, or a portion thereof, can be targeted. In some examples, SEQ ID NO: 103, or a portion thereof, can be targeted. In some examples, SEQ ID NO: 104, or a portion thereof, can be targeted. In some examples, SEQ ID NO: 105, or a portion thereof, can be targeted. In some examples, SEQ ID NO: 106, or a portion thereof, can be targeted. In some examples, SEQ ID NO: 107, or a portion thereof, can be targeted. In some examples, SEQ ID NO: 108, or a portion thereof, can be targeted. In some examples, SEQ ID NO: 109, or a portion thereof, can be targeted. In some examples, SEQ ID NO: 110, or a portion thereof, can be targeted. In some examples, SEQ ID NO: 111, or a portion thereof, can be targeted. In some examples, SEQ ID NO: 112, or a portion thereof, can be targeted. In some examples, SEQ ID NO: 113, or a portion thereof, can be targeted. In some examples, SEQ ID NO: 114, or a portion thereof, can be targeted. In some examples, SEQ ID NO: 115, or a portion thereof, can be targeted. In some examples, SEQ ID NO: 116, or a portion thereof, can be targeted. In some examples, SEQ ID NO: 117, or a portion thereof, can be targeted. In some examples, SEQ ID NO: 118, or a portion thereof, can be targeted. In some examples, SEQ ID NO: 119, or a portion thereof, can be targeted. In some examples, SEQ ID NO: 120, or a portion thereof, can be targeted. In some examples, SEQ ID NO: 121, or a portion thereof, can be targeted. In some examples, SEQ ID NO: 122, or a portion thereof, can be targeted. In some examples, SEQ ID NO: 123, or a portion thereof, can be targeted. In some examples, SEQ ID NO: 124, or a portion thereof, can be targeted. In some examples, SEQ ID NO: 125, or a portion thereof, can be targeted. In some examples, SEQ ID NO: 126, or a portion thereof, can be targeted. In some examples, SEQ ID NO: 127, or a portion thereof, can be targeted. In some examples, SEQ ID NO: 128, or a portion thereof, can be targeted. In some examples, SEQ ID NO: 129, or a portion thereof, can be targeted. In some examples, SEQ ID NO: 130, or a portion thereof, can be targeted. In some examples, SEQ ID NO: 131, or a portion thereof, can be targeted. In some examples, SEQ ID NO: 132, or a portion thereof, can be targeted. In some examples, SEQ ID NO: 133, or a portion thereof, can be targeted. In some examples, SEQ ID NO: 134, or a portion thereof, can be targeted. In some examples, SEQ ID NO: 135, or a portion thereof, can be targeted. In some examples, SEQ ID NO: 136, or a portion thereof, can be targeted. In some examples, SEQ ID NO: 137, or a portion thereof, can be targeted. In some examples, SEQ ID NO: 138, or a portion thereof, can be targeted. In some examples, SEQ ID NO: 139, or a portion thereof, can be targeted. In some examples, SEQ ID NO: 140, or a portion thereof, can be targeted. In some examples, SEQ ID NO: 141, or a portion thereof, can be targeted. In some examples, SEQ ID NO: 142, or a portion thereof, can be targeted. In some examples, SEQ ID NO: 143, or a portion thereof, can be targeted. In some examples, SEQ ID NO: 144, or a portion thereof, can be targeted. In some examples, SEQ ID NO: 145, or a portion thereof, can be targeted. In some examples, SEQ ID NO: 146, or a portion thereof, can be targeted. In some examples, SEQ ID NO: 147, or a portion thereof, can be targeted. In some examples, SEQ ID NO: 148, or a portion thereof, can be targeted. In some examples, SEQ ID NO: 149, or a portion thereof, can be targeted. In some examples, SEQ ID NO: 150, or a portion thereof, can be targeted. In some examples, SEQ ID NO: 151, or a portion thereof, can be targeted. In some examples, SEQ ID NO: 152, or a portion thereof, can be targeted. In some examples, SEQ ID NO: 153, or a portion thereof, can be targeted. In some examples, SEQ ID NO: 154, or a portion thereof, can be targeted. In some examples, SEQ ID NO: 155, or a portion thereof, can be targeted. In some examples, SEQ ID NO: 156, or a portion thereof, can be targeted. In some examples, SEQ ID NO: 157, or a portion thereof, can be targeted. In some examples, SEQ ID NO: 158, or a portion thereof, can be targeted. In some examples, SEQ ID NO: 159, or a portion thereof, can be targeted. In some examples, SEQ ID NO: 160, or a portion thereof, can be targeted. In some examples, SEQ ID NO: 161, or a portion thereof, can be targeted. In some examples, SEQ ID NO: 162, or a portion thereof, can be targeted. In some examples, SEQ ID NO: 163, or a portion thereof, can be targeted. In some examples, SEQ ID NO: 164, or a portion thereof, can be targeted. In some examples, SEQ ID NO: 165, or a portion thereof, can be targeted. In some examples, SEQ ID NO: 166, or a portion thereof, can be targeted. In some examples, SEQ ID NO: 167, or a portion thereof, can be targeted. In some examples, SEQ ID NO: 168, or a portion thereof, can be targeted. In some examples, SEQ ID NO: 169, or a portion thereof, can be targeted. In some examples, SEQ ID NO: 170, or a portion thereof, can be targeted. In some examples, SEQ ID NO: 171, or a portion thereof, can be targeted. In some examples, SEQ ID NO: 172, or a portion thereof, can be targeted. In some examples, SEQ ID NO: 173, or a portion thereof, can be targeted. In some examples, SEQ ID NO: 174, or a portion thereof, can be targeted. In some examples, SEQ ID NO: 175, or a portion thereof, can be targeted. In some examples, SEQ ID NO: 176, or a portion thereof, can be targeted. In some examples, SEQ ID NO: 177, or a portion thereof, can be targeted. In some examples, SEQ ID NO: 178, or a portion thereof, can be targeted. In some examples, SEQ ID NO: 179, or a portion thereof, can be targeted. In some examples, SEQ ID NO: 180, or a portion thereof, can be targeted. In some examples, SEQ ID NO: 181, or a portion thereof, can be targeted. In some examples, SEQ ID NO: 182, or a portion thereof, can be targeted. In some examples, SEQ ID NO: 183, or a portion thereof, can be targeted. In some examples, SEQ ID NO: 184, or a portion thereof, can be targeted. In some examples, SEQ ID NO: 185, or a portion thereof, can be targeted. In some examples, SEQ ID NO: 186, or a portion thereof, can be targeted. In some examples, SEQ ID NO: 187, or a portion thereof, can be targeted. In some examples, SEQ ID NO: 188, or a portion thereof, can be targeted. In some examples, SEQ ID NO: 189, or a portion thereof, can be targeted. In some examples, SEQ ID NO: 190, or a portion thereof, can be targeted. In some examples, SEQ ID NO: 191, or a portion thereof, can be targeted. In some examples, SEQ ID NO: 192, or a portion thereof, can be targeted. In some examples, SEQ ID NO: 193, or a portion thereof, can be targeted. In some examples, SEQ ID NO: 194, or a portion thereof, can be targeted. In some examples, SEQ ID NO: 195, or a portion thereof, can be targeted. In some examples, SEQ ID NO: 196, or a portion thereof, can be targeted. In some examples, SEQ ID NO: 197, or a portion thereof, can be targeted. In some examples, SEQ ID NO: 198, or a portion thereof, can be targeted. In some examples, SEQ ID NO: 199, or a portion thereof, can be targeted. In some examples, SEQ ID NO: 200, or a portion thereof, can be targeted. In some examples, SEQ ID NO: 201, or a portion thereof, can be targeted. In some examples, SEQ ID NO: 202, or a portion thereof, can be targeted. In some examples, SEQ ID NO: 203, or a portion thereof, can be targeted. In some examples, SEQ ID NO: 204, or a portion thereof, can be targeted. In some examples, SEQ ID NO: 205, or a portion thereof, can be targeted. In some examples, SEQ ID NO: 206, or a portion thereof, can be targeted. In some examples, SEQ ID NO: 207, or a portion thereof, can be targeted. In some examples, SEQ ID NO: 208, or a portion thereof, can be targeted. In some examples, SEQ ID NO: 209, or a portion thereof, can be targeted. In some examples, SEQ ID NO: 210, or a portion thereof, can be targeted. In some examples, SEQ ID NO: 211, or a portion thereof, can be targeted. In some examples, SEQ ID NO: 212, or a portion thereof, can be targeted. In some examples, SEQ ID NO: 213, or a portion thereof, can be targeted. In some examples, SEQ ID NO: 214, or a portion thereof, can be targeted. In some examples, SEQ ID NO: 215, or a portion thereof, can be targeted. In some examples, SEQ ID NO: 216, or a portion thereof, can be targeted. In some examples, SEQ ID NO: 217, or a portion thereof, can be targeted. In some examples, SEQ ID NO: 218, or a portion thereof, can be targeted. In some examples, SEQ ID NO: 219, or a portion thereof, can be targeted. In some examples, SEQ ID NO: 220, or a portion thereof, can be targeted. In some examples, SEQ ID NO: 221, or a portion thereof, can be targeted. In some examples, SEQ ID NO: 222, or a portion thereof, can be targeted. In some examples, SEQ ID NO: 223, or a portion thereof, can be targeted. In some examples, SEQ ID NO: 224, or a portion thereof, can be targeted. In some examples, SEQ ID NO: 225, or a portion thereof, can be targeted. In some examples, SEQ ID NO: 226, or a portion thereof, can be targeted. In some examples, SEQ ID NO: 227, or a portion thereof, can be targeted. In some examples, SEQ ID NO: 228, or a portion thereof, can be targeted. In some examples, SEQ ID NO: 229, or a portion thereof, can be targeted. In some examples, SEQ ID NO: 230, or a portion thereof, can be targeted. In some examples, SEQ ID NO: 231, or a portion thereof, can be targeted. In some examples, SEQ ID NO: 232, or a portion thereof, can be targeted. In some examples, SEQ ID NO: 234, or a portion thereof, can be targeted. In some examples, SEQ ID NO: 235, or a portion thereof, can be targeted. In some examples, SEQ ID NO: 236, or a portion thereof, can be targeted. In some examples, SEQ ID NO: 237, or a portion thereof, can be targeted. In some examples, SEQ ID NO: 238, or a portion thereof, can be targeted. In some examples, SEQ ID NO: 239, or a portion thereof, can be targeted. In some examples, SEQ ID NO: 240, or a portion thereof, can be targeted. In some examples, SEQ ID NO: 241, or a portion thereof, can be targeted. In some examples, SEQ ID NO: 242, or a portion thereof, can be targeted. In some examples, SEQ ID NO: 243, or a portion thereof, can be targeted. In some examples, SEQ ID NO: 244, or a portion thereof, can be targeted. In some examples, SEQ ID NO: 245, or a portion thereof, can be targeted. In some examples, SEQ ID NO: 246, or a portion thereof, can be targeted. In some examples, SEQ ID NO: 247, or a portion thereof, can be targeted. In some examples, SEQ ID NO: 248, or a portion thereof, can be targeted. In some examples, SEQ ID NO: 249, or a portion thereof, can be targeted. In some examples, SEQ ID NO: 250, or a portion thereof, can be targeted. In some examples, SEQ ID NO: 251, or a portion thereof, can be targeted. In some examples, SEQ ID NO: 252, or a portion thereof, can be targeted. In some examples, SEQ ID NO: 253, or a portion thereof, can be targeted. In some examples, SEQ ID NO: 254, or a portion thereof, can be targeted. In some examples, SEQ ID NO: 255, or a portion thereof, can be targeted. In some examples, SEQ ID NO: 256, or a portion thereof, can be targeted. In some examples, SEQ ID NO: 257, or a portion thereof, can be targeted. In some examples, SEQ ID NO: 258, or a portion thereof, can be targeted. In some examples, SEQ ID NO: 259, or a portion thereof, can be targeted. In some examples, SEQ ID NO: 260, or a portion thereof, can be targeted. In some examples, SEQ ID NO: 261, or a portion thereof, can be targeted. In some examples, SEQ ID NO: 262, or a portion thereof, can be targeted. In some examples, SEQ ID NO: 263, or a portion thereof, can be targeted. In some examples, SEQ ID NO: 264, or a portion thereof, can be targeted. In some examples, SEQ ID NO: 265, or a portion thereof, can be targeted. In some examples, SEQ ID NO: 266, or a portion thereof, can be targeted. In some examples, SEQ ID NO: 267, or a portion thereof, can be targeted. In some examples, SEQ ID NO: 268, or a portion thereof, can be targeted. In some examples, SEQ ID NO: 269, or a portion thereof, can be targeted. In some examples, SEQ ID NO: 270, or a portion thereof, can be targeted. In some examples, SEQ ID NO: 271, or a portion thereof, can be targeted. In some examples, SEQ ID NO: 272, or a portion thereof, can be targeted. In some examples, SEQ ID NO: 273, or a portion thereof, can be targeted. In some examples, SEQ ID NO: 274, or a portion thereof, can be targeted. In some examples, SEQ ID NO: 275, or a portion thereof, can be targeted. In some examples, SEQ ID NO: 276, or a portion thereof, can be targeted. In some examples, SEQ ID NO: 277, or a portion thereof, can be targeted. In some examples, SEQ ID NO: 278, or a portion thereof, can be targeted. In some examples, SEQ ID NO: 279, or a portion thereof, can be targeted. In some examples, SEQ ID NO: 280, or a portion thereof, can be targeted. In some examples, SEQ ID NO: 281, or a portion thereof, can be targeted. In some examples, SEQ ID NO: 282, or a portion thereof, can be targeted. In some examples, SEQ ID NO: 283, or a portion thereof, can be targeted. In some examples, SEQ ID NO: 284, or a portion thereof, can be targeted. In some examples, SEQ ID NO: 285, or a portion thereof, can be targeted. In some examples, SEQ ID NO: 286, or a portion thereof, can be targeted. In some examples, SEQ ID NO: 287, or a portion thereof, can be targeted. In some examples, SEQ ID NO: 289, or a portion thereof, can be targeted. In some examples, SEQ ID NO: 290, or a portion thereof, can be targeted. In some examples, SEQ ID NO: 291, or a portion thereof, can be targeted. In some examples, SEQ ID NO: 292, or a portion thereof, can be targeted. In some examples, SEQ ID NO: 293, or a portion thereof, can be targeted. In some examples, SEQ ID NO: 294, or a portion thereof, can be targeted. In some examples, SEQ ID NO: 295, or a portion thereof, can be targeted. In some examples, SEQ ID NO: 296, or a portion thereof, can be targeted. In some examples, SEQ ID NO: 297, or a portion thereof, can be targeted. In some examples, SEQ ID NO: 298, or a portion thereof, can be targeted. In some examples, SEQ ID NO: 299, or a portion thereof, can be targeted. In some examples, SEQ ID NO: 300, or a portion thereof, can be targeted. In some examples, SEQ ID NO: 301, or a portion thereof, can be targeted. In some examples, SEQ ID NO: 302, or a portion thereof, can be targeted. In some examples, SEQ ID NO: 303, or a portion thereof, can be targeted. In some examples, SEQ ID NO: 304, or a portion thereof, can be targeted. In some examples, SEQ ID NO: 305, or a portion thereof, can be targeted. In some examples, SEQ ID NO: 306, or a portion thereof, can be targeted. In some examples, SEQ ID NO: 307, or a portion thereof, can be targeted. In some examples, SEQ ID NO: 308, or a portion thereof, can be targeted. In some examples, SEQ ID NO: 309, or a portion thereof, can be targeted. In some examples, SEQ ID NO: 310, or a portion thereof, can be targeted. In some examples, SEQ ID NO: 311, or a portion thereof, can be targeted. In some examples, SEQ ID NO: 312, or a portion thereof, can be targeted. In some examples, SEQ ID NO: 313, or a portion thereof, can be targeted. In some examples, SEQ ID NO: 314, or a portion thereof, can be targeted. In some examples, SEQ ID NO: 315, or a portion thereof, can be targeted. In some examples, SEQ ID NO: 316, or a portion thereof, can be targeted. In some examples, SEQ ID NO: 317, or a portion thereof, can be targeted. In some examples, SEQ ID NO: 318, or a portion thereof, can be targeted. In some examples, SEQ ID NO: 319, or a portion thereof, can be targeted. In some examples, SEQ ID NO: 320, or a portion thereof, can be targeted. In some examples, SEQ ID NO: 321, or a portion thereof, can be targeted. In some examples, SEQ ID NO: 322, or a portion thereof, can be targeted. In some examples, SEQ ID NO: 323, or a portion thereof, can be targeted. In some examples, SEQ ID NO: 324, or a portion thereof, can be targeted. In some examples, SEQ ID NO: 325, or a portion thereof, can be targeted.

As described above, ABCC11 inhibitors are a potential class of pharmaceutically active agents that can be useful in treating a variety of conditions or symptoms. An example of such a symptom is osmidrosis. ABCC11 inhibitors can be administered in a variety of ways, including, but not limited to, oral, topical, intravenous, intrathecal, intradermal, and transdermal administration. Therefore, ABCC11 inhibitors can be used to treat osmidrosis symptoms both systemically and in targeted regions or areas of a subject's body.

For example, a subject may experience osmidrosis due to expression of the wildtype ABCC11 gene. Accordingly, an ABCC11 inhibitor can be administered as a first line of treatment to reduce odor. When odor is manifested in the skin, it may be desirable to apply treatment directly to the situs afflicted with these symptoms. For example, the situs can include the axillary region (e.g. armpits), the pectoral region (e.g. chest/breasts), or the genital region.

Some non-limiting examples of inhibitors or therapeutic agents can be those used for gene therapy. For example, in some cases, CRISPR-Cas9 systems can be employed. For example, by delivering a Cas9 nuclease complexed with a synthetic guide RNA into a cell, the cell's genome can be cut as a desired location, allowing existing genes to be removed and/or altered genes to be added. Thus, in some examples, a CRISPR-Cas9 system can be administered to an individual having a GG or GA genotype to remove this particular version of the ABCC11 gene and replace it with a version that includes the SNP version (538G→A, Gly180Arg, rs17822931) of the gene. In other examples, a therapeutic nucleotide including the rs17822931 SNP can be introduced into a target cell via a viral vector or via non-viral methods. Where viral vectors are used, any suitable viral vector can be employed. Non-limiting examples can include adenovirus, adeno-associated virus, retrovirus, lentivirus, herpes simplex, vaccinia, the like, or combinations thereof. Additionally, any suitable non-viral method can additionally or alternatively be employed. Non-limiting examples of non-viral methods can include electroporation, iontophoresis sonoporation, magnetofection, use of carriers (e.g. polymeric, dendritic, liposomic, etc.), gene gun, injection (including by arrays of microneedles) of naked or modified nucleotides, the like, or combinations thereof.

Other non-limiting examples of inhibitors or therapeutic agents can include siRNAs, miRNAs, morpholinos, ASOs, peptide nucleic acids, small molecule inhibitors, analogues thereof, derivatives thereof, the like, or combinations thereof. Generally, any therapeutic agent that can inhibit the expression of the ABCC11 gene or facilitate targeted degradation of the ABCC11 protein can be used. In some specific examples, the inhibitor can include an siRNA. In some additional examples, the inhibitor can include an miRNA. In yet additional examples, the inhibitor can include a morpholino. In still additional examples, the inhibitor can include an ASO. In some examples, the inhibitor can include a peptide nucleic acid. In some further examples, the inhibitor can include a small molecule inhibitor.

In some examples, the inhibitor can include an RNA sequence, such as an siRNA, miRNA, morpholino, ASO, analogues thereof, derivatives thereof, the like, or a combination thereof. In such examples, the RNA sequence can be administered to a target cell of a subject having osmidrosis. Target cells can include any suitable apocrine target cell. In some examples, the target cells can be or include any suitable ductal epithelial apocrine cell. In some examples, target cells can include axillary apocrine cells, pectoral apocrine cells, genital apocrine cells, or a combination thereof. The prepared inhibitory sequences can vary in length but generally are from about 15 to 31 bases in length. In some examples, these prepared sequences can be siRNAs. A variety of siRNAs can be used, such as one or more (i.e. any suitable combination) of those listed in Table 2 below:

TABLE 2

| | RNA Oligo Sequences* | RNA |
|---|---|---|
| ABCC11 Target | 21 nt guide (5′→3′) | SEQ ID |
| Sequence | 21 nt passenger (5′→3′) | NO: |
| SEQ ID NO: 2 | UGGUUUAUUCAAAUACACCGG | 326 |
| | GGUGUAUUUGAAUAAACCAGG | 327 |
| SEQ ID NO: 3 | UAUAGUAUGAUUUGCCAACCU | 328 |
| | GUUGGCAAAUCAUACUAUAGC | 329 |
| SEQ ID NO: 4 | AGCUAUAGUAUGAUUUGCCAA | 330 |
| | GGCAAAUCAUACUAUAGCUGA | 331 |
| SEQ ID NO: 5 | UCAGCUAUAGUAUGAUUUGCC | 332 |
| | CAAAUCAUACUAUAGCUGAAA | 333 |
| SEQ ID NO: 6 | UUCUUUCAGCUAUAGUAUGAU | 334 |
| | CAUACUAUAGCUGAAAGAAUU | 335 |
| SEQ ID NO: 7 | AGUUCCUGCCAAUUCUUUCAG | 336 |
| | GAAAGAAUUGGCAGGAACUGA | 337 |
| SEQ ID NO: 8 | UUUCAGUUCCUGCCAAUUCUU | 338 |
| | GAAUUGGCAGGAACUGAAAAU | 339 |
| SEQ ID NO: 9 | AGUCAUUUUCAGUUCCUGCCA | 340 |
| | GCAGGAACUGAAAAUGACUAG | 341 |
| SEQ ID NO: 10 | UAGUCAUUUUCAGUUCCUGCC | 342 |
| | CAGGAACUGAAAAUGACUAGG | 343 |
| SEQ ID NO: 11 | UCCUAGUCAUUUUCAGUUCCU | 344 |
| | GAACUGAAAAUGACUAGGAAG | 345 |
| SEQ ID NO: 12 | UCUUCCUAGUCAUUUUCAGUU | 346 |
| | CUGAAAAUGACUAGGAAGAGG | 347 |
| SEQ ID NO: 13 | UGUCGAUGCCACGAUUCACGA | 348 |
| | GUGAAUCGUGGCAUCGACAUA | 349 |
| SEQ ID NO: 14 | UAUGUCGAUGCCACGAUUCAC | 350 |
| | GAAUCGUGGCAUCGACAUAGG | 351 |
| SEQ ID NO: 15 | UCCUGAAACCAUGUCAUCGCC | 352 |
| | CGAUGACAUGGUUUCAGGACU | 353 |
| SEQ ID NO: 16 | UAAGUCCUGAAACCAUGUCAU | 354 |
| | GACAUGGUUUCAGGACUUAUU | 355 |
| SEQ ID NO: 17 | AAUAAGUCCUGAAACCAUGUC | 356 |
| | CAUGGUUUCAGGACUUAUUUA | 357 |
| SEQ ID NO: 18 | AUAAAUAAGUCCUGAAACCAU | 358 |
| | GGUUUCAGGACUUAUUUAUAA | 359 |
| SEQ ID NO: 19 | UAUAAAUAAGUCCUGAAACCA | 360 |
| | GUUUCAGGACUUAUUUAUAAA | 361 |
| SEQ ID NO: 20 | AGGUUUUAUAAAUAAGUCCUG | 362 |
| | GGACUUAUUUAUAAAACCUAU | 363 |
| SEQ ID NO: 21 | UAGGUUUUAUAAAUAAGUCCU | 364 |
| | GACUUAUUUAUAAAACCUAUA | 365 |
| SEQ ID NO: 22 | UAUAGGUUUUAUAAAUAAGUC | 366 |
| | CUUAUUUAUAAAACCUAUACU | 367 |
| SEQ ID NO: 23 | UUUCUCUCUUGCUGACUCCAG | 368 |
| | GGAGUCAGCAAGAGAGAAAUC | 369 |
| SEQ ID NO: 24 | UCUCAAGGCAGCAUCAUACUU | 370 |
| | GUAUGAUGCUGCCUUGAGAAC | 371 |
| SEQ ID NO: 25 | AGAACAGGCCAGCAUUGUCCA | 372 |
| | GACAAUGCUGGCCUGUUCUCC | 373 |
| SEQ ID NO: 26 | AAGCUUUGGAUCAUGAGCGGG | 374 |
| | CGCUCAUGAUCCAAAGCUUAC | 375 |
| SEQ ID NO: 27 | UAAGCUUUGGAUCAUGAGCGG | 376 |
| | GCUCAUGAUCCAAAGCUUACG | 377 |
| SEQ ID NO: 28 | UCUAAGCGACUCCGUAAGCUU | 378 |
| | GCUUACGGAGUCGCUUAGAUG | 379 |
| SEQ ID NO: 29 | AUCUAAGCGACUCCGUAAGCU | 380 |
| | CUUACGGAGUCGCUUAGAUGA | 381 |
| SEQ ID NO: 30 | AUGGUGUUCUCAUCUAAGCGA | 382 |
| | GCUUAGAUGAGAACACCAUCC | 383 |
| SEQ ID NO: 31 | UUUGUCUGAGGCAUCAUGGAC | 384 |
| | CCAUGAUGCCUCAGACAAAAA | 385 |
| SEQ ID NO: 32 | UUUUGUCUGAGGCAUCAUGGA | 386 |
| | CAUGAUGCCUCAGACAAAAAU | 387 |
| SEQ ID NO: 33 | UGGACAUUUUUGUCUGAGGCA | 388 |
| | CCUCAGACAAAAAUGUCCAAA | 389 |
| SEQ ID NO: 34 | UUGGACAUUUUUGUCUGAGGC | 390 |
| | CUCAGACAAAAAUGUCCAAAG | 391 |
| SEQ ID NO: 35 | AAGCCUUUGGACAUUUUUGUC | 392 |
| | CAAAAAUGUCCAAAGGCUUCA | 393 |
| SEQ ID NO: 36 | UUCCCAAAGGCGGUGAAGCCU | 394 |
| | GCUUCACCGCCUUUGGGAAGA | 395 |
| SEQ ID NO: 37 | UUCUUCUUCCCAAAGGCGGUG | 396 |
| | CCGCCUUUGGGAAGAAGAAGU | 397 |
| SEQ ID NO: 38 | ACUUCUUCUUCCCAAAGGCGG | 398 |
| | GCCUUUGGGAAGAAGAAGUCU | 399 |
| SEQ ID NO: 39 | AGACUUCUUCUUCCCAAAGGC | 400 |
| | CUUUGGGAAGAAGAAGUCUCA | 401 |
| SEQ ID NO: 40 | ACUGAAGCUUUUUCAAUCCCU | 402 |
| | GGAUUGAAAAAGCUUCAGUGC | 403 |
| SEQ ID NO: 41 | UCAGCAUCACCAGAAGCACUG | 404 |
| | GUGCUUCUGGUGAUGCUGAGG | 405 |
| SEQ ID NO: 42 | UCUGGAACCUCAGCAUCACCA | 406 |
| | GUGAUGCUGAGGUUCCAGAGA | 407 |
| SEQ ID NO: 43 | UUGUUCUCUGGAACCUCAGCA | 408 |
| | CUGAGGUUCCAGAGAACAAGG | 409 |
| SEQ ID NO: 44 | AACCUUGUUCUCUGGAACCUC | 410 |
| | GGUUCCAGAGAACAAGGUUGA | 411 |
| SEQ ID NO: 45 | AAUCAACCUUGUUCUCUGGAA | 412 |
| | CCAGAGAACAAGGUUGAUUUU | 413 |
| SEQ ID NO: 46 | AAAUCAACCUUGUUCUCUGGA | 414 |
| | CAGAGAACAAGGUUGAUUUUC | 415 |
| SEQ ID NO: 47 | UCGAAAAUCAACCUUGUUCUC | 416 |
| | GAACAAGGUUGAUUUUCGAUG | 417 |
| SEQ ID NO: 48 | AAGUGCAUCGAAAAUCAACCU | 418 |
| | GUUGAUUUUCGAUGCACUUCU | 419 |
| SEQ ID NO: 49 | AGCAGAUGCCCAGAAGUGCAU | 420 |
| | GCACUUCUGGGCAUCUGCUUC | 421 |
| SEQ ID NO: 50 | AAGCAGAUGCCCAGAAGUGCA | 422 |
| | CACUUCUGGGCAUCUGCUUCU | 423 |
| SEQ ID NO: 51 | AAUAUUGGCCCGAGUACACUG | 424 |
| | GUGUACUCGGGCCAAUAUUGA | 425 |
| SEQ ID NO: 52 | UGGUAUAAUCAAUAUUGGCCC | 426 |
| | GCCAAUAUUGAUUAUACCAAA | 427 |
| SEQ ID NO: 53 | UUGGUAUAAUCAAUAUUGGCC | 428 |
| | CCAAUAUUGAUUAUACCAAAG | 429 |
| SEQ ID NO: 54 | UUUGGUAUAAUCAAUAUUGGC | 430 |
| | CAAUAUUGAUUAUACCAAAGA | 431 |
| SEQ ID NO: 55 | AAUAUUCCAGGAUCUUUGGUA | 432 |
| | CCAAAGAUCCUGGAAUAUUCA | 433 |
| SEQ ID NO: 56 | ACUCCAUGGACAGCAUUCCCC | 434 |
| | GGAAUGCUGUCCAUGGAGUGG | 435 |
| SEQ ID NO: 57 | AGACUUCACGCAUUCGGAGAG | 436 |
| | CUCCGAAUGCGUGAAGUCUCU | 437 |
| SEQ ID NO: 58 | AGAGACUUCACGCAUUCGGAG | 438 |
| | CCGAAUGCGUGAAGUCUCUGA | 439 |
| SEQ ID NO: 59 | UCAGAGACUUCACGCAUUCGG | 440 |
| | GAAUGCGUGAAGUCUCUGAGU | 441 |
| SEQ ID NO: 60 | AAACUCAGAGACUUCACGCAU | 442 |
| | GCGUGAAGUCUCUGAGUUUCU | 443 |
| SEQ ID NO: 61 | AGAAACUCAGAGACUUCACGC | 444 |
| | GUGAAGUCUCUGAGUUUCUCC | 445 |
| SEQ ID NO: 62 | AUCCAACUGGAGGAGAAACUC | 446 |
| | GUUUCUCCUCCAGUUGGAUCA | 447 |
| SEQ ID NO: 63 | UGGUUGAUGAUCCAACUGGAG | 448 |
| | CCAGUUGGAUCAUCAACCAAC | 449 |
| SEQ ID NO: 64 | UUGGUUGAUGAUCCAACUGGA | 450 |
| | CAGUUGGAUCAUCAACCAACG | 451 |
| SEQ ID NO: 65 | AGGCAAAGGAGGAAACAGCUG | 452 |
| | GCUGUUUCCUCCUUUGCCUUU | 453 |
| SEQ ID NO: 66 | AAGGCAAAGGAGGAAACAGCU | 454 |
| | CUGUUUCCUCCUUUGCCUUUG | 455 |
| SEQ ID NO: 67 | UUCUCAAAGGCAAAGGAGGAA | 456 |
| | CCUCCUUUGCCUUUGAGAAGC | 457 |
| SEQ ID NO: 68 | UGGAUGAGCUUCUCAAAGGCA | 458 |
| | CCUUUGAGAAGCUCAUCCAAU | 459 |
| SEQ ID NO: 69 | UAAAUUGGAUGAGCUUCUCAA | 460 |
| | GAGAAGCUCAUCCAAUUUAAG | 461 |
| SEQ ID NO: 70 | AGACUUAAAUUGGAUGAGCUU | 462 |
| | GCUCAUCCAAUUUAAGUCUGU | 463 |
| SEQ ID NO: 71 | UACAGACUUAAAUUGGAUGAG | 464 |
| | CAUCCAAUUUAAGUCUGUAAU | 465 |
| SEQ ID NO: 72 | UAUUACAGACUUAAAUUGGAU | 466 |
| | CCAAUUUAAGUCUGUAAUACA | 467 |
| SEQ ID NO: 73 | ACAUCACCGGUGAAGAAGCUG | 468 |
| | GCUUCUUCACCGGUGAUGUAA | 469 |
| SEQ ID NO: 74 | UACAUCACCGGUGAAGAAGCU | 470 |
| | CUUCUUCACCGGUGAUGUAAA | 471 |
| SEQ ID NO: 75 | ACAGGUAGUUUACAUCACCGG | 472 |
| | GGUGAUGUAAACUACCUGUUU | 473 |
| SEQ ID NO: 76 | AACAGGUAGUUUACAUCACCG | 474 |
| | GUGAUGUAAACUACCUGUUUG | 475 |
| SEQ ID NO: 77 | AUAGCACACCCCUUCAAACAG | 476 |
| | GUUUGAAGGGGUGUGCUAUGG | 477 |
| SEQ ID NO: 78 | AGUACUAGGGGUCCAUAGCAC | 478 |
| | GCUAUGGACCCCUAGUACUGA | 479 |
| SEQ ID NO: 79 | AAAUGCUGCAGAUGACCAGCG | 480 |
| | CUGGUCAUCUGCAGCAUUUCU | 481 |
| SEQ ID NO: 80 | AGAAAUGCUGCAGAUGACCAG | 482 |
| | GGUCAUCUGCAGCAUUUCUUC | 483 |
| SEQ ID NO: 81 | AAGAAAUGCUGCAGAUGACCA | 484 |
| | GUCAUCUGCAGCAUUUCUUCC | 485 |
| SEQ ID NO: 82 | AAGUAGGAAGAAAUGCUGCAG | 486 |
| | GCAGCAUUUCUUCCUACUUCA | 487 |
| SEQ ID NO: 83 | AUGAAGUAGGAAGAAAUGCUG | 488 |
| | GCAUUUCUUCCUACUUCAUUA | 489 |
| SEQ ID NO: 84 | AAUGAAGUAGGAAGAAAUGCU | 490 |
| | CAUUUCUUCCUACUUCAUUAU | 491 |
| SEQ ID NO: 85 | UAUCCAAUAAUGAAGUAGGAA | 492 |
| | CCUACUUCAUUAUUGGAUACA | 493 |
| SEQ ID NO: 86 | AGUGUAUCCAAUAAUGAAGUA | 494 |
| | CUUCAUUAUUGGAUACACUGC | 495 |
| SEQ ID NO: 87 | AUAAGAUGGCAAUAAAUGCAG | 496 |
| | GCAUUUAUUGCCAUCUUAUGC | 497 |
| SEQ ID NO: 88 | AGGAGAUAGCAUAAGAUGGCA | 498 |
| | CCAUCUUAUGCUAUCUCCUGG | 499 |
| SEQ ID NO: 89 | UGGGAAAACCAGGAGAUAGCA | 500 |
| | CUAUCUCCUGGUUUUCCCACU | 501 |
| SEQ ID NO: 90 | UUCUUGUCAUGAAUACCGCCA | 502 |
| | GCGGUAUUCAUGACAAGAAUG | 503 |
| SEQ ID NO: 91 | AUUCUUGUCAUGAAUACCGCC | 504 |
| | CGGUAUUCAUGACAAGAAUGG | 505 |
| SEQ ID NO: 92 | UGAUGCUGAGCCUUCACAGCC | 506 |
| | CUGUGAAGGCUCAGCAUCACA | 507 |
| SEQ ID NO: 93 | UCAGAUGUGUGAUGCUGAGCC | 508 |
| | CUCAGCAUCACACAUCUGAGG | 509 |
| SEQ ID NO: 94 | AUGCAAGUGAGAACUUCACUG | 510 |
| | GUGAAGUUCUCACUUGCAUUA | 511 |
| SEQ ID NO: 95 | UAAUGCAAGUGAGAACUUCAC | 512 |
| | GAAGUUCUCACUUGCAUUAAG | 513 |
| SEQ ID NO: 96 | UCAGCUUAAUGCAAGUGAGAA | 514 |
| | CUCACUUGCAUUAAGCUGAUU | 515 |
| SEQ ID NO: 97 | AAUCAGCUUAAUGCAAGUGAG | 516 |
| | CACUUGCAUUAAGCUGAUUAA | 517 |
| SEQ ID NO: 98 | UUAAUCAGCUUAAUGCAAGUG | 518 |
| | CUUGCAUUAAGCUGAUUAAAA | 519 |
| SEQ ID NO: 99 | AUUUUAAUCAGCUUAAUGCAA | 520 |
| | GCAUUAAGCUGAUUAAAAUGU | 521 |
| SEQ ID NO: 100 | UGUGUACAUUUUAAUCAGCUU | 522 |
| | GCUGAUUAAAAUGUACACAUG | 523 |
| SEQ ID NO: 101 | AUGUGUACAUUUUAAUCAGCU | 524 |
| | CUGAUUAAAAUGUACACAUGG | 525 |
| SEQ ID NO: 102 | UGGUUUCUCCCAUGUGUACAU | 526 |
| | GUACACAUGGGAGAAACCAUU | 527 |
| SEQ ID NO: 103 | UCUGCAAAUGGUUUCUCCCAU | 528 |
| | GGGAGAAACCAUUUGCAGAAA | 529 |
| SEQ ID NO: 104 | UUCUGCAAAUGGUUUCUCCCA | 530 |
| | GGAGAAACCAUUUGCAGAAAU | 531 |
| SEQ ID NO: 105 | UUUCUGCAAAUGGUUUCUCCC | 532 |
| | GAGAAACCAUUUGCAGAAAUC | 533 |
| SEQ ID NO: 106 | UCAAUGAUUUCUGCAAAUGGU | 534 |
| | CAUUUGCAGAAAUCAUUGAAG | 535 |
| SEQ ID NO: 107 | UUAGGUCUUCAAUGAUUUCUG | 536 |
| | GAAAUCAUUGAAGACCUAAGA | 537 |
| SEQ ID NO: 108 | UUUCCUUCCUUCUUAGGUCUU | 538 |
| | GACCUAAGAAGGAAGGAAAGG | 539 |
| SEQ ID NO: 109 | UCCUUUCCUUCCUUCUUAGGU | 540 |
| | CUAAGAAGGAAGGAAAGGAAA | 541 |
| SEQ ID NO: 110 | AGUUUCCUUUCCUUCCUUCUU | 542 |
| | GAAGGAAGGAAAGGAAACUAU | 543 |
| SEQ ID NO: 111 | AAUAGUUUCCUUUCCUUCCUU | 544 |
| | GGAAGGAAAGGAAACUAUUGG | 545 |
| SEQ ID NO: 112 | UCUCCAAUAGUUUCCUUUCCU | 546 |
| | GAAAGGAAACUAUUGGAGAAG | 547 |
| SEQ ID NO: 113 | ACAAGGUUAUACUUGUCAGGC | 548 |
| | CUGACAAGUAUAACCUUGUUC | 549 |
| SEQ ID NO: 114 | AUGAACAAGGUUAUACUUGUC | 550 |
| | CAAGUAUAACCUUGUUCAUCA | 551 |
| SEQ ID NO: 115 | AUGAUGAACAAGGUUAUACUU | 552 |
| | GUAUAACCUUGUUCAUCAUCC | 553 |
| SEQ ID NO: 116 | AGGAUGUGUGGAUGAGAACCC | 554 |
| | GUUCUCAUCCACACAUCCUUA | 555 |
| SEQ ID NO: 117 | UCAGCUUUAAGGAUGUGUGGA | 556 |
| | CACACAUCCUUAAAGCUGAAA | 557 |
| SEQ ID NO: 118 | UUUCAGCUUUAAGGAUGUGUG | 558 |
| | CACAUCCUUAAAGCUGAAACU | 559 |
| SEQ ID NO: 119 | AGUUUCAGCUUUAAGGAUGUG | 560 |
| | CAUCCUUAAAGCUGAAACUCA | 561 |
| SEQ ID NO: 120 | UGUGAGUUUCAGCUUUAAGGA | 562 |
| | CUUAAAGCUGAAACUCACAGC | 563 |
| SEQ ID NO: 121 | UGCUGAAGGCCAUUGACGCUG | 564 |
| | GCGUCAAUGGCCUUCAGCAUG | 565 |
| SEQ ID NO: 122 | AUGCUGAAGGCCAUUGACGCU | 566 |
| | CGUCAAUGGCCUUCAGCAUGC | 567 |
| SEQ ID NO: 123 | UUCAAGGAGGCCAGCAUGCUG | 568 |
| | GCAUGCUGGCCUCCUUGAAUC | 569 |
| SEQ ID NO: 124 | UGCAAUAGGCACAAAGAACAC | 570 |
| | GUUCUUUGUGCCUAUUGCAGU | 571 |
| SEQ ID NO: 125 | ACCUUUGACUGCAAUAGGCAC | 572 |
| | GCCUAUUGCAGUCAAAGGUCU | 573 |
| SEQ ID NO: 126 | AGACCUUUGACUGCAAUAGGC | 574 |
| | CUAUUGCAGUCAAAGGUCUCA | 575 |
| SEQ ID NO: 127 | AAUUCGUGAGACCUUUGACUG | 576 |
| | GUCAAAGGUCUCACGAAUUCC | 577 |
| SEQ ID NO: 128 | UCUUGAACCUCAUCACUGCAG | 578 |
| | GCAGUGAUGAGGUUCAAGAAG | 579 |
| SEQ ID NO: 129 | UUCUUGAACCUCAUCACUGCA | 580 |
| | CAGUGAUGAGGUUCAAGAAGU | 581 |
| SEQ ID NO: 130 | ACUUCUUGAACCUCAUCACUG | 582 |
| | GUGAUGAGGUUCAAGAAGUUU | 583 |
| SEQ ID NO: 131 | AAACUUCUUGAACCUCAUCAC | 584 |
| | GAUGAGGUUCAAGAAGUUUUU | 585 |
| SEQ ID NO: 132 | AGGAAAAACUUCUUGAACCUC | 586 |
| | GGUUCAAGAAGUUUUUCCUCC | 587 |
| SEQ ID NO: 133 | UUGUAAUGUCUGGACAUAGAA | 588 |
| | CUAUGUCCAGACAUUACAAGA | 589 |
| SEQ ID NO: 134 | AAAGACCAGAGCUUUGCUGGG | 590 |
| | CAGCAAAGCUCUGGUCUUUGA | 591 |
| SEQ ID NO: 135 | UCAAAGACCAGAGCUUUGCUG | 592 |
| | GCAAAGCUCUGGUCUUUGAGG | 593 |
| SEQ ID NO: 136 | AGAAGCAUGCCCGUUCCUCUC | 594 |
| | GAGGAACGGGCAUGCUUCUGA | 595 |
| SEQ ID NO: 137 | AUCUCUAGGCCUGGUCAUCCC | 596 |
| | GAUGACCAGGCCUAGAGAUGC | 597 |
| SEQ ID NO: 138 | UGAUCUUGUGCAACUCUGGGC | 598 |
| | CCAGAGUUGCACAAGAUCAAC | 599 |
| SEQ ID NO: 139 | UUGAUCUUGUGCAACUCUGGG | 600 |
| | CAGAGUUGCACAAGAUCAACC | 601 |
| SEQ ID NO: 140 | ACAUCAUCCCCUUGGACACCA | 602 |
| | GUGUCCAAGGGGAUGAUGUUA | 603 |
| SEQ ID NO: 141 | UUACCACUCCCCGUGUUGCCG | 604 |
| | GCAACACGGGGAGUGGUAAGA | 605 |
| SEQ ID NO: 142 | ACAACAGGCUGCUCUUACCAC | 606 |
| | GGUAAGAGCAGCCUGUUGUCA | 607 |
| SEQ ID NO: 143 | AUGUUCUCCCUGAUGUUCCCG | 608 |
| | GGAACAUCAGGGAGAACAUCC | 609 |
| SEQ ID NO: 144 | UCCAAAGGGCAGAAGUUCCAG | 610 |
| | GGAACUUCUGCCCUUUGGAGA | 611 |
| SEQ ID NO: 145 | UGUCAUGUCUCCAAAGGGCAG | 612 |
| | GCCCUUUGGAGACAUGACAGA | 613 |
| SEQ ID NO: 146 | UCUGUCAUGUCUCCAAAGGGC | 614 |
| | CCUUUGGAGACAUGACAGAGA | 615 |
| SEQ ID NO: 147 | AAUGCACUCCUCAAAAAUGUG | 616 |
| | CAUUUUUGAGGAGUGCAUUAA | 617 |
| SEQ ID NO: 148 | AAAAUUCUAAGUACUGCAGCU | 618 |
| | CUGCAGUACUUAGAAUUUUGU | 619 |
| SEQ ID NO: 149 | ACAAAAUUCUAAGUACUGCAG | 620 |
| | GCAGUACUUAGAAUUUUGUGG | 621 |
| SEQ ID NO: 150 | UGAUCUGGCCACAAAAUUCUA | 622 |
| | GAAUUUUGUGGCCAGAUCAUU | 623 |
| SEQ ID NO: 151 | UCCAACAAAAUGAUCUGGCCA | 624 |
| | GCCAGAUCAUUUUGUUGGAAA | 625 |
| SEQ ID NO: 152 | UUCCAACAAAAUGAUCUGGCC | 626 |
| | CCAGAUCAUUUUGUUGGAAAA | 627 |
| SEQ ID NO: 153 | UUUCCAACAAAAUGAUCUGGC | 628 |
| | CAGAUCAUUUUGUUGGAAAAU | 629 |
| SEQ ID NO: 154 | AUUUUCCAACAAAAUGAUCUG | 630 |
| | GAUCAUUUUGUUGGAAAAUGG | 631 |
| SEQ ID NO: 155 | ACAGAUUUUCCCAUUUUCCAA | 632 |
| | GGAAAAUGGGAAAAUCUGUGA | 633 |
| SEQ ID NO: 156 | UCCAUUUUCACAGAUUUUCCC | 634 |
| | GAAAAUCUGUGAAAAUGGAAC | 635 |
| SEQ ID NO: 157 | UUAACUCACUGUGAGUUCCAU | 636 |
| | GGAACUCACAGUGAGUUAAUG | 637 |
| SEQ ID NO: 158 | AUUAACUCACUGUGAGUUCCA | 638 |
| | GAACUCACAGUGAGUUAAUGC | 639 |
| SEQ ID NO: 159 | UGCAUUAACUCACUGUGAGUU | 640 |
| | CUCACAGUGAGUUAAUGCAGA | 641 |
| SEQ ID NO: 160 | UCUGCAUUAACUCACUGUGAG | 642 |
| | CACAGUGAGUUAAUGCAGAAA | 643 |
| SEQ ID NO: 161 | UUUCUGCAUUAACUCACUGUG | 644 |
| | CAGUGAGUUAAUGCAGAAAAA | 645 |
| SEQ ID NO: 162 | UUUUUCUGCAUUAACUCACUG | 646 |
| | GUGAGUUAAUGCAGAAAAAGG | 647 |
| SEQ ID NO: 163 | AUAUUUCCCCUUUUUCUGCAU | 648 |
| | GCAGAAAAAGGGGAAAUAUGC | 649 |
| SEQ ID NO: 164 | UAAGUUGGGCAUAUUUCCCCU | 650 |
| | GGGAAAUAUGCCCAACUUAUC | 651 |
| SEQ ID NO: 165 | AUAAGUUGGGCAUAUUUCCCC | 652 |
| | GGAAAUAUGCCCAACUUAUCC | 653 |
| SEQ ID NO: 166 | AUCUUCUGGAUAAGUUGGGCA | 654 |
| | CCCAACUUAUCCAGAAGAUGC | 655 |
| SEQ ID NO: 167 | UUCCUUGUGCAUCUUCUGGAU | 656 |
| | CCAGAAGAUGCACAAGGAAGC | 657 |
| SEQ ID NO: 168 | UGCUAUCUUUGCUGUGUCCUG | 658 |
| | GGACACAGCAAAGAUAGCAGA | 659 |
| SEQ ID NO: 169 | UCUACCUUUGGCUUCUCUGCU | 660 |
| | CAGAGAAGCCAAAGGUAGAAA | 661 |
| SEQ ID NO: 170 | ACUUUCUACCUUUGGCUUCUC | 662 |
| | GAAGCCAAAGGUAGAAAGUCA | 663 |
| SEQ ID NO: 171 | AGCAUUUCCGUUGAGAGACUC | 664 |
| | GUCUCUCAACGGAAAUGCUGU | 665 |
| SEQ ID NO: 172 | ACAGCAUUUCCGUUGAGAGAC | 666 |
| | CUCUCAACGGAAAUGCUGUGC | 667 |
| SEQ ID NO: 173 | ACUCAAGGAGCCUUCUUCCAU | 668 |
| | GGAAGAAGGCUCCUUGAGUUG | 669 |
| SEQ ID NO: 174 | AACUCAAGGAGCCUUCUUCCA | 670 |
| | GAAGAAGGCUCCUUGAGUUGG | 671 |
| SEQ ID NO: 175 | UGCAAGAGACCAUGUAACCUC | 672 |
| | GGUUACAUGGUCUCUUGCAUA | 673 |
| SEQ ID NO: 176 | AUGCAAGAGACCAUGUAACCU | 674 |
| | GUUACAUGGUCUCUUGCAUAA | 675 |
| SEQ ID NO: 177 | AAUUAUGCAAGAGACCAUGUA | 676 |
| | CAUGGUCUCUUGCAUAAUUUU | 677 |
| SEQ ID NO: 178 | AGAAAAUUAUGCAAGAGACCA | 678 |
| | GUCUCUUGCAUAAUUUUCUUC | 679 |
| SEQ ID NO: 179 | AAGAAGAAAAUUAUGCAAGAG | 680 |
| | CUUGCAUAAUUUUCUUCUUCG | 681 |
| SEQ ID NO: 180 | ACGAAGAAGAAAAUUAUGCAA | 682 |
| | GCAUAAUUUUCUUCUUCGUGG | 683 |
| SEQ ID NO: 181 | UUAAGAAGACGAUCAGCACCA | 684 |
| | GUGCUGAUCGUCUUCUUAACG | 685 |
| SEQ ID NO: 182 | UCGUUAAGAAGACGAUCAGCA | 686 |
| | CUGAUCGUCUUCUUAACGAUC | 687 |
| SEQ ID NO: 183 | UGAAGAUCGUUAAGAAGACGA | 688 |
| | GUCUUCUUAACGAUCUUCAGC | 689 |
| SEQ ID NO: 184 | AGGAUUGUCUGCAAUGUUGCC | 690 |
| | CAACAUUGCAGACAAUCCUCA | 691 |
| SEQ ID NO: 185 | UUGAGGAUUGUCUGCAAUGUU | 692 |
| | CAUUGCAGACAAUCCUCAACU | 693 |
| SEQ ID NO: 186 | ACAGUUGAGGAUUGUCUGCAA | 694 |
| | GCAGACAAUCCUCAACUGUCC | 695 |
| SEQ ID NO: 187 | UGGUAGAAGGACAGUUGAGGA | 696 |
| | CUCAACUGUCCUUCUACCAGC | 697 |
| SEQ ID NO: 188 | UGACCUUGGUGAAAAUCCCUG | 698 |
| | GGGAUUUUCACCAAGGUCACG | 699 |
| SEQ ID NO: 189 | UAAAGAGCUUGUUGUGCAGGG | 700 |
| | CUGCACAACAAGCUCUUUAAC | 701 |
| SEQ ID NO: 190 | UGUUAAAGAGCUUGUUGUGCA | 702 |
| | CACAACAAGCUCUUUAACAAG | 703 |
| SEQ ID NO: 191 | AACCUUGUUAAAGAGCUUGUU | 704 |
| | CAAGCUCUUUAACAAGGUUUU | 705 |
| SEQ ID NO: 192 | UGUCAAAGAAACUCAUGGGGC | 706 |
| | CCCAUGAGUUUCUUUGACACC | 707 |
| SEQ ID NO: 193 | UAUUGGGAUGGUGUCAAAGAA | 708 |
| | CUUUGACACCAUCCCAAUAGG | 709 |
| SEQ ID NO: 194 | UCAAAAGCCGGCCUAUUGGGA | 710 |
| | CCAAUAGGCCGGCUUUUGAAC | 711 |
| SEQ ID NO: 195 | UUCAAAAGCCGGCCUAUUGGG | 712 |
| | CAAUAGGCCGGCUUUUGAACU | 713 |
| SEQ ID NO: 196 | AAAAGAUGGGCAAGAGCUGGU | 714 |
| | CAGCUCUUGCCCAUCUUUUCA | 715 |
| SEQ ID NO: 197 | UGAAAAGAUGGGCAAGAGCUG | 716 |
| | GCUCUUGCCCAUCUUUUCAGA | 717 |
| SEQ ID NO: 198 | UAACAGGAUAUAUGGAGACAG | 718 |
| | GUCUCCAUAUAUCCUGUUAAU | 719 |
| SEQ ID NO: 199 | AUUAACAGGAUAUAUGGAGAC | 720 |
| | CUCCAUAUAUCCUGUUAAUGG | 721 |
| SEQ ID NO: 200 | UUAUGGCUCCCAUUAACAGGA | 722 |
| | CUGUUAAUGGGAGCCAUAAUC | 723 |
| SEQ ID NO: 201 | AUAACCAUGAUUAUGGCUCCC | 724 |
| | GAGCCAUAAUCAUGGUUAUUU | 725 |
| SEQ ID NO: 202 | AAAUAACCAUGAUUAUGGCUC | 726 |
| | GCCAUAAUCAUGGUUAUUUGC | 727 |
| SEQ ID NO: 203 | AAUGAAGCAAAUAACCAUGAU | 728 |
| | CAUGGUUAUUUGCUUCAUUUA | 729 |
| SEQ ID NO: 204 | AUAAAUGAAGCAAAUAACCAU | 730 |
| | GGUUAUUUGCUUCAUUUAUUA | 731 |
| SEQ ID NO: 205 | AAUAAAUGAAGCAAAUAACCA | 732 |
| | GUUAUUUGCUUCAUUUAUUAU | 733 |
| SEQ ID NO: 206 | AUCAUAUAAUAAAUGAAGCAA | 734 |
| | GCUUCAUUUAUUAUAUGAUGU | 735 |
| SEQ ID NO: 207 | AUAGUUCUCCAGUCUCUUGAA | 736 |
| | CAAGAGACUGGAGAACUAUAG | 737 |
| SEQ ID NO: 208 | UAAAGGAGACCGGCUAUAGUU | 738 |
| | CUAUAGCCGGUCUCCUUUAUU | 739 |
| SEQ ID NO: 209 | AGGAUGUGGGAGAAUAAAGGA | 740 |
| | CUUUAUUCUCCCACAUCCUCA | 741 |
| SEQ ID NO: 210 | AGAGAAUUGAGGAUGUGGGAG | 742 |
| | CCCACAUCCUCAAUUCUCUGC | 743 |
| SEQ ID NO: 211 | UUUCCAUAGACAUGGAUGGAG | 744 |
| | CCAUCCAUGUCUAUGGAAAAA | 745 |
| SEQ ID NO: 212 | UUUUCCAUAGACAUGGAUGGA | 746 |
| | CAUCCAUGUCUAUGGAAAAAC | 747 |
| SEQ ID NO: 213 | AGUUUUUCCAUAGACAUGGAU | 748 |
| | CCAUGUCUAUGGAAAAACUGA | 749 |
| SEQ ID NO: 214 | UGAAGUCUUCAGUUUUUCCAU | 750 |
| | GGAAAAACUGAAGACUUCAUC | 751 |
| SEQ ID NO: 215 | AUGAAGUCUUCAGUUUUUCCA | 752 |
| | GAAAAACUGAAGACUUCAUCA | 753 |
| SEQ ID NO: 216 | UAAACUGGCUGAUGAAGUCUU | 754 |
| | GACUUCAUCAGCCAGUUUAAG | 755 |
| SEQ ID NO: 217 | AUCAGUCAGCCUCUUAAACUG | 756 |
| | GUUUAAGAGGCUGACUGAUGC | 757 |
| SEQ ID NO: 218 | UAUUCUGCGCAUCAGUCAGCC | 758 |
| | CUGACUGAUGCGCAGAAUAAC | 759 |
| SEQ ID NO: 219 | AAGAUAGAAACAACAGCAGGU | 760 |
| | CUGCUGUUGUUUCUAUCUUCC | 761 |
| SEQ ID NO: 220 | UGGAAGAUAGAAACAACAGCA | 762 |
| | CUGUUGUUUCUAUCUUCCACA | 763 |
| SEQ ID NO: 221 | UGUGGAAGAUAGAAACAACAG | 764 |
| | GUUGUUUCUAUCUUCCACACG | 765 |
| SEQ ID NO: 222 | AUGAUCUCCAGCCUCAAUGCC | 766 |
| | CAUUGAGGCUGGAGAUCAUGA | 767 |
| SEQ ID NO: 223 | UGCCAAAAGCCACGAACAGGG | 768 |
| | CUGUUCGUGGCUUUUGGCAUU | 769 |
| SEQ ID NO: 224 | AGGAAAUGCCAAAAGCCACGA | 770 |
| | GUGGCUUUUGGCAUUUCCUCC | 771 |
| SEQ ID NO: 225 | UGACUUUAAAGGAGUAGGGGG | 772 |
| | CCCUACUCCUUUAAAGUCAUG | 773 |
| SEQ ID NO: 226 | AUGACUUUAAAGGAGUAGGGG | 774 |
| | CCUACUCCUUUAAAGUCAUGG | 775 |
| SEQ ID NO: 227 | UGAACUGUGCCUCUGUCUCCA | 776 |
| | GAGACAGAGGCACAGUUCACG | 777 |
| SEQ ID NO: 228 | UCUACAGCCGUGAACUGUGCC | 778 |
| | CACAGUUCACGGCUGUAGAGA | 779 |
| SEQ ID NO: 229 | UCUCUACAGCCGUGAACUGUG | 780 |
| | CAGUUCACGGCUGUAGAGAGG | 781 |
| SEQ ID NO: 230 | UGCAGUAUCCUCUCUACAGCC | 782 |
| | CUGUAGAGAGGAUACUGCAGU | 783 |
| SEQ ID NO: 231 | UUCAUGUACUGCAGUAUCCUC | 784 |
| | GGAUACUGCAGUACAUGAAGA | 785 |
| SEQ ID NO: 232 | ACAUCUUCAUGUACUGCAGUA | 786 |
| | CUGCAGUACAUGAAGAUGUGU | 787 |
| SEQ ID NO: 233 | 000 | |
| SEQ ID NO: 234 | ACACAUCUUCAUGUACUGCAG | 788 |
| | GCAGUACAUGAAGAUGUGUGU | 789 |
| SEQ ID NO: 235 | AACUUGUGCCUUCCAUGUGUA | 790 |
| | CACAUGGAAGGCACAAGUUGU | 791 |
| SEQ ID NO: 236 | ACAACUUGUGCCUUCCAUGUG | 792 |
| | CAUGGAAGGCACAAGUUGUCC | 793 |
| SEQ ID NO: 237 | AUGAUUUCCCCAUGCUGUGGC | 794 |
| | CACAGCAUGGGGAAAUCAUAU | 795 |
| SEQ ID NO: 238 | UCCUGAAAUAUGAUUUCCCCA | 796 |
| | GGGAAAUCAUAUUUCAGGAUU | 797 |
| SEQ ID NO: 239 | AUCCUGAAAUAUGAUUUCCCC | 798 |
| | GGAAAUCAUAUUUCAGGAUUA | 799 |
| SEQ ID NO: 240 | AAUCCUGAAAUAUGAUUUCCC | 800 |
| | GAAAUCAUAUUUCAGGAUUAU | 801 |
| SEQ ID NO: 241 | AUUUCAUGUGAUAAUCCUGAA | 802 |
| | CAGGAUUAUCACAUGAAAUAC | 803 |
| SEQ ID NO: 242 | UGUAUUUCAUGUGAUAAUCCU | 804 |
| | GAUUAUCACAUGAAAUACAGA | 805 |
| SEQ ID NO: 243 | UGUCUCUGUAUUUCAUGUGAU | 806 |
| | CACAUGAAAUACAGAGACAAC | 807 |
| SEQ ID NO: 244 | UGUGUUGUCUCUGUAUUUCAU | 808 |
| | GAAAUACAGAGACAACACACC | 809 |
| SEQ ID NO: 245 | AAUGUCCACGCCGUCAAUGAG | 810 |
| | CAUUGACGGCGUGGACAUUUG | 811 |
| SEQ ID NO: 246 | AGAGCUUGGACCGCAAGUCCU | 812 |
| | GACUUGCGGUCCAAGCUCUCA | 813 |
| SEQ ID NO: 247 | AUCACUGAGAGCUUGGACCGC | 814 |
| | GGUCCAAGCUCUCAGUGAUCC | 815 |
| SEQ ID NO: 248 | AUCUUGAGGGAUCACUGAGAG | 816 |
| | CUCAGUGAUCCCUCAAGAUCC | 817 |
| SEQ ID NO: 249 | UCUGAUGGUUCCUGAGAGCAG | 818 |
| | GCUCUCAGGAACCAUCAGAUU | 819 |
| SEQ ID NO: 250 | AGGUUGAAUCUGAUGGUUCCU | 820 |
| | GAACCAUCAGAUUCAACCUAG | 821 |
| SEQ ID NO: 251 | UCUAGGUUGAAUCUGAUGGUU | 822 |
| | CCAUCAGAUUCAACCUAGAUC | 823 |
| SEQ ID NO: 252 | AUCUAGGUUGAAUCUGAUGGU | 824 |
| | CAUCAGAUUCAACCUAGAUCC | 825 |
| SEQ ID NO: 253 | UCAGUGUGACGGUCAAAGGGA | 826 |
| | CCUUUGACCGUCACACUGACC | 827 |
| SEQ ID NO: 254 | AGGAAUGUCCUCUCCAAGGCA | 828 |
| | CCUUGGAGAGGACAUUCCUGA | 829 |
| SEQ ID NO: 255 | UUGAGAUGGCCUUGGUCAGGA | 830 |
| | CUGACCAAGGCCAUCUCAAAG | 831 |
| SEQ ID NO: 256 | AGCUUUUUGGGGAACUUUGAG | 832 |
| | CAAAGUUCCCCAAAAAGCUGC | 833 |
| SEQ ID NO: 257 | UGUAUGCAGCUUUUUGGGGAA | 834 |
| | CCCCAAAAAGCUGCAUACAGA | 835 |
| SEQ ID NO: 258 | UCUGUAUGCAGCUUUUUGGGG | 836 |
| | CCAAAAAGCUGCAUACAGAUG | 837 |
| SEQ ID NO: 259 | AUCUGUAUGCAGCUUUUUGGG | 838 |
| | CAAAAAGCUGCAUACAGAUGU | 839 |
| SEQ ID NO: 260 | AGUUUCCACCGUUUUCCACCA | 840 |
| | GUGGAAAACGGUGGAAACUUC | 841 |
| SEQ ID NO: 261 | AGAAGUUUCCACCGUUUUCCA | 842 |
| | GAAAACGGUGGAAACUUCUCU | 843 |
| SEQ ID NO: 262 | UUGGAGUUGCGAAGCACAGCC | 844 |
| | CUGUGCUUCGCAACUCCAAGA | 845 |
| SEQ ID NO: 263 | UGAUCUUGGAGUUGCGAAGCA | 846 |
| | CUUCGCAACUCCAAGAUCAUC | 847 |
| SEQ ID NO: 264 | AGGAUGAUCUUGGAGUUGCGA | 848 |
| | GCAACUCCAAGAUCAUCCUUA | 849 |
| SEQ ID NO: 265 | AAGGAUGAUCUUGGAGUUGCG | 850 |
| | CAACUCCAAGAUCAUCCUUAU | 851 |
| SEQ ID NO: 266 | UCGAUAAGGAUGAUCUUGGAG | 852 |
| | CCAAGAUCAUCCUUAUCGAUG | 853 |
| SEQ ID NO: 267 | AUCGAUAAGGAUGAUCUUGGA | 854 |
| | CAAGAUCAUCCUUAUCGAUGA | 855 |
| SEQ ID NO: 268 | UUCAUCGAUAAGGAUGAUCUU | 856 |
| | GAUCAUCCUUAUCGAUGAAGC | 857 |
| SEQ ID NO: 269 | UCUGUCUCCAUGUCAAUGGAG | 858 |
| | CCAUUGACAUGGAGACAGACA | 859 |
| SEQ ID NO: 270 | UCACAGUUCAGCACAGUGGUG | 860 |
| | CCACUGUGCUGAACUGUGACC | 861 |
| SEQ ID NO: 271 | UCCCAUUGCCCAUAACCAGGA | 862 |
| | CUGGUUAUGGGCAAUGGGAAG | 863 |
| SEQ ID NO: 272 | UCUACCACCUUCCCAUUGCCC | 864 |
| | GCAAUGGGAAGGUGGUAGAAU | 865 |
| SEQ ID NO: 273 | UUCUACCACCUUCCCAUUGCC | 866 |
| | CAAUGGGAAGGUGGUAGAAUU | 867 |
| SEQ ID NO: 274 | UCAAAUUCUACCACCUUCCCA | 868 |
| | GGAAGGUGGUAGAAUUUGAUC | 869 |
| SEQ ID NO: 275 | AUCUCAGUGAAGAAGUGGCUG | 870 |
| | GCCACUUCUUCACUGAGAUAA | 871 |
| SEQ ID NO: 276 | UAUCUCAGUGAAGAAGUGGCU | 872 |
| | CCACUUCUUCACUGAGAUAAG | 873 |
| SEQ ID NO: 277 | UUAUCUCAGUGAAGAAGUGGC | 874 |
| | CACUUCUUCACUGAGAUAAGG | 875 |
| SEQ ID NO: 278 | UCUCCUUAUCUCAGUGAAGAA | 876 |
| | CUUCACUGAGAUAAGGAGAUG | 877 |
| SEQ ID NO: 279 | ACAUCUCCUUAUCUCAGUGAA | 878 |
| | CACUGAGAUAAGGAGAUGUGG | 879 |
| SEQ ID NO: 280 | AUGAAGUCUCCACAUCUCCUU | 880 |
| | GGAGAUGUGGAGACUUCAUGG | 881 |
| SEQ ID NO: 281 | UCCAUGAAGUCUCCACAUCUC | 882 |
| | GAUGUGGAGACUUCAUGGAGG | 883 |
| SEQ ID NO: 282 | AGACUGUGGGCCUCGAAGCUG | 884 |
| | GCUUCGAGGCCCACAGUCUGC | 885 |
| SEQ ID NO: 283 | AAGAAGGUCGCAGACUGUGGG | 886 |
| | CACAGUCUGCGACCUUCUUGU | 887 |
| SEQ ID NO: 284 | AUCUCCAAACAAGAAGGUCGC | 888 |
| | GACCUUCUUGUUUGGAGAUGA | 889 |
| SEQ ID NO: 285 | UCAUCUCCAAACAAGAAGGUC | 890 |
| | CCUUCUUGUUUGGAGAUGAGA | 891 |
| SEQ ID NO: 286 | AGGAGAAGUUCUCAUCUCCAA | 892 |
| | GGAGAUGAGAACUUCUCCUGG | 893 |
| SEQ ID NO: 287 | AUUUACCCCUGCUUCCAGGAG | 894 |
| | CCUGGAAGCAGGGGUAAAUGU | 895 |
| SEQ ID NO: 288 | 000 | |
| SEQ ID NO: 289 | UACAUUUACCCCUGCUUCCAG | 896 |
| | GGAAGCAGGGGUAAAUGUAGG | 897 |
| SEQ ID NO: 290 | UUUCCAUCCAGCAAUCCCCAC | 898 |
| | GGGGAUUGCUGGAUGGAAACC | 899 |
| SEQ ID NO: 291 | UAUUCCAGGGUUUCCAUCCAG | 900 |
| | GGAUGGAAACCCUGGAAUAGG | 901 |
| SEQ ID NO: 292 | UAGCCUAUUCCAGGGUUUCCA | 902 |
| | GAAACCCUGGAAUAGGCUACU | 903 |
| SEQ ID NO: 293 | UCAAGUAGCCUAUUCCAGGGU | 904 |
| | CCUGGAAUAGGCUACUUGAUG | 905 |
| SEQ ID NO: 294 | AUCAAGUAGCCUAUUCCAGGG | 906 |
| | CUGGAAUAGGCUACUUGAUGG | 907 |
| SEQ ID NO: 295 | UGGUUCUGGGGUUCUAAGGUC | 908 |
| | CCUUAGAACCCCAGAACCAUC | 909 |
| SEQ ID NO: 296 | AUGGUUCUGGGGUUCUAAGGU | 910 |
| | CUUAGAACCCCAGAACCAUCU | 911 |
| SEQ ID NO: 297 | UGUCUUAGAUGGUUCUGGGGU | 912 |
| | CCCAGAACCAUCUAAGACAUG | 913 |
| SEQ ID NO: 298 | AUGUCUUAGAUGGUUCUGGGG | 914 |
| | CCAGAACCAUCUAAGACAUGG | 915 |
| SEQ ID NO: 299 | AAUCCCAUGUCUUAGAUGGUU | 916 |
| | CCAUCUAAGACAUGGGAUUCA | 917 |
| SEQ ID NO: 300 | ACUGAAUCCCAUGUCUUAGAU | 918 |
| | CUAAGACAUGGGAUUCAGUGA | 919 |
| SEQ ID NO: 301 | AUGAUCACUGAAUCCCAUGUC | 920 |
| | CAUGGGAUUCAGUGAUCAUGU | 921 |
| SEQ ID NO: 302 | ACCACAUGAUCACUGAAUCCC | 922 |
| | GAUUCAGUGAUCAUGUGGUUC | 923 |
| SEQ ID NO: 303 | AAAGGAGAACCACAUGAUCAC | 924 |
| | GAUCAUGUGGUUCUCCUUUUA | 925 |
| SEQ ID NO: 304 | UUAAAAGGAGAACCACAUGAU | 926 |
| | CAUGUGGUUCUCCUUUUAACU | 927 |
| SEQ ID NO: 305 | AAGUUAAAAGGAGAACCACAU | 928 |
| | GUGGUUCUCCUUUUAACUUAC | 929 |
| SEQ ID NO: 306 | UGUAAGUUAAAAGGAGAACCA | 930 |
| | GUUCUCCUUUUAACUUACAUG | 931 |
| SEQ ID NO: 307 | UCAGCAUGUAAGUUAAAAGGA | 932 |
| | CUUUUAACUUACAUGCUGAAU | 933 |
| SEQ ID NO: 308 | AAAUUAUUCAGCAUGUAAGUU | 934 |
| | CUUACAUGCUGAAUAAUUUUA | 935 |
| SEQ ID NO: 309 | UAUAAAAUUAUUCAGCAUGUA | 936 |
| | CAUGCUGAAUAAUUUUAUAAU | 937 |
| SEQ ID NO: 310 | UAUUAUAAAAUUAUUCAGCAU | 938 |
| | GCUGAAUAAUUUUAUAAUAAG | 939 |
| SEQ ID NO: 311 | UUAUUAUAAAAUUAUUCAGCA | 940 |
| | CUGAAUAAUUUUAUAAUAAGG | 941 |
| SEQ ID NO: 312 | AAAACUAUAAGCUUUUACCUU | 942 |
| | GGUAAAAGCUUAUAGUUUUCU | 943 |
| SEQ ID NO: 313 | AGAUCAGAAAACUAUAAGCUU | 944 |
| | GCUUAUAGUUUUCUGAUCUGU | 945 |
| SEQ ID NO: 314 | AACACUUCUAACACAGAUCAG | 946 |
| | GAUCUGUGUUAGAAGUGUUGC | 947 |
| SEQ ID NO: 315 | UUUGCAACACUUCUAACACAG | 948 |
| | GUGUUAGAAGUGUUGCAAAUG | 949 |
| SEQ ID NO: 316 | ACAGCAUUUGCAACACUUCUA | 950 |
| | GAAGUGUUGCAAAUGCUGUAC | 951 |
| SEQ ID NO: 317 | UCAGUACAGCAUUUGCAACAC | 952 |
| | GUUGCAAAUGCUGUACUGACU | 953 |
| SEQ ID NO: 318 | AAGUCAGUACAGCAUUUGCAA | 954 |
| | GCAAAUGCUGUACUGACUUUG | 955 |
| SEQ ID NO: 319 | AAAGUCAGUACAGCAUUUGCA | 956 |
| | CAAAUGCUGUACUGACUUUGU | 957 |
| SEQ ID NO: 320 | UUUACAAAGUCAGUACAGCAU | 958 |
| | GCUGUACUGACUUUGUAAAAU | 959 |
| SEQ ID NO: 321 | UUUUACAAAGUCAGUACAGCA | 960 |
| | CUGUACUGACUUUGUAAAAUA | 961 |
| SEQ ID NO: 322 | UAUUUUACAAAGUCAGUACAG | 962 |
| | GUACUGACUUUGUAAAAUAUA | 963 |
| SEQ ID NO: 323 | UUAUAUUUUACAAAGUCAGUA | 964 |
| | CUGACUUUGUAAAAUAUAAAA | 965 |
| SEQ ID NO: 324 | UUUUAUAUUUUACAAAGUCAG | 966 |
| | GACUUUGUAAAAUAUAAAACU | 967 |
| SEQ ID NO: 325 | AGUUUUAUAUUUUACAAAGUC | 968 |
| | CUUUGUAAAAUAUAAAACUAA | 969 |

*It is noted that the particular sequences listed in this table do not include any 3′ nucleotide overhangs. However, this is not intended to preclude the use of suitable 3′ nucleotide overhangs. The use of any suitable number and variety of 3′ nucleotide overhangs is contemplated. Thus, any suitable 3′ overhangs can be used with the guide strands and the passenger strands listed in Table 2.

As described above, in some examples, one or more siRNA inhibitors listed in Table 2 can be used to inhibit expression of the ABCC11 gene. In one example, the ABCC11 inhibitor can include a sequence listed in Table 2, or a compliment thereof. Such sequence can further include any required delivery components to create a deliverable construct, including relevant promotors, viral vectors, etc. In some examples, one or more siRNA inhibitors having a guide strand listed in Table 2, or a guide strand at least 90% or 95% homologous thereto, can be used to inhibit expression of the ABCC11 gene. In some examples, two or more, three or more, four or more, five or more, or ten or more siRNA inhibitors having a guide strand listed in Table 2, or a guide strand at least 90% or 95% homologous thereto, can be used to inhibit expression of the ABCC11 gene. In some examples, the one or more siRNA inhibitors can include a guide strand having a sequence of SEQ ID NO: 326, or a sequence that is at least 90% or 95% homologous thereto. In some examples, the one or more siRNA inhibitors can include a guide strand having a sequence of SEQ ID NO: 328, or a sequence that is at least 90% or 95% homologous thereto. In some examples, the one or more siRNA inhibitors can include a guide strand having a sequence of SEQ ID NO: 330, or a sequence that is at least 90% or 95% homologous thereto. In some examples, the one or more siRNA inhibitors can include a guide strand having a sequence of SEQ ID NO: 332, or a sequence that is at least 90% or 95% homologous thereto. In some examples, the one or more siRNA inhibitors can include a guide strand having a sequence of SEQ ID NO: 334, or a sequence that is at least 90% or 95% homologous thereto. In some examples, the one or more siRNA inhibitors can include a guide strand having a sequence of SEQ ID NO: 336, or a sequence that is at least 90% or 95% homologous thereto. In some examples, the one or more siRNA inhibitors can include a guide strand having a sequence of SEQ ID NO: 338, or a sequence that is at least 90% or 95% homologous thereto. In some examples, the one or more siRNA inhibitors can include a guide strand having a sequence of SEQ ID NO: 340, or a sequence that is at least 90% or 95% homologous thereto. In some examples, the one or more siRNA inhibitors can include a guide strand having a sequence of SEQ ID NO: 342, or a sequence that is at least 90% or 95% homologous thereto. In some examples, the one or more siRNA inhibitors can include a guide strand having a sequence of SEQ ID NO: 344, or a sequence that is at least 90% or 95% homologous thereto. In some examples, the one or more siRNA inhibitors can include a guide strand having a sequence of SEQ ID NO: 346, or a sequence that is at least 90% or 95% homologous thereto. In some examples, the one or more siRNA inhibitors can include a guide strand having a sequence of SEQ ID NO: 348, or a sequence that is at least 90% or 95% homologous thereto. In some examples, the one or more siRNA inhibitors can include a guide strand having a sequence of SEQ ID NO: 350, or a sequence that is at least 90% or 95% homologous thereto. In some examples, the one or more siRNA inhibitors can include a guide strand having a sequence of SEQ ID NO: 352, or a sequence that is at least 90% or 95% homologous thereto. In some examples, the one or more siRNA inhibitors can include a guide strand having a sequence of SEQ ID NO: 354, or a sequence that is at least 90% or 95% homologous thereto. In some examples, the one or more siRNA inhibitors can include a guide strand having a sequence of SEQ ID NO: 356, or a sequence that is at least 90% or 95% homologous thereto. In some examples, the one or more siRNA inhibitors can include a guide strand having a sequence of SEQ ID NO: 358, or a sequence that is at least 90% or 95% homologous thereto. In some examples, the one or more siRNA inhibitors can include a guide strand having a sequence of SEQ ID NO: 360, or a sequence that is at least 90% or 95% homologous thereto. In some examples, the one or more siRNA inhibitors can include a guide strand having a sequence of SEQ ID NO: 362, or a sequence that is at least 90% or 95% homologous thereto. In some examples, the one or more siRNA inhibitors can include a guide strand having a sequence of SEQ ID NO: 364, or a sequence that is at least 90% or 95% homologous thereto. In some examples, the one or more siRNA inhibitors can include a guide strand having a sequence of SEQ ID NO: 366, or a sequence that is at least 90% or 95% homologous thereto. In some examples, the one or more siRNA inhibitors can include a guide strand having a sequence of SEQ ID NO: 368, or a sequence that is at least 90% or 95% homologous thereto. In some examples, the one or more siRNA inhibitors can include a guide strand having a sequence of SEQ ID NO: 370, or a sequence that is at least 90% or 95% homologous thereto. In some examples, the one or more siRNA inhibitors can include a guide strand having a sequence of SEQ ID NO: 372, or a sequence that is at least 90% or 95% homologous thereto. In some examples, the one or more siRNA inhibitors can include a guide strand having a sequence of SEQ ID NO: 374, or a sequence that is at least 90% or 95% homologous thereto. In some examples, the one or more siRNA inhibitors can include a guide strand having a sequence of SEQ ID NO: 376, or a sequence that is at least 90% or 95% homologous thereto. In some examples, the one or more siRNA inhibitors can include a guide strand having a sequence of SEQ ID NO: 378, or a sequence that is at least 90% or 95% homologous thereto. In some examples, the one or more siRNA inhibitors can include a guide strand having a sequence of SEQ ID NO: 380, or a sequence that is at least 90% or 95% homologous thereto. In some examples, the one or more siRNA inhibitors can include a guide strand having a sequence of SEQ ID NO: 382, or a sequence that is at least 90% or 95% homologous thereto. In some examples, the one or more siRNA inhibitors can include a guide strand having a sequence of SEQ ID NO: 384, or a sequence that is at least 90% or 95% homologous thereto. In some examples, the one or more siRNA inhibitors can include a guide strand having a sequence of SEQ ID NO: 386, or a sequence that is at least 90% or 95% homologous thereto. In some examples, the one or more siRNA inhibitors can include a guide strand having a sequence of SEQ ID NO: 388, or a sequence that is at least 90% or 95% homologous thereto. In some examples, the one or more siRNA inhibitors can include a guide strand having a sequence of SEQ ID NO: 390, or a sequence that is at least 90% or 95% homologous thereto. In some examples, the one or more siRNA inhibitors can include a guide strand having a sequence of SEQ ID NO: 392, or a sequence that is at least 90% or 95% homologous thereto. In some examples, the one or more siRNA inhibitors can include a guide strand having a sequence of SEQ ID NO: 394, or a sequence that is at least 90% or 95% homologous thereto. In some examples, the one or more siRNA inhibitors can include a guide strand having a sequence of SEQ ID NO: 396, or a sequence that is at least 90% or 95% homologous thereto. In some examples, the one or more siRNA inhibitors can include a guide strand having a sequence of SEQ ID NO: 398, or a sequence that is at least 90% or 95% homologous thereto. In some examples, the one or more siRNA inhibitors can include a guide strand having a sequence of SEQ ID NO: 400, or a sequence that is at least 90% or 95% homologous thereto. In some examples, the one or more siRNA inhibitors can include a guide strand having a sequence of SEQ ID NO: 402, or a sequence that is at least 90% or 95% homologous thereto. In some examples, the one or more siRNA inhibitors can include a guide strand having a sequence of SEQ ID NO: 404, or a sequence that is at least 90% or 95% homologous thereto. In some examples, the one or more siRNA inhibitors can include a guide strand having a sequence of SEQ ID NO: 406, or a sequence that is at least 90% or 95% homologous thereto. In some examples, the one or more siRNA inhibitors can include a guide strand having a sequence of SEQ ID NO: 408, or a sequence that is at least 90% or 95% homologous thereto. In some examples, the one or more siRNA inhibitors can include a guide strand having a sequence of SEQ ID NO: 410, or a sequence that is at least 90% or 95% homologous thereto. In some examples, the one or more siRNA inhibitors can include a guide strand having a sequence of SEQ ID NO: 412, or a sequence that is at least 90% or 95% homologous thereto. In some examples, the one or more siRNA inhibitors can include a guide strand having a sequence of SEQ ID NO: 414, or a sequence that is at least 90% or 95% homologous thereto. In some examples, the one or more siRNA inhibitors can include a guide strand having a sequence of SEQ ID NO: 416, or a sequence that is at least 90% or 95% homologous thereto. In some examples, the one or more siRNA inhibitors can include a guide strand having a sequence of SEQ ID NO: 418, or a sequence that is at least 90% or 95% homologous thereto. In some examples, the one or more siRNA inhibitors can include a guide strand having a sequence of SEQ ID NO: 420, or a sequence that is at least 90% or 95% homologous thereto. In some examples, the one or more siRNA inhibitors can include a guide strand having a sequence of SEQ ID NO: 422, or a sequence that is at least 90% or 95% homologous thereto. In some examples, the one or more siRNA inhibitors can include a guide strand having a sequence of SEQ ID NO: 424, or a sequence that is at least 90% or 95% homologous thereto. In some examples, the one or more siRNA inhibitors can include a guide strand having a sequence of SEQ ID NO: 426, or a sequence that is at least 90% or 95% homologous thereto. In some examples, the one or more siRNA inhibitors can include a guide strand having a sequence of SEQ ID NO: 428, or a sequence that is at least 90% or 95% homologous thereto. In some examples, the one or more siRNA inhibitors can include a guide strand having a sequence of SEQ ID NO: 430, or a sequence that is at least 90% or 95% homologous thereto. In some examples, the one or more siRNA inhibitors can include a guide strand having a sequence of SEQ ID NO: 432, or a sequence that is at least 90% or 95% homologous thereto. In some examples, the one or more siRNA inhibitors can include a guide strand having a sequence of SEQ ID NO: 434, or a sequence that is at least 90% or 95% homologous thereto. In some examples, the one or more siRNA inhibitors can include a guide strand having a sequence of SEQ ID NO: 436, or a sequence that is at least 90% or 95% homologous thereto. In some examples, the one or more siRNA inhibitors can include a guide strand having a sequence of SEQ ID NO: 438, or a sequence that is at least 90% or 95% homologous thereto. In some examples, the one or more siRNA inhibitors can include a guide strand having a sequence of SEQ ID NO: 440, or a sequence that is at least 90% or 95% homologous thereto. In some examples, the one or more siRNA inhibitors can include a guide strand having a sequence of SEQ ID NO: 442, or a sequence that is at least 90% or 95% homologous thereto. In some examples, the one or more siRNA inhibitors can include a guide strand having a sequence of SEQ ID NO: 444, or a sequence that is at least 90% or 95% homologous thereto. In some examples, the one or more siRNA inhibitors can include a guide strand having a sequence of SEQ ID NO: 446, or a sequence that is at least 90% or 95% homologous thereto. In some examples, the one or more siRNA inhibitors can include a guide strand having a sequence of SEQ ID NO: 448, or a sequence that is at least 90% or 95% homologous thereto. In some examples, the one or more siRNA inhibitors can include a guide strand having a sequence of SEQ ID NO: 450, or a sequence that is at least 90% or 95% homologous thereto. In some examples, the one or more siRNA inhibitors can include a guide strand having a sequence of SEQ ID NO: 452, or a sequence that is at least 90% or 95% homologous thereto. In some examples, the one or more siRNA inhibitors can include a guide strand having a sequence of SEQ ID NO: 454, or a sequence that is at least 90% or 95% homologous thereto. In some examples, the one or more siRNA inhibitors can include a guide strand having a sequence of SEQ ID NO: 456, or a sequence that is at least 90% or 95% homologous thereto. In some examples, the one or more siRNA inhibitors can include a guide strand having a sequence of SEQ ID NO: 458, or a sequence that is at least 90% or 95% homologous thereto. In some examples, the one or more siRNA inhibitors can include a guide strand having a sequence of SEQ ID NO: 460, or a sequence that is at least 90% or 95% homologous thereto. In some examples, the one or more siRNA inhibitors can include a guide strand having a sequence of SEQ ID NO: 462, or a sequence that is at least 90% or 95% homologous thereto. In some examples, the one or more siRNA inhibitors can include a guide strand having a sequence of SEQ ID NO: 464, or a sequence that is at least 90% or 95% homologous thereto. In some examples, the one or more siRNA inhibitors can include a guide strand having a sequence of SEQ ID NO: 466, or a sequence that is at least 90% or 95% homologous thereto. In some examples, the one or more siRNA inhibitors can include a guide strand having a sequence of SEQ ID NO: 468, or a sequence that is at least 90% or 95% homologous thereto. In some examples, the one or more siRNA inhibitors can include a guide strand having a sequence of SEQ ID NO: 470, or a sequence that is at least 90% or 95% homologous thereto. In some examples, the one or more siRNA inhibitors can include a guide strand having a sequence of SEQ ID NO: 472, or a sequence that is at least 90% or 95% homologous thereto. In some examples, the one or more siRNA inhibitors can include a guide strand having a sequence of SEQ ID NO: 474, or a sequence that is at least 90% or 95% homologous thereto. In some examples, the one or more siRNA inhibitors can include a guide strand having a sequence of SEQ ID NO: 476, or a sequence that is at least 90% or 95% homologous thereto. In some examples, the one or more siRNA inhibitors can include a guide strand having a sequence of SEQ ID NO: 478, or a sequence that is at least 90% or 95% homologous thereto. In some examples, the one or more siRNA inhibitors can include a guide strand having a sequence of SEQ ID NO: 480, or a sequence that is at least 90% or 95% homologous thereto. In some examples, the one or more siRNA inhibitors can include a guide strand having a sequence of SEQ ID NO: 482, or a sequence that is at least 90% or 95% homologous thereto. In some examples, the one or more siRNA inhibitors can include a guide strand having a sequence of SEQ ID NO: 484, or a sequence that is at least 90% or 95% homologous thereto. In some examples, the one or more siRNA inhibitors can include a guide strand having a sequence of SEQ ID NO: 486, or a sequence that is at least 90% or 95% homologous thereto. In some examples, the one or more siRNA inhibitors can include a guide strand having a sequence of SEQ ID NO: 488, or a sequence that is at least 90% or 95% homologous thereto. In some examples, the one or more siRNA inhibitors can include a guide strand having a sequence of SEQ ID NO: 490, or a sequence that is at least 90% or 95% homologous thereto. In some examples, the one or more siRNA inhibitors can include a guide strand having a sequence of SEQ ID NO: 492, or a sequence that is at least 90% or 95% homologous thereto. In some examples, the one or more siRNA inhibitors can include a guide strand having a sequence of SEQ ID NO: 494, or a sequence that is at least 90% or 95% homologous thereto. In some examples, the one or more siRNA inhibitors can include a guide strand having a sequence of SEQ ID NO: 496, or a sequence that is at least 90% or 95% homologous thereto. In some examples, the one or more siRNA inhibitors can include a guide strand having a sequence of SEQ ID NO: 498, or a sequence that is at least 90% or 95% homologous thereto. In some examples, the one or more siRNA inhibitors can include a guide strand having a sequence of SEQ ID NO: 500, or a sequence that is at least 90% or 95% homologous thereto. In some examples, the one or more siRNA inhibitors can include a guide strand having a sequence of SEQ ID NO: 502, or a sequence that is at least 90% or 95% homologous thereto. In some examples, the one or more siRNA inhibitors can include a guide strand having a sequence of SEQ ID NO: 504, or a sequence that is at least 90% or 95% homologous thereto. In some examples, the one or more siRNA inhibitors can include a guide strand having a sequence of SEQ ID NO: 506, or a sequence that is at least 90% or 95% homologous thereto. In some examples, the one or more siRNA inhibitors can include a guide strand having a sequence of SEQ ID NO: 508, or a sequence that is at least 90% or 95% homologous thereto. In some examples, the one or more siRNA inhibitors can include a guide strand having a sequence of SEQ ID NO: 510, or a sequence that is at least 90% or 95% homologous thereto. In some examples, the one or more siRNA inhibitors can include a guide strand having a sequence of SEQ ID NO: 512, or a sequence that is at least 90% or 95% homologous thereto. In some examples, the one or more siRNA inhibitors can include a guide strand having a sequence of SEQ ID NO: 514, or a sequence that is at least 90% or 95% homologous thereto. In some examples, the one or more siRNA inhibitors can include a guide strand having a sequence of SEQ ID NO: 516, or a sequence that is at least 90% or 95% homologous thereto. In some examples, the one or more siRNA inhibitors can include a guide strand having a sequence of SEQ ID NO: 518, or a sequence that is at least 90% or 95% homologous thereto. In some examples, the one or more siRNA inhibitors can include a guide strand having a sequence of SEQ ID NO: 520, or a sequence that is at least 90% or 95% homologous thereto. In some examples, the one or more siRNA inhibitors can include a guide strand having a sequence of SEQ ID NO: 522, or a sequence that is at least 90% or 95% homologous thereto. In some examples, the one or more siRNA inhibitors can include a guide strand having a sequence of SEQ ID NO: 524, or a sequence that is at least 90% or 95% homologous thereto. In some examples, the one or more siRNA inhibitors can include a guide strand having a sequence of SEQ ID NO: 526, or a sequence that is at least 90% or 95% homologous thereto. In some examples, the one or more siRNA inhibitors can include a guide strand having a sequence of SEQ ID NO: 528, or a sequence that is at least 90% or 95% homologous thereto. In some examples, the one or more siRNA inhibitors can include a guide strand having a sequence of SEQ ID NO: 530, or a sequence that is at least 90% or 95% homologous thereto. In some examples, the one or more siRNA inhibitors can include a guide strand having a sequence of SEQ ID NO: 532, or a sequence that is at least 90% or 95% homologous thereto. In some examples, the one or more siRNA inhibitors can include a guide strand having a sequence of SEQ ID NO: 534, or a sequence that is at least 90% or 95% homologous thereto. In some examples, the one or more siRNA inhibitors can include a guide strand having a sequence of SEQ ID NO: 536, or a sequence that is at least 90% or 95% homologous thereto. In some examples, the one or more siRNA inhibitors can include a guide strand having a sequence of SEQ ID NO: 538, or a sequence that is at least 90% or 95% homologous thereto. In some examples, the one or more siRNA inhibitors can include a guide strand having a sequence of SEQ ID NO: 540, or a sequence that is at least 90% or 95% homologous thereto. In some examples, the one or more siRNA inhibitors can include a guide strand having a sequence of SEQ ID NO: 542, or a sequence that is at least 90% or 95% homologous thereto. In some examples, the one or more siRNA inhibitors can include a guide strand having a sequence of SEQ ID NO: 544, or a sequence that is at least 90% or 95% homologous thereto. In some examples, the one or more siRNA inhibitors can include a guide strand having a sequence of SEQ ID NO: 546, or a sequence that is at least 90% or 95% homologous thereto. In some examples, the one or more siRNA inhibitors can include a guide strand having a sequence of SEQ ID NO: 548, or a sequence that is at least 90% or 95% homologous thereto. In some examples, the one or more siRNA inhibitors can include a guide strand having a sequence of SEQ ID NO: 550, or a sequence that is at least 90% or 95% homologous thereto. In some examples, the one or more siRNA inhibitors can include a guide strand having a sequence of SEQ ID NO: 552, or a sequence that is at least 90% or 95% homologous thereto. In some examples, the one or more siRNA inhibitors can include a guide strand having a sequence of SEQ ID NO: 554, or a sequence that is at least 90% or 95% homologous thereto. In some examples, the one or more siRNA inhibitors can include a guide strand having a sequence of SEQ ID NO: 556, or a sequence that is at least 90% or 95% homologous thereto. In some examples, the one or more siRNA inhibitors can include a guide strand having a sequence of SEQ ID NO: 558, or a sequence that is at least 90% or 95% homologous thereto. In some examples, the one or more siRNA inhibitors can include a guide strand having a sequence of SEQ ID NO: 560, or a sequence that is at least 90% or 95% homologous thereto. In some examples, the one or more siRNA inhibitors can include a guide strand having a sequence of SEQ ID NO: 562, or a sequence that is at least 90% or 95% homologous thereto. In some examples, the one or more siRNA inhibitors can include a guide strand having a sequence of SEQ ID NO: 564, or a sequence that is at least 90% or 95% homologous thereto. In some examples, the one or more siRNA inhibitors can include a guide strand having a sequence of SEQ ID NO: 566, or a sequence that is at least 90% or 95% homologous thereto. In some examples, the one or more siRNA inhibitors can include a guide strand having a sequence of SEQ ID NO: 568, or a sequence that is at least 90% or 95% homologous thereto. In some examples, the one or more siRNA inhibitors can include a guide strand having a sequence of SEQ ID NO: 570, or a sequence that is at least 90% or 95% homologous thereto. In some examples, the one or more siRNA inhibitors can include a guide strand having a sequence of SEQ ID NO: 572, or a sequence that is at least 90% or 95% homologous thereto. In some examples, the one or more siRNA inhibitors can include a guide strand having a sequence of SEQ ID NO: 574, or a sequence that is at least 90% or 95% homologous thereto. In some examples, the one or more siRNA inhibitors can include a guide strand having a sequence of SEQ ID NO: 576, or a sequence that is at least 90% or 95% homologous thereto. In some examples, the one or more siRNA inhibitors can include a guide strand having a sequence of SEQ ID NO: 578, or a sequence that is at least 90% or 95% homologous thereto. In some examples, the one or more siRNA inhibitors can include a guide strand having a sequence of SEQ ID NO: 580, or a sequence that is at least 90% or 95% homologous thereto. In some examples, the one or more siRNA inhibitors can include a guide strand having a sequence of SEQ ID NO: 582, or a sequence that is at least 90% or 95% homologous thereto. In some examples, the one or more siRNA inhibitors can include a guide strand having a sequence of SEQ ID NO: 584, or a sequence that is at least 90% or 95% homologous thereto. In some examples, the one or more siRNA inhibitors can include a guide strand having a sequence of SEQ ID NO: 586, or a sequence that is at least 90% or 95% homologous thereto. In some examples, the one or more siRNA inhibitors can include a guide strand having a sequence of SEQ ID NO: 588, or a sequence that is at least 90% or 95% homologous thereto. In some examples, the one or more siRNA inhibitors can include a guide strand having a sequence of SEQ ID NO: 590, or a sequence that is at least 90% or 95% homologous thereto. In some examples, the one or more siRNA inhibitors can include a guide strand having a sequence of SEQ ID NO: 592, or a sequence that is at least 90% or 95% homologous thereto. In some examples, the one or more siRNA inhibitors can include a guide strand having a sequence of SEQ ID NO: 594, or a sequence that is at least 90% or 95% homologous thereto. In some examples, the one or more siRNA inhibitors can include a guide strand having a sequence of SEQ ID NO: 596, or a sequence that is at least 90% or 95% homologous thereto. In some examples, the one or more siRNA inhibitors can include a guide strand having a sequence of SEQ ID NO: 598, or a sequence that is at least 90% or 95% homologous thereto. In some examples, the one or more siRNA inhibitors can include a guide strand having a sequence of SEQ ID NO: 600, or a sequence that is at least 90% or 95% homologous thereto. In some examples, the one or more siRNA inhibitors can include a guide strand having a sequence of SEQ ID NO: 602, or a sequence that is at least 90% or 95% homologous thereto. In some examples, the one or more siRNA inhibitors can include a guide strand having a sequence of SEQ ID NO: 604, or a sequence that is at least 90% or 95% homologous thereto. In some examples, the one or more siRNA inhibitors can include a guide strand having a sequence of SEQ ID NO: 606, or a sequence that is at least 90% or 95% homologous thereto. In some examples, the one or more siRNA inhibitors can include a guide strand having a sequence of SEQ ID NO: 608, or a sequence that is at least 90% or 95% homologous thereto. In some examples, the one or more siRNA inhibitors can include a guide strand having a sequence of SEQ ID NO: 610, or a sequence that is at least 90% or 95% homologous thereto. In some examples, the one or more siRNA inhibitors can include a guide strand having a sequence of SEQ ID NO: 612, or a sequence that is at least 90% or 95% homologous thereto. In some examples, the one or more siRNA inhibitors can include a guide strand having a sequence of SEQ ID NO: 614, or a sequence that is at least 90% or 95% homologous thereto. In some examples, the one or more siRNA inhibitors can include a guide strand having a sequence of SEQ ID NO: 616, or a sequence that is at least 90% or 95% homologous thereto. In some examples, the one or more siRNA inhibitors can include a guide strand having a sequence of SEQ ID NO: 618, or a sequence that is at least 90% or 95% homologous thereto. In some examples, the one or more siRNA inhibitors can include a guide strand having a sequence of SEQ ID NO: 620, or a sequence that is at least 90% or 95% homologous thereto. In some examples, the one or more siRNA inhibitors can include a guide strand having a sequence of SEQ ID NO: 622, or a sequence that is at least 90% or 95% homologous thereto. In some examples, the one or more siRNA inhibitors can include a guide strand having a sequence of SEQ ID NO: 624, or a sequence that is at least 90% or 95% homologous thereto. In some examples, the one or more siRNA inhibitors can include a guide strand having a sequence of SEQ ID NO: 626, or a sequence that is at least 90% or 95% homologous thereto. In some examples, the one or more siRNA inhibitors can include a guide strand having a sequence of SEQ ID NO: 628, or a sequence that is at least 90% or 95% homologous thereto. In some examples, the one or more siRNA inhibitors can include a guide strand having a sequence of SEQ ID NO: 630, or a sequence that is at least 90% or 95% homologous thereto. In some examples, the one or more siRNA inhibitors can include a guide strand having a sequence of SEQ ID NO: 632, or a sequence that is at least 90% or 95% homologous thereto. In some examples, the one or more siRNA inhibitors can include a guide strand having a sequence of SEQ ID NO: 634, or a sequence that is at least 90% or 95% homologous thereto. In some examples, the one or more siRNA inhibitors can include a guide strand having a sequence of SEQ ID NO: 636, or a sequence that is at least 90% or 95% homologous thereto. In some examples, the one or more siRNA inhibitors can include a guide strand having a sequence of SEQ ID NO: 638, or a sequence that is at least 90% or 95% homologous thereto. In some examples, the one or more siRNA inhibitors can include a guide strand having a sequence of SEQ ID NO: 640, or a sequence that is at least 90% or 95% homologous thereto. In some examples, the one or more siRNA inhibitors can include a guide strand having a sequence of SEQ ID NO: 642, or a sequence that is at least 90% or 95% homologous thereto. In some examples, the one or more siRNA inhibitors can include a guide strand having a sequence of SEQ ID NO: 644, or a sequence that is at least 90% or 95% homologous thereto. In some examples, the one or more siRNA inhibitors can include a guide strand having a sequence of SEQ ID NO: 646, or a sequence that is at least 90% or 95% homologous thereto. In some examples, the one or more siRNA inhibitors can include a guide strand having a sequence of SEQ ID NO: 648, or a sequence that is at least 90% or 95% homologous thereto. In some examples, the one or more siRNA inhibitors can include a guide strand having a sequence of SEQ ID NO: 650, or a sequence that is at least 90% or 95% homologous thereto. In some examples, the one or more siRNA inhibitors can include a guide strand having a sequence of SEQ ID NO: 652, or a sequence that is at least 90% or 95% homologous thereto. In some examples, the one or more siRNA inhibitors can include a guide strand having a sequence of SEQ ID NO: 654, or a sequence that is at least 90% or 95% homologous thereto. In some examples, the one or more siRNA inhibitors can include a guide strand having a sequence of SEQ ID NO: 656, or a sequence that is at least 90% or 95% homologous thereto. In some examples, the one or more siRNA inhibitors can include a guide strand having a sequence of SEQ ID NO: 658, or a sequence that is at least 90% or 95% homologous thereto. In some examples, the one or more siRNA inhibitors can include a guide strand having a sequence of SEQ ID NO: 660, or a sequence that is at least 90% or 95% homologous thereto. In some examples, the one or more siRNA inhibitors can include a guide strand having a sequence of SEQ ID NO: 662, or a sequence that is at least 90% or 95% homologous thereto. In some examples, the one or more siRNA inhibitors can include a guide strand having a sequence of SEQ ID NO: 664, or a sequence that is at least 90% or 95% homologous thereto. In some examples, the one or more siRNA inhibitors can include a guide strand having a sequence of SEQ ID NO: 666, or a sequence that is at least 90% or 95% homologous thereto. In some examples, the one or more siRNA inhibitors can include a guide strand having a sequence of SEQ ID NO: 668, or a sequence that is at least 90% or 95% homologous thereto. In some examples, the one or more siRNA inhibitors can include a guide strand having a sequence of SEQ ID NO: 670, or a sequence that is at least 90% or 95% homologous thereto. In some examples, the one or more siRNA inhibitors can include a guide strand having a sequence of SEQ ID NO: 672, or a sequence that is at least 90% or 95% homologous thereto. In some examples, the one or more siRNA inhibitors can include a guide strand having a sequence of SEQ ID NO: 674, or a sequence that is at least 90% or 95% homologous thereto. In some examples, the one or more siRNA inhibitors can include a guide strand having a sequence of SEQ ID NO: 676, or a sequence that is at least 90% or 95% homologous thereto. In some examples, the one or more siRNA inhibitors can include a guide strand having a sequence of SEQ ID NO: 678, or a sequence that is at least 90% or 95% homologous thereto. In some examples, the one or more siRNA inhibitors can include a guide strand having a sequence of SEQ ID NO: 680, or a sequence that is at least 90% or 95% homologous thereto. In some examples, the one or more siRNA inhibitors can include a guide strand having a sequence of SEQ ID NO: 682, or a sequence that is at least 90% or 95% homologous thereto. In some examples, the one or more siRNA inhibitors can include a guide strand having a sequence of SEQ ID NO: 684, or a sequence that is at least 90% or 95% homologous thereto. In some examples, the one or more siRNA inhibitors can include a guide strand having a sequence of SEQ ID NO: 686, or a sequence that is at least 90% or 95% homologous thereto. In some examples, the one or more siRNA inhibitors can include a guide strand having a sequence of SEQ ID NO: 688, or a sequence that is at least 90% or 95% homologous thereto. In some examples, the one or more siRNA inhibitors can include a guide strand having a sequence of SEQ ID NO: 690, or a sequence that is at least 90% or 95% homologous thereto. In some examples, the one or more siRNA inhibitors can include a guide strand having a sequence of SEQ ID NO: 692, or a sequence that is at least 90% or 95% homologous thereto. In some examples, the one or more siRNA inhibitors can include a guide strand having a sequence of SEQ ID NO: 694, or a sequence that is at least 90% or 95% homologous thereto. In some examples, the one or more siRNA inhibitors can include a guide strand having a sequence of SEQ ID NO: 696, or a sequence that is at least 90% or 95% homologous thereto. In some examples, the one or more siRNA inhibitors can include a guide strand having a sequence of SEQ ID NO: 698, or a sequence that is at least 90% or 95% homologous thereto. In some examples, the one or more siRNA inhibitors can include a guide strand having a sequence of SEQ ID NO: 700, or a sequence that is at least 90% or 95% homologous thereto. In some examples, the one or more siRNA inhibitors can include a guide strand having a sequence of SEQ ID NO: 702, or a sequence that is at least 90% or 95% homologous thereto. In some examples, the one or more siRNA inhibitors can include a guide strand having a sequence of SEQ ID NO: 704, or a sequence that is at least 90% or 95% homologous thereto. In some examples, the one or more siRNA inhibitors can include a guide strand having a sequence of SEQ ID NO: 706, or a sequence that is at least 90% or 95% homologous thereto. In some examples, the one or more siRNA inhibitors can include a guide strand having a sequence of SEQ ID NO: 708, or a sequence that is at least 90% or 95% homologous thereto. In some examples, the one or more siRNA inhibitors can include a guide strand having a sequence of SEQ ID NO: 710, or a sequence that is at least 90% or 95% homologous thereto. In some examples, the one or more siRNA inhibitors can include a guide strand having a sequence of SEQ ID NO: 712, or a sequence that is at least 90% or 95% homologous thereto. In some examples, the one or more siRNA inhibitors can include a guide strand having a sequence of SEQ ID NO: 714, or a sequence that is at least 90% or 95% homologous thereto. In some examples, the one or more siRNA inhibitors can include a guide strand having a sequence of SEQ ID NO: 716, or a sequence that is at least 90% or 95% homologous thereto. In some examples, the one or more siRNA inhibitors can include a guide strand having a sequence of SEQ ID NO: 718, or a sequence that is at least 90% or 95% homologous thereto. In some examples, the one or more siRNA inhibitors can include a guide strand having a sequence of SEQ ID NO: 720, or a sequence that is at least 90% or 95% homologous thereto. In some examples, the one or more siRNA inhibitors can include a guide strand having a sequence of SEQ ID NO: 722, or a sequence that is at least 90% or 95% homologous thereto. In some examples, the one or more siRNA inhibitors can include a guide strand having a sequence of SEQ ID NO: 724, or a sequence that is at least 90% or 95% homologous thereto. In some examples, the one or more siRNA inhibitors can include a guide strand having a sequence of SEQ ID NO: 726, or a sequence that is at least 90% or 95% homologous thereto. In some examples, the one or more siRNA inhibitors can include a guide strand having a sequence of SEQ ID NO: 728, or a sequence that is at least 90% or 95% homologous thereto. In some examples, the one or more siRNA inhibitors can include a guide strand having a sequence of SEQ ID NO: 730, or a sequence that is at least 90% or 95% homologous thereto. In some examples, the one or more siRNA inhibitors can include a guide strand having a sequence of SEQ ID NO: 732, or a sequence that is at least 90% or 95% homologous thereto. In some examples, the one or more siRNA inhibitors can include a guide strand having a sequence of SEQ ID NO: 734, or a sequence that is at least 90% or 95% homologous thereto. In some examples, the one or more siRNA inhibitors can include a guide strand having a sequence of SEQ ID NO: 736, or a sequence that is at least 90% or 95% homologous thereto. In some examples, the one or more siRNA inhibitors can include a guide strand having a sequence of SEQ ID NO: 738, or a sequence that is at least 90% or 95% homologous thereto. In some examples, the one or more siRNA inhibitors can include a guide strand having a sequence of SEQ ID NO: 740, or a sequence that is at least 90% or 95% homologous thereto. In some examples, the one or more siRNA inhibitors can include a guide strand having a sequence of SEQ ID NO: 742, or a sequence that is at least 90% or 95% homologous thereto. In some examples, the one or more siRNA inhibitors can include a guide strand having a sequence of SEQ ID NO: 744, or a sequence that is at least 90% or 95% homologous thereto. In some examples, the one or more siRNA inhibitors can include a guide strand having a sequence of SEQ ID NO: 746, or a sequence that is at least 90% or 95% homologous thereto. In some examples, the one or more siRNA inhibitors can include a guide strand having a sequence of SEQ ID NO: 748, or a sequence that is at least 90% or 95% homologous thereto. In some examples, the one or more siRNA inhibitors can include a guide strand having a sequence of SEQ ID NO: 750, or a sequence that is at least 90% or 95% homologous thereto. In some examples, the one or more siRNA inhibitors can include a guide strand having a sequence of SEQ ID NO: 752, or a sequence that is at least 90% or 95% homologous thereto. In some examples, the one or more siRNA inhibitors can include a guide strand having a sequence of SEQ ID NO: 754, or a sequence that is at least 90% or 95% homologous thereto. In some examples, the one or more siRNA inhibitors can include a guide strand having a sequence of SEQ ID NO: 756, or a sequence that is at least 90% or 95% homologous thereto. In some examples, the one or more siRNA inhibitors can include a guide strand having a sequence of SEQ ID NO: 758, or a sequence that is at least 90% or 95% homologous thereto. In some examples, the one or more siRNA inhibitors can include a guide strand having a sequence of SEQ ID NO: 760, or a sequence that is at least 90% or 95% homologous thereto. In some examples, the one or more siRNA inhibitors can include a guide strand having a sequence of SEQ ID NO: 762, or a sequence that is at least 90% or 95% homologous thereto. In some examples, the one or more siRNA inhibitors can include a guide strand having a sequence of SEQ ID NO: 764, or a sequence that is at least 90% or 95% homologous thereto. In some examples, the one or more siRNA inhibitors can include a guide strand having a sequence of SEQ ID NO: 766, or a sequence that is at least 90% or 95% homologous thereto. In some examples, the one or more siRNA inhibitors can include a guide strand having a sequence of SEQ ID NO: 768, or a sequence that is at least 90% or 95% homologous thereto. In some examples, the one or more siRNA inhibitors can include a guide strand having a sequence of SEQ ID NO: 770, or a sequence that is at least 90% or 95% homologous thereto. In some examples, the one or more siRNA inhibitors can include a guide strand having a sequence of SEQ ID NO: 772, or a sequence that is at least 90% or 95% homologous thereto. In some examples, the one or more siRNA inhibitors can include a guide strand having a sequence of SEQ ID NO: 774, or a sequence that is at least 90% or 95% homologous thereto. In some examples, the one or more siRNA inhibitors can include a guide strand having a sequence of SEQ ID NO: 776, or a sequence that is at least 90% or 95% homologous thereto. In some examples, the one or more siRNA inhibitors can include a guide strand having a sequence of SEQ ID NO: 778, or a sequence that is at least 90% or 95% homologous thereto. In some examples, the one or more siRNA inhibitors can include a guide strand having a sequence of SEQ ID NO: 780, or a sequence that is at least 90% or 95% homologous thereto. In some examples, the one or more siRNA inhibitors can include a guide strand having a sequence of SEQ ID NO: 782, or a sequence that is at least 90% or 95% homologous thereto. In some examples, the one or more siRNA inhibitors can include a guide strand having a sequence of SEQ ID NO: 784, or a sequence that is at least 90% or 95% homologous thereto. In some examples, the one or more siRNA inhibitors can include a guide strand having a sequence of SEQ ID NO: 786, or a sequence that is at least 90% or 95% homologous thereto. In some examples, the one or more siRNA inhibitors can include a guide strand having a sequence of SEQ ID NO: 788, or a sequence that is at least 90% or 95% homologous thereto. In some examples, the one or more siRNA inhibitors can include a guide strand having a sequence of SEQ ID NO: 790, or a sequence that is at least 90% or 95% homologous thereto. In some examples, the one or more siRNA inhibitors can include a guide strand having a sequence of SEQ ID NO: 792, or a sequence that is at least 90% or 95% homologous thereto. In some examples, the one or more siRNA inhibitors can include a guide strand having a sequence of SEQ ID NO: 794, or a sequence that is at least 90% or 95% homologous thereto. In some examples, the one or more siRNA inhibitors can include a guide strand having a sequence of SEQ ID NO: 796, or a sequence that is at least 90% or 95% homologous thereto. In some examples, the one or more siRNA inhibitors can include a guide strand having a sequence of SEQ ID NO: 798, or a sequence that is at least 90% or 95% homologous thereto. In some examples, the one or more siRNA inhibitors can include a guide strand having a sequence of SEQ ID NO: 800, or a sequence that is at least 90% or 95% homologous thereto. In some examples, the one or more siRNA inhibitors can include a guide strand having a sequence of SEQ ID NO: 802, or a sequence that is at least 90% or 95% homologous thereto. In some examples, the one or more siRNA inhibitors can include a guide strand having a sequence of SEQ ID NO: 804, or a sequence that is at least 90% or 95% homologous thereto. In some examples, the one or more siRNA inhibitors can include a guide strand having a sequence of SEQ ID NO: 806, or a sequence that is at least 90% or 95% homologous thereto. In some examples, the one or more siRNA inhibitors can include a guide strand having a sequence of SEQ ID NO: 808, or a sequence that is at least 90% or 95% homologous thereto. In some examples, the one or more siRNA inhibitors can include a guide strand having a sequence of SEQ ID NO: 810, or a sequence that is at least 90% or 95% homologous thereto. In some examples, the one or more siRNA inhibitors can include a guide strand having a sequence of SEQ ID NO: 812, or a sequence that is at least 90% or 95% homologous thereto. In some examples, the one or more siRNA inhibitors can include a guide strand having a sequence of SEQ ID NO: 814, or a sequence that is at least 90% or 95% homologous thereto. In some examples, the one or more siRNA inhibitors can include a guide strand having a sequence of SEQ ID NO: 816, or a sequence that is at least 90% or 95% homologous thereto. In some examples, the one or more siRNA inhibitors can include a guide strand having a sequence of SEQ ID NO: 818, or a sequence that is at least 90% or 95% homologous thereto. In some examples, the one or more siRNA inhibitors can include a guide strand having a sequence of SEQ ID NO: 820, or a sequence that is at least 90% or 95% homologous thereto. In some examples, the one or more siRNA inhibitors can include a guide strand having a sequence of SEQ ID NO: 822, or a sequence that is at least 90% or 95% homologous thereto. In some examples, the one or more siRNA inhibitors can include a guide strand having a sequence of SEQ ID NO: 824, or a sequence that is at least 90% or 95% homologous thereto. In some examples, the one or more siRNA inhibitors can include a guide strand having a sequence of SEQ ID NO: 826, or a sequence that is at least 90% or 95% homologous thereto. In some examples, the one or more siRNA inhibitors can include a guide strand having a sequence of SEQ ID NO: 828, or a sequence that is at least 90% or 95% homologous thereto. In some examples, the one or more siRNA inhibitors can include a guide strand having a sequence of SEQ ID NO: 830, or a sequence that is at least 90% or 95% homologous thereto. In some examples, the one or more siRNA inhibitors can include a guide strand having a sequence of SEQ ID NO: 832, or a sequence that is at least 90% or 95% homologous thereto. In some examples, the one or more siRNA inhibitors can include a guide strand having a sequence of SEQ ID NO: 834, or a sequence that is at least 90% or 95% homologous thereto. In some examples, the one or more siRNA inhibitors can include a guide strand having a sequence of SEQ ID NO: 836, or a sequence that is at least 90% or 95% homologous thereto. In some examples, the one or more siRNA inhibitors can include a guide strand having a sequence of SEQ ID NO: 838, or a sequence that is at least 90% or 95% homologous thereto. In some examples, the one or more siRNA inhibitors can include a guide strand having a sequence of SEQ ID NO: 840, or a sequence that is at least 90% or 95% homologous thereto. In some examples, the one or more siRNA inhibitors can include a guide strand having a sequence of SEQ ID NO: 842, or a sequence that is at least 90% or 95% homologous thereto. In some examples, the one or more siRNA inhibitors can include a guide strand having a sequence of SEQ ID NO: 844, or a sequence that is at least 90% or 95% homologous thereto. In some examples, the one or more siRNA inhibitors can include a guide strand having a sequence of SEQ ID NO: 846, or a sequence that is at least 90% or 95% homologous thereto. In some examples, the one or more siRNA inhibitors can include a guide strand having a sequence of SEQ ID NO: 848, or a sequence that is at least 90% or 95% homologous thereto. In some examples, the one or more siRNA inhibitors can include a guide strand having a sequence of SEQ ID NO: 850, or a sequence that is at least 90% or 95% homologous thereto. In some examples, the one or more siRNA inhibitors can include a guide strand having a sequence of SEQ ID NO: 852, or a sequence that is at least 90% or 95% homologous thereto. In some examples, the one or more siRNA inhibitors can include a guide strand having a sequence of SEQ ID NO: 854, or a sequence that is at least 90% or 95% homologous thereto. In some examples, the one or more siRNA inhibitors can include a guide strand having a sequence of SEQ ID NO: 856, or a sequence that is at least 90% or 95% homologous thereto. In some examples, the one or more siRNA inhibitors can include a guide strand having a sequence of SEQ ID NO: 858, or a sequence that is at least 90% or 95% homologous thereto. In some examples, the one or more siRNA inhibitors can include a guide strand having a sequence of SEQ ID NO: 860, or a sequence that is at least 90% or 95% homologous thereto. In some examples, the one or more siRNA inhibitors can include a guide strand having a sequence of SEQ ID NO: 862, or a sequence that is at least 90% or 95% homologous thereto. In some examples, the one or more siRNA inhibitors can include a guide strand having a sequence of SEQ ID NO: 864, or a sequence that is at least 90% or 95% homologous thereto. In some examples, the one or more siRNA inhibitors can include a guide strand having a sequence of SEQ ID NO: 866, or a sequence that is at least 90% or 95% homologous thereto. In some examples, the one or more siRNA inhibitors can include a guide strand having a sequence of SEQ ID NO: 868, or a sequence that is at least 90% or 95% homologous thereto. In some examples, the one or more siRNA inhibitors can include a guide strand having a sequence of SEQ ID NO: 870, or a sequence that is at least 90% or 95% homologous thereto. In some examples, the one or more siRNA inhibitors can include a guide strand having a sequence of SEQ ID NO: 872, or a sequence that is at least 90% or 95% homologous thereto. In some examples, the one or more siRNA inhibitors can include a guide strand having a sequence of SEQ ID NO: 874, or a sequence that is at least 90% or 95% homologous thereto. In some examples, the one or more siRNA inhibitors can include a guide strand having a sequence of SEQ ID NO: 876, or a sequence that is at least 90% or 95% homologous thereto. In some examples, the one or more siRNA inhibitors can include a guide strand having a sequence of SEQ ID NO: 878, or a sequence that is at least 90% or 95% homologous thereto. In some examples, the one or more siRNA inhibitors can include a guide strand having a sequence of SEQ ID NO: 880, or a sequence that is at least 90% or 95% homologous thereto. In some examples, the one or more siRNA inhibitors can include a guide strand having a sequence of SEQ ID NO: 882, or a sequence that is at least 90% or 95% homologous thereto. In some examples, the one or more siRNA inhibitors can include a guide strand having a sequence of SEQ ID NO: 884, or a sequence that is at least 90% or 95% homologous thereto. In some examples, the one or more siRNA inhibitors can include a guide strand having a sequence of SEQ ID NO: 886, or a sequence that is at least 90% or 95% homologous thereto. In some examples, the one or more siRNA inhibitors can include a guide strand having a sequence of SEQ ID NO: 888, or a sequence that is at least 90% or 95% homologous thereto. In some examples, the one or more siRNA inhibitors can include a guide strand having a sequence of SEQ ID NO: 890, or a sequence that is at least 90% or 95% homologous thereto. In some examples, the one or more siRNA inhibitors can include a guide strand having a sequence of SEQ ID NO: 892, or a sequence that is at least 90% or 95% homologous thereto. In some examples, the one or more siRNA inhibitors can include a guide strand having a sequence of SEQ ID NO: 894, or a sequence that is at least 90% or 95% homologous thereto. In some examples, the one or more siRNA inhibitors can include a guide strand having a sequence of SEQ ID NO: 896, or a sequence that is at least 90% or 95% homologous thereto. In some examples, the one or more siRNA inhibitors can include a guide strand having a sequence of SEQ ID NO: 898, or a sequence that is at least 90% or 95% homologous thereto. In some examples, the one or more siRNA inhibitors can include a guide strand having a sequence of SEQ ID NO: 900, or a sequence that is at least 90% or 95% homologous thereto. In some examples, the one or more siRNA inhibitors can include a guide strand having a sequence of SEQ ID NO: 902, or a sequence that is at least 90% or 95% homologous thereto. In some examples, the one or more siRNA inhibitors can include a guide strand having a sequence of SEQ ID NO: 904, or a sequence that is at least 90% or 95% homologous thereto. In some examples, the one or more siRNA inhibitors can include a guide strand having a sequence of SEQ ID NO: 906, or a sequence that is at least 90% or 95% homologous thereto. In some examples, the one or more siRNA inhibitors can include a guide strand having a sequence of SEQ ID NO: 908, or a sequence that is at least 90% or 95% homologous thereto. In some examples, the one or more siRNA inhibitors can include a guide strand having a sequence of SEQ ID NO: 910, or a sequence that is at least 90% or 95% homologous thereto. In some examples, the one or more siRNA inhibitors can include a guide strand having a sequence of SEQ ID NO: 912, or a sequence that is at least 90% or 95% homologous thereto. In some examples, the one or more siRNA inhibitors can include a guide strand having a sequence of SEQ ID NO: 914, or a sequence that is at least 90% or 95% homologous thereto. In some examples, the one or more siRNA inhibitors can include a guide strand having a sequence of SEQ ID NO: 916, or a sequence that is at least 90% or 95% homologous thereto. In some examples, the one or more siRNA inhibitors can include a guide strand having a sequence of SEQ ID NO: 918, or a sequence that is at least 90% or 95% homologous thereto. In some examples, the one or more siRNA inhibitors can include a guide strand having a sequence of SEQ ID NO: 920, or a sequence that is at least 90% or 95% homologous thereto. In some examples, the one or more siRNA inhibitors can include a guide strand having a sequence of SEQ ID NO: 922, or a sequence that is at least 90% or 95% homologous thereto. In some examples, the one or more siRNA inhibitors can include a guide strand having a sequence of SEQ ID NO: 924, or a sequence that is at least 90% or 95% homologous thereto. In some examples, the one or more siRNA inhibitors can include a guide strand having a sequence of SEQ ID NO: 926, or a sequence that is at least 90% or 95% homologous thereto. In some examples, the one or more siRNA inhibitors can include a guide strand having a sequence of SEQ ID NO: 928, or a sequence that is at least 90% or 95% homologous thereto. In some examples, the one or more siRNA inhibitors can include a guide strand having a sequence of SEQ ID NO: 930, or a sequence that is at least 90% or 95% homologous thereto. In some examples, the one or more siRNA inhibitors can include a guide strand having a sequence of SEQ ID NO: 932, or a sequence that is at least 90% or 95% homologous thereto. In some examples, the one or more siRNA inhibitors can include a guide strand having a sequence of SEQ ID NO: 934, or a sequence that is at least 90% or 95% homologous thereto. In some examples, the one or more siRNA inhibitors can include a guide strand having a sequence of SEQ ID NO: 936, or a sequence that is at least 90% or 95% homologous thereto. In some examples, the one or more siRNA inhibitors can include a guide strand having a sequence of SEQ ID NO: 938, or a sequence that is at least 90% or 95% homologous thereto. In some examples, the one or more siRNA inhibitors can include a guide strand having a sequence of SEQ ID NO: 940, or a sequence that is at least 90% or 95% homologous thereto. In some examples, the one or more siRNA inhibitors can include a guide strand having a sequence of SEQ ID NO: 942, or a sequence that is at least 90% or 95% homologous thereto. In some examples, the one or more siRNA inhibitors can include a guide strand having a sequence of SEQ ID NO: 944, or a sequence that is at least 90% or 95% homologous thereto. In some examples, the one or more siRNA inhibitors can include a guide strand having a sequence of SEQ ID NO: 946, or a sequence that is at least 90% or 95% homologous thereto. In some examples, the one or more siRNA inhibitors can include a guide strand having a sequence of SEQ ID NO: 948, or a sequence that is at least 90% or 95% homologous thereto. In some examples, the one or more siRNA inhibitors can include a guide strand having a sequence of SEQ ID NO: 950, or a sequence that is at least 90% or 95% homologous thereto. In some examples, the one or more siRNA inhibitors can include a guide strand having a sequence of SEQ ID NO: 952, or a sequence that is at least 90% or 95% homologous thereto. In some examples, the one or more siRNA inhibitors can include a guide strand having a sequence of SEQ ID NO: 954, or a sequence that is at least 90% or 95% homologous thereto. In some examples, the one or more siRNA inhibitors can include a guide strand having a sequence of SEQ ID NO: 956, or a sequence that is at least 90% or 95% homologous thereto. In some examples, the one or more siRNA inhibitors can include a guide strand having a sequence of SEQ ID NO: 958, or a sequence that is at least 90% or 95% homologous thereto. In some examples, the one or more siRNA inhibitors can include a guide strand having a sequence of SEQ ID NO: 960, or a sequence that is at least 90% or 95% homologous thereto. In some examples, the one or more siRNA inhibitors can include a guide strand having a sequence of SEQ ID NO: 962, or a sequence that is at least 90% or 95% homologous thereto. In some examples, the one or more siRNA inhibitors can include a guide strand having a sequence of SEQ ID NO: 964, or a sequence that is at least 90% or 95% homologous thereto. In some examples, the one or more siRNA inhibitors can include a guide strand having a sequence of SEQ ID NO: 966, or a sequence that is at least 90% or 95% homologous thereto. In some examples, the one or more siRNA inhibitors can include a guide strand having a sequence of SEQ ID NO: 968, or a sequence that is at least 90% or 95% homologous thereto.

Alternatively to or in combination with one or more of the guide strands listed in Table 2, one or more of the following guide strands, or a strand 90% or 95% homologous thereto, can also be employed in the present methods and/or compositions to inhibit expression of the ABCC11 gene: GUUUCAGGACUUAUUUAUA (SEQ ID NO: 970), CCUACUUCAUUAUUGGAUA (SEQ ID NO: 971), GUCCUGUCCUUAAUGGUGA (SEQ ID NO: 972), and CAAAGAUCCUGGAAUAUUC (SEQ ID NO: 973). In some examples, the one or more siRNA inhibitors can include a guide strand having a sequence of SEQ ID NO: 970, or a sequence that is at least 90% or 95% homologous thereto. In some examples, the one or more siRNA inhibitors can include a guide strand having a sequence of SEQ ID NO: 971, or a sequence that is at least 90% or 95% homologous thereto. In some examples, the one or more siRNA inhibitors can include a guide strand having a sequence of SEQ ID NO: 972, or a sequence that is at least 90% or 95% homologous thereto. In some examples, the one or more siRNA inhibitors can include a guide strand having a sequence of SEQ ID NO: 973, or a sequence that is at least 90% or 95% homologous thereto.

It is also noted that one or more of the guide strands listed above, or a portion thereof, can also be used as an ASO. In some examples, one or more of the guide strands listed above, or the portion thereof, can be modified with phosphorothioate linkages in place of phosphodiester bonds to increase the stability of the single stranded RNA for use as an ASO. In some examples, one or more of the guide strands listed above, or the portion thereof, can be modified to include a 2′-O-methyl group, 2′-fluoro group, 2′-O-methoxyethyl group, the like, or a combination thereof to increase the stability of the single stranded RNA for use as an ASO. In some examples, one or more of the guide strands, or the portion thereof, can be modified with locked nucleic acid (LNA), which contains a methylene bridge between the 2′ and 4′ position of the ribose to increase the stability of the single stranded RNA for use as an ASO. Any suitable combination of these modifications, or other similar, related, or suitable modification, can be employed with one or more of the guide strands listed above, or the portion thereof, to prepare a suitable ASO for use in inhibiting expression of the ABCC11 gene. In some examples, a 15-19 nucleotide portion of one or more of the guide strands listed above can be employed as an ASO to inhibit expression of the ABCC11 gene.

It is also noted that any RNA inhibitor described herein can include the modifications listed above with respect to ASOs, or similar modifications, to increase the stability thereof. In some examples, the RNA sequences can include modifications to either the phosphate-sugar backbone or the base. For example, the phosphodiester linkages of the RNA can be modified to include at least one of a nitrogen, sulfur, or heteroatom. Likewise, in some examples, bases can be modified to block the activity of adenosine deaminase. It is further noted that the RNA sequence can be prepared by any suitable method. In some examples, the RNA sequence can be produced enzymatically. In other examples, the RNA sequence can be produced by partial or total organic synthesis. In some examples, additional moieties can be included to facilitate self-delivery of the RNA sequence, such a lipids, sugars (e.g. N-acetylgalactosamine (GalNAc)), ligands, peptides, cholesterol, the like, or combinations thereof to facilitate cellular uptake of the RNA inhibitor. Thus, in some examples, the RNA inhibitor can include self-delivery modifications so as to not require the addition of transfection reagents and aids.

The RNA sequences of the present invention can be administered in a variety of forms. For example, in some cases, RNA sequences can be administered as hybridized double stranded complementary RNA (dsRNA), as single stranded RNA (ssRNA), as a single hairpin molecule of RNA (shRNA), as ribozymes, as DNA antisense (AS), as nucleic acid mimics such as peptide nucleic acids or mopholinos, or in any other suitable form. Whether administered as dsRNA, ssRNA, shRNA, or AS, there are a variety of mechanisms by which the RNA/DNA sequences of the present invention can be delivered to a subject.

Suitable delivery mechanisms include but are not limited to injections, including intradermal injection using single needles and needle arrays, topical formulations, such as lotions, creams, gels, ointments, jellies (such as petroleum jelly), adhesives, pastes, liquids, soaps, shampoos, transdermal patches, films, electrophoresis, sonoporation, iontophoresis, nanoparticles, the like, or combinations thereof. In one aspect, the specific carrier utilized in the production of a formation may be selected because of its positive impact on skin. For example, in some cases, carriers that moisturize, hydrate, or otherwise benefit the skin can be used. In yet other examples, carriers that absorb moisture from the skin can be beneficial. This can help eliminate an environment in which odor-producing bacterial thrive. Thus, in some examples, the carrier can include a water-absorbing component (i.e. dessicant).

In further detail, in some examples, a therapeutically effective amount of an RNA inhibitor can be administered via injection, such as intramuscular injection, intravenous injection, subcutaneous injection, intrathecal injection, intradermal injection, transdermal injection or the like. In such examples, the pharmaceutically acceptable carrier can include a variety of components, such as water, a solubilizing or dispersing agent, a tonicity agent, a pH adjuster or buffering agent, a preservative, a chelating agent, a bulking agent, the like, or a combination thereof.

In some examples, an injectable therapeutic composition can include a solubilizing or dispersing agent. Non-limiting examples of solubilizing or dispersing agents can include polyoxyethylene sorbitan monooleates, lecithin, polyoxyethylene polyoxypropylene co-polymers, propylene glycol, glycerin, ethanol, polyethylene glycols, sorbitol, dimethylacetamide, polyethoxylated castor oils, n-lactamide, cyclodextrins, caboxymethyl cellulose, acacia, gelatin, methyl cellulose, polyvinyl pyrrolidone, the like, or combinations thereof.

In some examples, an injectable therapeutic composition can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. The tonicity agent can be used to provide an appropriate tonicity of the therapeutic composition. In one aspect, the tonicity of the therapeutic composition can be from about 250 to about 350 milliosmoles/liter (mOsm/L). In another aspect, the tonicity of the therapeutic composition can be from about 277 to about 310 mOsm/L.

In some examples, an injectable therapeutic composition can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8.

In some examples, an injectable therapeutic composition can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In some examples, an injectable therapeutic composition can include a chelating agent. Non-limiting examples of chelating agents can include ethylenediaminetetra acetic acid, calcium, calcium disodium, versetamide, calteridol, diethylenetriaminepenta acetic acid, the like, or combinations thereof.

In some examples, an injectable therapeutic composition can include a bulking agent. Non-limiting examples of bulking agents can include sucrose, lactose, trehalose, mannitol, sorbitol, glucose, rafinose, glycine, histidine, polyvinyl pyrrolidone, the like, or combinations thereof.

In one example, a method of treating osmidrosis in a subject is disclosed. The method can comprise administering to the subject a therapeutically effective amount of an ABCC11 inhibitor, wherein the ABCC11 inhibitor is delivered to the subject via injection.

In some examples, a therapeutically effective amount of the RNA inhibitor can be administered via a microneedle array. Such microneedle arrays can include a base portion and a plurality of microneedles attached to, or projecting from the surface of the base portion. In some examples, the base portion can be a polymer layer. The microneedles can be applied to a skin surface of a subject in a manner sufficient to embed the microneedles into the skin surface. In some embodiments, the base portion of the microneedle array can then be separated from the microneedles such that the microneedles remain embedded in the skin surface and the base portion can be removed from the skin surface. In such examples, the microneedles can be maintained in the skin surface until the microneedles are absorbed by the subject. In other embodiments, the base portion and the microneedles can remain connected.

The microneedles of the microneedle array can have any suitable length. In some examples, the microneedles can have a length from about 1 μm to about 10,000 μm. In yet other examples, the microneedles can have a length from about 50 μm to about 1,000 μm. In still other examples, the microneedles can have a length from about 75 μm to about 500 μm.

The microneedle array can have any suitable number of microneedles, depending on the size and distribution of the microneedles. In some examples, the microneedle array can have from about 1 microneedle to about 25,000,000 microneedles. In some examples, the microneedle array can have from about 10 microneedles to about 200 microneedles. In yet other examples, the microneedle array can have from about 50 microneedles to about 500 microneedles. In yet other examples, the microneedle array can have from about 100 microneedles to about 1000 microneedles. In yet other examples, the microneedle array can have from about 500 microneedles to about 50,000 microneedles. In still additional examples, the microneedle array can have from about 10,000 microneedles to about 10,000,000 microneedles.

The microneedle arrays can have a variety of distributions of microneedles. For example, in some cases, the microneedles can be spaced on the base portion at a density of from about 1 microneedle per square centimeter (cm²) to about 2500 microneedles per cm². In other examples, the microneedles can be spaced on the base portion at a density of from about 10 microneedles per cm² to about 100 microneedles per cm². In other examples, the microneedles can be spaced on the base portion at a density of from about 50 microneedles per cm² to about 200 microneedles per cm². In yet other examples, the microneedles can be spaced on the base portion at a density of from about 100 microneedles per cm² to about 1000 microneedles per cm². In still other examples, the microneedles can be spaced on the base portion at a density of from about 500 microneedles per cm² to about 2500 microneedles per cm².

The microneedle array can be fabricated to simultaneously apply needles to a range of skin surface areas. For example, the microneedle arrays can be fabricated as a continuous sheet, which can optionally be sub-divided into smaller unit doses. In some examples, the microneedle array unit dose can be fabricated to have a surface area or to cover a skin surface area from 1 mm² to 20 cm² or from 10 cm² to 80 cm². In yet other examples, the microneedle array unit dose can be fabricated to have a surface area or to cover a skin surface area from 50 cm² to 150 cm², or from 100 cm² to 1 m². In one specific embodiment, the unit dose can have a surface area or cover a skin surface area from 1 cm² to 350 cm². Unit dose size can be preselected to be appropriate for treating the skin surface of a particular body part, such as the palm of the hand, the sole of the foot, or the front or back torso, for example. Further, the flexible sheets of microneedles can be cut into shapes convenient for application to a selected body part. Thus, the microneedle array can have the shape of a circle, an oval, a triangle, a square, a rectangle, a trapezoid, a rhombus, a crescent, a polygonal shape, or any other suitable shape for a particular application. Alternatively, a preselected shape can be dispensed as the base layer and needles subsequently produced from that base layer of a preselected shape.

In some examples, the microneedles of the microneedle arrays can be made of bioabsorbable/biodegradable materials and, in some further examples, can also include materials that can hydrate to form an intradermal and/or subcutaneous depot upon administration. Non-limiting examples of bioabsorbable/biodegradable materials that can be used include polyvinyl alcohol, polyvinylpyrrolidone, carbomers, polyacrylic acid, polyoxyethylene/polyoxypropylene copolymers, other copolymers, albumins, casein, zein, collagen, other proteins, glucose, sucrose, maltose, trehalose, amylose, dextrose, fructose, mannose, galactose, other sugars, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, other sugar alcohols, chondroitin and/or other glycosaminoglycans, inulin, starches, acacia gum, agar, carboxymethyl cellulose, methyl cellulose, ethyl cellulose, alginates, carrageenan, cassia gums, cellulose gums, chitin, chitosan, curdlan, gelatin, dextran, fibrin, fulcelleran, gellan gum, ghatti gum, guar gum, tragacanth, karaya gum, locust bean gum, pectin, starch, tara gum, xanthan gum, and other polysaccharides, and functionalized derivatives of any of the above, copolymers thereof, or mixtures thereof. The bioabsorbable/biodegradable materials are generally only limited by the ability to create a viscous solution in a solvent that can volatilize during formation of the fiber-like needle structure, and/or the property of drying to form a glassy or non-crystalline solid.

In one example, a method of treating osmidrosis in a targeted region of a subject is disclosed. The method can comprise administering to the subject a therapeutically effective amount of an ABCC11 inhibitor, wherein the ABCC11 inhibitor is delivered to the subject via a microneedle array.

In one aspect, a topical formulation containing a therapeutically effective amount of an ABCC11 inhibitor can be used to treat the symptoms of osmidrosis at a targeted localized region or area of subject's skin by direct application thereto. Further, the topical formulations can be formulated for local and/or systemic delivery of one or more components of the therapeutic composition. Where the therapeutic composition is formulated for topical or transdermal administration, the pharmaceutically acceptable carrier can include a variety of components suitable for forming a suspension, dispersion, lotion, cream, ointment, gel, foam, patch, powder, paste, sponge, shampoo, jellies (such as petroleum jelly), adhesives, pastes, liquids, soaps, the like, or a combination thereof. Non-limiting examples can include a solubilizer, an emulsifier, a dispersant, a thickener, an emollient, a pH adjuster, a tonicity agent, a preservative, an adhesive, a penetration enhancer, the like, or a combination thereof.

Non-limiting examples of solubilizers and/or emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof. In some examples, the solubilizer can also include a hydrocarbon or fatty substance, such as petrolatum, microcrystalline wax, paraffin wax, mineral oil, ceresi, coconut oil, bees wax, olive oil, lanolin, peanut oil, spermaceti wax, sesame oil, almond oil, hydrogenated castor oils, cotton seed oil, soybean oil, corn oil, hydrogenated sulfated castor oils, cetyl alcohol, stearyl alcohol, oleyl alcohol, lauryl alcohol, myristyl alcohol, stearic acid, oleic acid, palmitic acid, lauraic acid, ethyl oleate, isopropyl myristicate, the like, or combinations thereof. In some examples, the solubilizer can include a silicon, such as polydimethylsiloxanes, methicones, dimethylpropylsiloxanes, methyl phenyl polysiloxanes, steryl esters of dimethyl polysiloxanes, ethoxylated dimethicones, ethoxylated methicones, the like, or combinations thereof.

In some additional examples, the therapeutic composition can include a dispersant and/or thickening agent, such as polyacrylic acids (e.g. Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof.

In some examples, the therapeutic composition can include an emollient, such as aloe vera, lanolin, urea, petrolatum, shea butter, cocoa butter, mineral oil, paraffin, beeswax, squalene, jojoba oil, coconut oil, sesame oil, almond oil, cetyl alcohol, stearyl alcohol, olive oil, oleic acid, triethylhexanoin, glycerol, sorbitol, propylene glycol, cyclomethicone, dimethicone, the like, or combinations thereof. A wide range of emollient additives are known in the art and any of these may be included in the present compositions. The emollient component may provide multiple advantages, which include but are not limited to improving the cosmetic feel and appearance of the formulation during application and after drying. Generally, inclusion of emollient materials is understood by those versed in the art to suppress evaporation rate and to reduce the chemical potential of the drug-solvent system in regard to percutaneous absorption. In one embodiment, the emollient can be present in the formulation in an amount from 0.1 wt % to 10 wt %. In another embodiment, the emollient can be present in the formulation in an amount from 0.1 wt % to 5 wt %. In another embodiment, the emollient can be present in the formulation in an amount from 0.5 wt % to 3 wt %.

In some examples, the topical or transdermal composition can include an adhesive, such as acrylic adhesives, polyisobutylene adhesives, silicon adhesives, hydrogel adhesives, the like, or combinations thereof.

In some examples, the topical or transdermal composition can include a penetration enhancer, such as ethanol, propylene glycol, oleic acid and other fatty acids, azone, terpenes, terpenoids, bile acids, isopropyl myristate and other fatty esters, dimethyl sulphoxides, N-methyl-2-pyrrolidone and other pyrrolidones, the like, or combinations thereof.

The pH adjusters, tonicity agents, and preservatives can also be included in the topical or transdermal therapeutic composition, such as those pH adjusters and buffering agents, tonicity agents, and preservative agents listed above, or any other suitable pH adjusters, buffering agent, tonicity agent, or preservative for a particular formulation and/or use thereof. In some examples, the topical or transdermal therapeutic composition can also include fumed silica, mica, talc, titanium dioxide, kaolin, aluminum glycinate, ethylenediaminetetraacetic acid, fragrances, colorants, other components as described above, the like, or combinations thereof.

In some specific examples, the topical or transdermal delivery system can be in the form of aqueous lotions or creams. These topical or transdermal delivery systems can be such that following application to a skin surface, the skin surface is dry, or substantially dry, to the touch within about 1 minute to about 5 minutes. In one embodiment, the following application of the transdermal delivery system to a skin surface, the skin surface is dry, or substantially dry, to the touch within about 1 minute to about 2 minutes. In another embodiment, following application to a skin surface, the skin surface is dry, or substantially dry, to the touch in less than about 1 minute. In one embodiment, the formulation of the present invention can be substantially free of triglycerides, waxes, or liquid surfactants that, following application to a skin surface and being allowed to dry, are left behind on the skin surface (i.e. leave a residue). Following drying, the topical or transdermal delivery system of the present disclosure typically does not leave a residue on the skin surface. This is advantageous in that the risk of transfer of the substances, particularly the siRNA, from the skin is significantly reduced as compared to other non-aqueous formulations (e.g. ointments). Further, by reducing superficial residue on the skin surface, the presence of materials that might solubilize siRNA locally at the skin surface without assisting their transport onto or into the skin is reduced, which tendency might otherwise act to compromise the efficacy of the composition. For instance, if a triglyceride residue remained at the surface of the skin while the other components evaporated or absorbed into the skin, the residual triglyceride would be likely to dissolve a fraction of the siRNA active ingredient, which would therefore be less available to be delivered by the percutaneous absorbing portions of the formulation, as topically applied triglycerides are not understood to penetrate significantly into the skin.

The compositional make-up of the topical or transdermal delivery systems disclosed herein can be such that they have a low yield stress value (e.g. dynes/cm²), which allows it to be readily applied to sensitive skin areas without requiring substantial pressure for rubbing or spreading. Nonetheless, the yield stress value of the compositions is still high enough to provide for convenient, localized, and non-messy application. This is particularly advantageous in that many conditions that can be treated with formulations of the present invention often result in tender or sensitive skin. Accordingly, the transdermal delivery systems described herein can provide for better patient compliance.

In some specific examples, the topical delivery vehicle can comprise a polymer having surfactant properties, a polymer having thickening properties, a solvent for solubilizing the ABCC11 inhibitor, a glycol, a C₁₀-C₂₀ fatty acid, a base, and water.

Polymers having surfactant properties (surfactant polymers) can include a wide array of surfactant or emulsifying polymers that are known in the art. Non-limiting examples of polymers having surfactant or emulsifying properties include, but are not limited to hydrophobically modified polyacrylic acid commercially under the tradename Pemulen™ TR-I and TR-2 by Lubrizol Corp., water-soluble or water-swellable copolymers based on acrylamidoalkyl sulfonic acid and cyclic N-vinylcarboxamides commercially available under the tradename Aristoflex® AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation and a homopolymer of acrylamidoalkyl sulfonic acid commercially available under the tradename Granthix APP by Grant Industries, Inc. Another class of notable polymeric emulsifier includes hydrophobically-modified, crosslinked, anionic acrylic copolymers, including random polymers, but may also exist in other forms such as block, star, graft, and the like. In one embodiment, the hydrophobically modified, crosslinked, anionic acrylic copolymer may be synthesized from at least one acidic monomer and at least one hydrophobic ethylenically unsaturated monomer. Examples of suitable acidic monomers include those ethylenically unsaturated acid monomers that may be neutralized by a base. Examples of suitable hydrophobic ethylenically unsaturated monomers include those that contain a hydrophobic chain having a carbon chain length of at least about 3 carbon atoms.

Other materials that may be suitable polymeric surfactants can include ethylene oxide/propylene oxide block copolymers, sold under the trade name PLURONIC®, available from BASF Corporation of Parsippany, NJ., modified cellulose polymers such as those modified cellulose polymers described by the trade name KLUCEL®, available from Hercules Corporation of Wilmington, DE. Particularly notable embodiments of the invention are compositions that include hydrophobically modified polyacrylic acid, acrylamidoalkyl sulfonic acid, cyclic N-vinylcarboxamides, acrylamidoalkyl sulfonic acid, hydrophobically modified methacrylic acid, a homopolymer of acrylamidoalkyl sulfonic acid, or combinations thereof as polymeric emulsifiers; and monomeric anionic surfactants, monomeric amphoteric surfactants, or combinations thereof as foaming agents. More particularly notable embodiments of the invention are compositions that include hydrophobically modified polyacrylic acid; water-soluble or water-swellable copolymers based on acrylamidoalkyl sulfonic acid, cyclic N-vinylcarboxamides; water-soluble or water-swellable copolymers based on acrylamidoalkyl sulfonic acid, hydrophobically modified methacrylic acid; a homopolymer of acrylamidoalkyl sulfonic acid, or combinations thereof as polymeric emulsifiers, and include a betaine as the foaming surfactant. Especially notable embodiments of the invention are compositions that include copolymers based on acrylamidoalkylsulfonic acids and cyclic N-vinylcarboxamides and/or linear N-vinylcarboxamides (e.g., Aristoflex® AVC and Aristoflex® HMB from Clariant Corporation) as polymeric emulsifiers and a betaine as foaming surfactant.

Polymers having surfactant properties can enhance the ability of a formulation to support highly loaded emulsions of low polarity oils, and it has been discovered that it is in some circumstances possible to extend this capability to form emulsions of an intermediate polarity material as well. In some embodiments, the surfactant polymer can comprise about 0.01 wt % to about 3 wt %. In one embodiment, the surfactant polymer can comprise about 0.1 wt % to about 1.0 wt % of the formulations of the present invention. In one embodiment, the surfactant polymer can comprise about 0.1 wt % to about 0.5 wt % of the total formulation. In another embodiment, the surfactant polymer can comprise about 0.15 wt % to about 0.3 wt % of the total formulation.

The formulations of the present invention also can include a polymer having thickening properties (thickening polymer). In one embodiment, the polymer having thickening properties can be a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C₁₀-C₃₀ alkyl acrylate crosspolymers, and combinations thereof. In some embodiments, the polymer having thickening properties can comprise about 0.1 wt % to about 3 wt %. In another embodiment, polymers having thickening properties can be present in amounts of 0.4 wt % to about 1.0 wt % of the total composition. In one embodiment, the polymer having thickening properties comprises about 0.5 wt % to about 0.75 wt % of the total composition. The thickening polymer can be mixed with the surfactant polymer and water as a component of an aqueous phase.

In some embodiments, the formulations of the present invention can also include a base or buffer system, which is present in the formulation to neutralize and/or activate the thickening polymer in order to facilitate the formation of a composition having the desirable rheological qualities. Any base or buffer system known in the art and suitable for use in a skin contact application can be used. In one embodiment, the base can include triethanolamine, such as solutions of 10% triethanolamine (TEA), tetrasodium ethylenediaminetetraacetic acid (EDTA), alkali metal hydroxides like sodium hydroxide (NaOH), salts of weak acids such as ammonium lactate, sodium citrate, sodium ascorbate, or mixtures thereof. The base component also provides utility in that the pH of the overall composition may be adjusted to a range favorable for minimizing irritation of the skin due to pH effects. In some embodiments formulations of the present invention can also include an acid or the acid component of a buffer system, and any acid known in the art and appropriate for human skin contact may be used. Examples of acids useful in the present formulation and commonly used to adjust pH of topical formulations include but are not limited to: citric acid, lactic acid, ascorbic acid, and hydrochloric acid, and combinations of these and similar acids. Generally the pH of the formulations of the present invention can be between about 5.0 and about 7.0.

The formulations of the present invention can also include a glycol and/or glycol ether. Non-limiting examples of glycols and glycol ethers can be selected from butylene glycol, propylene glycol, diethylene glycol (Transcutol), triethylene glycol, ethylene glycol monomethyl ether, or other glycols and glycol ethers, and combinations thereof. The formulations can also include a C₁₀-C₂₀ fatty acid. Non-limiting examples of C₁₀-C₂₀ fatty acid can include oleic acid, arachidonic acid, linoleic acid, linolenic acid, or other fatty acids or combinations of fatty acids, and preferably unsaturated cis conformation fatty acids. Without being bound to any particular interpretation, such conformations are understood to disrupt superficial packing of the structured lipids of the stratum corneum, thereby promoting fluidization of these lipids and thus enhancing the diffusion of the drug and/or solvent into the skin, and are believed to play this role in the present formulation. In one embodiment, the C₁₀-C₂₀ fatty acid can be oleic acid.

In one example, a method of treating osmidrosis in a targeted region of a subject is disclosed. The method can comprise topically administering to the subject a therapeutically effective amount of an ABCC11 inhibitor, wherein the ABCC11 inhibitor is delivered to the subject via a topical or transdermal delivery vehicle.

The effectiveness of osmidrosis inhibition can depend on the particular RNA inhibitor as well as the amount of inhibiting RNA administered to the subject. Other biologically-related factors may also be important in determining the effectiveness of the inhibitors. In some examples, therapeutically effective amounts of RNA sequences can be from about 0.01 mg per squared cm of body surface area per day to about 50 mg per squared cm of body surface area per day. In other examples, therapeutically effective amounts of RNA sequences can be from about 0.05 mg per squared cm of body surface area per day to about 20 mg per squared cm of body surface area per day. In yet other examples, therapeutically effective amounts of RNA sequences can be from about 0.1 mg per squared cm of body surface area per day to about 10 mg per squared cm of body surface area per day.

Various factors can affect an appropriate amount of an ABCC11 inhibitor to ameliorate osmidrosis symptoms in a subject. Such factors can include the specific ABCC11 inhibitor or inhibitors being used, type or extent of odor-producing condition experienced by the subject, the age and weight of the subject, as well as various other physical and genetic factors, other medications being used to treat the patient, and many other factors known by those skilled in the relevant arts. As a result, there are a range of therapeutically effective amounts that can be used for treating the osmidrosis of a subject, which can depend on the above listed factors and others. In one aspect, a therapeutically effective amount can be an osmidrosis-reducing amount. In another aspect, a therapeutically effective amount can be an odor-removal or odor-reducing amount.

In some examples, a therapeutically effective amount can include an amount from about 0.01 mg to about 100 mg per day. In another aspect, a therapeutically effective amount can include an amount from about 0.1 mg per day to about 50 mg per day. In another aspect, a therapeutically effective amount can include an amount from about 0.2 mg to about 20 mg per day. In yet a further aspect, a therapeutically effective amount can include an amount from about 0.2 mg to about 1 mg per day.

In one embodiment, the amount of ABCC11 inhibitor that is therapeutically effective may be sufficient to provide a reduction of osmidrosis severity; delay of onset of odor following stimuli; accelerate removal of odor following stimuli; etc. When applied to a target region that also includes a condition or disease that causes the osmidrosis symptoms, the amount of ABCC11 inhibitor can also be sufficient to provide prolonged reduction of osmidrosis.

As previously mentioned, the therapeutically effective amount of an ABCC11 inhibitor present pharmaceutically acceptable carrier can vary depending on the particular ABCC11 inhibitor being used, the mode of administration being employed, the severity of the condition, the particular subject being treated, etc. In one embodiment, the delivery system can include from about 0.0001 wt % to about 20 wt % of an ABCC11 inhibitor. In another embodiment, the delivery system can include about 0.0005 wt % to about 10 wt % of the formulation. In another embodiment, the ABCC11 inhibitor can comprise about 0.001 wt % to about 5 wt % of the formulation. In another embodiment, the ABCC11 inhibitor can comprise about 0.005 to about 1 wt % of the formulation. In still a further embodiment, the ABCC11 inhibitor can comprise about 0.01 wt % to about 0.5 wt % of the formulation. In one embodiment, the ABCC11 inhibitor can comprise about 0.05 wt % to about 0.1 wt % of the formulation. In some specific examples, the ABCC11 inhibitor can comprise about 0.0001 wt % to about 0.001 wt %, about 0.001 wt % to about 0.01 wt %, or from about 0.005 wt % to about 0.05 wt %. In one example, the ABCC11 inhibitor can be an siRNA.

In some examples, a therapeutically effective amount of the inhibitor or therapeutic agent can be an amount sufficient to inhibit expression of an ABCC11 gene in a target cell of the subject to an osmidrosis-reducing level. In some examples, an osmidrosis-reducing level of expression can be at least 30% lower than baseline. In additional examples, an osmidrosis-reducing level of expression can be at least 40% lower than baseline. In yet additional examples, an osmidrosis-reducing level of expression can be at least 50% lower than baseline. In still additional examples, an osmidrosis-reducing level of expression can be at least 60% lower than baseline. In further examples, an osmidrosis-reducing level of expression can be at least 65%, 67%, or 69% lower than baseline.

As previously mentioned, in some examples, the RNA inhibitors can be modified to enable passive uptake of the inhibitor. In other words, in some examples, the inhibitor can be modified for self-delivery (e.g. Accell siRNAs, or the like) without the need for electroporation, viral-mediated delivery of the inhibitor, liposomic/polymeric carriers, or the like. In yet other examples, cationic liposomes or polymeric carriers can be employed to facilitate transfection of the inhibitor into a target cell. In still other examples, electroporation or the like can be employed to facilitate transfection into a target cell. In other examples, the RNA inhibitor can be delivered via viral-mediated delivery. Where this is the case, any suitable viral vector can be employed, such as lentivirus, retrovirus, adenovirus, adeno-associated virus, the like, or a combination thereof.

In some examples, an additional therapeutic agent can be included in the composition and/or administered contemporaneously with the therapeutic agent. Non-limiting examples can include antimicrobials (e.g. antibacterials, antifungals, antivirals, antiparasitics, etc.) or agents that reduce overall sweating such as antiperspirants and botulinum toxin or other toxins.

In some specific examples, the composition can include an antibacterial agent. Non-limiting examples of antibacterial agents can include triclosan, triclocarban, chloroxylenol, dicloxacillin, cephalexin, cefuroxime, clindamycin, bacitracin, polymixin B, neomycin, gentamicin, mupirocin, the like, or combinations thereof. Alternatively, one or more antibacterial agents, such as those listed above, can be administered separately from the compositions described herein, but as part of a method of treating osmidrosis. For example, in some cases, it can be desirable to administer the composition locally, whereas it can be desirable to administer the antibacterial agent systemically, or vice versa.

In yet other examples, the composition can include an antiperspirant. Non-limiting examples of antiperspirants can include any one of aluminum chlorohydrate, aluminum chloride, aluminum hydroxide, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrate polyethylene glycol, aluminum sesquichlorohydrate propylene glycol, aluminum-zirconium octachlorohydrate, aluminum-zirconium octachlorohydrex glycine, aluminum-zirconium pentachlorohydrate, aluminum-zirconium pentachlorohydrex glycine, aluminum-zirconium tetrachlorohydrate, aluminum-zirconium tetrachlorohydrex glycine, aluminum-zirconium trichlorohydrate, aluminum-zirconium trichlorohydrex glycine, potassium aluminum sulfate, aluminum undecylenoyl collagen amino acid, sodium aluminum lactate, aluminum sulfate, sodium aluminum chlorohydroxylactate, aluminum bromohydrate, aluminum chlorohydroxyallantoinate, zinc chloride, zinc sulfocarbolate, zinc sulfate, zirconium chlorohydrate, the like, or any suitable combinations thereof.

In some other examples, the composition can further include a toxin. Non-limiting examples of toxins can include any one of botulinum toxin, cyanotoxins, such as anatoxin-a, lyngbyatoxin-a, aplysiatoxins, and other toxins produced by cyanobacteria; dinotoxins, such as saxitoxins and gonyautoxins, and other toxins produced by dinoflagellates; necrotoxins, causing necrosis or cell death, such as toxins found in the brown recluse spider, venom of the rattlesnake and other vipers, pore forming toxins of necrotizing fasciitis bacteria; neurotoxins, including toxins that disrupt ion channel conductance, comprising tetrodotoxin, chlorotoxin, conotoxin, botulinum toxin, tetanus toxin, anatoxin, bungarotoxin, carambotoxin, curare poisons, and those found in the venom of the black widow spider, jellyfish, elapid snakes, venomous fish, mollusks, and amphibians, coral and some algae; myotoxins found in snake and lizard venoms; cytotoxins, such as ricin, apitoxin, and mycotoxins, including aflatoxins, ochratoxins, citrinin, ergot toxins, patulin, fumonisins, trichothecenes, zearalenone, beauvercin, enniatins, butenolide, equisetin, fusarins, batroxobins, batrachotoxins, cobrotoxins, crotamines, didemnins, deltorphins, exendins, gephyrotoxin, hannalgesins, histrionicotoxins, opitoxins, phycotoxins, scorpion toxins (such as scorpion β-toxins, etc.), spider toxins (such as co-agatoxins, psalmotoxins, etc.), the like, or any suitable combination thereof.

The present methods and compositions can be illustrated by a number of non-exclusive embodiments as follows:

A method of treating an osmidrosis condition in a subject can include administering a therapeutic agent in an amount that is effective to inhibit expression of an ABCC11 gene in a target cell of the subject to an osmidrosis-reducing level.

In some examples, the osmidrosis condition includes axillary osmidrosis, pectoral osmidrosis, genital osmidrosis, or a combination thereof.

In some examples, the osmidrosis condition includes axillary oxmidrosis.

In some examples, the osmidrosis condition includes pectoral osmidrosis.

In some examples, the osmidrosis condition includes genital osmidrosis.

In some examples, administration is performed locally at a situs of the condition.

In some examples, the situs includes one or more of the axillary region, the chest region, and the genital region.

In some examples, the situs includes the axillary region.

In some examples, the situs includes the chest/pectoral region.

In some examples, the situs includes the genital region.

In some examples, administration is performed via injection, microneedle array, topical administration, transdermal administration, or a combination thereof.

In some examples, administration is performed via injection.

In some examples, administration is performed via microneedle array.

In some examples, administration is performed via topical administration.

In some examples, administration is performed via transdermal administration.

In some examples, the therapeutic agent is configured to inhibit expression of the ABCC11 gene in the target cell via gene therapy.

In some examples, the therapeutic agent is a member selected from the group consisting of a CRISPR/Cas9 system, a therapeutic polynucleotide including a rs17822931 single-nucleotide polymorphism (SNP), and a combination thereof.

In some examples, the therapeutic agent is a member selected from the group consisting of small interfering RNAs (siRNAs), micro RNAs (miRNAs), morpholinos, antisense oligonucleotides (ASOs), peptide nucleic acids, small molecule inhibitors, and combinations thereof.

In some examples, the therapeutic agent is administered in an amount of from about 0.01 mg to about 100 mg per dose.

In some examples, the therapeutic agent includes a self-delivery modification to facilitate uptake by the target cell.

In some examples, the self-delivery modification includes one or more of lipids, cholesterol, natural ligands, peptides, and chemical modifications.

In some examples, the therapeutic agent is an siRNA.

In some examples, the siRNA includes a sequence that is at least 90% homologous to any one of SEQ ID NOs: 326 through 973, or a segment thereof having at least 13consecutive nucleotides of any one of said sequences (i.e. SEQ ID NOs: 326 through 973).

In some examples, the siRNA includes a sequence that is at least 90% homologous to any one of SEQ ID NOs: 326 through 973, or a segment thereof having at least 15consecutive nucleotides of any one of said sequences (i.e. SEQ ID NOs: 326 through 973).

In some examples, the siRNA includes a sequence that is at least 90% homologous to any one of SEQ ID NOs: 326 through 973, or a segment thereof having at least 17 consecutive nucleotides of any one of said sequences (i.e. SEQ ID NOs: 326 through 973).

In some examples, the siRNA includes a sequence that is at least 90% homologous to any one of SEQ ID NOs: 326 through 973, or a segment thereof having at least 19 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 13 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 15 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 17 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 19 consecutive nucleotides of any one of said sequences (i.e. SEQ ID NOs: 326 through 973).

In some examples, the siRNA has a sequence that is at least 90% homologous to SEQ ID NO: 970, SEQ ID NO: 971, SEQ ID NO: 972, or SEQ ID NO: 973.

In some examples, the siRNA has a sequence that is at least 95% homologous to SEQ ID NO: 970, SEQ ID NO: 971, SEQ ID NO: 972, or SEQ ID NO: 973.

In some examples, the therapeutic agent is configured to target one or more gene sequences individually selected from SEQ ID NOs: 2 through 325 to inhibit expression of the ABCC11 gene.

In some examples, the target cell is an apocrine cell.

In some examples, the target cell is an axillary apocrine cell.

In some examples, the target cell is a pectoral apocrine cell.

In some examples, the target cell is a genital apocrine cell.

In some examples, the osmodrosis-reducing level of expression is at least 30% lower than baseline.

In some examples, the osmodrosis-reducing level of expression is at least 40% lower than baseline.

In some examples, the osmodrosis-reducing level of expression is at least 50% lower than baseline.

In some examples, the osmodrosis-reducing level of expression is at least 60% lower than baseline.

In some examples, the osmodrosis-reducing level of expression is at least 65% lower than baseline.

In some examples, a therapeutic composition for treating an osmidrosis condition in a subject can include a therapeutically effective amount of an ABCC11 gene-inhibiting agent and a pharmaceutically acceptable carrier.

In some examples, the amount of therapeutic agent is an amount sufficient to reduce expression of the ABCC11 gene to a level at least 30% below baseline.

In some examples, the amount of therapeutic agent is an amount sufficient to reduce expression of the ABCC11 gene to a level at least 40% below baseline.

In some examples, the amount of therapeutic agent is an amount sufficient to reduce expression of the ABCC11 gene to a level at least 50% below baseline.

In some examples, the amount of therapeutic agent is an amount sufficient to reduce expression of the ABCC11 gene to a level at least 60% below baseline.

In some examples, the amount of therapeutic agent is an amount sufficient to reduce expression of the ABCC11 gene to a level at least 65% below baseline.

In some examples, the therapeutic agent is a member selected from the group consisting of a CRISPR/Cas9 system, a therapeutic polynucleotide including a rs17822931 single-nucleotide polymorphism (SNP), and a combination thereof.

In some examples, the therapeutic agent is a member selected from the group consisting of: small interfering RNAs (siRNAs), micro RNAs (miRNAs), morpholinos, antisense oligonucleotides (ASOs), peptide nucleic acids, small molecule inhibitors, and combinations thereof.

In some examples, the therapeutic agent includes a self-delivery modification to facilitate uptake by the target cell.

In some examples, the self-delivery modification includes one or more of a lipid, cholesterol, a natural ligand, a peptide, and a chemical modification.

In some examples, the therapeutic agent includes an siRNA.

In some examples, the siRNA includes a sequence that is at least 90% homologous to any one of SEQ ID NOs: 326 through 973, or a segment thereof having at least 13 consecutive nucleotides of any one of said sequences (i.e. SEQ ID NOs: 326 through 973).

In some examples, the siRNA includes a sequence that is at least 90% homologous to any one of SEQ ID NOs: 326 through 973, or a segment thereof having at least 15 consecutive nucleotides of any one of said sequences (i.e. SEQ ID NOs: 326 through 973).

In some examples, the siRNA includes a sequence that is at least 90% homologous to any one of SEQ ID NOs: 326 through 973, or a segment thereof having at least 17 consecutive nucleotides of any one of said sequences (i.e. SEQ ID NOs: 326 through 973).

In some examples, the siRNA includes a sequence that is at least 90% homologous to any one of SEQ ID NOs: 326 through 973, or a segment thereof having at least 19 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 13 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 15 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 17 consecutive nucleotides of any one of said sequences (i.e. SEQ ID NOs: 326 through 973).

In some examples, the siRNA includes a sequence that is at least 95% homologous to any one of SEQ ID NOs: 326 through 973, or a segment thereof having at least 19 consecutive nucleotides of any one of said sequences (i.e. SEQ ID NOs: 326 through 973).

In some examples, the siRNA has a sequence that is at least 90% homologous to SEQ ID NO: 970, SEQ ID NO: 971, SEQ ID NO: 972, or SEQ ID NO: 973.

In some examples, the siRNA has a sequence that is at least 95% homologous to SEQ ID NO: 970, SEQ ID NO: 971, SEQ ID NO: 972, or SEQ ID NO: 973.

In some examples, the therapeutic agent is present in the composition in an amount from about 0.0001 wt % to about 20 wt %.

In some examples, the pharmaceutically acceptable carrier is formulated for injection.

In some examples, the pharmaceutically acceptable carrier is formulated as a microneedle array.

In some examples, the pharmaceutically acceptable carrier is formulated as a topical or transdermal delivery system.

In some examples, the composition further includes an additional therapeutic agent.

In some examples, the additional therapeutic agent is a member selected from the group consisting of: an antimicrobial, an antiperspirant, a toxin, and combinations thereof.

### EXAMPLE

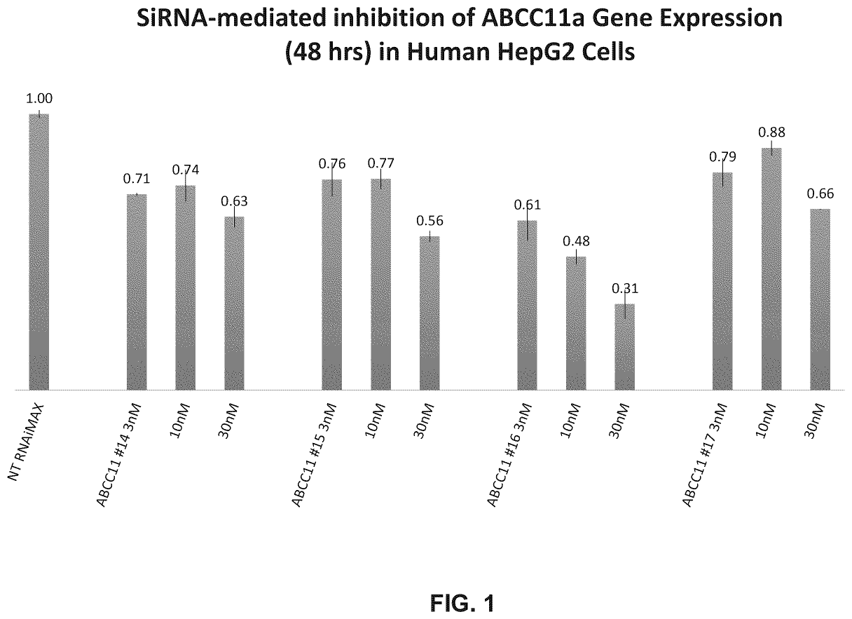

Human HepG2 liver cells (seeded onto 96 well plates at 0.3×10⁵ cells per well from stock cultures from an ˜80% confluent T75 tissue culture flask (cultured in RPMI supplemented with 10% fetal bovine serum, 1 mM sodium pyruvate, pen/strep antibiotics and glutamine as well as 1× MEM NEAA solution) were transfected (using RNAiMax, ThermoFisher) with 3 nM, 10 nM, or 30 nM of each of four distinct siRNAs (containing Accell self-delivery and stability modifications proprietary to GE Life Sciences/Dharmacon Division) targeting ABCC11. After a 48-hour incubation period in a 37° C. CO₂ incubator, cells were harvested, RNA extracted and subjected to RT-qPCR using TaqMan primer/probe sets (ABCC11 probe cat #Hs01090768_m1 ABCC11 FAM and hGAPDH probe cat # Hs99999905_m1 GAPDH FAM). The results are illustrated in FIG. 1. The resulting data were normalized to GAPDH and demonstrate that ABCC11 #16 siRNA treatment results in 69% inhibition from baseline levels. Further, each of the siRNAs employed in this study resulted in at least 34% inhibition from baseline levels at an amount of 30 nM.

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A method of treating an osmidrosis condition in a subject, comprising:
administering to a sweat gland of the subject, a therapeutic agent comprising a small interfering RNA (siRNA) in an amount that is effective to inhibit expression of an ATP-Binding Cassette Protein C11 (ABCC11) gene in a target cell of the sweat gland to an osmidrosis-reducing level, wherein the siRNA includes a sequence that is at least 90% homologous to any one of SEQ ID NOs: 326 through 969, or a segment thereof having at least 15 consecutive nucleotides of any one of said sequences.

2. The method of claim 1, wherein the osmidrosis condition includes axillary osmidrosis, pectoral osmidrosis, genital osmidrosis, or a combination thereof.

3. The method of claim 1, wherein sweat gland is one or more of an eccrine gland or an apocrine gland.

4. The method of claim 3, wherein the situs includes one or more of an axillary region, a chest region, and a genital region.

5. The method of claim 1, wherein administration is performed via injection, microneedle array, topical administration, transdermal administration, or a combination thereof.

6. The method of claim 1, wherein the therapeutic agent is administered in an amount of from about 0.01 mg to about 100 mg per dose.

7. The method of claim 1, wherein the therapeutic agent includes a self-delivery modification to facilitate uptake by the target cell.

8. The method of claim 7, wherein the self-delivery modification includes one or more of lipids, cholesterol, natural ligands, peptides, and chemical modifications.

9. The method of claim 1, wherein the therapeutic agent further includes an siRNA guide strand that has a sequence that is at least 90% homologous to SEQ ID NO: 970, SEQ ID NO: 971, SEQ ID NO: 972, or SEQ ID NO: 973, and wherein said siRNA guide strand is present in an amount that is effective to inhibit expression of an ATP-Binding Cassette Protein C11 (ABCC11) gene in a target cell of the subject to an osmidrosis-reducing level.

10. The method of claim 1, wherein the osmidrosis-reducing level of expression is at least 30% lower than baseline.

11. A therapeutic composition for treating an osmidrosis condition in a subject, comprising:
a therapeutically effective amount of an ABCC11 gene-inhibiting agent; and
a pharmaceutically acceptable carrier.

12. The composition of claim 11, wherein the amount of therapeutic agent is an amount sufficient to reduce expression of the ABCC11 gene to a level at least 30% below baseline.

13. The composition of claim 11, wherein the therapeutic agent is a member selected from the group consisting of: small interfering RNAs (siRNAs), micro RNAs (miRNAs), morpholinos, antisense oligonucleotides (ASOs), peptide nucleic acids, small molecule inhibitors, and combinations thereof.

14. The composition of claim 11, wherein the therapeutic agent includes a self-delivery modification to facilitate uptake by the target cell.

15. The composition of claim 14, wherein the self-delivery modification includes one or more of a lipid, cholesterol, a natural ligand, a peptide, and a chemical modification.

16. The composition of claim 11, wherein the therapeutic agent includes an siRNA.

17. The composition of claim 16, wherein the siRNA includes a sequence that is at least 90% homologous to any one of SEQ ID NOs: 326 through 973, or a segment thereof having at least 15 consecutive nucleotides of any one of said sequences.

18. The composition of claim 16, wherein the siRNA has a sequence that is at least 90% homologous to SEQ ID NO: 970, SEQ ID NO: 971, SEQ ID NO: 972, or SEQ ID NO: 973.

19. The composition of claim 11, wherein the therapeutic agent is present in the composition in an amount from about 0.0001 wt % to about 20 wt %.
